(19) United States
(12) Patent Application Publication
(10) Pub. No.: US 2003/0004172 A1
(43) Pub. Date: Jan. 2, 2003

(54) Fused pyrimidinone matrix metalloproteinase inhibitors

(76) Inventors: William Glen Harter, Chelsea, MI (US); Jie Jack Li, Ann Arbor, MI (US); Daniel Fred Ortwine, Saline, MI (US); Kevon Ray Shuler, Chelsea, MI (US); Wen-Song Yue, Ypsilanti, MI (US)

Correspondence Address: Claude F. Purchase, Jr., Warner-Lambert Company, 2800 Plymouth Road, Ann Arbor, MI, 48105, US

(21) Appl. No.: 10/075,073

(22) Filed: Feb. 13, 2002

Related U.S. Application Data
(60) Provisional application No. 60/268,756, filed on Feb. 14, 2001.

(51) Int. Cl. A61K 31/522; A61K 31/519; C07D 491/02; C07D 473/02

(52) U.S. Cl. 514/260.1; 514/263.34; 514/263.33; 514/265.1; 544/256; 544/267; 544/271; 544/280

(57) ABSTRACT

Selective MMP-13 inhibitors are fused pyrimidinones of the formula

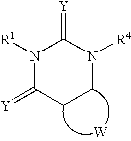

or a pharmaceutically acceptable salt thereof, wherein:

W, together with the carbon atoms to which it is attached, form a 5-membered ring diradical

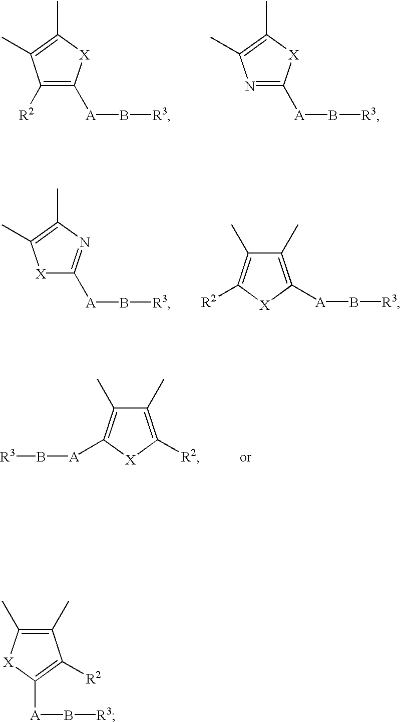

X is O, S, SO, SO₂, NR⁵, or CH₂;

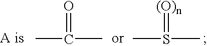

B is O or NR⁵; or

A and B are taken together to form —C≡C—;

R¹, R⁴, and R⁵ are hydrogen, alkyl, alkenyl, alkynyl, (CH₂)ₙ aryl, (CH₂)ₙ cycloalkyl, C₁-C₆ alkanoyl, or (CH₂)ₙ heteroaryl;

R² and R³ are hydrogen, alkyl, alkenyl, alkynyl CN, NO₂, NR⁴R⁵, (CH₂)ₙ cycloalkyl, (CH₂)ₙ aryl, or (CH₂)ₙ heteroaryl;

R² may further be halo;

n is an integer of from 0 to 5; and

R⁴ and R⁵ when taken together with a nitrogen to which they are both attached complete a 3- to 8-membered ring containing carbon atoms and optionally containing O, S, or N, and substituted or unsubstituted;

with the proviso that R¹ and R³ are not both selected from hydrogen and C₁-C₆ alkyl.

### CROSS-REFERENCE TO RELATED APPLICATIONS

This application claims benefit of priority from United States provisional application number 60/268,756, filed Feb. 14, 2001.

### FIELD OF THE INVENTION

This invention relates to fused bicyclic pyrimidinones that inhibit matrix metalloproteinase enzymes and thus are useful for treating diseases resulting from tissue breakdown, such as heart disease, multiple sclerosis, arthritis, atherosclerosis, and osteoporosis.

### BACKGROUND OF THE INVENTION

Matrix metalloproteinases (sometimes referred to as MMPs) are naturally-occurring enzymes found in most mammals. Over-expression and activation of MMPs or an imbalance between MMPs and inhibitors of MMPs, have been suggested as factors in the pathogenesis of diseases characterized by the breakdown of extracellular matrix or connective tissues.

Stromelysin-1 and gelatinase A are members of the matrix metalloproteinases (MMP) family. Other members include fibroblast collagenase (MMP-1), neutrophil collagenase (MMP-8), gelatinase B (92 kDa gelatinase) (MMP-9), stromelysin-2 (MMP-10), stromelysin-3 (MMP-11), matrilysin (MMP-7), collagenase 3 (MMP-13), TNF-alpha converting enzyme (TACE), and other newly discovered membrane-associated matrix metalloproteinases (Sato H., Takino T., Okada Y., Cao J., Shinagawa A., Yamamoto E., and Seiki M., Nature, 1994;370:61-65). These enzymes have been implicated with a number of diseases which result from breakdown of connective tissue, including such diseases as rheumatoid arthritis, osteoarthritis, osteoporosis, periodontitis, multiple sclerosis, gingivitis, corneal epidermal and gastric ulceration, atherosclerosis, neointimal proliferation which leads to restenosis and ischemic heart failure, stroke, renal disease, macular degeneration, and tumor metastasis. A method for preventing and treating these and other diseases is now recognized to be by inhibiting metalloproteinase enzymes, thereby curtailing and/or eliminating the breakdown of connective tissues that results in the disease states.

The catalytic zinc in matrix metalloproteinases is typically the focal point for inhibitor design. The modification of substrates by introducing zinc chelating groups has generated potent inhibitors such as peptide hydroxamates and thiol-containing peptides. Peptide hydroxamates and the natural endogenous inhibitors of MMPs (TIMPs) have been used successfully to treat animal models of cancer and inflammation. MMP inhibitors have also been used to prevent and treat congestive heart failure and other cardiovascular diseases, U.S. Pat. No. 5,948,780.

A major limitation on the use of currently known MMP inhibitors is their lack of specificity for any particular enzyme. Recent data has established that specific MMP enzymes are associated with some diseases, with no effect on others. The MMPs are generally categorized based on their substrate specificity, and indeed the collagenase subfamily of MMP-1, MMP-8, and MMP-13 selectively cleave native interstitial collagens, and thus are associated only with diseases linked to such interstitial collagen tissue. This is evidenced by the recent discovery that MMP-13 alone is over expressed in breast carcinoma, while MMP-1 alone is over expressed in papillary carcinoma (see Chen et al., J. Am. Chem. Soc., 2000;122:9648-9654).

There appears to be few selective inhibitors of MMP-13 reported. A compound named WAY-170523 has been reported by Chen et al., supra., 2000, and a few other compounds are reported in PCT International patent application publication Number WO 01/63244 A1, as allegedly selective inhibitors of MMP-13. Further, U.S. Pat. No. 6,088,243 discloses inhibitors of MMP-13. However, no selective or nonselective inhibitor of MMP-13 has been approved and marketed for the treatment of any disease in any mammal. Accordingly, the need continues to find new low molecular weight compounds that are potent and selective MMP inhibitors, and that have an acceptable therapeutic index of toxicity/potency to make them amenable for use clinically in the prevention and treatment of the associated disease states. An object of this invention is to provide a group of selective MMP-13 inhibitor compounds characterized as being fused bicyclic pyrimidinones.

### SUMMARY OF THE INVENTION

This invention provides bicyclic pyrimidinones that are inhibitors of matrix metalloproteinase enzymes, and especially MMP-13. The invention is more particularly directed to compounds defined by Formula I

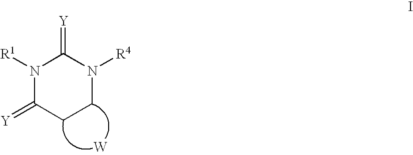

or a pharmaceutically acceptable salt thereof; wherein:

W, together with the carbon atoms to which it is attached, form a 5-membered ring diradical

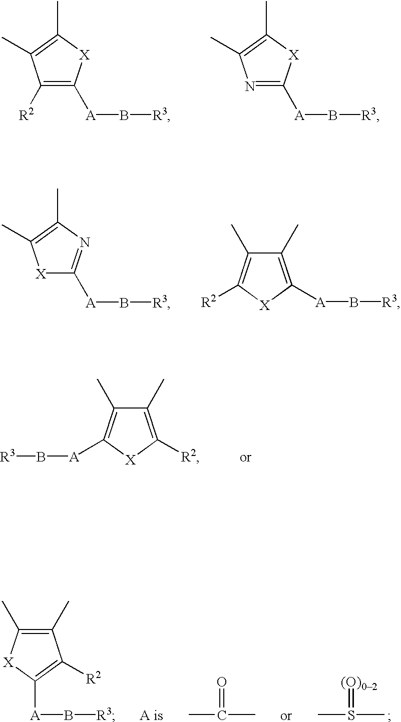

A is

B is O or NR⁵; or

A and B are taken together to form —C≡C—;

X is O, S, SO, SO₂, NR⁵, or CH₂;

each Y independently is O or S;

R¹, R⁴, and R⁵ independently are hydrogen, C₁-C₆ alkyl, C₂-C₆ alkenyl, C₂-C₆ alkynyl, (CH₂)ₙ cycloalkyl, (CH₂)ₙ heterocyclic, C₁-C₆ alkanoyl, (CH₂)ₙ aryl, or (CH₂)ₙ heteroaryl;

R² and R³ independently are hydrogen, C₁-C₆ alkyl, C₂-C₆ alkenyl, C₂-C₆ alkynyl, CN, NO₂, NR⁴R⁵, (CH₂)ₙ cycloalkyl, (CH₂)ₙ aryl, or (CH₂)ₙ heteroaryl; CONR⁴R⁵, or COR⁶;

R² may further be halo;

n is an integer of from 0 to 5;

R⁴ and R⁵ when taken together with the nitrogen to which they are attached complete a 3- to 8-membered ring containing carbon atoms and optionally containing O, S, or N, and substituted or unsubstituted;

with the proviso that R¹ and R³ are not both selected from: hydrogen and C₁-C₆ alkyl.

Another invention embodiment is compounds that are thieno[2,3-d]pyrimidinones of Formula II

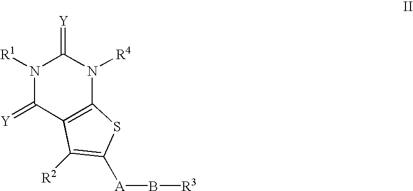

or a pharmaceutically acceptable salt thereof, wherein A, B, R¹, R², R³, R⁴, and Y are as defined above.

Another invention embodiment is compounds that of Formula II, or a pharmaceutically acceptable salt thereof, wherein—A—B— is —C≡C—,

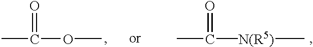

wherein R⁵ is as defined above for Formula I.

Another invention embodiment is compounds that have Formula III

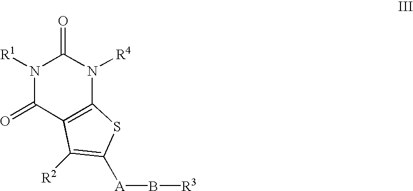

or a pharmaceutically acceptable salt thereof, wherein A, B, R¹, R², and R⁴ are as defined above, and R³ is (CH₂)ₙ aryl, (CH₂)ₙ cycloalkyl, or (CH₂)ₙ heteroaryl.

Another invention embodiment is compounds that are compounds of Formula III, or a pharmaceutically acceptable salt thereof, wherein R³ is (CH₂)ₙ aryl, (CH₂)ₙ cycloalkyl, or (CH₂)ₙ heteroaryl, and—A—B— is —C≡C—.

Another invention embodiment is compounds that are pyrimidinone MMP-inhibitors of Formula IV

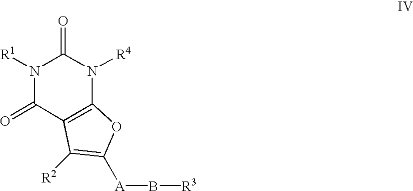

or a pharmaceutically acceptable salt thereof, wherein A, B, R¹, R², R³, and R⁴ are as defined above.

Another invention embodiment are pyrimidinone compounds provided by this invention that have Formula V

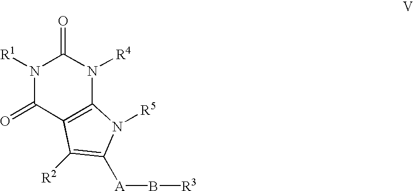

or a pharmaceutically acceptable salt thereof, wherein A, B, R¹, R², R³, R⁴, and R⁵ are as defined above.

Another invention embodiment is compounds of Formula VI-IX:

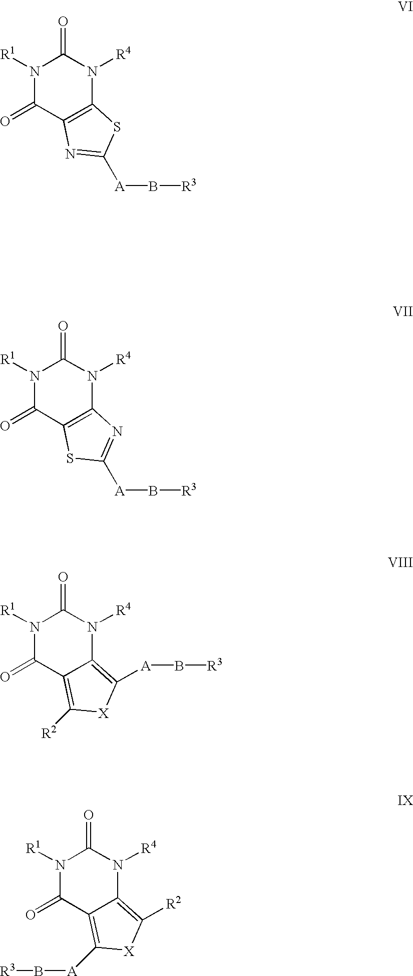

or a pharmaceutically acceptable salt thereof, wherein A, B, X, R¹, R², R³, and R⁴ are as defined above.

Another embodiment of the invention is a compound of Formula X

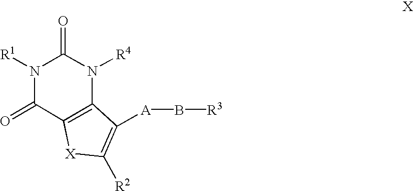

or a pharmaceutically acceptable salt thereof, wherein R¹-R⁴, A, B, and X are as defined above.

Another invention embodiment is a compound, or a pharmaceutically acceptable salt thereof, in any one of the above formulas wherein R⁴ in the above formulas is hydrogen, methyl, or trifluoromethyl.

Another invention embodiment is a compound, or a pharmaceutically acceptable salt thereof, in any one of the above Formulas wherein R⁴ is methyl or trifluoromethyl.

Another invention embodiment is a compound, or a pharmaceutically acceptable salt thereof, in any one of the above formulas wherein R¹ in the above formulas is (CH₂)ₙ cycloalkyl, (CH₂)ₙ aryl, (CH₂)ₙ heterocyclic, or (CH₂)ₙ heteroaryl, wherein n is as defined above for Formula I.

Another invention embodiment is a compound, or a pharmaceutically acceptable salt thereof, in any one of the above formulas wherein R² in the above Formulas is hydrogen or fluoro.

Another invention embodiment is a compound, or a pharmaceutically acceptable salt thereof, in any one of the above formulas wherein n in the above Formulas is 1.

Another invention embodiment is a compound, or a pharmaceutically acceptable salt thereof, in any one of the above Formulas

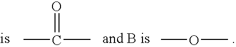

where A

Another invention embodiment is a compound, or a pharmaceutically acceptable salt thereof, of any one of the above Formulas which comprises a combination of any two or more embodiments selected from:

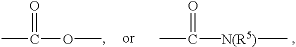

A—B is —C≡C—,

wherein R⁵ is as defined above for Formula I;

R¹ and R³ independently are (CH₂)ₙ cycloalkyl, (CH₂)ₙ aryl, (CH₂)ₙ heterocyclic, or (CH₂)ₙ heteroaryl, wherein n is as defined above for Formula I;

R² is hydrogen or fluoro;

R⁴ is methyl or trifluoromethyl; and

n is 1.

Another embodiment of the invention is a compound of Formula XI

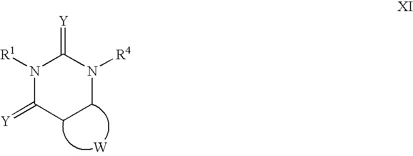

or a pharmaceutically acceptable salt thereof, wherein:

W, together with the carbon atoms to which it is attached, form a 5-membered ring diradical

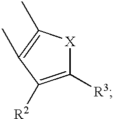

each Y independently is O or S;

X is S, O, or NR⁵;

R¹, R⁴, and R⁵ independently are hydrogen, C₁—C₆ alkyl, C₂-C₆ alkenyl, C₂-C₆ alkynyl, (CH₂)ₙ cycloalkyl, (CH₂)ₙ heterocyclic, C₁-C₆ alkanoyl, (CH₂)ₙ aryl, or (CH₂)ₙ heteroaryl;

R² is hydrogen, C₁-C₆ alkyl, C₂-C₆ alkenyl, C₂-C₆ alkynyl, CN, NO₂, NR⁴R⁵, (CH₂)ₙ cycloalkyl, (CH₂)ₙ aryl, or (CH₂)ₙ heteroaryl;

R³ is hydrogen, halo, C₁-C₆ alkyl, C₂-C₆ alkenyl, CN, NO₂, NR⁴R⁵, (CH₂)_{q} cycloalkyl, (CH₂)_{q} aryl, or (CH₂)_{q} heteroaryl;

n is 0, 1, or 2;

q is 2, 3, or 4; and

R⁴ and R⁵ when taken together with the nitrogen to which they are attached complete a 3- to 8-membered ring containing carbon atoms and optionally containing O, S, or N, and substituted or unsubstituted;

with the proviso that R¹ and R³ are not both selected from: hydrogen and C₁-C₆ alkyl.

Another invention embodiment is the compound of Formula XI, or a pharmaceutically acceptable salt thereof, wherein Y is O and X is S.

Another invention embodiment is the compound of Formula XI, or a pharmaceutically acceptable salt thereof, wherein Y is O and X is O.

Another invention embodiment is the compound of Formula XI, or a pharmaceutically acceptable salt thereof, wherein Y is O and X is NR⁵, wherein R⁵ is hydrogen, C₁-C₆ alkyl, C₂-C₆ alkenyl, C₂-C₆ alkynyl, (CH₂)ₙ cycloalkyl, (CH₂)ₙ heterocyclic, C₁-C₆ alkanoyl, (CH₂)ₙ aryl, or (CH₂)ₙ heteroaryl.

The compounds of this invention typically will be named according to the following numbering system

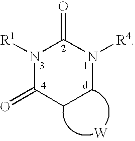

The compound of the formula

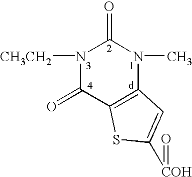

will be named 3-ethyl-1-methyl-2,4-dioxo-1,2,3,4-tetrahydro-thieno[2,3-d]pyrimidine-6-carboxylic acid.

Another invention embodiment are compounds provided by this invention selected from:

3-Benzyl-2,4-dioxo-1,2,3,4-tetrahydro-thieno[2,3-d]pyrimidine-6-carboxylic acid benzyl ester; and

3-Benzyl-1-methyl-2,4-dioxo-1,2,3,4-tetrahydro-thieno[2,3-d]pyrimidine-6-carboxylic acid benzyl ester.

Another invention embodiment is a compound selected from:

3-(4-Pyridyl)-2,4-dioxo-1,2,3,4-tetrahydro-thieno[2,3-d]pyrimidine-6-carboxylic acid benzyl ester;

3-(4-Pyridyl)-1-methyl-2,4-dioxo-1,2,3,4-tetrahydro-thieno[2,3-d]-pyrimidine-6-carboxylic acid benzyl ester;

3-Benzyl-2,4-dioxo-1,2,3,4-tetrahydro-thieno[2,3-d]pyrimidine-6-carboxylic acid (4-pyridyl)ester;

3-Benzyl-1-methyl-2,4-dioxo-1,2,3,4-tetrahydro-thieno[2,3-d]pyrimidine-6-carboxylic acid (4-pyridyl)ester;

3-(4-Pyridyl)-2,4-dioxo-1,2,3,4-tetrahydro-thieno[2,3-d]pyrimidine-6-carboxylic acid (4-pyridyl)ester;

3-(4-Pyridyl)-1-methyl-2,4-dioxo-1,2,3,4-tetrahydro-thieno[2,3-d]-pyrimidine-6-carboxylic acid (4-pyridyl)ester;

3-Benzyl-2,4-dioxo-1,2,3,4-tetrahydro-thieno[2,3-d]pyrimidine-6-carboxylic acid piperoyl ester;

3-Benzyl-1-methyl-2,4-dioxo-1,2,3,4-tetrahydro-thieno[2,3-d]pyrimidine-6-carboxylic acid piperoyl ester;

3-Piperoyl-2,4-dioxo-1,2,3,4-tetrahydro-thieno[2,3-d]pyrimidine-6-carboxylic acid piperoyl ester;

3-Piperoyl-1-methyl-2,4-dioxo-1,2,3,4-tetrahydro-thieno[2,3-d]-pyrimidine-6-carboxylic acid piperoyl ester;

3-Benzyl-1-methyl-2,4-dioxo-1,2,3,4-tetrahydro-furo[2,3-d]pyrimidine-6-carboxylic acid benzyl ester;

3-Benzyl-1-methyl-2,4-dioxo-1,2,3,4-tetrahydro-1H-pyrrolo[2,3-d]-pyrimidine-6-carboxylic acid benzyl ester;

3-Benzyl-1,7-dimethyl-2,4-dioxo-1,2,3,4-tetrahydro-1H-pyrrolo[2,3-d]-pyrimidine-6-carboxylic acid benzyl ester;

3-Benzyl-1,7-dimethyl-2,4-dioxo-1,2,3,4-tetrahydro-1H-pyrrolo[2,3-d]-pyrimidine-6-carboxylic acid benzofuran-6-ylmethyl ester;

3-Benzyl-1-dimethyl-2,4-dioxo-1,2,3,4-tetrahydro-1H-pyrrolo[2,3-d]-pyrimidine-6-carboxylic acid benzofuran-6-ylmethyl ester;

3-Benzyl-1-methyl-2,4-dioxo-1,2,3,4-tetrahydro-furo[2,3-d]pyrimidine-6-carboxylic acid benzofuran-6-ylmethyl ester;

3-Benzyl-1-methyl-2,4-dioxo-1,2,3,4-tetrahydro-thieno[2,3-d]pyrimidine-6-carboxylic acid benzofuran-6-ylmethyl ester;

3-Benzyl-1,7-dimethyl-2,4-dioxo-1,2,3,4-tetrahydro-1H-pyrrolo[2,3-d]-pyrimidine-6-carboxylic acid benzothiophene-6-ylmethyl ester;

3-Benzyl-1-dimethyl-2,4-dioxo-1,2,3,4-tetrahydro-1H-pyrrolo[2,3-d]-pyrimidine-6-carboxylic acid benzothiophene-6-ylmethyl ester;

3-Benzyl-1-methyl-2,4-dioxo-1,2,3,4-tetrahydro-furo[2,3-d]pyrimidine-6-carboxylic acid benzothiophene-6-ylmethyl ester; and

3-Benzyl-1-methyl-2,4-dioxo-1,2,3,4-tetrahydro-thieno[2,3-d]pyrimidine-6-carboxylic acid benzothiophene-6-ylmethyl ester.

Another invention embodiment is a compound selected from:

3-(3-Methoxy-benzyl)-1-methyl-2,4-dioxo-1,2,3,4-tetrahydro-thieno[2,3-d]pyrimidine-6-carboxylic acid benzyl ester;

3-(4-Chloro-benzyl)-1-methyl-2,4-dioxo-1,2,3,4-tetrahydro-thieno[2,3-d]pyrimidine-6-carboxylic acid benzyl ester;

3-(4-Cyano-benzyl)-1-methyl-2,4-dioxo-1,2,3,4-tetrahydro-thieno[2,3-d]pyrimidine-6-carboxylic acid benzyl ester;

3-(4-Fluoro-benzyl)-1-methyl-2,4-dioxo-1,2,3,4-tetrahydro-thieno[2,3-d]pyrimidine-6-carboxylic acid benzyl ester;

3-(3-Chloro-benzyl)-1-methyl-2,4-dioxo-1,2,3,4-tetrahydro-thieno[2,3-d]pyrimidine-6-carboxylic acid benzyl ester;

1-Methyl-3-(2-methyl-benzyl)-2,4-dioxo-1,2,3,4-tetrahydro-thieno[2,3-d]pyrimidine-6-carboxylic acid benzyl ester;

1-Methyl-3-(4-methyl-benzyl)-2,4-dioxo-1,2,3,4-tetrahydro-thieno[2,3-d]pyrimidine-6-carboxylic acid benzyl ester;

3-(4-Carboxy-benzyl)-1-methyl-2,4-dioxo-1,2,3,4-tetrahydro-thieno[2,3-d]pyrimidine-6-carboxylic acid benzyl ester;

1-Methyl-2,4-dioxo-3-(3-trifluoromethyl-benzyl)-1,2,3,4-tetrahydro-thieno[2,3-d]pyrimidine-6-carboxylic acid benzyl ester;

3-Biphenyl-4-ylmethyl-1-methyl-2,4-dioxo-1,2,3,4-tetrahydro-thieno[2,3-d]pyrimidine-6-carboxylic acid benzyl ester;

1-Methyl-2,4-dioxo-3-(2-trifluoromethyl-benzyl)-1,2,3,4-tetrahydro-thieno[2,3-d]pyrimidine-6-carboxylic acid benzyl ester;

3-(3-Cyano-benzyl)-1-methyl-2,4-dioxo-1,2,3,4-tetrahydro-thieno[2,3-d]pyrimidine-6-carboxylic acid benzyl ester;

3-(2-Cyano-benzyl)-1-methyl-2,4-dioxo-1,2,3,4-tetrahydro-thieno[2,3-d]pyrimidine-6-carboxylic acid benzyl ester;

1-Methyl-2,4-dioxo-3-(4-trifluoromethyl-benzyl)-1,2,3,4-tetrahydro-thieno[2,3-d]pyrimidine-6-carboxylic acid benzyl ester;

3-[2-Hydroxy-3-(naphthalen-1-yloxy)-propyl]-1-methyl-2,4-dioxo-1,2,3,4-tetrahydro-thieno[2,3-d]pyrimidine-6-carboxylic acid benzyl ester;

3-(3-Chloro-benzyl)-1-methyl-2,4-dioxo-1,2,3,4-tetrahydro-thieno[2,3-d]pyrimidine-6-carboxylic acid benzyl ester;

1-Methyl-3-naphthalen-1-ylmethyl-2,4-dioxo-1,2,3,4-tetrahydro-thieno[2,3-d]pyrimidine-6-carboxylic acid benzyl ester;

3-(6-Chloro-benzo[1,3]dioxol-5-ylmethyl)-1-methyl-2,4-dioxo-1,2,3,4-tetrahydro-thieno[2,3-d]pyrimidine-6-carboxylic acid benzyl ester;

1-Methyl-2,4-dioxo-3-(4-oxo-4-thiophen-2-yl-butyl)-1,2,3,4-tetrahydro-thieno[2,3-d]pyrimidine-6-carboxylic acid benzyl ester;

1-Methyl-2,4-dioxo-3-pyridin-4-ylmethyl-1,2,3,4-tetrahydro-thieno[2,3-d]pyrimidine-6-carboxylic acid benzyl ester;

1-Methyl-2,4-dioxo-3-(4-m-tolyloxy-butyl)-1,2,3,4-tetrahydro-thieno[2,3-d]pyrimidine-6-carboxylic acid benzyl ester;

3-(3,5-Dimethyl-isoxazol-4-ylmethyl)-1-methyl-2,4-dioxo-1,2,3,4-tetrahydro-thieno[2,3-d]pyrimidine-6-carboxylic acid benzyl ester;

3-Dioxo-1,3-dihydro-isoindol-2-ylmethyl)-1-methyl-2,4-dioxo-1,2,3,4-tetrahydro-thieno[2,3-d]pyrimidine-6-carboxylic acid benzyl ester;

3-Dihydro-benzo[1,4]dioxin-2-ylmethyl)-1-methyl-2,4-dioxo-1,2,3,4-tetrahydro-thieno[2,3-d]pyrimidine-6-carboxylic acid benzyl ester;

1-Methyl-2,4-dioxo-3-pyridin-2-ylmethyl-1,2,3,4-tetrahydro-thieno[2,3-d]pyrimidine-6-carboxylic acid benzyl ester;

3-[2-(2,5-Dimethoxy-phenyl)-2-oxo-ethyl]-1-methyl-2,4-dioxo-1,2,3,4-tetrahydro-thieno[2,3-d]pyrimidine-6-carboxylic acid benzyl ester;

3-Benzyloxymethyl-1-methyl-2,4-dioxo-1,2,3,4-tetrahydro-thieno[2,3-d]pyrimidine-6-carboxylic acid benzyl ester;

1-Methyl-2,4-dioxo-3-(4-m-tolyloxy-butyl)-1,2,3,4-tetrahydro-thieno[2,3-d]pyrimidine-6-carboxylic acid benzyl ester;

1-Methyl-2,4-dioxo-3-(2-phenylmethanesulfonyl-ethyl)-1,2,3,4-tetrahydro-thieno[2,3-d]pyrimidine-6-carboxylic acid benzyl ester;

3-(4-Amino-6-phenylamino-[1,3,5]triazin-2-ylmethyl)-1-methyl-2,4-dioxo-1,2,3,4-tetrahydro-thieno[2,3-d]pyrimidine-6-carboxylic acid benzyl ester;

3-[4-(4-Fluoro-phenyl)-4-oxo-butyl]-1-methyl-2,4-dioxo-1,2,3,4-tetrahydro-thieno[2,3-d]pyrimidine-6-carboxylic acid benzyl ester;

3-[4-(1,3-Dioxo-1,3-dihydro-isoindol-2-yl)-butyl]-1-methyl-2,4-dioxo-1,2,3,4-tetrahydro-thieno[2,3-d]pyrimidine-6-carboxylic acid benzyl ester;

1-Methyl-2,4-dioxo-3-(4-phenoxy-butyl)-1,2,3,4-tetrahydro-thieno[2,3-d]pyrimidine-6-carboxylic acid benzyl ester;

1-Methyl-2,4-dioxo-3-(4-oxo-4-phenyl-butyl)-1,2,3,4-tetrahydro-thieno[2,3-d]pyrimidine-6-carboxylic acid benzyl ester;

1-Methyl-2,4-dioxo-3-(2-phenoxy-ethyl)-1,2,3,4-tetrahydro-thieno[2,3-d]pyrimidine-6-carboxylic acid benzyl ester;

3-{3-[4-(3-Chloro-phenyl)-piperazin-1-yl]-propyl}-1-methyl-2,4-dioxo-1,2,3,4-tetrahydro-thieno[2,3-d]pyrimidine-6-carboxylic acid benzyl ester;

3-[1-Bromo-2-(1H-indol-3-yl)-ethyl]-1-methyl-2,4-dioxo-1,2,3,4-tetrahydro-thieno[2,3-d]pyrimidine-6-carboxylic acid benzyl ester;

3-(2-Benzenesulfinyl-ethyl)-1-methyl-2,4-dioxo-1,2,3,4-tetrahydro-thieno[2,3-d]pyrimidine-6-carboxylic acid benzyl ester;

3-[3-(3-Fluoro-phenylcarbamoyl)-propyl]-1-methyl-2,4-dioxo-1,2,3,4-tetrahydro-thieno[2,3-d]pyrimidine-6-carboxylic acid benzyl ester;

1-Methyl-2,4-dioxo-3-[2-(2-trifluoromethyl-phenylcarbamoyl)-ethyl]-1,2,3,4-tetrahydro-thieno[2,3-d]pyrimidine-6-carboxylic acid benzyl ester;

3-[2-(4-Methoxy-phenyl)-ethyl]-1-methyl-2,4-dioxo-1,2,3,4-tetrahydro-thieno[2,3-d]pyrimidine-6-carboxylic acid benzyl ester;

3-[2-(4-Chloro-2-nitro-phenoxy)-ethyl]-1-methyl-2,4-dioxo-1,2,3,4-tetrahydro-thieno[2,3-d]pyrimidine-6-carboxylic acid benzyl ester;

1-Methyl-3-(5-nitro-furan-2-ylmethyl)-2,4-dioxo-1,2,3,4-tetrahydro-thieno[2,3-d]pyrimidine-6-carboxylic acid benzyl ester;

3-(1-Benzyl-1H-imidazol-2-ylmethyl)-1-methyl-2,4-dioxo-1,2,3,4-tetrahydro-thieno[2,3-d]pyrimidine-6-carboxylic acid benzyl ester;

3-[3-(Benzyl-methyl-amino)-propyl]-1-methyl-2,4-dioxo-1,2,3,4-tetrahydro-thieno[2,3-d]pyrimidine-6-carboxylic acid benzyl ester;

3-(Bis-trifluoromethyl-benzyl)-1-methyl-2,4-dioxo-1,2,3,4-tetrahydro-thieno[2,3-d]pyrimidine-6-carboxylic acid benzyl ester;

3-[3-(2-Bromo-4-methyl-phenoxy)-propyl]-1-methyl-2,4-dioxo-1,2,3,4-tetrahydro-thieno[2,3-d]pyrimidine-6-carboxylic acid benzyl ester;

3-Benzenesulfonylmethyl-1-methyl-2,4-dioxo-1,2,3,4-tetrahydro-thieno[2,3-d]pyrimidine-6-carboxylic acid benzyl ester;

3-[2-(4-Chloro-benzenesulfonyl)-ethyl]-1-methyl-2,4-dioxo-1,2,3,4-tetrahydro-thieno[2,3-d]pyrimidine-6-carboxylic acid benzyl ester;

3-Benzo[1,3]dioxol-5-ylmethyl-1-methyl-2,4-dioxo-1,2,3,4-tetrahydro-thieno[2,3-d]pyrimidine-6-carboxylic acid benzyl ester;

3-(3-Iodo-benzyl)-1-methyl-2,4-dioxo-1,2,3,4-tetrahydro-thieno[2,3-d]pyrimidine-6-carboxylic acid benzyl ester;

1-Methyl-2,4-dioxo-3-(4-trifluoromethoxy-benzyl)-1,2,3,4-tetrahydro-thieno[2,3-d]pyrimidine-6-carboxylic acid benzyl ester;

3-(4-Acetoxy-butyl)-1-methyl-2,4-dioxo-1,2,3,4-tetrahydro-thieno[2,3-d]pyrimidine-6-carboxylic acid benzyl ester;

3-(4-Methanesulfonyl-benzyl)-1-methyl-2,4-dioxo-1,2,3,4-tetrahydro-thieno[2,3-d]pyrimidine-6-carboxylic acid benzyl ester;

1-Methyl-2,4-dioxo-3-(4-[1,2,3]thiadiazol-4-yl-benzyl)-1,2,3,4-tetrahydro-thieno[2,3-d]pyrimidine-6-carboxylic acid benzyl ester;

3-(5-Methoxycarbonyl-furan-2-ylmethyl)-1-methyl-2,4-dioxo-1,2,3,4-tetrahydro-thieno[2,3-d]pyrimidine-6-carboxylic acid benzyl ester;

3-(2-Carboxy-ethyl)-1-methyl-2,4-dioxo-1,2,3,4-tetrahydro-thieno[2,3-d]pyrimidine-6-carboxylic acid benzyl ester;

1-Methyl-2,4-dioxo-3-(3-pyrrol-1-yl-propyl)-1,2,3,4-tetrahydro-thieno[2,3-d]pyrimidine-6-carboxylic acid benzyl ester;

3-(3-Carboxy-propyl)-1-methyl-2,4-dioxo-1,2,3,4-tetrahydro-thieno[2,3-d]pyrimidine-6-carboxylic acid benzyl ester;

3-(2-Cyano-ethyl)-1-methyl-2,4-dioxo-1,2,3,4-tetrahydro-thieno[2,3-d]pyrimidine-6-carboxylic acid benzyl ester;

3-(3-Ethoxycarbonyl-furan-2-ylmethyl)-1-methyl-2,4-dioxo-1,2,3,4-tetrahydro-thieno[2,3-d]pyrimidine-6-carboxylic acid benzyl ester;

3-(3-Amino-propyl)-1-methyl-2,4-dioxo-1,2,3,4-tetrahydro-thieno[2,3-d]pyrimidine-6-carboxylic acid benzyl ester;

3-(3-Cyano-propyl)-1-methyl-2,4-dioxo-1,2,3,4-tetrahydro-thieno[2,3-d]pyrimidine-6-carboxylic acid benzyl ester;

3-(2-Hydroxy-ethyl)-1-methyl-2,4-dioxo-1,2,3,4-tetrahydro-thieno[2,3-d]pyrimidine-6-carboxylic acid benzyl ester;

3-(2-Carboxy-hexyl)-1-methyl-2,4-dioxo-1,2,3,4-tetrahydro-thieno[2,3-d]pyrimidine-6-carboxylic acid benzyl ester;

1-Methyl-2,4-dioxo-3-(2,2,2-trifluoro-ethyl)-1,2,3,4-tetrahydro-thieno[2,3-d]pyrimidine-6-carboxylic acid benzyl ester;

1-Methyl-2,4-dioxo-3-(2,2,2-trifluoro-ethyl)-1,2,3,4-tetrahydro-thieno[2,3-d]pyrimidine-6-carboxyl carboxylic acid benzyl ester;

Iodomethyl-1-methyl-2,4-dioxo-1,2,3,4-tetrahydro-thieno[2,3-d]pyrimidine-6-carboxylic acid benzyl ester;

3-(2-Fluoro-ethyl)-1-methyl-2,4-dioxo-1,2,3,4-tetrahydro-thieno[2,3-d]pyrimidine-6-carboxylic acid benzyl ester;

Methyl-2,4-dioxo-3-(tetrahydro-furan-2-ylmethyl)-1,2,3,4-tetrahydro-thieno[2,3-d]pyrimidine-6-carboxylic acid benzyl ester;

3-[1-(4-Carboxy-phenyl)-ethyl]-1-methyl-2,4-dioxo-1,2,3,4-tetrahydro-thieno[2,3-d]pyrimidine-6-carboxylic acid benzyl ester;

3-(Hex-5-enyl)-1-methyl-2,4-dioxo-1,2,3,4-tetrahydro-thieno[2,3-d]pyrimidine-6-carboxylic acid benzyl ester;

3-(2-Ethyl-butyl)-1-methyl-2,4-dioxo-1,2,3,4-tetrahydro-thieno[2,3-d]pyrimidine-6-carboxylic acid benzyl ester;

1-Methyl-2,4-dioxo-3-(2,2,2-trifluoro-ethyl)-1,2,3,4-tetrahydro-thieno[2,3-d]pyrimidine-6-carboxyl pyrimidine acid benzyl ester;

3-(Diethoxy-phosphorylmethyl)-1-methyl-2,4-dioxo-1,2,3,4-tetrahydro-thieno[2,3-d]pyrimidine-6-carboxylic acid benzyl ester;

3-But-2-ynyl-1-methyl-2,4-dioxo-1,2,3,4-tetrahydro-thieno[2,3-d]pyrimidine-6-carboxylic acid benzyl ester;

Bromo-ethyl)-1-methyl-2,4-dioxo-1,2,3,4-tetrahydro-thieno[2,3-d]pyrimidine-6-carboxylic acid;

1-Methyl-2,4-dioxo-3-[2-(tetrahydro-pyran-2-yloxy)-ethyl]-1,2,3,4-tetrahydro-thieno[2,3-d]pyrimidine-6-carboxylic acid benzyl ester;

1-Methyl-2,4-dioxo-3-propyl-1,2,3,4-tetrahydro-thieno[2,3-d]pyrimidine-6-carboxylic acid benzyl ester;

3-(2-Acetoxy-ethyl)-1-methyl-2,4-dioxo-1,2,3,4-tetrahydro-thieno[2,3-d]pyrimidine-6-carboxylic acid;

3-Butyl-1-methyl-2,4-dioxo-1,2,3,4-tetrahydro-thieno[2,3-d]pyrimidine-6-carboxylic acid benzyl ester;

3-Isobutyl-1-methyl-2,4-dioxo-1,2,3,4-tetrahydro-thieno[2,3-d]pyrimidine-6-carboxylic acid benzyl ester;

3-Ethyl-1-methyl-2,4-dioxo-1,2,3,4-tetrahydro-thieno[2,3-d]pyrimidine-6-carboxylic acid benzyl ester;

3-(3-Bromo-propyl)-1-methyl-2,4-dioxo-1,2,3,4-tetrahydro-thieno[2,3-d]pyrimidine-6-carboxylic acid benzyl ester;

3-Cyclohexylmethyl-1-methyl-2,4-dioxo-1,2,3,4-tetrahydro-thieno[2,3-d]pyrimidine-6-carboxylic acid benzyl ester;

3-(2-Ethylamino-ethyl)-1-methyl-2,4-dioxo-1,2,3,4-tetrahydro-thieno[2,3-d]pyrimidine-6-carboxylic acid benzyl ester;

3-Cyclobutylmethyl-1-methyl-2,4-dioxo-1,2,3,4-tetrahydro-thieno[2,3-d]pyrimidine-6-carboxylic acid benzyl ester;

3-((R)-3-Hydroxy-2-methyl-propyl)-1-methyl-2,4-dioxo-1,2,3,4-tetrahydro-thieno[2,3-d]pyrimidine-6-carboxylic acid benzyl ester;

3-(4-Hydroxy-butyl)-1-methyl-2,4-dioxo-1,2,3,4-tetrahydro-thieno[2,3-d]pyrimidine-6-carboxylic acid benzyl ester;

3-(2-Ethoxy-ethyl)-1-methyl-2,4-dioxo-1,2,3,4-tetrahydro-thieno[2,3-d]pyrimidine-6-carboxylic acid benzyl ester;

3-Isobutyl-1-methyl-2,4-dioxo-1,2,3,4-tetrahydro-thieno[2,3-d]pyrimidine-6-carboxylic acid benzyl ester;

3-(2-Chloro-ethyl)-1-methyl-2,4-dioxo-1,2,3,4-tetrahydro-thieno[2,3-d]pyrimidine-6-carboxylic acid benzyl ester;

1-Methyl-3-(3-methyl-but-2-enyl)-2,4-dioxo-1,2,3,4-tetrahydro-thieno[2,3-d]pyrimidine-6-carboxylic acid benzyl ester;

3-Allyl-1-methyl-2,4-dioxo-1,2,3,4-tetrahydro-thieno[2,3-d]pyrimidine-6-carboxylic acid benzyl ester;

3-(2,2-Dimethoxy-ethyl)-1-methyl-2,4-dioxo-1,2,3,4-tetrahydro-thieno[2,3-d]pyrimidine-6-carboxylic acid benzyl ester;

1-Methyl-3-oxiranylmethyl-2,4-dioxo-1,2,3,4-tetrahydro-thieno[2,3-d]pyrimidine-6-carboxylic acid benzyl ester;

1-Methyl-2,4-dioxo-3-propyl-1,2,3,4-tetrahydro-thieno[2,3-d]pyrimidine-6-carboxylic acid benzyl ester;

3-Benzo[1,2,5]oxadiazol-5-ylmethyl-1-methyl-2,4-dioxo-1,2,3,4-tetrahydro-thieno[2,3-d]pyrimidine-6-carboxylic acid benzyl ester;

3-(3-Hydroxy-2,2-dimethyl-propyl)-1-methyl-2,4-dioxo-1,2,3,4-tetrahydro-thieno[2,3-d]pyrimidine-6-carboxylic acid benzyl ester;

3-(2-Carboxy-ethyl)-1-methyl-2,4-dioxo-1,2,3,4-tetrahydro-thieno[2,3-d]pyrimidine-6-carboxylic acid benzyl ester;

3-Propyl-1-methyl-2,4-dioxo-1,2,3,4-tetrahydro-thieno[2,3-d]pyrimidine-6-carboxylic benzyl ester;

1-Methyl-2,4-dioxo-3-(4-sulfamoyl-benzyl)-1,2,3,4-tetrahydro-thieno[2,3-d]pyrimidine-6-carboxylic acid benzyl ester;

1-Methyl-3-(4-methylsulfamoyl-benzyl)-2,4-dioxo-1,2,3,4-tetrahydro-thieno[2,3-d]pyrimidine-6-carboxylic acid benzyl ester;

3-(4-Dimethylsulfamoyl-benzyl)-1-methyl-2,4-dioxo-1,2,3,4-tetrahydro-thieno[2,3-d]pyrimidine-6-carboxylic acid benzyl ester;

3-(4-Methanesulfonylamino-benzyl)-1-methyl-2,4-dioxo-1,2,3,4-tetrahydro-thieno[2,3-d]pyrimidine-6-carboxylic acid benzyl ester;

3-[4-(Methanesulfonyl-methyl-amino)-benzyl]-1-methyl-2,4-dioxo-1,2,3,4-tetrahydro-thieno[2,3-d]pyrimidine-6-carboxylic acid benzyl ester;

3-(4-Acetylamino-benzyl)-1-methyl-2,4-dioxo-1,2,3,4-tetrahydro-thieno[2,3-d]pyrimidine-6-carboxylic acid benzyl ester;

3-[4-(Acetyl-methyl-amino)-benzyl]-1-methyl-2,4-dioxo-1,2,3,4-tetrahydro-thieno[2,3-d]pyrimidine-6-carboxylic acid benzyl ester;

3-(4-Dimethylamino-benzyl)-1-methyl-2,4-dioxo-1,2,3,4-tetrahydro-thieno[2,3-d]pyrimidine-6-carboxylic acid benzyl ester;

1-Methyl-3-(4-methylamino-benzyl)-2,4-dioxo-1,2,3,4-tetrahydro-thieno[2,3-d]pyrimidine-6-carboxylic acid benzyl ester;

3-(4-Carbamoyl-benzyl)-1-methyl-2,4-dioxo-1,2,3,4-tetrahydro-thieno[2,3-d]pyrimidine-6-carboxylic acid benzyl ester;

3-(4-Dimethylcarbamoyl-benzyl)-1-methyl-2,4-dioxo-1,2,3,4-tetrahydro-thieno[2,3-d]pyrimidine-6-carboxylic acid benzyl ester;

3-(4-Carboxy-benzyl)-1-methyl-2,4-dioxo-1,2,3,4-tetrahydro-thieno[2,3-d]pyrimidine-6-carboxylic acid benzyl ester;

3-(4-Methoxycarbonyl-benzyl)-1-methyl-2,4-dioxo-1,2,3,4-tetrahydro-thieno[2,3-d]pyrimidine-6-carboxylic acid benzyl ester;

3-{4-[Bis-(2-hydroxy-ethyl)-amino]-benzyl}-1-1-methyl-2,4-dioxo-1,2,3,4-tetrahydro-thieno[2,3-d]pyrimidine-6-carboxylic acid benzyl ester;

3-(3,5-Dimethoxy-benzyl)-1-methyl-2,4-dioxo-1,2,3,4-tetrahydro-thieno[2,3-d]pyrimidine-6-carboxylic acid benzyl ester;

3-(4-tert-Butyl-benzyl)-1-methyl-2,4-dioxo-1,2,3,4-tetrahydro-thieno[2,3-d]pyrimidine-6-carboxylic acid benzyl ester;

1-Methyl-2,4-dioxo-3-(4-trifluoromethoxy-benzyl)-1,2,3,4-tetrahydro-thieno[2,3-d]pyrimidine-6-carboxylic acid benzyl ester;

3-(4-Methanesulfonyl-benzyl)-1-methyl-2,4-dioxo-1,2,3,4-tetrahydro-thieno[2,3-d]pyrimidine-6-carboxylic acid benzyl ester;

2,4-Dioxo-3-[1,3,4]thiadiazol-2-ylmethyl-1,2,3,4-tetrahydro-thieno[2,3-d]pyrimidine-6-carboxylic acid benzyl ester;

3-Isoxazol-3-ylmethyl-2,4-dioxo-1,2,3,4-tetrahydro-thieno[2,3-d]pyrimidine-6-carboxylic acid benzyl ester;

3-Oxazol-2-ylmethyl-2,4-dioxo-1,2,3,4-tetrahydro-thieno[2,3-d]pyrimidine-6-carboxylic acid benzyl ester;

2,4-Dioxo-3-thiazol-2-ylmethyl-1,2,3,4-tetrahydro-thieno[2,3-d]pyrimidine-6-carboxylic acid benzyl ester;

3-(1H-Imidazol-2-ylmethyl)-2,4-dioxo-1,2,3,4-tetrahydro-thieno[2,3-d]pyrimidine-6-carboxylic acid benzyl ester;

3-(1-Methyl-1H-imidazol-2-ylmethyl)-2,4-dioxo-1,2,3,4-tetrahydro-thieno[2,3-d]pyrimidine-6-carboxylic acid benzyl ester;

3-(1-Methyl-1H-pyrrol-2-ylmethyl)-2,4-dioxo-1,2,3,4-tetrahydro-thieno[2,3-d]pyrimidine-6-carboxylic acid benzyl ester;

2,4-Dioxo-3-(1H-pyrrol-2-ylmethyl)-1,2,3,4-tetrahydro-thieno[2,3-d]pyrimidine-6-carboxylic acid benzyl ester;

2,4-Dioxo-3-(1H-pyrrol-2-ylmethyl)-1,2,3,4-tetrahydro-thieno[2,3-d]pyrimidine-6-carboxylic acid benzyl ester;

2,4-Dioxo-3-thiophen-2-ylmethyl-1,2,3,4-tetrahydro-thieno[2,3-d]pyrimidine-6-carboxylic acid benzyl ester;

2,4-Dioxo-3-[1,2,3,4]tetrazin-5-ylmethyl-1,2,3,4-tetrahydro-thieno[2,3-d]pyrimidine-6-carboxylic acid benzyl ester;

2,4-Dioxo-3-[1,2,4,5]tetrazin-3-ylmethyl-1,2,3,4-tetrahydro-thieno[2,3-d]pyrimidine-6-carboxylic acid benzyl ester;

3-(1-Methyl-piperidin-4-ylmethyl)-2,4-dioxo-1,2,3,4-tetrahydro-thieno[2,3-d]pyrimidine-6-carboxylic acid benzyl ester;

2,4-Dioxo-3-pyrimidin-2-ylmethyl-1,2,3,4-tetrahydro-thieno[2,3-d]pyrimidine-6-carboxylic acid benzyl ester;

2,4-Dioxo-3-(2H-pyran-2-ylmethyl)-1,2,3,4-tetrahydro-thieno[2,3-d]pyrimidine-6-carboxylic acid benzyl ester;

3-(1H-Imidazo[4,5-b]pyridin-2-ylmethyl)-2,4-dioxo-1,2,3,4-tetrahydro-thieno[2,3-d]pyrimidine-6-carboxylic acid benzyl ester;

3-(1H-Benzoimidazol-2-ylmethyl)-2,4-dioxo-1,2,3,4-tetrahydro-thieno[2,3-d]pyrimidine-6-carboxylic acid benzyl ester;

3-Benzo[b]thiophen-2-ylmethyl-2,4-dioxo-1,2,3,4-tetrahydro-thieno[2,3-d]pyrimidine-6-carboxylic acid benzyl ester;

2,4-Dioxo-3-quinolin-2-ylmethyl-1,2,3,4-tetrahydro-thieno[2,3-d]pyrimidine-6-carboxylic acid benzyl ester;

3-(2H-Chromen-2-ylmethyl)-2,4-dioxo-1,2,3,4-tetrahydro-thieno[2,3-d]pyrimidine-6-carboxylic acid benzyl ester;

3-(1H-Benzoimidazol-2-ylmethyl)-2,4-dioxo-1,2,3,4-tetrahydro-thieno[2,3-d]pyrimidine-6-carboxylic acid benzyl ester;

3-(1-Methyl-1H-benzoimidazol-2-ylmethyl)-2,4-dioxo-1,2,3,4-tetrahydro-thieno[2,3-d]pyrimidine-6-carboxylic acid benzyl ester;

3-(1H-Indol-2-ylmethyl)-2,4-dioxo-1,2,3,4-tetrahydro-thieno[2,3-d]pyrimidine-6-carboxylic acid benzyl ester;

3-Benzyl-1-methyl-2,4-dioxo-1,2,3,4-tetrahydro-thieno[2,3-d]pyrimidine-6-carboxylic acid furan-3-ylmethyl ester;

3-Benzyl-1-methyl-2,4-dioxo-1,2,3,4-tetrahydro-thieno[2,3-d]pyrimidine-6-carboxylic acid 1-ethyl-propyl ester;

3-Benzyl-1-methyl-2,4-dioxo-1,2,3,4-tetrahydro-thieno[2,3-d]pyrimidine-6-carboxylic acid 1,1-dioxo-tetrahydro-116-thiophen-3-yl ester;

3-Benzyl-1-methyl-2,4-dioxo-1,2,3,4-tetrahydro-thieno[2,3-d]pyrimidine-6-carboxylic acid 4-hydroxy-benzyl ester;

3-Benzyl-1-methyl-2,4-dioxo-1,2,3,4-tetrahydro-thieno[2,3-d]pyrimidine-6-carboxylic acid 1-oxy-pyridin-4-ylmethyl ester;

3-Benzyl-1-methyl-2,4-dioxo-1,2,3,4-tetrahydro-thieno[2,3-d]pyrimidine-6-carboxylic acid but-3-enyl ester;

3-Benzyl-1-methyl-2,4-dioxo-1,2,3,4-tetrahydro-thieno[2,3-d]pyrimidine-6-carboxylic acid 3-diethylamino-propyl ester;

3-Benzyl-1-methyl-2,4-dioxo-1,2,3,4-tetrahydro-thieno[2,3-d]pyrimidine-6-carboxylic acid 1-cyano-1-phenyl-methyl ester;

3-Benzyl-1-methyl-2,4-dioxo-1,2,3,4-tetrahydro-thieno[2,3-d]pyrimidine-6-carboxylic acid 3-amino-benzyl ester;

3-Benzyl-1-methyl-2,4-dioxo-1,2,3,4-tetrahydro-thieno[2,3-d]pyrimidine-6-carboxylic acid 3-methoxy-benzyl ester;

3-Benzyl-1-methyl-2,4-dioxo-1,2,3,4-tetrahydro-thieno[2,3-d]pyrimidine-6-carboxylic acid 1-oxy-pyridin-3-ylmethyl ester;

3-Benzyl-1-methyl-2,4-dioxo-1,2,3,4-tetrahydro-thieno[2,3-d]pyrimidine-6-carboxylic acid 2-ethoxy-ethyl ester;

3-Benzyl-1-methyl-2,4-dioxo-1,2,3,4-tetrahydro-thieno[2,3-d]pyrimidine-6-carboxylic acid thiophen-2-ylmethyl ester;

3-Benzyl-1-methyl-2,4-dioxo-1,2,3,4-tetrahydro-thieno[2,3-d]pyrimidine-6-carboxylic acid 2,6-dichloro-benzyl ester;

3-Benzyl-1-methyl-2,4-dioxo-1,2,3,4-tetrahydro-thieno[2,3-d]pyrimidine-6-carboxylic acid dimethylamino-methyl-ethyl ester;

3-Benzyl-1-methyl-2,4-dioxo-1,2,3,4-tetrahydro-thieno[2,3-d]pyrimidine-6-carboxylic acid 2,2-diphenyl-ethyl ester;

3-Benzyl-1-methyl-2,4-dioxo-1,2,3,4-tetrahydro-thieno[2,3-d]pyrimidine-6-carboxylic acid 2-pyridin -2-yl-ethyl ester;

3-Benzyl-1-methyl-2,4-dioxo-1,2,3,4-tetrahydro-thieno[2,3-d]pyrimidine-6-carboxylic acid 2-ethanesulfonyl-ethyl ester;

3-Benzyl-1-methyl-2,4-dioxo-1,2,3,4-tetrahydro-thieno[2,3-d]pyrimidine-6-carboxylic acid diethylamino-methyl-ethyl ester;

3-Benzyl-1-methyl-2,4-dioxo-1,2,3,4-tetrahydro-thieno[2,3-d]pyrimidine-6-carboxylic acid dimethylamino-methyl-propyl ester;

3-Benzyl-1-methyl-2,4-dioxo-1,2,3,4-tetrahydro-thieno[2,3-d]pyrimidine-6-carboxylic acid 2-(2-chloro-phenoxy)-ethyl ester;

3-Benzyl-1-methyl-2,4-dioxo-1,2,3,4-tetrahydro-thieno[2,3-d]pyrimidine-6-carboxylic acid 2-(2-ethoxy-ethoxy)-ethyl ester;

3-Benzyl-1-methyl-2,4-dioxo-1,2,3,4-tetrahydro-thieno[2,3-d]pyrimidine-6-carboxylic acid 2-hydroxy-benzyl ester;

1-Methyl-2,4-dioxo-1,2,3,4-tetrahydro-thieno[2,3-d]pyrimidine-6-carboxylic acid 2-morpholin-4-yl-ethyl ester;

3-Benzyl-1-methyl-2,4-dioxo-1,2,3,4-tetrahydro-thieno[2,3-d]pyrimidine-6-carboxylic acid 2-(1,3-dioxo-1,3-dihydro-isoindol-2-yl)-ethyl ester;

3-Benzyl-1-methyl-2,4-dioxo-1,2,3,4-tetrahydro-thieno[2,3-d]pyrimidine-6-carboxylic acid 2,3-dihydro-benzo[1,4]dioxin-2-ylmethyl ester;

3-Benzyl-1-methyl-2,4-dioxo-1,2,3,4-tetrahydro-thieno[2,3-d]pyrimidine-6-carboxylic acid 1-methyl-piperidin-4-yl ester;

3-Benzyl-1-methyl-2,4-dioxo-1,2,3,4-tetrahydro-thieno[2,3-d]pyrimidine-6-carboxylic acid 2-(4-hydroxy-phenyl)-ethyl ester;

3-Benzyl-1-methyl-2,4-dioxo-1,2,3,4-tetrahydro-thieno[2,3-d]pyrimidine-6-carboxylic acid 2-cyano-ethyl ester;

3-Benzyl-1-methyl-2,4-dioxo-1,2,3,4-tetrahydro-thieno[2,3-d]pyrimidine-6-carboxylic acid hexyl ester;

3-Benzyl-1-methyl-2,4-dioxo-1,2,3,4-tetrahydro-thieno[2,3-d]pyrimidine-6-carboxylic acid 4-fluoro-benzyl ester;

3-Benzyl-1-methyl-2,4-dioxo-1,2,3,4-tetrahydro-thieno[2,3-d]pyrimidine-6-carboxylic acid 3-hydroxy-6-methyl-pyridin-2-ylmethyl ester;

3-Benzyl-1-methyl-2,4-dioxo-1,2,3,4-tetrahydro-thieno[2,3-d]pyrimidine-6-carboxylic acid 2-benzyloxy-ethyl ester;

3-Benzyl-1-methyl-2,4-dioxo-1,2,3,4-tetrahydro-thieno[2,3-d]pyrimidine-6-carboxylic acid 2-methoxy-benzyl ester;

3-Benzyl-1-methyl-2,4-dioxo-1,2,3,4-tetrahydro-thieno[2,3-d]pyrimidine-6-carboxylic acid 4-methoxy-benzyl ester;

3-Benzyl-1-methyl-2,4-dioxo-1,2,3,4-tetrahydro-thieno[2,3-d]pyrimidine-6-carboxylic acid 2,2,2-trifluoro-ethyl ester;

3-Benzyl-1-methyl-2,4-dioxo-1,2,3,4-tetrahydro-thieno[2,3-d]pyrimidine-6-carboxylic acid 2,2,2-trichloro-ethyl ester;

3-Benzyl-1-methyl-2,4-dioxo-1,2,3,4-tetrahydro-thieno[2,3-d]pyrimidine-6-carboxylic acid pyridin-3-ylmethyl ester;

3-Benzyl-1-methyl-2,4-dioxo-1,2,3,4-tetrahydro-thieno[2,3-d]pyrimidine-6-carboxylic acid pyridin-4-ylmethyl ester;

3-Benzyl-1-methyl-2,4-dioxo-1,2,3,4-tetrahydro-thieno[2,3-d]pyrimidine-6-carboxylic acid 3-pyridin-3-yl-propyl ester;

3-Benzyl-1-methyl-2,4-dioxo-1,2,3,4-tetrahydro-thieno[2,3-d]pyrimidine-6-carboxylic acid;

3-Benzyl-1-methyl-2,4-dioxo-1,2,3,4-tetrahydro-thieno[2,3-d]pyrimidine-6-carboxylic acid 2-phenoxy-ethyl ester;

3-Benzyl-1-methyl-2,4-dioxo-1,2,3,4-tetrahydro-thieno[2,3-d]pyrimidine-6-carboxylic acid 1,3-dimethyl-butyl ester;

3-Benzyl-1-methyl-2,4-dioxo-1,2,3,4-tetrahydro-thieno[2,3-d]pyrimidine-6-carboxylic acid 2-methyl-benzyl ester;

3-Benzyl-1-methyl-2,4-dioxo-1,2,3,4-tetrahydro-thieno[2,3-d]pyrimidine-6-carboxylic acid 1-phenyl-ethyl ester;

3-Benzyl-1-methyl-2,4-dioxo-1,2,3,4-tetrahydro-thieno[2,3-d]pyrimidine-6-carboxylic acid 1-benzyl-piperidin-4-yl ester;

3-Benzyl-1-methyl-2,4-dioxo-1,2,3,4-tetrahydro-thieno[2,3-d]pyrimidine-6-carboxylic acid propyl ester;

3-Benzyl-1-methyl-2,4-dioxo-1,2,3,4-tetrahydro-thieno[2,3-d]pyrimidine-6-carboxylic acid methyl ester;

3-Benzyl-1-methyl-2,4-dioxo-1,2,3,4-tetrahydro-thieno[2,3-d]pyrimidine-6-carboxylic acid 2-trifluoromethyl-benzyl ester;

3-Benzyl-1-methyl-2,4-dioxo-1,2,3,4-tetrahydro-thieno[2,3-d]pyrimidine-6-carboxylic acid 2-p-tolyl-ethyl ester;

3-Benzyl-1-methyl-2,4-dioxo-1,2,3,4-tetrahydro-thieno[2,3-d]pyrimidine-6-carboxylic acid 4-trifluoromethyl-benzyl ester;

3-Benzyl-1-methyl-2,4-dioxo-1,2,3,4-tetrahydro-thieno[2,3-d]pyrimidine-6-carboxylic acid tetrahydro-furan-2-ylmethyl ester;

3-Benzyl-1-methyl-2,4-dioxo-1,2,3,4-tetrahydro-thieno[2,3-d]pyrimidine-6-carboxylic acid octahydro-inden-5-yl ester;

3-Benzyl-1-methyl-2,4-dioxo-1,2,3,4-tetrahydro-thieno[2,3-d]pyrimidine-6-carboxylic acid 4-amino-benzyl ester;

3-Benzyl-1-methyl-2,4-dioxo-1,2,3,4-tetrahydro-thieno[2,3-d]pyrimidine-6-carboxylic acid 2-aziridin-1-yl-ethyl ester;

3-Benzyl-1-methyl-2,4-dioxo-1,2,3,4-tetrahydro-thieno[2,3-d]pyrimidine-6-carboxylic acid 3-methyl-but-2-enyl ester;

3-Benzyl-1-methyl-2,4-dioxo-1,2,3,4-tetrahydro-thieno[2,3-d]pyrimidine-6-carboxylic acid benzyl ester;

3-Benzyl-1-methyl-2,4-dioxo-1,2,3,4-tetrahydro-thieno[2,3-d]pyrimidine-6-carboxylic acid trifluoro-trifluoromethyl-ethyl ester;

3-Benzyl-1-methyl-2,4-dioxo-1,2,3,4-tetrahydro-thieno[2,3-d]pyrimidine-6-carboxylic acid phenethyl ester;

3-Benzyl-1-methyl-2,4-dioxo-1,2,3,4-tetrahydro-thieno[2,3-d]pyrimidine-6-carboxylic acid 2-methoxy-ethyl ester;

3-Benzyl-1-methyl-2,4-dioxo-1,2,3,4-tetrahydro-thieno[2,3-d]pyrimidine-6-carboxylic acid biphenyl-4-ylmethyl ester;

3-Benzyl-1-methyl-2,4-dioxo-1,2,3,4-tetrahydro-thieno[2,3-d]pyrimidine-6-carboxylic acid 2-chloro-6-fluoro-benzyl ester;

3-Benzyl-1-methyl-2,4-dioxo-1,2,3,4-tetrahydro-thieno[2,3-d]pyrimidine-6-carboxylic acid tetrahydro-pyran-4-yl ester;

3-Benzyl-1-methyl-2,4-dioxo-1,2,3,4-tetrahydro-thieno[2,3-d]pyrimidine-6-carboxylic acid 3-ethyl-oxetan-3-ylmethyl ester;

3-Benzyl-1-methyl-2,4-dioxo-1,2,3,4-tetrahydro-thieno[2,3-d]pyrimidine-6-carboxylic acid butyl ester;

3-Benzyl-1-methyl-2,4-dioxo-1,2,3,4-tetrahydro-thieno[2,3-d]pyrimidine-6-carboxylic acid 2-(2-hydroxy-phenyl)-ethyl ester;

3-Benzyl-1-methyl-2,4-dioxo-1,2,3,4-tetrahydro-thieno[2,3-d]pyrimidine-6-carboxylic acid 2-(4-fluoro-phenyl)-ethyl ester;

3-Benzyl-1-methyl-2,4-dioxo-1,2,3,4-tetrahydro-thieno[2,3-d]pyrimidine-6-carboxylic acid cyclopropylmethyl ester;

3-Benzyl-1-methyl-2,4-dioxo-1,2,3,4-tetrahydro-thieno[2,3-d]pyrimidine-6-carboxylic acid 4-ethyl-benzyl ester;

3-Benzyl-1-methyl-2,4-dioxo-1,2,3,4-tetrahydro-thieno[2,3-d]pyrimidine-6-carboxylic acid (S)-1-phenyl-ethyl ester;

3-Benzyl-1-methyl-2,4-dioxo-1,2,3,4-tetrahydro-thieno[2,3-d]pyrimidine-6-carboxylic acid 2,6-difluoro-benzyl ester;

3-Benzyl-1-methyl-2,4-dioxo-1,2,3,4-tetrahydro-thieno[2,3-d]pyrimidine-6-carboxylic acid cyclobutyl methyl ester;

3-Benzyl-1-methyl-2,4-dioxo-1,2,3,4-tetrahydro-thieno[2,3-d]pyrimidine-6-carboxylic acid 2-pyridin-4-yl-ethyl ester;

3-Benzyl-1-methyl-2,4-dioxo-1,2,3,4-tetrahydro-thieno[2,3-d]pyrimidine-6-carboxylic acid 3-hydroxy-cyclopentyl ester;

3-Benzyl-1-methyl-2,4-dioxo-1,2,3,4-tetrahydro-thieno[2,3-d]pyrimidine-6-carboxylic acid 1-pentafluorophenyl-ethyl ester;

3-Benzyl-1-methyl-2,4-dioxo-1,2,3,4-tetrahydro-thieno[2,3-d]pyrimidine-6-carboxylic acid 2-benzyloxycarbonylamino-ethyl ester; and

3-Benzyl-1-methyl-2,4-dioxo-1,2,3,4-tetrahydro-thieno[2,3-d]pyrimidine-6-carboxylic acid ethyl ester.

Another invention embodiment is any compound of the above formulas wherein

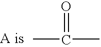

and B is NR⁵, wherein R⁵ is as defined above.

Another invention embodiment is a compound selected from:

3-Benzyl-1-methyl-2,4-dioxo-1,2,3,4-tetrahydro-thieno[2,3-d]pyrimidine-6-carboxylic acid (2-pyridin-4-yl-ethyl)-amide;

3-Benzyl-1-methyl-2,4-dioxo-1,2,3,4-tetrahydro-thieno[2,3-d]pyrimidine-6-carboxylic acid (2-morpholin-4-yl-ethyl)-amide;

3-Benzyl-1-methyl-2,4-dioxo-1,2,3,4-tetrahydro-thieno[2,3-d]pyrimidine-6-carboxylic acid 4-methyl-benzylamide;

3-Benzyl-1-methyl-2,4-dioxo-1,2,3,4-tetrahydro-thieno[2,3-d]pyrimidine-6-carboxylic acid sec-butylamide;

3-Benzyl-1-methyl-2,4-dioxo-1,2,3,4-tetrahydro-thieno[2,3-d]pyrimidine-6-carboxylic acid cyclopentylamide;

3-Benzyl-1-methyl-2,4-dioxo-1,2,3,4-tetrahydro-thieno[2,3-d]pyrimidine-6-carboxylic acid cyclopropylamide;

3-Benzyl-1-methyl-2,4-dioxo-1,2,3,4-tetrahydro-thieno[2,3-d]pyrimidine-6-carboxylic acid cyanomethyl-amide;

3-Benzyl-1-methyl-2,4-dioxo-1,2,3,4-tetrahydro-thieno[2,3-d]pyrimidine-6-carboxylic acid cyclohexylamide;

3-Benzyl-1-methyl-2,4-dioxo-1,2,3,4-tetrahydro-thieno[2,3-d]pyrimidine-6-carboxylic acid 3-methyl-benzylamide;

3-Benzyl-1-methyl-2,4-dioxo-1,2,3,4-tetrahydro-thieno[2,3-d]pyrimidine-6-carboxylic acid (3-ethoxy-propyl)-amide;

3-Benzyl-1-methyl-2,4-dioxo-1,2,3,4-tetrahydro-thieno[2,3-d]pyrimidine-6-carboxylic acid 2-chloro-benzylamide;

3-Benzyl-1-methyl-2,4-dioxo-1,2,3,4-tetrahydro-thieno[2,3-d]pyrimidine-6-carboxylic acid 2-methyl-benzylamide;

3-Benzyl-1-methyl-2,4-dioxo-1,2,3,4-tetrahydro-thieno[2,3-d]pyrimidine-6-carboxylic acid (2,2-diphenyl-ethyl)-amide;

3-Benzyl-1-methyl-2,4-dioxo-1,2,3,4-tetrahydro-thieno[2,3-d]pyrimidine-6-carboxylic acid (pyridin-3-ylmethyl)-amide;

3-Benzyl-1-methyl-2,4-dioxo-1,2,3,4-tetrahydro-thieno[2,3-d]pyrimidine-6-carboxylic acid cyclopropylmethyl-amide;

3-Benzyl-1-methyl-2,4-dioxo-1,2,3,4-tetrahydro-thieno[2,3-d]pyrimidine-6-carboxylic acid (1-ethyl-pyrrolidin-2-ylmethyl)-amide;

3-Benzyl-1-methyl-2,4-dioxo-1,2,3,4-tetrahydro-thieno[2,3-d]pyrimidine-6-carboxylic acid (pyridin-2-ylmethyl)-amide;

3-Benzyl-1-methyl-2,4-dioxo-1,2,3,4-tetrahydro-thieno[2,3-d]pyrimidine-6-carboxylic acid[2-(3,4-dimethoxy-phenyl)-ethyl]-amide;

3-Benzyl-1-methyl-2,4-dioxo-1,2,3,4-tetrahydro-thieno[2,3-d]pyrimidine-6-carboxylic acid (furan-2-ylmethyl)-amide;

3-Benzyl-1-methyl-2,4-dioxo-1,2,3,4-tetrahydro-thieno[2,3-d]pyrimidine-6-carboxylic acid 2-fluoro-benzylamide;

3-Benzyl-1-methyl-2,4-dioxo-1,2,3,4-tetrahydro-thieno[2,3-d]pyrimidine-6-carboxylic acid (2-bromo-ethyl)-amide;

3-Benzyl-1-methyl-2,4-dioxo-1,2,3,4-tetrahydro-thieno[2,3-d]pyrimidine-6-carboxylic acid 4-sulfamoyl-benzylamide;

3-Benzyl-1-methyl-2,4-dioxo-1,2,3,4-tetrahydro-thieno[2,3-d]pyrimidine-6-carboxylic acid 3-methoxy-benzylamide;

3-Benzyl-1-methyl-2,4-dioxo-1,2,3,4-tetrahydro-thieno[2,3-d]pyrimidine-6-carboxylic acid 4-methoxy-benzylamide;

3-Benzyl-1-methyl-2,4-dioxo-1,2,3,4-tetrahydro-thieno[2,3-d]pyrimidine-6-carboxylic acid phenethyl-amide;

(S)-2-{[1-(3-Benzyl-1-methyl-2,4-dioxo-1,2,3,4-tetrahydro-thieno[2,3-d]pyrimidin-6-yl)-methanoyl]-amino}-propionic acid;

3-Benzyl-1-methyl-2,4-dioxo-1,2,3,4-tetrahydro-thieno[2,3-d]pyrimidine-6-carboxylic acid (1-phenyl-ethyl)-amide;

3-Benzyl-1-methyl-2,4-dioxo-1,2,3,4-tetrahydro-thieno[2,3-d]pyrimidine-6-carboxylic acid 2-methoxy-benzylamide;

3-Benzyl-1-methyl-2,4-dioxo-1,2,3,4-tetrahydro-thieno[2,3-d]pyrimidine-6-carboxylic acid benzylamide;

3-Benzyl-1-methyl-2,4-dioxo-1,2,3,4-tetrahydro-thieno[2,3-d]pyrimidine-6-carboxylic acid 3-bromo-benzylamide;

3-Benzyl-1-methyl-2,4-dioxo-1,2,3,4-tetrahydro-thieno[2,3-d]pyrimidine-6-carboxylic acid[2-(4-sulfamoyl-phenyl)-ethyl]-amide;

2-{[1-(3-Benzyl-1-methyl-2,4-dioxo-1,2,3,4-tetrahydro-thieno[2,3-d]pyrimidin-6-yl)-methanoyl]-amino}-3-phenyl-propionic acid methyl ester;

3-Benzyl-1-methyl-2,4-dioxo-1,2,3,4-tetrahydro-thieno[2,3-d]pyrimidine-6-carboxylic acid (3-imidazol-1-yl-propyl)-amide;

3-Benzyl-1-methyl-2,4-dioxo-1,2,3,4-tetrahydro-thieno[2,3-d]pyrimidine-6-carboxylic acid[2-(2-methoxy-phenyl)-ethyl]-amide;

3-Benzyl-1-methyl-2,4-dioxo-1,2,3,4-tetrahydro-thieno[2,3-d]pyrimidine-6-carboxylic acid 4-trifluoromethyl-benzylamide;

3-Benzyl-1-methyl-2,4-dioxo-1,2,3,4-tetrahydro-thieno[2,3-d]pyrimidine-6-carboxylic acid 4-amino-benzylamide;

3-Benzyl-1-methyl-2,4-dioxo-1,2,3,4-tetrahydro-thieno[2,3-d]pyrimidine-6-carboxylic acid[2-(4-fluoro-phenyl)-ethyl]-amide; and

3-Benzyl-1-methyl-2,4-dioxo-1,2,3,4-tetrahydro-thieno[2,3-d]pyrimidine-6-carboxylic acid ((R)-2-hydroxy-1-methyl-ethyl)-amide.

Another invention embodiment is a compound selected from:

3-Benzyl-1-methyl-2,4-dioxo-1,2,3,4-tetrahydro-thieno[2,3-d]pyrimidine-6-carboxylic acid benzofuran-5-ylmethyl ester;

(3-{[1-(3-Benzyl-1-methyl-2,4-dioxo-1,2,3,4-tetrahydro-thieno[2,3-d]pyrimidin-6-yl)-methanoyl]-amino}-propyl)-carbamic acid tert-butyl ester;

3-Benzyl-1-methyl-2,4-dioxo-1,2,3,4-tetrahydro-thieno[2,3-d]pyrimidine-6-carboxylic acid benzofuran-2-ylmethyl ester;

3-Benzyl-2,4-dioxo-1,2,3,4-tetrahydro-thieno[2,3-d]pyrimidine-6-carboxylic acid thiophen-3-ylmethyl ester;

3-Benzyl-1-methyl-2,4-dioxo-1,2,3,4-tetrahydro-thieno[2,3-d]pyrimidine-6-carboxylic acid 3H-[1,2,3]oxathiazol-5-ylmethyl ester;

3-Benzyl-2,4-dioxo-1,2,3,4-tetrahydro-thieno[2,3-d]pyrimidine-6-carboxylic acid 3H-[1,2,3]oxathiazol-5-ylmethyl ester;

3-Benzyl-2,4-dioxo-1,2,3,4-tetrahydro-thieno[2,3-d]pyrimidine-6-carboxylic acid[1,4,2]dioxazol-3-ylmethyl ester;

3-Benzyl-1-methyl-2,4-dioxo-1,2,3,4-tetrahydro-thieno[2,3-d]pyrimidine-6-carboxylic acid[1,4,2]dioxazol-3-ylmethyl ester;

3-Benzyl-2,4-dioxo-1,2,3,4-tetrahydro-thieno[2,3-d]pyrimidine-6-carboxylic acid furazan-3-ylmethyl ester;

3-Benzyl-1-methyl-2,4-dioxo-1,2,3,4-tetrahydro-thieno[2,3-d]pyrimidine-6-carboxylic acid furazan-3-ylmethyl ester;

3-Benzyl-1-methyl-2,4-dioxo-1,2,3,4-tetrahydro-thieno[2,3-d]pyrimidine-6-carboxylic acid[1,2,4]oxadiazol-5-ylmethyl ester;

3-Benzyl-2,4-dioxo-1,2,3,4-tetrahydro-thieno[2,3-d]pyrimidine-6-carboxylic acid[1,2,4]oxadiazol-5-ylmethyl ester;

3-Benzyl-2,4-dioxo-1,2,3,4-tetrahydro-thieno[2,3-d]pyrimidine-6-carboxylic acid 3H-[1,2,3]triazol-4-ylmethyl ester;

3-Benzyl-1-methyl-2,4-dioxo-1,2,3,4-tetrahydro-thieno[2,3-d]pyrimidine-6-carboxylic acid 3H-[1,2,3]triazol-4-ylmethyl ester;

3-Benzyl-1-methyl-2,4-dioxo-1,2,3,4-tetrahydro-thieno[2,3-d]pyrimidine-6-carboxylic acid 2H-[1,2,4]triazol-3-ylmethyl ester;

3-Benzyl-2,4-dioxo-1,2,3,4-tetrahydro-thieno[2,3-d]pyrimidine-6-carboxylic acid 2H-[1,2,4]triazol-3-ylmethyl ester;

3-Benzyl-2,4-dioxo-1,2,3,4-tetrahydro-thieno[2,3-d]pyrimidine-6-carboxylic acid isoxazol-5-ylmethyl ester;

3-Benzyl-1-methyl-2,4-dioxo-1,2,3,4-tetrahydro-thieno[2,3-d]pyrimidine-6-carboxylic acid isoxazol-5-ylmethyl ester;

3-Benzyl-1-methyl-2,4-dioxo-1,2,3,4-tetrahydro-thieno[2,3-d]pyrimidine-6-carboxylic acid oxazol-2-ylmethyl ester;

3-Benzyl-2,4-dioxo-1,2,3,4-tetrahydro-thieno[2,3-d]pyrimidine-6-carboxylic acid oxazol-2-ylmethyl ester;

3-Benzyl-2,4-dioxo-1,2,3,4-tetrahydro-thieno[2,3-d]pyrimidine-6-carboxylic acid isothiazol-5-ylmethyl ester;

3-Benzyl-1-methyl-2,4-dioxo-1,2,3,4-tetrahydro-thieno[2,3-d]pyrimidine-6-carboxylic acid isothiazol-5-ylmethyl ester;

3-Benzyl-2,4-dioxo-1,2,3,4-tetrahydro-thieno[2,3-d]pyrimidine-6-carboxylic acid thiazol-2-ylmethyl ester;

3-Benzyl-1-methyl-2,4-dioxo-1,2,3,4-tetrahydro-thieno[2,3-d]pyrimidine-6-carboxylic acid thiazol-2-ylmethyl ester;

3-Benzyl-1-methyl-2,4-dioxo-1,2,3,4-tetrahydro-thieno[2,3-d]pyrimidine-6-carboxylic acid 1H-imidazol-2-ylmethyl ester;

3-Benzyl-2,4-dioxo-1,2,3,4-tetrahydro-thieno[2,3-d]pyrimidine-6-carboxylic acid 2H-imidazol-2-ylmethyl ester;

3-Benzyl-2,4-dioxo-1,2,3,4-tetrahydro-thieno[2,3-d]pyrimidine-6-carboxylic acid 1H-pyrazol-3-ylmethyl ester;

3-Benzyl-1-methyl-2,4-dioxo-1,2,3,4-tetrahydro-thieno[2,3-d]pyrimidine-6-carboxylic acid 2H-pyrazol-3-ylmethyl ester;

3-Benzyl-1-methyl-2,4-dioxo-1,2,3,4-tetrahydro-thieno[2,3-d]pyrimidine-6-carboxylic acid 1H-pyrrol-2-ylmethyl ester;

3-Benzyl-2,4-dioxo-1,2,3,4-tetrahydro-thieno[2,3-d]pyrimidine-6-carboxylic acid 2H-pyrrol-2-ylmethyl ester;

3-Furazan-3-ylmethyl-2,4-dioxo-1,2,3,4-tetrahydro-thieno[2,3-d]pyrimidine-6-carboxylic acid benzyl ester;

3-Benzyl-1-methyl-2,4-dioxo-1,2,3,4-tetrahydro-thieno[2,3-d]pyrimidine-6-carboxylic acid 2H-chromen-2-ylmethyl ester;

3-Benzyl-1-methyl-2,4-dioxo-1,2,3,4-tetrahydro-thieno[2,3-d]pyrimidine-6-carboxylic acid 2H-thiochromen-2-ylmethyl ester;

3-Benzyl-2,4-dioxo-1,2,3,4-tetrahydro-thieno[2,3-d]pyrimidine-6-carboxylic acid 2H-thiochromen-2-ylmethyl ester;

3-Benzyl-2,4-dioxo-1,2,3,4-tetrahydro-thieno[2,3-d]pyrimidine-6-carboxylic acid[1,3,4]thiadiazol-2-ylmethyl ester;

3-Benzyl-1-methyl-2,4-dioxo-1,2,3,4-tetrahydro-thieno[2,3-d]pyrimidine-6-carboxylic acid[1,3,4]thiadiazol-2-ylmethyl ester;

3-Benzyl-2,4-dioxo-1,2,3,4-tetrahydro-thieno[2,3-d]pyrimidine-6-carboxylic acid 1H-benzoimidazol-5-ylmethyl ester;

3-Benzyl-1-methyl-2,4-dioxo-1,2,3,4-tetrahydro-thieno[2,3-d]pyrimidine-6-carboxylic acid 1H-benzoimidazol-5-ylmethyl ester;

3-Benzyl-1-methyl-2,4-dioxo-1,2,3,4-tetrahydro-thieno[2,3-d]pyrimidine-6-carboxylic acid 1H-benzoimidazol-2-ylmethyl ester;

3-Benzyl-2,4-dioxo-1,2,3,4-tetrahydro-thieno[2,3-d]pyrimidine-6-carboxylic acid 1H-benzoimidazol-2-ylmethyl ester;

3-Benzyl-2,4-dioxo-1,2,3,4-tetrahydro-thieno[2,3-d]pyrimidine-6-carboxylic acid 1H-indol-2-ylmethyl ester;

3-Benzyl-1-methyl-2,4-dioxo-1,2,3,4-tetrahydro-thieno[2,3-d]pyrimidine-6-carboxylic acid 1H-indol-2-ylmethyl ester;

3-Benzyl-1-methyl-2,4-dioxo-1,2,3,4-tetrahydro-thieno[2,3-d]pyrimidine-6-carboxylic acid 1H-indol-5-ylmethyl ester;

3-Benzyl-2,4-dioxo-1,2,3,4-tetrahydro-thieno[2,3-d]pyrimidine-6-carboxylic acid 1H-indol-5-ylmethyl ester;

3-Benzyl-2,4-dioxo-1,2,3,4-tetrahydro-thieno[2,3-d]pyrimidine-6-carboxylic acid 2,3-dihydro-benzofuran-5-ylmethyl ester; and

3-Benzyl-1-methyl-2,4-dioxo-1,2,3,4-tetrahydro-thieno[2,3-d]pyrimidine-6-carboxylic acid 2,3-dihydro-benzofuran-5-ylmethyl ester.

Another invention embodiment is a compound selected from:

4-{6-[3-(4-Methoxy-phenyl)-prop-1-ynyl]-1-methyl-2,4-dioxo-1,4-dihydro-2H-thieno[2,3-d]pyrimidine-3-ylmethyl}-benzoic acid;

3-(4-Methanesulfonyl-benzyl)-6-[3-(4-methoxy-phenyl)-prop-1-ynyl]-1-methyl-1H-thieno[2,3-d]pyrimidine-2,4-dione;

4-{6-[3-(3-Methoxy-phenyl)-prop-1-ynyl]-1-methyl-2,4-dioxo-1,4-dihydro-2H-thieno[2,3-d]pyrimidine-3-ylmethyl}-benzoic acid;

3-(4-Methanesulfonyl-benzyl)-6-[3-(3-methoxy-phenyl)-prop-1-ynyl]-1-methyl-1H-thieno[2,3-d]pyrimidine-2,4-dione;

4-[1-Methyl-2,4-dioxo-6-(3-pyridine-4-yl-prop-1-ynyl)-1,4-dihydro-2H-thieno[2,3-d]pyrimidine-3-ylmethyl]-benzoic acid;

3-(4-Methanesulfonyl-benzyl)-1-6-(3-pyridin-4-yl-prop-1-ynyl)-1H-thieno[2,3-d]pyrimidine-2,4-dione;

4-[1-Methyl-2,4-dioxo-6-(3-pyridine-3-yl-prop-1-ynyl)-1,4-dihydro-2H-thieno[2,3-d]pyrimidine-3-ylmethyl]-benzoic acid;

3-(4-Methanesulfonyl-benzyl)-1-6-(3-pyridin-3-yl-prop-1-ynyl)-1H-thieno[2,3-d]pyrimidine-2,4-dione;

4-{6-[3-(4-Fluoro-phenyl)-prop-1-ynyl]-1-methyl-2,4-dioxo-1,4-dihydro-2H-thieno[2,3-d]pyrimidine-3-ylmethyl}-benzoic acid 6-[3-(4-Fluoro-phenyl)-prop-1-ynyl]-3-(4-methanesulfonyl-benzyl)-1-methyl-1H-thieno[2,3-d]pyrimidine-2,4-dione;

4-{6-[3-(3-Fluoro-phenyl)-prop-1-ynyl]-1-methyl-2,4-dioxo-1,4-dihydro-2H-thieno[2,3-d]pyrimidine-3-ylmethyl}-benzoic acid;

6-[3-(3-Fluoro-phenyl)-prop-1-ynyl]-3-(4-methanesulfonyl-benzyl)-1-methyl-1H-thieno[2,3-d]pyrimidine-2,4-dione;

4-{6-[3-(4-Chloro-phenyl)-prop-1-ynyl]-1-methyl-2,4-dioxo-1,4-dihydro-2H-thieno[2,3-d]pyrimidine-3-ylmethyl}-benzoic acid;

6-[3-(4-Chloro-phenyl)-prop-1-ynyl]-3-(4-methanesulfonyl-benzyl)-1-methyl-1H-thieno[2,3-d]pyrimidine-2,4-dione;

4-{6-[3-(3-Chloro-phenyl)-prop-1-ynyl]-1-methyl-2,4-dioxo-1,4-dihydro-2H-thieno[2,3-d]pyrimidine-3-ylmethyl}-benzoic acid;

6-[3-(3-Chloro-phenyl)-prop-1-ynyl]-3-(4-methanesulfonyl-benzyl)-1-methyl-1H-thieno[2,3-d]pyrimidine-2,4-dione;

4-{6-[3-(4-Bromo-phenyl)-prop-1-ynyl]-1-methyl-2,4-dioxo-1,4-dihydro-2H-thieno[2,3-d]pyrimidine-3-ylmethyl}-benzoic acid;

6-[3-(4-Bromo-phenyl)-prop-1-ynyl]-3-(4-methanesulfonyl-benzyl)-1-methyl-1H-thieno[2,3-d]pyrimidine-2,4-dione;

4-{6-[3-(3-Bromo-phenyl)-prop-1-ynyl]-1-methyl-2,4-dioxo-1,4-dihydro-2H-thieno[2,3-d]pyrimidine-3-ylmethyl}-benzoic acid;

6-[3-(3-Bromo-phenyl)-prop-1-ynyl]-3-(4-methanesulfonyl-benzyl)-1-methyl-1H-thieno[2,3-d]pyrimidine-2,4-dione;

4-{1-Methyl-6-[3-(4-nitro-phenyl)-prop-1-ynyl]-2,4-dioxo-1,4-dihydro-2H-thieno[2,3-d]pyrimidine-3-ylmethyl}-benzoic acid;

3-(4-Methanesulfonyl-benzyl)-1-methyl-6-[3-(4-nitro-phenyl)-prop-1-ynyl)-1H-thieno[2,3-d]pyrimidine-2,4-dione;

4-{6-[3-(2-Methoxy-pyridin-4-yl)-prop-1-ynyl]-1-methyl-2,4-dioxo-1,4-dihydro-2H-thieno[2,3-d]pyrimidine-3-ylmethyl}-benzoic acid;

3-(4-Methanesulfonyl-benzyl)-6-[3-(2-methoxy-pyridin-4-yl)-prop-1-ynyl]-1-methyl-1H-thieno[2,3-d]pyrimidine-2,4-dione;

4-{1-Methyl-6-[3-(4-methylsulfanyl-phenyl)-prop-1-ynyl]-2,4-dioxo-1,4-dihydro-2H-thieno[2,3-d]pyrimidine-3-ylmethyl}-benzoic acid;

3-(4-Methanesulfonyl-benzyl)-1-methyl-6-[3-(4-methylsulfanyl-phenyl)-prop-1-ynyl]-1H-thieno[2,3-d]pyrimidine-2,4-dione;

4-{1-Methyl-6-[3-(3-methylsulfanyl-phenyl)-prop-1-ynyl]-2,4-dioxo-1,4-dihydro-2H-thieno[2,3-d]pyrimidine-3-ylmethyl}-benzoic acid;

3-(4-Methanesulfonyl-benzyl)-1-methyl-6-[3-(3-methylsulfanyl-phenyl)-prop-1-ynyl]-1H-thieno[2,3-d]pyrimidine-2,4-dione;

4-[1-Methyl-2,4-dioxo-6-(3-p-tolyl-prop-1-ynyl)-1,4-dihydro-2H-thieno[2,3-d]pyrimidin-3-ylmethyl]benzoic acid;

3-(4-Methanesulfonyl-benzyl)-1-methyl-6-(3-p-tolyl-prop-1-ynyl)-1 H-thieno[2,3-d]pyrimidine-2,4-dione;

4-[1-Methyl-2,4-dioxo-6-(3-m-tolyl-prop-1-ynyl)-1,4-dihydro-2H-thieno[2,3-d]pyrimidin-3-ylmethyl]benzoic acid;

3-(4-Methanesulfonyl-benzyl)-1-methyl-6-(3-m-tolyl-prop-1-ynyl)-1H-thieno[2,3-d]pyrimidine-2,4-dione;

3-Benzyl-6-[3-(4-methoxy-phenyl)-prop-1-ynyl]-1-methyl-1H-thieno[2,3-d]pyrimidine-2,4-dione;

3-Benzyl-6-[3-(3-methoxy-phenyl)-prop-1-ynyl]-1-methyl-1H-thieno[2,3-d]pyrimidine-2,4-dione;

3-Benzyl-1-methyl-6-(3-pyridin-4-yl-prop-1-ynyl)-1H-thieno[2,3-d]pyrimidine-2,4-dione;

3-Benzyl-1-methyl-6-(3-pyridin-3-yl-prop-1-ynyl)-1H-thieno[2,3-d]pyrimidine-2,4-dione;

3-Benzyl-6-[3-(4-fluoro-phenyl)-prop-1-ynyl]-1-methyl-1H-thieno[2,3-d]pyrimidine-2,4-dione;

3-Benzyl-6-[3-(3-fluoro-phenyl)-prop-1-ynyl]-1-methyl-1H-thieno[2,3-d]pyrimidine-2,4-dione;

3-Benzyl-6-[3-(4-chloro-phenyl)-prop-1-ynyl]-1-methyl-1H-thieno[2,3-d]pyrimidine-2,4-dione;

3-Benzyl-6-[3-(3-chloro-phenyl)-prop-1-ynyl]-1-methyl-1H-thieno[2,3-d]pyrimidine-2,4-dione;

3-Benzyl-6-[3-(4-bromo-phenyl)-prop-1-ynyl]-1-methyl-1H-thieno[2,3-d]pyrimidine-2,4-dione;

3-Benzyl-6-[3-(3-bromo-phenyl)-prop-1-ynyl]-1-methyl-1H-thieno[2,3-d]pyrimidine-2,4-dione;

3-Benzyl-6-[3-(2-methoxy-pyridin-4-yl)-prop-1-ynyl]-1-methyl-1H-thieno[2,3-d]pyrimidine-2,4-dione;

3-Benzyl-1-methyl-6-[3-(4-methylsulfanyl-phenyl)-prop-1-ynyl]-1H-thieno[2,3-d]pyrimidine-2,4-dione;

3-Benzyl-1-methyl-6-[3-(3-methylsulfanyl-phenyl)-prop-1-ynyl]-1H-thieno[2,3-d]pyrimidine-2,4-dione;

3-Benzyl-1-methyl-6-(3-p-tolyl-prop-1-ynyl)-1H-thieno[2,3-d]pyrimidine-2,4-dione;

3-Benzyl-1-methyl-6-(3-m-tolyl-prop-1-ynyl)-1H-thieno[2,3-d]pyrimidine-2,4-dione;

3-(3-Fluoro-benzyl)-6-[3-(4-methoxy-phenyl)-prop-1-ynyl]-1-methyl-1H-thieno[2,3-d]pyrimidine-2,4-dione;

3-(3-Fluoro-benzyl)-6-[3-(3-methoxy-phenyl)-prop-1-ynyl]-1-methyl-1H-thieno[2,3-d]pyrimidine-2,4-dione;

3-(3-Fluoro-benzyl)-1-methyl-6-(3-pyridine-4-yl-prop-1-ynyl)-1H-thieno[2,3-d]pyrimidine-2,4-dione;

3-(3-Fluoro-benzyl)-1-methyl-6-(3-pyridine-3-yl-prop-1-ynyl)-1H-thieno[2,3-d]pyrimidine-2,4-dione;

3-(3-Fluoro-benzyl)-6-[3-(4-fluoro-phenyl)-prop-1-ynyl]-1-methyl-1H-thieno[2,3-d]pyrimidine-2,4-dione;

3-(3-Fluoro-benzyl)-6-[3-(3-fluoro-phenyl)-prop-1-ynyl]-1-methyl-1H-thieno[2,3-d]pyrimidine-2,4-dione;

6-[3-(4-Chloro-phenyl)-prop-1-ynyl]-3-(3-fluoro-benzyl)-1-methyl-1H-thieno[2,3-d]pyrimidine-2,4-dione;

6-[3-(3-Chloro-phenyl)-prop-1-ynyl]-3-(3-fluoro-benzyl)-1-methyl-1H-thieno[2,3-d]pyrimidine-2,4-dione;

6-[3-(4-Bromo-phenyl)-prop-1-ynyl]-3-(3-fluoro-benzyl)-1-methyl-1H-thieno[2,3-d]pyrimidine-2,4-dione;

6-[3-(3-Bromo-phenyl)-prop-1-ynyl]-3-(3-fluoro-benzyl)-1-methyl-1H-thieno[2,3-d]pyrimidine-2,4-dione;

3-(3-Fluoro-benzyl)-6-[3-(2-methoxy-pyridin-4-yl)-prop-1-ynyl]-1-methyl-1H-thieno[2,3-d]pyrimidine-2,4-dione;

3-(3-Fluoro-benzyl)-1-methyl-6-[3-(4-methylsulfanyl-phenyl)-prop-1-ynyl]-1H-thieno[2,3-d]pyrimidine-2,4-dione;

3-(3-Fluoro-benzyl)-1-methyl-6-[3-(3-methylsulfanyl-phenyl)-prop-1-ynyl]-1H-thieno[2,3-d]pyrimidine-2,4-dione;

3-(3-Fluoro-benzyl)-1-methyl-6-(3-p-tolyl-prop-1-ynyl)-1H-thieno[2,3-d]pyrimidine-2,4-dione; and

3-(3-Fluoro-benzyl)-1-methyl-6-(3-m-tolyl-prop-1-ynyl)-1H-thieno[2,3-d]pyrimidine-2,4-dione.

Another invention embodiment is a compound selected from:

3-(3-Methoxycarbonyl-benzyl)-1-methyl-2,4-dioxo-1,2,3,4-tetrahydro-thieno[2,3-d]pyrimidine-6-carboxylic acid benzyl ester;

3-(3-Methoxycarbonyl-benzyl)-1-methyl-2,4-dioxo-1,2,3,4-tetrahydro-thieno[2,3-d]pyrimidine-6-carboxylic acid benzyl ester;

3-Benzofuran-5-ylmethyl-1-methyl-2,4-dioxo-1,2,3,4-tetrahydro-thieno[2,3-d]pyrimidine-6-carboxylic acid benzyl ester;

1-Methyl-3-(4-methyl-benzyl)-2,4-dioxo-1,2,3,4-tetrahydro-thieno[2,3-d]pyrimidine-6-carboxylic acid benzyl ester;

3-(4-Acetylamino-benzyl)-1-methyl-2,4-dioxo-1,2,3,4-tetrahydro-thieno[2,3-d]pyrimidine-6-carboxylic acid benzyl ester;

1-Methyl-2,4-dioxo-3-(4-vinyl-benzyl)-1,2,3,4-tetrahydro-thieno[2,3-d]pyrimidine-6-carboxylic acid benzyl ester;

1-Methyl-2,4-dioxo-3-(4-sulfamoyl-benzyl)-1,2,3,4-tetrahydro-thieno[2,3-d]pyrimidine-6-carboxylic acid benzyl ester;

3-(4-Bromo-benzyl)-2,4-dioxo-1,2,3,4-tetrahydro-thieno[2,3-d]pyrimidine-6-carboxylic acid pyridin-4-ylmethyl ester;

1-Methyl-2,4-dioxo-3-phenethyl-1,2,3,4-tetrahydro-thieno[2,3-d]pyrimidine-6-carboxylic acid benzyl ester;

1-Methyl-2,4-dioxo-3-[4-(2H-tetrazol-5-yl)-benzyl]-1,2,3,4-tetrahydro-thieno[2,3-d]pyrimidine-6-carboxylic acid benzyl ester;

3-(4-Fluoro-benzyl)-2,4-dioxo-1,2,3,4-tetrahydro-thieno[2,3-d]pyrimidine-6-carboxylic acid pyridin-4-ylmethyl ester;

3-(4-tert-Butyoxycarbonyl-benzyl)-1-methyl-2,4-dioxo-1,2,3,4-tetrahydro-thieno[2,3-d]pyrimidine-6-carboxylic acid benzyl ester;

3-(4-tert-Butyoxycarbonyl-benzyl)-1-methyl-2,4-dioxo-1,2,3,4-tetrahydro-thieno[2,3-d]pyrimidine-6-carboxylic acid;

4-[6-(4-Fluoro-benzylcarbamoyl)-1-methyl-2,4-dioxo-1,4-dihydro-2H-thieno[2,3-d]pyrimidin-3-ylmethyl]-benzoic acid;

4-[6-(4-Dimethylamino-benzylcarbamoyl)-1-methyl-2,4-dioxo-1,4-dihydro-2H-thieno[2,3-d]pyrimidin-3-ylmethyl]-benzoic acid, compound with trifluoro-acetic acid;

4-[6-(2-Ethoxy-benzylcarbamoyl)-1-methyl-2,4-dioxo-1,2,3,4-tetrahydro-thieno[2,3-d]pyrimidine-3-ylmethyl]-benzoic acid;

1-Methyl-2,4-dioxo-1,2,3,4-tetrahydro-thieno[2,3-d]pyrimidine-6-carboxylic acid;

1-Methyl-2,4-dioxo-1,2,3,4-tetrahydro-thieno[2,3-d]pyrimidine-6-carboxylic acid 3-methoxy-benzylamide;

1-Methyl-2,4-dioxo-3-[4-(1H-tetrazol-5-yl)-benzyl]-1,2,3,4-tetrahydro-thieno[2,3-d]pyrimidine-6-carboxylic acid 3-methoxy-benzylamide;

1-Methyl-3-[4-(morpholine-4-sulfonyl)-benzyl]-2,4-dioxo-1,2,3,4-tetrahydro-thieno[2,3-d]pyrimidine-6-carboxylic acid 3-methoxy-benzylamide;

1-Methyl-3-[4-(morpholine-4-carbonyl)-benzyl]-2,4-dioxo-1,2,3,4-tetrahydro-thieno[2,3-d]pyrimidine-6-carboxylic acid 3-methoxy-benzylamide;

3-But-2-ynyl-1-methyl-2,4-dioxo-1,2,3,4-tetrahydro-thieno[2,3-d]pyrimidine-6-carboxylic acid 3-methoxy-benzylamide;

1-Methyl-2,4-dioxo-3-[3-(1H-tetrazol-5-yl)-benzyl]-1,2,3,4-tetrahydro-thieno[2,3-d]pyrimidine-6-carboxylic acid 3-methoxy-benzylamide;

3-(4-Cyano-benzyl)-1-methyl-2,4-dioxo-1,2,3,4-tetrahydro-thieno[2,3-d]pyrimidine-6-carboxylic acid 3-methoxy-benzylamide;

{4-[6-(3-Methoxy-benzylcarbamoyl)-1-methyl-2,4-dioxo-1,4-dihydro-2H-thieno[2,3-d]pyrimidin-3-ylmethyl]-phenyl}-acetic acid;

3-[2-(2,4-Dichloro-benzenesulfonyl)-ethyl]-1-methyl-2,4-dioxo-1,2,3,4-tetrahydro-thieno[2,3-d]pyrimidine-6-carboxylic acid 3-methoxy-benzylamide;

3-(4-Methanesulfonyl-benzyl)-1-methyl-2,4-dioxo-1,2,3,4-tetrahydro-thieno[2,3-d]pyrimidine-6-carboxylic acid 3-methoxy-benzylamide;

1-Methyl-2,4-dioxo-3-(4-sulfamoyl-benzyl)-1,2,3,4-tetrahydro-thieno[2,3-d]pyrimidine-6-carboxylic acid 3-methoxy-benzylamide;

3-Benzyl-1-methyl-2,4-dioxo-1,2,3,4-tetrahydro-thieno[2,3-d]pyrimidine-6-carboxylic acid (2-methoxy-pyridin-4-ylmethyl)-amide;

1-Methyl-3-(4-methylsulfamoyl-benzyl)-2,4-dioxo-1,2,3,4-tetrahydro-thieno[2,3-d]pyrimidine-6-carboxylic acid 3-methoxy-benzylamide;

3-(4-Isopropylsulfamoyl-benzyl)-1-methyl-2,4-dioxo-1,2,3,4-tetrahydro-thieno[2,3-d]pyrimidine-6-carboxylic acid 3-methoxy-benzylamide;

1-Methyl-2,4-dioxo-3-[4-(pyrrolidine-1-sulfonyl)-benzyl]-1,2,3,4-tetrahydro-thieno[2,3-d]pyrimidine-6-carboxylic acid 3-methoxy-benzylamide; and

1-Methyl-3-[4-(4-methyl-piperidine-1-sulfonyl)-benzyl]-2,4-dioxo-1,2,3,4-tetrahydro-thieno[2,3-d]pyrimidine-6-carboxylic acid 3-methoxy-benzylamide.

Another invention embodiment is a compound selected from:

3-Benzyl-1-methyl-2,4-dioxo-1,2,3,4-tetrahydro-thieno[2,3-d]pyrimidine-6-carboxylic acid benzofuran-2-ylmethyl ester;

3-(4-Bromo-benzyl)-2,4-dioxo-1,2,3,4-tetrahydro-thieno[2,3-d]pyrimidine-6-carboxylic acid pyridin-4-ylmethyl ester;

3-Benzyl-2,4-dioxo-1,2,3,4-tetrahydro-thieno[2,3-d]pyrimidine-6-carboxylic acid 4-methoxy-benzyl ester;

4-{1-Methyl-2,4-dioxo-6-[(pyridin-4-ylmethyl)-carbamoyl]-1,4-dihydro-2H-thieno[2,3-d]pyrimidin-3-ylmethyl}-benzoic acid, compound with trifluoro-acetic acid;

4-[6-(4-Methoxy-benzylcarbamoyl)-1-methyl-2,4-dioxo-1,4-dihydro-2H-thieno[2,3-d]pyrimidin-3-ylmethyl]-benzoic acid;

4-[6-(3,4-Dimethoxy-benzylcarbamoyl)-1-methyl-2,4-dioxo-1,4-dihydro-2H-thieno[2,3-d]pyrimidin-3-ylmethyl]-benzoic acid tert-butyl ester;

4-[6-(3,4-Dimethoxy-benzylcarbamoyl)-1-methyl-2,4-dioxo-1,4-dihydro-2H-thieno[2,3-d]pyrimidin-3-ylmethyl]-benzoic acid;

4-[6-(4-Bromo-benzylcarbamoyl)-1-methyl-2,4-dioxo-1,4-dihydro-2H-thieno[2,3-d]pyrimidin-3-ylmethyl]-benzoic acid;

4-[6-(4-Bromo-benzylcarbamoyl)-1-methyl-2,4-dioxo-1,4-dihydro-2H-thieno[2,3-d]pyrimidin-3-ylmethyl]-benzoic acid tert-butyl ester;

4-[6-(3,5-Bis-trifluoromethyl-benzylcarbamoyl)-1-methyl-2,4-dioxo-1,4-dihydro-2H-thieno[2,3-d]pyrimidin-3-ylmethyl]-benzoic acid;

4-[6-(4-Chloro-benzylcarbamoyl)-1-methyl-2,4-dioxo-1,4-dihydro-2H-thieno[2,3-d]pyrimidin-3-ylmethyl]-benzoic acid;

4-[1-Methyl-2,4-dioxo-6-(4-sulfamoyl-benzylcarbamoyl)-1,4-dihydro-2H-thieno[2,3-d]pyrimidin-3-ylmethyl]-benzoic acid;

3-(4-Fluoro-benzyl)-1-methyl-2,4-dioxo-1,2,3,4-tetrahydro-thieno[2,3-d]pyrimidine-6-carboxylic acid 4-methoxy-benzylamide;

3-(4-Iodo-benzyl)-1-methyl-2,4-dioxo-1,2,3,4-tetrahydro-thieno[2,3-d]pyrimidine-6-carboxylic acid 3-methoxy-benzylamide;

3-(4-Dimethylsulfamoyl-benzyl)-1-methyl-2,4-dioxo-1,2,3,4-tetrahydro-thieno[2,3-d]pyrimidine-6-carboxylic acid 4-methoxy-benzylamide;

3-(3-Methoxy-benzyl)-1-methyl-2,4-dioxo-1,2,3,4-tetrahydro-thieno[2,3-d]pyrimidine-6-carboxylic acid 4-methoxy-benzylamide;

3-(4-Cyano-benzyl)-1-methyl-2,4-dioxo-1,2,3,4-tetrahydro-thieno[2,3-d]pyrimidine-6-carboxylic acid 4-methoxy-benzylamide;

3-(4-Acetylamino-benzyl)-1-methyl-2,4-dioxo-1,2,3,4-tetrahydro-thieno[2,3-d]pyrimidine-6-carboxylic acid 3-methoxy-benzylamide;

5-[6-(3-Methoxy-benzylcarbamoyl)-1-methyl-2,4-dioxo-1,4-dihydro-2H-thieno[2,3-d]pyrimidin-3-ylmethyl]-furan-2-carboxylic acid ethyl ester;

3-(4-Cyano-benzyl)-2,4-dioxo-1,2,3,4-tetrahydro-thieno[2,3-d]pyrimidine-6-carboxylic acid 3-methoxy-benzyl ester;

1-Methyl-2,4-dioxo-3-[4-(5-thioxo-4,5-dihydro-1,2,4-oxadiazol-3-yl)-benzyl]-1,2,3,4-tetrahydro-thieno[2,3-d]pyrimidine-6-carboxylic acid 4-methoxy-benzylamide;

4-[6-(3-Methoxy-benzylcarbamoyl)-1-methyl-2,4-dioxo-1,4-dihydro-2H-thieno[2,3-d]pyrimidin-3-ylmethyl]-benzoic acid 2-dimethylamino-ethyl ester;

3-Cyclohexylmethyl-1-methyl-2,4-dioxo-1,2,3,4-tetrahydro-thieno[2,3-d]pyrimidine-6-carboxylic acid 4-methoxy-benzylamide;

3-Cyclohexylmethyl-1-methyl-2,4-dioxo-1,2,3,4-tetrahydro-thieno[2,3-d]pyrimidine-6-carboxylic acid 4-methoxy-benzylamide;

3-Benzyl-2,4-dioxo-1,2,3,4-tetrahydro-thieno[2,3-d]pyrimidine-6-carboxylic acid furan-3-ylmethyl ester;

3-Benzyl-1-methyl-2,4-dioxo-1,2,3,4-tetrahydro-thieno[2,3-d]pyrimidine-6-carboxylic acid pentafluorophenylmethyl ester;

3-Benzyl-1-ethyl-2,4-dioxo-1,2,3,4-tetrahydro-thieno[2,3-d]pyrimidine-6-carboxylic acid benzyl ester;

3-Benzyl-1-cyclopropylmethyl-2,4-dioxo-1,2,3,4-tetrahydro-thieno[2,3-d]pyrimidine-6-carboxylic acid benzyl ester;

3-Benzyl-1-methyl-2,4-dioxo-1,2,3,4-tetrahydro-thieno[2,3-d]pyrimidine-6-carboxylic acid (pyridin-4-ylmethyl)-amide;

3-Benzyl-1-methyl-2,4-dioxo-1,2,3,4-tetrahydro-thieno[2,3-d]pyrimidine-6-carboxylic acid 3-bromo-benzyl ester;

4-[6-(3-Difluoromethoxy-benzylcarbamoyl)-1-methyl-2,4-dioxo-1,4-dihydro-2H-thieno[2,3-d]pyrimidin-3-ylmethyl]-benzoic acid;

4-[6-(3-Difluoromethoxy-benzylcarbamoyl)-1-methyl-2,4-dioxo-1,4-dihydro-2H-thieno[2,3-d]pyrimidin-3-ylmethyl]-benzoic acid tert-butyl ester;

4-[6-(3-Methoxy-benzylcarbamoyl)-1-methyl-2,4-dioxo-1,4-dihydro-2H-thieno[2,3-d]pyrimidin-3-ylmethyl]-benzoic acid;

4-[6-(4-Methanesulfonyl-benzylcarbamoyl)-1-methyl-2,4-dioxo-1,4-dihydro-2H-thieno[2,3-d]pyrimidin-3-ylmethyl]-benzoic acid tert-butyl ester;

4-[6-(4-Methanesulfonyl-benzylcarbamoyl)-1-methyl-2,4-dioxo-1,4-dihydro-2H-thieno[2,3-d]pyrimidin-3-ylmethyl]-benzoic acid;

4-[1-Methyl-2,4-dioxo-6-(2-pyridin-4-yl-ethylcarbamoyl)-1,4-dihydro-2H-thieno[2,3-d]pyrimidin-3-ylmethyl]-benzoic acid;

1-Methyl-2,4-dioxo-3-(4-trifluoromethoxy-benzyl)-1,2,3,4-tetrahydro-thieno[2,3-d]pyrimidine-6-carboxylic acid 3-methoxy-benzylamide;

4-[6-(3-Methoxy-benzylcarbamoyl)-1-methyl-2,4-dioxo-1,4-dihydro-2H-thieno[2,3-d]pyrimidin-3-ylmethyl]-benzoic acid methyl ester;

3-(2,3-Dihydro-benzofuran-6-ylmethyl)-1-methyl-2,4-dioxo-1,2,3,4-tetrahydro-thieno[2,3-d]pyrimidine-6-carboxylic acid 3-methoxy-benzylamide;

1-Methyl-3-(2-methyl-thiazol-5-ylmethyl)-2,4-dioxo-1,2,3,4-tetrahydro-thieno[2,3-d]pyrimidine-6-carboxylic acid 3-methoxy-benzylamide;

1-Methyl-2,4-dioxo-3-[4-(1H-tetrazol-5-yl)-benzyl]-1,2,3,4-tetrahydro-thieno[2,3-d]pyrimidine-6-carboxylic acid 4-fluoro-benzylamide;

3-Benzyl-2-methoxy-4-oxo-3,4-dihydro-thieno[2,3-d]pyrimidine-6-carboxylic acid benzyl ester;

4-[6-(3-Methoxy-benzylcarbamoyl)-1-methyl-2,4-dioxo-1,4-dihydro-2H-thieno[2,3-d]pyrimidin-3-ylmethyl]-benzoic acid 2,2-dimethyl-propionyloxymethyl ester;

4-[6-(3-Methoxy-benzylcarbamoyl)-1-methyl-2,4-dioxo-1,4-dihydro-2H-thieno[2,3-d]pyrimidin-3-ylmethyl]-cyclohexanecarboxylic acid;

4-[6-(3-Methoxy-benzylcarbamoyl)-1-methyl-2,4-dioxo-1,4-dihydro-2H-thieno[2,3-d]pyrimidin-3-ylmethyl]-cyclohexanecarboxylic acid methyl ester;

1-{4-[6-(3-Methoxy-benzylcarbamoyl)-1-methyl-2,4-dioxo-1,4-dihydro-2H-thieno[2,3-d]pyrimidin-3-ylmethyl]-phenyl}-cyclopropanecarboxylic acid methyl ester;

1-{4-[6-(3-Methoxy-benzylcarbamoyl)-1-methyl-2,4-dioxo-1,4-dihydro-2H-thieno[2,3-d]pyrimidin-3-ylmethyl]-phenyl}-cyclopropanecarboxylic acid tert-butyl ester;

1-{4-[6-(3-Methoxy-benzylcarbamoyl)-1-methyl-2,4-dioxo-1,4-dihydro-2H-thieno[2,3-d]pyrimidin-3-ylmethyl]-phenyl}-cyclopropanecarboxylic acid;

2-{4-[6-(3-Methoxy-benzylcarbamoyl)-1-methyl-2,4-dioxo-1,4-dihydro-2H-thieno[2,3-d]pyrimidin-3-ylmethyl]-phenoxy}-2-methyl-propionic acid tert-butyl ester;

2-{4-[6-(3-Methoxy-benzylcarbamoyl)-1-methyl-2,4-dioxo-1,4-dihydro-2H-thieno[2,3-d]pyrimidin-3-ylmethyl]-phenoxy}-2-methyl-propionic acid;

3-Benzyl-1-methyl-2,4-dioxo-1,2,3,4-tetrahydro-furo[2,3-d]pyrimidine-6-carboxylic acid benzyl ester;

3-(3-Methoxy-benzyl)-1-methyl-2,4-dioxo-1,2,3,4-tetrahydro-thieno[2,3-d]pyrimidine-6-carboxylic acid benzyl ester;

3-Benzyl-1-methyl-2,4-dioxo-1,2,3,4-tetrahydro-thieno[2,3-d]pyrimidine-6-carboxylic acid benzyl ester;

3-Biphenyl-4-ylmethyl-1-methyl-2,4-dioxo-1,2,3,4-tetrahydro-thieno[2,3-d]pyrimidine-6-carboxylic acid benzyl ester;

3-(4-Methanesulfonyl-benzyl)-1-methyl-2,4-dioxo-1,2,3,4-tetrahydro-thieno[2,3-d]pyrimidine-6-carboxylic acid benzyl ester;

3-(4-Methanesulfonyl-benzyl)-1-methyl-2,4-dioxo-1,2,3,4-tetrahydro-thieno[2,3-d]pyrimidine-6-carboxylic acid benzyl ester;

1-Methyl-3-(4-methyl-benzyl)-2,4-dioxo-1,2,3,4-tetrahydro-thieno[2,3-d]pyrimidine-6-carboxylic acid benzyl ester;

1-Methyl-2,4-dioxo-3-phenethyl-1,2,3,4-tetrahydro-thieno[2,3-d]pyrimidine-6-carboxylic acid benzyl ester;

3-(4-Amino-6-phenylamino-1,3,5-triazin-2-ylmethyl)-1-methyl-2,4-dioxo-1,2,3,4-tetrahydro-thieno[2,3-d]pyrimidine-6-carboxylic acid benzyl ester;

1-Methyl-2,4-dioxo-3-(4-trifluoromethyl-benzyl)-1,2,3,4-tetrahydro-thieno[2,3-d]pyrimidine-6-carboxylic acid benzyl ester;

3-(6-Cyano-hexyl)-1-methyl-2,4-dioxo-1,2,3,4-tetrahydro-thieno[2,3-d]pyrimidine-6-carboxylic acid benzyl ester;

3-[2-(2,5-Dimethoxy-phenyl)-2-oxo-ethyl]-1-methyl-2,4-dioxo-1,2,3,4-tetrahydro-thieno[2,3-d]pyrimidine-6-carboxylic acid benzyl ester;

3-(3-Iodo-benzyl)-1-methyl-2,4-dioxo-1,2,3,4-tetrahydro-thieno[2,3-d]pyrimidine-6-carboxylic acid benzyl ester;

1-Methyl-2,4-dioxo-3-(3-trifluoromethyl-benzyl)-1,2,3,4-tetrahydro-thieno[2,3-d]pyrimidine-6-carboxylic acid benzyl ester;

3-(2,4-Bis-trifluoromethyl-benzyl)-1-methyl-2,4-dioxo-1,2,3,4-tetrahydro-thieno[2,3-d]pyrimidine-6-carboxylic acid benzyl ester;

3-[2-(1,3-Dioxo-1,3-dihydro-isoindol-2-yl)-ethyl]-1-methyl-2,4-dioxo-1,2,3,4-tetrahydro-thieno[2,3-d]pyrimidine-6-carboxylic acid benzyl ester;

3-[2-(1,3-Dioxo-1,3-dihydro-isoindol-2-yl)-ethyl]-1-methyl-2,4-dioxo-1,2,3,4-tetrahydro-thieno[2,3-d]pyrimidine-6-carboxylic acid benzyl ester;

3-(2-Carboxy-allyl)-1-methyl-2,4-dioxo-1,2,3,4-tetrahydro-thieno[2,3-d]pyrimidine-6-carboxylic acid benzyl ester;

3-(2-Carboxy-allyl)-1-methyl-2,4-dioxo-1,2,3,4-tetrahydro-thieno[2,3-d]pyrimidine-6-carboxylic acid benzyl ester;

3-(3-Amino-propyl)-1-methyl-2,4-dioxo-1,2,3,4-tetrahydro-thieno[2,3-d]pyrimidine-6-carboxylic acid benzyl ester;

3-(1,3-Dioxo-1,3-dihydro-isoindol-2-ylmethyl)-1-methyl-2,4-dioxo-1,2,3,4-tetrahydro-thieno[2,3-d]pyrimidine-6-carboxylic acid benzyl ester;

3-(4-Fluoro-benzyl)-1-methyl-2,4-dioxo-1,2,3,4-tetrahydro-thieno[2,3-d]pyrimidine-6-carboxylic acid benzyl ester;

1-Methyl-3-oxiranylmethyl-2,4-dioxo-1,2,3,4-tetrahydro-thieno[2,3-d]pyrimidine-6-carboxylic acid benzyl ester;

1-Methyl-3-((S)-2-methyl-butyl)-2,4-dioxo-1,2,3,4-tetrahydro-thieno[2,3-d]pyrimidine-6-carboxylic acid benzyl ester;

1-Methyl-2,4-dioxo-3-(4-phenoxy-butyl)-1,2,3,4-tetrahydro-thieno[2,3-d]pyrimidine-6-carboxylic acid benzyl ester;

3-(2-Cyano-benzyl)-1-methyl-2,4-dioxo-1,2,3,4-tetrahydro-thieno[2,3-d]pyrimidine-6-carboxylic acid benzyl ester;

1-Methyl-2,4-dioxo-3-(3-phenoxy-propyl)-1,2,3,4-tetrahydro-thieno[2,3-d]pyrimidine-6-carboxylic acid benzyl ester;

3-Hex-5-enyl-1-methyl-2,4-dioxo-1,2,3,4-tetrahydro-thieno[2,3-d]pyrimidine-6-carboxylic acid benzyl ester;

1-Methyl-2,4-dioxo-3-pyridin-3-ylmethyl-1,2,3,4-tetrahydro-thieno[2,3-d]pyrimidine-6-carboxylic acid benzyl ester;

3-[2-Hydroxy-3-(naphthalen-1-yloxy)-propyl]-1-methyl-2,4-dioxo-1,2,3,4-tetrahydro-thieno[2,3-d]pyrimidine-6-carboxylic acid benzyl ester;

1,3-Dimethyl-2,4-dioxo-1,2,3,4-tetrahydro-thieno[2,3-d]pyrimidine-6-carboxylic acid benzyl ester;

3-Cyclobutylmethyl-1-methyl-2,4-dioxo-1,2,3,4-tetrahydro-thieno[2,3-d]pyrimidine-6-carboxylic acid benzyl ester;

3-Allyl-1-methyl-2,4-dioxo-1,2,3,4-tetrahydro-thieno[2,3-d]pyrimidine-6-carboxylic acid benzyl ester;

1-Methyl-2,4-dioxo-3-prop-2-ynyl-1,2,3,4-tetrahydro-thieno[2,3-d]pyrimidine-6-carboxylic acid benzyl ester;

3-But-2-ynyl-1-methyl-2,4-dioxo-1,2,3,4-tetrahydro-thieno[2,3-d]pyrimidine-6-carboxylic acid benzyl ester;

1-Methyl-2,4-dioxo-3-(2-phenoxy-ethyl)-1,2,3,4-tetrahydro-thieno[2,3-d]pyrimidine-6-carboxylic acid benzyl ester;

1-Methyl-2,4-dioxo-3-(2-phenoxy-ethyl)-1,2,3,4-tetrahydro-thieno[2,3-d]pyrimidine-6-carboxylic acid benzyl ester;

3-((R)-3-Hydroxy-2-methyl-propyl)-1-methyl-2,4-dioxo-1,2,3,4-tetrahydro-thieno[2,3-d]pyrimidine-6-carboxylic acid benzyl ester;

3-Isobutyl-1-methyl-2,4-dioxo-1,2,3,4-tetrahydro-thieno[2,3-d]pyrimidine-6-carboxylic acid benzyl ester;

3-(6-Chloro-pyridin-3-ylmethyl)-1-methyl-2,4-dioxo-1,2,3,4-tetrahydro-thieno[2,3-d]pyrimidine-6-carboxylic acid benzyl ester;

3-(2-Benzenesulfonylmethyl-benzyl)-1-methyl-2,4-dioxo-1,2,3,4-tetrahydro-thieno[2,3-d]pyrimidine-6-carboxylic acid benzyl ester;

1-Methyl-3-naphthalen-1-ylmethyl-2,4-dioxo-1,2,3,4-tetrahydro-thieno[2,3-d]pyrimidine-6-carboxylic acid benzyl ester;

1-Methyl-2,4-dioxo-3-(2-trifluoromethyl-benzyl)-1,2,3,4-tetrahydro-thieno[2,3-d]pyrimidine-6-carboxylic acid benzyl ester;

3-(3-Chloro-benzyl)-1-methyl-2,4-dioxo-1,2,3,4-tetrahydro-thieno[2,3-d]pyrimidine-6-carboxylic acid benzyl ester;

3-(4-Methoxycarbonyl-butyl)-1-methyl-2,4-dioxo-1,2,3,4-tetrahydro-thieno[2,3-d]pyrimidine-6-carboxylic acid benzyl ester;

3-Ethyl-1-methyl-2,4-dioxo-1,2,3,4-tetrahydro-thieno[2,3-d]pyrimidine-6-carboxylic acid benzyl ester;

3-Benzyl-1-methyl-2,4-dioxo-1,2,3,4-tetrahydro-thieno[2,3-d]pyrimidine-6-carboxylic acid 4-fluoro-benzyl ester;

3-[2-(4-Chloro-benzenesulfonyl)-ethyl]-1-methyl-2,4-dioxo-1,2,3,4-tetrahydro-thieno[2,3-d]pyrimidine-6-carboxylic acid benzyl ester;

3-(2-Acetoxy-ethyl)-1-methyl-2,4-dioxo-1,2,3,4-tetrahydro-thieno[2,3-d]pyrimidine-6-carboxylic acid benzyl ester;

3-Benzyl-1-methyl-2,4-dioxo-1,2,3,4-tetrahydro-thieno[2,3-d]pyrimidine-6-carboxylic acid 2-phenoxy-ethyl ester;

3-Benzyl-1-methyl-2,4-dioxo-1,2,3,4-tetrahydro-thieno[2,3-d]pyrimidine-6-carboxylic acid benzyl ester;

3-Benzyl-1-methyl-2,4-dioxo-1,2,3,4-tetrahydro-thieno[2,3-d]pyrimidine-6-carboxylic acid benzylamide;

3-Benzyl-1-methyl-2,4-dioxo-1,2,3,4-tetrahydro-thieno[2,3-d]pyrimidine-6-carboxylic acid 2,6-dichloro-benzyl ester;

3-Benzyl-1-methyl-2,4-dioxo-1,2,3,4-tetrahydro-thieno[2,3-d]pyrimidine-6-carboxylic acid butyl ester;

3-Benzyl-1-methyl-2,4-dioxo-1,2,3,4-tetrahydro-thieno[2,3-d]pyrimidine-6-carboxylic acid 2,3-dihydro-1,4-benzodioxin-2-ylmethyl ester;

3-Benzyl-1-methyl-2,4-dioxo-1,2,3,4-tetrahydro-thieno[2,3-d]pyrimidine-6-carboxylic acid 2-diethylamino-1-methyl-ethyl ester;

3-Benzyl-1-methyl-2,4-dioxo-1,2,3,4-tetrahydro-thieno[2,3-d]pyrimidine-6-carboxylic acid 4-fluoro-benzyl ester;

3-Benzyl-1-methyl-2,4-dioxo-1,2,3,4-tetrahydro-thieno[2,3-d]pyrimidine-6-carboxylic acid 4-isopropyl-benzyl ester;

3-Benzyl-1-methyl-2,4-dioxo-1,2,3,4-tetrahydro-thieno[2,3-d]pyrimidine-6-carboxylic acid 2-p-tolyl-ethyl ester;

3-Benzyl-1-methyl-2,4-dioxo-1,2,3,4-tetrahydro-thieno[2,3-d]pyrimidine-6-carboxylic acid 4-trifluoromethyl-benzyl ester;

3-Benzyl-1-methyl-2,4-dioxo-1,2,3,4-tetrahydro-thieno[2,3-d]pyrimidine-6-carboxylic acid cyclobutylmethyl ester;

3-Benzyl-1-methyl-2,4-dioxo-1,2,3,4-tetrahydro-thieno[2,3-d]pyrimidine-6-carboxylic acid 2,6-difluoro-benzyl ester;

3-Benzyl-1-methyl-2,4-dioxo-1,2,3,4-tetrahydro-thieno[2,3-d]pyrimidine-6-carboxylic acid 2-(2-hydroxy-phenyl)-ethyl ester;

3-Benzyl-1-methyl-2,4-dioxo-1,2,3,4-tetrahydro-thieno[2,3-d]pyrimidine-6-carboxylic acid 2-(2-hydroxy-phenyl)-ethyl ester;

3-Benzyl-1-methyl-2,4-dioxo-1,2,3,4-tetrahydro-thieno[2,3-d]pyrimidine-6-carboxylic acid 1-methyl-piperidin-4-yl ester;

3-Benzyl-1-methyl-2,4-dioxo-1,2,3,4-tetrahydro-thieno[2,3-d]pyrimidine-6-carboxylic acid 1-methyl-piperidin-4-yl ester;

3-Benzyl-1-methyl-2,4-dioxo-1,2,3,4-tetrahydro-thieno[2,3-d]pyrimidine-6-carboxylic acid pyridin-3-ylmethyl ester;

3-Benzyl-1-methyl-2,4-dioxo-1,2,3,4-tetrahydro-thieno[2,3-d]pyrimidine-6-carboxylic acid 3-pyridin-3-yl-propyl ester;

3-Benzyl-1-methyl-2,4-dioxo-1,2,3,4-tetrahydro-thieno[2,3-d]pyrimidine-6-carboxylic acid 2-dimethylamino-1-methyl-ethyl ester;

3-Benzyl-1-methyl-2,4-dioxo-1,2,3,4-tetrahydro-thieno[2,3-d]pyrimidine-6-carboxylic acid 4-methoxy-benzyl ester;

3-Benzyl-1-methyl-2,4-dioxo-1,2,3,4-tetrahydro-thieno[2,3-d]pyrimidine-6-carboxylic acid tetrahydro-pyran-4-yl ester;

3-Benzyl-1-methyl-2,4-dioxo-1,2,3,4-tetrahydro-thieno[2,3-d]pyrimidine-6-carboxylic acid 2,2,2-trifluoro-1-trifluoromethyl-ethyl ester;

3-Benzyl-1-methyl-2,4-dioxo-1,2,3,4-tetrahydro-thieno[2,3-d]pyrimidine-6-carboxylic acid 2-trifluoromethyl-benzyl ester;

3-Benzyl-1-methyl-2,4-dioxo-1,2,3,4-tetrahydro-thieno[2,3-d]pyrimidine-6-carboxylic acid 2-benzyloxy-ethyl ester;

3-Benzyl-1-methyl-2,4-dioxo-1,2,3,4-tetrahydro-thieno[2,3-d]pyrimidine-6-carboxylic acid 2,2,2-trichloro-ethyl ester;

3-Benzyl-1-methyl-2,4-dioxo-1,2,3,4-tetrahydro-thieno[2,3-d]pyrimidine-6-carboxylic acid phenethyl ester;

3-Benzyl-1-methyl-2,4-dioxo-1,2,3,4-tetrahydro-thieno[2,3-d]pyrimidine-6-carboxylic acid 3-ethyl-oxetan-3-ylmethyl ester;

3-Benzyl-1-methyl-2,4-dioxo-1,2,3,4-tetrahydro-thieno[2,3-d]pyrimidine-6-carboxylic acid 2-morpholin-4-yl-ethyl ester;

3-Benzyl-1-methyl-2,4-dioxo-1,2,3,4-tetrahydro-thieno[2,3-d]pyrimidine-6-carboxylic acid 2-pyrrolidin-1-yl-ethyl ester;

3-Benzyl-1-methyl-2,4-dioxo-1,2,3,4-tetrahydro-thieno[2,3-d]pyrimidine-6-carboxylic acid 2-pyrrolidin-1-yl-ethyl ester;

3-Benzyl-1-methyl-2,4-dioxo-1,2,3,4-tetrahydro-thieno[2,3-d]pyrimidine-6-carboxylic acid 2-(2-ethoxy-ethoxy)-ethyl ester;

3-Benzyl-1-methyl-2,4-dioxo-1,2,3,4-tetrahydro-thieno[2,3-d]pyrimidine-6-carboxylic acid tetrahydro-pyran-2-ylmethyl ester;

3-Benzyl-1-methyl-2,4-dioxo-1,2,3,4-tetrahydro-thieno[2,3-d]pyrimidine-6-carboxylic acid 4-nitro-benzyl ester;

3-Benzyl-1-methyl-2,4-dioxo-1,2,3,4-tetrahydro-thieno[2,3-d]pyrimidine-6-carboxylic acid pentyl ester;

3-Benzyl-1-methyl-2,4-dioxo-1,2,3,4-tetrahydro-thieno[2,3-d]pyrimidine-6-carboxylic acid 3-phenyl-propyl ester;

3-Benzyl-1-methyl-2,4-dioxo-1,2,3,4-tetrahydro-thieno[2,3-d]pyrimidine-6-carboxylic acid 3-phenoxy-benzyl ester;

3-Benzyl-1-methyl-2,4-dioxo-1,2,3,4-tetrahydro-thieno[2,3-d]pyrimidine-6-carboxylic acid 3,5-dimethoxy-benzyl ester;

3-Benzyl-1-methyl-2,4-dioxo-1,2,3,4-tetrahydro-thieno[2,3-d]pyrimidine-6-carboxylic acid 3-methyl-butyl ester;

3-Benzyl-1-methyl-2,4-dioxo-1,2,3,4-tetrahydro-thieno[2,3-d]pyrimidine-6-carboxylic acid 4-chloro-benzyl ester;

3-Benzyl-1-methyl-2,4-dioxo-1,2,3,4-tetrahydro-thieno[2,3-d]pyrimidine-6-carboxylic acid 1-ethyl-piperidin-3-yl ester;

3-Benzyl-1-methyl-2,4-dioxo-1,2,3,4-tetrahydro-thieno[2,3-d]pyrimidine-6-carboxylic acid 4-benzyloxy-benzyl ester;

3-Benzyl-1-methyl-2,4-dioxo-1,2,3,4-tetrahydro-thieno[2,3-d]pyrimidine-6-carboxylic acid isobutyl ester;

3-Benzyl-1-methyl-2,4-dioxo-1,2,3,4-tetrahydro-thieno[2,3-d]pyrimidine-6-carboxylic acid 3-(4-methoxy-phenyl)-propyl ester;

3-Benzyl-1-methyl-2,4-dioxo-1,2,3,4-tetrahydro-thieno[2,3-d]pyrimidine-6-carboxylic acid 2-chloro-6-fluoro-benzyl ester;

3-Benzyl-1-methyl-2,4-dioxo-1,2,3,4-tetrahydro-thieno[2,3-d]pyrimidine-6-carboxylic acid (S)-(tetrahydro-furan-3-yl)ester;

3-Benzyl-1-methyl-2,4-dioxo-1,2,3,4-tetrahydro-thieno[2,3-d]pyrimidine-6-carboxylic acid 3-methoxy-benzyl ester;

3-Benzyl-1-methyl-2,4-dioxo-1,2,3,4-tetrahydro-thieno[2,3-d]pyrimidine-6-carboxylic acid 3-methoxy-benzyl ester;

3-Benzyl-1-methyl-2,4-dioxo-1,2,3,4-tetrahydro-thieno[2,3-d]pyrimidine-6-carboxylic acid 3-pyridin-2-yl-propyl ester;

3-Benzyl-1-methyl-2,4-dioxo-1,2,3,4-tetrahydro-thieno[2,3-d]pyrimidine-6-carboxylic acid 2-piperidin-2-yl-ethyl ester;

3-Benzyl-1-methyl-2,4-dioxo-1,2,3,4-tetrahydro-thieno[2,3-d]pyrimidine-6-carboxylic acid 5-bromo-2-methoxy-benzyl ester;

3-Benzyl-1-methyl-2,4-dioxo-1,2,3,4-tetrahydro-thieno[2,3-d]pyrimidine-6-carboxylic acid cycloheptylmethyl ester;

3-Benzyl-1-methyl-2,4-dioxo-1,2,3,4-tetrahydro-thieno[2,3-d]pyrimidine-6-carboxylic acid 1,2,3,4-tetrahydro-naphthalen-1-yl ester;

3-Benzyl-1-methyl-2,4-dioxo-1,2,3,4-tetrahydro-thieno[2,3-d]pyrimidine-6-carboxylic acid (S)-1-pyrrolidin-2-ylmethyl ester;

3-Benzyl-1-methyl-2,4-dioxo-1,2,3,4-tetrahydro-thieno[2,3-d]pyrimidine-6-carboxylic acid 3-chloro-benzyl ester;

3-Benzyl-1-methyl-2,4-dioxo-1,2,3,4-tetrahydro-thieno[2,3-d]pyrimidine-6-carboxylic acid 1,3-benzodioxol-5-ylmethyl ester;

3-Benzyl-1-methyl-2,4-dioxo-1,2,3,4-tetrahydro-thieno[2,3-d]pyrimidine-6-carboxylic acid 4-methylsulfanyl-benzyl ester;

3-Benzyl-1-methyl-2,4-dioxo-1,2,3,4-tetrahydro-thieno[2,3-d]pyrimidine-6-carboxylic acid 4-methylsulfanyl-benzyl ester;

3-Benzyl-1-methyl-2,4-dioxo-1,2,3,4-tetrahydro-thieno[2,3-d]pyrimidine-6-carboxylic acid 3,4-dichloro-benzyl ester;

3-Benzyl-1-methyl-2,4-dioxo-1,2,3,4-tetrahydro-thieno[2,3-d]pyrimidine-6-carboxylic acid 3,3-diphenyl-propyl ester;

3-Benzyl-1-methyl-2,4-dioxo-1,2,3,4-tetrahydro-thieno[2,3-d]pyrimidine-6-carboxylic acid 2-pyridin-2-yl-ethyl ester;

3-Benzyl-1-methyl-2,4-dioxo-1,2,3,4-tetrahydro-thieno[2,3-d]pyrimidine-6-carboxylic acid furan-3-ylmethyl ester;

3-Benzyl-1-methyl-2,4-dioxo-1,2,3,4-tetrahydro-thieno[2,3-d]pyrimidine-6-carboxylic acid but-3-enyl ester;

3-Benzyl-1-methyl-2,4-dioxo-1,2,3,4-tetrahydro-thieno[2,3-d]pyrimidine-6-carboxylic acid 2-cyano-ethyl ester;

3-Benzyl-1-methyl-2,4-dioxo-1,2,3,4-tetrahydro-thieno[2,3-d]pyrimidine-6-carboxylic acid 2-ethoxy-ethyl ester;

3-Benzyl-1-methyl-2,4-dioxo-1,2,3,4-tetrahydro-thieno[2,3-d]pyrimidine-6-carboxylic acid cyano-phenyl-methyl ester;

3-Benzyl-1-methyl-2,4-dioxo-1,2,3,4-tetrahydro-thieno[2,3-d]pyrimidine-6-carboxylic acid 4-trifluoromethyl-benzylamide;

3-Benzyl-1-methyl-2,4-dioxo-1,2,3,4-tetrahydro-thieno[2,3-d]pyrimidine-6-carboxylic acid 4-methyl-benzylamide;

3-Benzyl-1-methyl-2,4-dioxo-1,2,3,4-tetrahydro-thieno[2,3-d]pyrimidine-6-carboxylic acid phenethyl-amide;

3-Benzyl-1-methyl-2,4-dioxo-1,2,3,4-tetrahydro-thieno[2,3-d]pyrimidine-6-carboxylic acid cyclopropylamide;

1-Methyl-1,2,3,4-tetrahydro-thieno[2,3-d]pyrimidine-6-carboxylic acid 4-methoxy-benzylamide;

1-Methyl-2,4-dioxo-3-(4-sulfamoyl-benzyl)-1,2,3,4-tetrahydro-thieno[2,3-d]pyrimidine-6-carboxylic acid 4-methoxy-benzylamide;

1-Methyl-2,4-dioxo-1,2,3,4-tetrahydro-thieno[2,3-d]pyrimidine-6-carboxylic acid3-methoxy-benzylamide;

1-Methyl-2,4-dioxo-3-(3-oxo-3-phenyl-propyl)-1,2,3,4-tetrahydro-thieno[2,3-d]pyrimidine-6-carboxylic acid 3-methoxy-benzylamide;

3-[4-(N-Hydroxycarbamimidoyl)-benzyl]-1-methyl-2,4-dioxo-1,2,3,4-tetrahydro-thieno[2,3-d]pyrimidine-6-carboxylic acid 4-methoxy-benzylamide;

1-Methyl-2,4-dioxo-3-[4-(5-oxo-4,5-dihydro-1,2,4-oxadiazol-3-yl)-benzyl]-1,2,3,4-tetrahydro-thieno[2,3-d]pyrimidine-6-carboxylic acid 4-methoxy-benzylamide;

1-Methyl-2,4-dioxo-3-[4-(5-thioxo-4,5-dihydro-1,2,4-oxadiazol-3-yl)-benzyl]-1,2,3,4-tetrahydro-thieno[2,3-d]pyrimidine-6-carboxylic acid 4-methoxy-benzylamide;

4-(5-Isopropyl-2H-pyrazol-3-yl)-pyridine;

3-Cyanomethyl-1-methyl-2,4-dioxo-1,2,3,4-tetrahydro-thieno[2,3-d]pyrimidine-6-carboxylic acid 3-methoxy-benzylamide;

(E)-4-[6-(4-Methoxy-benzylcarbamoyl)-1-methyl-2,4-dioxo-1,4-dihydro-2H-thieno[2,3-d]pyrimidin-3-yl]-but-2-enoic acid methyl ester;

(E)-4-[6-(4-Methoxy-benzylcarbamoyl)-1-methyl-2,4-dioxo-1,4-dihydro-2H-thieno[2,3-d]pyrimidin-3-yl]-but-2-enoic acid;

3-(2-Benzenesulfonyl-ethyl)-1-methyl-2,4-dioxo-1,2,3,4-tetrahydro-thieno[2,3-d]pyrimidine-6-carboxylic acid 4-methoxy-benzylamide;

2-Methoxy-4-[6-(4-methoxy-benzylcarbamoyl)-1-methyl-2,4-dioxo-1,4-dihydro-2H-thieno[2,3-d]pyrimidin-3-ylmethyl]-benzoic acid methyl ester;

3-(2-Methoxymethyl-1,1,3-trioxo-2,3-dihydro-1H-1l⁶-1,2-benzisothiazol-6-ylmethyl)-1-methyl-2,4-dioxo-1,2,3,4-tetrahydro-thieno[2,3-d]pyrimidine-6-carboxylic acid 4-methoxy-benzylamide;

1-Methyl-3-oct-2-ynyl-2,4-dioxo-1,2,3,4-tetrahydro-thieno[2,3-d]pyrimidine-6-carboxylic acid 4-methoxy-benzylamide;

3-[2-(4-Chloro-benzenesulfonylamino)-ethyl]-1-methyl-2,4-dioxo-1,2,3,4-tetrahydro-thieno[2,3-d]pyrimidine-6-carboxylic acid 4-methoxy-benzylamide;

3-[2-(4-Bromo-phenoxy)-ethyl]-1-methyl-2,4-dioxo-1,2,3,4-tetrahydro-thieno[2,3-d]pyrimidine-6-carboxylic acid 4-methoxy-benzylamide;

3-[2-(4-Bromo-phenoxy)-ethyl]-1-methyl-2,4-dioxo-1,2,3,4-tetrahydro-thieno[2,3-d]pyrimidine-6-carboxylic acid 3-methoxy-benzylamide;

3-[2-(4-Fluoro-phenoxy)-ethyl]-1-methyl-2,4-dioxo-1,2,3,4-tetrahydro-thieno[2,3-d]pyrimidine-6-carboxylic acid 4-methoxy-benzylamide;

3-[2-(4-Chloro-benzenesulfonyl)-ethyl]-1-methyl-2,4-dioxo-1,2,3,4-tetrahydro-thieno[2,3-d]pyrimidine-6-carboxylic acid 4-methoxy-benzylamide;

3-[2-(4-Fluoro-phenoxy)-ethyl]-1-methyl-2,4-dioxo-1,2,3,4-tetrahydro-thieno[2,3-d]pyrimidine-6-carboxylic acid 3-methoxy-benzylamide;

1-Methyl-2,4-dioxo-1,2,3,4-tetrahydro-thieno[2,3-d]pyrimidine-6-carboxylic acid (2-methoxy-pyridin-4-ylmethyl)-amide;

3-Cyclohexylmethyl-1-methyl-2,4-dioxo-1,2,3,4-tetrahydro-thieno[2,3-d]pyrimidine-6-carboxylic acid 3-methoxy-benzylamide;

4-[6-(4-Methoxy-benzylcarbamoyl)-1-methyl-2,4-dioxo-1,4-dihydro-2H-thieno[2,3-d]pyrimidin-3-ylmethyl]-2-methyl-benzoic acid methyl ester;

4-[6-(4-Methoxy-benzylcarbamoyl)-1-methyl-2,4-dioxo-1,4-dihydro-2H-thieno[2,3-d]pyrimidin-3-ylmethyl]-benzoic acid methyl ester;

2-Methoxy-4-[6-(3-methoxy-benzylcarbamoyl)-1-methyl-2,4-dioxo-1,4-dihydro-2H-thieno[2,3-d]pyrimidin-3-ylmethyl]-benzoic acid methyl ester;

4-[6-(3-Methoxy-benzylcarbamoyl)-1-methyl-2,4-dioxo-1,4-dihydro-2H-thieno[2,3-d]pyrimidin-3-ylmethyl]-2-methyl-benzoic acid methyl ester;

1-Methyl-2,4-dioxo-3-(3-oxo-3-phenyl-propyl)-1,2,3,4-tetrahydro-thieno[2,3-d]pyrimidine-6-carboxylic acid 4-methoxy-benzylamide;

3-[2-(4-Chloro-phenoxy)-ethyl]-1-methyl-2,4-dioxo-1,2,3,4-tetrahydro-thieno[2,3-d]pyrimidine-6-carboxylic acid 3-methoxy-benzylamide;

1-Methyl-2,4-dioxo-3-[2-(3-trifluoromethyl-benzenesulfonyl)-ethyl]-1,2,3,4-tetrahydro-thieno[2,3-d]pyrimidine-6-carboxylic acid 3-methoxy-benzylamide;

1-Methyl-2,4-dioxo-3-[2-(3-trifluoromethyl-benzenesulfonyl)-ethyl]-1,2,3,4-tetrahydro-thieno[2,3-d]pyrimidine-6-carboxylic acid 4-methoxy-benzylamide;

3-[2-(4-Chloro-benzenesulfonyl)-ethyl]-1-methyl-2,4-dioxo-1,2,3,4-tetrahydro-thieno[2,3-d]pyrimidine-6-carboxylic acid 3-methoxy-benzylamide; and

3-(2-Amino-ethyl)-1-methyl-2,4-dioxo-1,2,3,4-tetrahydro-thieno[2,3-d]pyrimidine-6-carboxylic acid 4-methoxy-benzylamide.

Another invention embodiment is a compound selected from:

1-Methyl-2,4-dioxo-1,2,3,4-tetrahydro-thieno[2,3-d]pyrimidine-6-carboxylic acid;

4-(6-Carbamoyl 1-methyl-2,4-dioxo-1,4-dihydro-2H-thieno[2,3-d]pyrimidin-3-ylmethyl)-2-methyl-benzoic acid;

4-(6-Carbamoyl-1-methyl-2,4-dioxo-1,4-dihydro-2H-thieno[2,3-d]pyrimidin-3-ylmethyl)-2-methyl-benzoic acid methyl ester;

4-[6-(3-Hydroxy-benzylcarbamoyl)-1-methyl-2,4-dioxo-1,4-dihydro-2H-thieno[2,3-d]pyrimidin-3-ylmethyl]-2-methyl-benzoic acid;

4-(6-Carbamoyl-1-methyl-2,4-dioxo-1,4-dihydro-2H-thieno[2,3-d]pyrimidin-3-ylmethyl)-2-hydroxy-benzoic acid;

3-(2-Amino-ethyl)-1-methyl-2,4-dioxo-1,2,3,4-tetrahydro-thieno[2,3-d]pyrimidine-6-carboxylic acid 3-methoxy-benzylamide; and

4-(2,5-Di-pyridin-4-yl-thiophen-3-yl)-benzaldehyde.

Another invention embodiment is a compound selected from:

1-Methyl-2,4-dioxo-3-(1-phenyl-ethyl)-1,2,3,4-tetrahydro-thieno[2,3-d]pyrimidine-6-carboxylic acid 4-methoxy-benzylamide;

1-Methyl-2,4-dioxo-3-(3-oxo-3-phenyl-propyl)-1,2,3,4-tetrahydro-thieno[2,3-d]pyrimidine-6-carboxylic acid 4-methoxy-benzylamide;

3-((S)-3,7-Dimethyl-oct-6-enyl)-1-methyl-2,4-dioxo-1,2,3,4-tetrahydro-thieno[2,3-d]pyrimidine-6-carboxylic acid 4-methoxy-benzylamide;

3-(2-Ethyl-hexyl)-1-methyl-2,4-dioxo-1,2,3,4-tetrahydro-thieno[2,3-d]pyrimidine-6-carboxylic acid 4-methoxy-benzylamide;

3-(5-Cyano-pentyl)-1-methyl-2,4-dioxo-1,2,3,4-tetrahydro-thieno[2,3-d]pyrimidine-6-carboxylic acid 4-methoxy-benzylamide;

3-(E)-But-2-enyl-1-methyl-2,4-dioxo-1,2,3,4-tetrahydro-thieno[2,3-d]pyrimidine-6-carboxylic acid 4-methoxy-benzylamide;

1-Methyl-3-(2-naphthalen-1-yl-ethyl)-2,4-dioxo-1,2,3,4-tetrahydro-thieno[2,3-d]pyrimidine-6-carboxylic acid 4-methoxy-benzylamide;

1-Methyl-2,4-dioxo-3-(E)-pent-2-enyl-1,2,3,4-tetrahydro-thieno[2,3-d]pyrimidine-6-carboxylic acid 4-methoxy-benzylamide;

1-Methyl-2,4-dioxo-3-(2-phenylsulfanyl-ethyl)-1,2,3,4-tetrahydro-thieno[2,3-d]pyrimidine-6-carboxylic acid 4-methoxy-benzylamide;

3-sec-Butyl-1-methyl-2,4-dioxo-1,2,3,4-tetrahydro-thieno[2,3-d]pyrimidine-6-carboxylic acid 4-methoxy-benzylamide;

1-Methyl-3-(2-methyl-allyl)-2,4-dioxo-1,2,3,4-tetrahydro-thieno[2,3-d]pyrimidine-6-carboxylic acid 4-methoxy-benzylamide;

3-(1-Ethyl-propyl)-1-methyl-2,4-dioxo-1,2,3,4-tetrahydro-thieno[2,3-d]pyrimidine-6-carboxylic acid 4-methoxy-benzylamide;

1-Methyl-2,4-dioxo-3-pent-2-ynyl-1,2,3,4-tetrahydro-thieno[2,3-d]pyrimidine-6-carboxylic acid 4-methoxy-benzylamide;

3-(2-Benzenesulfonyl-ethyl)-1-methyl-2,4-dioxo-1,2,3,4-tetrahydro-thieno[2,3-d]pyrimidine-6-carboxylic acid 4-methoxy-benzylamide;

1-Methyl-3-(3-methyl-but-2-enyl)-2,4-dioxo-1,2,3,4-tetrahydro-thieno[2,3-d]pyrimidine-6-carboxylic acid 4-methoxy-benzylamide;

3-[2-(4-Fluoro-benzenesulfonyl)-ethyl]-1-methyl-2,4-dioxo-1,2,3,4-tetrahydro-thieno[2,3-d]pyrimidine-6-carboxylic acid 4-methoxy-benzylamide;

1-Methyl-2,4-dioxo-3-[2-(toluene-4-sulfonyl)-ethyl]-1,2,3,4-tetrahydro-thieno[2,3-d]pyrimidine-6-carboxylic acid 4-methoxy-benzylamide;

3-[3-(4-Fluoro-phenyl)-3-oxo-propyl]-1-methyl-2,4-dioxo-1,2,3,4-tetrahydro-thieno[2,3-d]pyrimidine-6-carboxylic acid 4-methoxy-benzylamide;

3-[3-(4-Chloro-phenyl)-3-oxo-propyl]-1-methyl-2,4-dioxo-1,2,3,4-tetrahydro-thieno[2,3-d]pyrimidine-6-carboxylic acid 4-methoxy-benzylamide;

3-(2-Benzoylamino-ethyl)-1-methyl-2,4-dioxo-1,2,3,4-tetrahydro-thieno[2,3-d]pyrimidine-6-carboxylic acid 4-methoxy-benzylamide;

3-[2-(4-Bromo-phenoxy)-ethyl]-1-methyl-2,4-dioxo-1,2,3,4-tetrahydro-thieno[2,3-d]pyrimidine-6-carboxylic acid 4-methoxy-benzylamide;

3-Benzofurazan-5-ylmethyl-1-methyl-2,4-dioxo-1,2,3,4-tetrahydro-thieno[2,3-d]pyrimidine-6-carboxylic acid 4-methoxy-benzylamide;

1-Methyl-2,4-dioxo-3-(2-phenoxy-ethyl)-1,2,3,4-tetrahydro-thieno[2,3-d]pyrimidine-6-carboxylic acid 4-methoxy-benzylamide;

{5-[6-(4-Methoxy-benzylcarbamoyl)-1-methyl-2,4-dioxo-1,4-dihydro-2H-thieno[2,3-d]pyrimidin-3-ylmethyl]-isoxazol-3-yl}-carbamic acid methyl ester;

3-Benzyloxycarbonylamino-5-[6-(4-methoxy-benzylcarbamoyl)-1-methyl-2,4-dioxo-1,4-dihydro-2H-thieno[2,3-d]pyrimidin-3-yl]-4-oxo-pentanoic acid tert-butyl ester;

3-[2-(4-Chloro-phenylsulfanyl)-ethyl]-1-methyl-2,4-dioxo-1,2,3,4-tetrahydro-thieno[2,3-d]pyrimidine-6-carboxylic acid 3-methoxy-benzylamide;

1-Methyl-2,4-dioxo-3-(1-phenyl-ethyl)-1,2,3,4-tetrahydro-thieno[2,3-d]pyrimidine-6-carboxylic acid 3-methoxy-benzylamide;

1-Methyl-2,4-dioxo-3-(E)-pent-2-enyl-1,2,3,4-tetrahydro-thieno[2,3-d]pyrimidine-6-carboxylic acid 3-methoxy-benzylamide;

3-(2-Ethyl-hexyl)-1-methyl-2,4-dioxo-1,2,3,4-tetrahydro-thieno[2,3-d]pyrimidine-6-carboxylic acid 3-methoxy-benzylamide;

1-Methyl-2,4-dioxo-3-(2-phenylmethanesulfonyl-ethyl)-1,2,3,4-tetrahydro-thieno[2,3-d]pyrimidine-6-carboxylic acid 3-methoxy-benzylamide;

3-(5-Cyano-pentyl)-1-methyl-2,4-dioxo-1,2,3,4-tetrahydro-thieno[2,3-d]pyrimidine-6-carboxylic acid 3-methoxy-benzylamide;

3-(E)-But-2-enyl-1-methyl-2,4-dioxo-1,2,3,4-tetrahydro-thieno[2,3-d]pyrimidine-6-carboxylic acid 3-methoxy-benzylamide;

1-Methyl-3-(2-naphthalen-1-yl-ethyl)-2,4-dioxo-1,2,3,4-tetrahydro-thieno[2,3-d]pyrimidine-6-carboxylic acid 3-methoxy-benzylamide;

1-Methyl-2,4-dioxo-3-(E)-pent-2-enyl-1,2,3,4-tetrahydro-thieno[2,3-d]pyrimidine-6-carboxylic acid 3-methoxy-benzylamide;

1-Methyl-2,4-dioxo-3-(2-phenylsulfanyl-ethyl)-1,2,3,4-tetrahydro-thieno[2,3-d]pyrimidine-6-carboxylic acid 3-methoxy-benzylamide;

3-sec-Butyl-1-methyl-2,4-dioxo-1,2,3,4-tetrahydro-thieno[2,3-d]pyrimidine-6-carboxylic acid 3-methoxy-benzylamide;

1-Methyl-3-(2-methyl-allyl)-2,4-dioxo-1,2,3,4-tetrahydro-thieno[2,3-d]pyrimidine-6-carboxylic acid 3-methoxy-benzylamide;

3-(1-Ethyl-propyl)-1-methyl-2,4-dioxo-1,2,3,4-tetrahydro-thieno[2,3-d]pyrimidine-6-carboxylic acid 3-methoxy-benzylamide;

1-Methyl-2,4-dioxo-3-pent-2-ynyl-1,2,3,4-tetrahydro-thieno[2,3-d]pyrimidine-6-carboxylic acid 3-methoxy-benzylamide;

1-Methyl-3-(3-methyl-but-2-enyl)-2,4-dioxo-1,2,3,4-tetrahydro-thieno[2,3-d]pyrimidine-6-carboxylic acid 3-methoxy-benzylamide;

1-Methyl-2,4-dioxo-3-[2-(toluene-4-sulfonyl)-ethyl]-1,2,3,4-tetrahydro-thieno[2,3-d]pyrimidine-6-carboxylic acid 3-methoxy-benzylamide;

3-(2-Benzoylamino-ethyl)-1-methyl-2,4-dioxo-1,2,3,4-tetrahydro-thieno[2,3-d]pyrimidine-6-carboxylic acid 3-methoxy-benzylamide;

3-[2-(4-Bromo-phenoxy)-ethyl]-1-methyl-2,4-dioxo-1,2,3,4-tetrahydro-thieno[2,3-d]pyrimidine-6-carboxylic acid 3-methoxy-benzylamide;

3-Benzofurazan-5-ylmethyl-1-methyl-2,4-dioxo-1,2,3,4-tetrahydro-thieno[2,3-d]pyrimidine-6-carboxylic acid 3-methoxy-benzylamide;

1-Methyl-2,4-dioxo-3-(2-phenoxy-ethyl)-1,2,3,4-tetrahydro-thieno[2,3-d]pyrimidine-6-carboxylic acid 3-methoxy-benzylamide;

{5-[6-(3-Methoxy-benzylcarbamoyl)-1-methyl-2,4-dioxo-1,4-dihydro-2H-thieno[2,3-d]pyrimidin-3-ylmethyl]-isoxazol-3-yl}-carbamic acid methyl ester; and

3-Benzyloxycarbonylamino-5-[6-(3-methoxy-benzylcarbamoyl)-1-methyl-2,4-dioxo-1,4-dihydro-2H-thieno[2,3-d]pyrimidin-3-yl]-4-oxo-pentanoic acid tert-butyl ester.

Another invention embodiment is a compound selected from:

3-Benzyl-1-methyl-2,4-dioxo-1,2,3,4-tetrahydro-thieno[2,3-d]pyrimidine-6-carboxylic acid methyl ester;

3-(4-Bromo-benzyl)-5-methyl-2,4-dioxo-1,2,3,4-tetrahydro-thieno[2,3-d]pyrimidine-6-carboxylic acid benzyl ester;

3-(4-Fluoro-benzyl)-5-methyl-2,4-dioxo-1,2,3,4-tetrahydro-thieno[2,3-d]pyrimidine-6-carboxylic acid benzyl ester;

3-Benzyl-1-methyl-2,4-dioxo-1,2,3,4-tetrahydro-thieno[2,3-d]pyrimidine-6-carboxylic acid pyridin-4-ylmethyl ester;

3-Benzyl-1-methyl-2,4-dioxo-1,2,3,4-tetrahydro-thieno[2,3-d]pyrimidine-6-carboxylic acid benzo[b]thiophen-2-ylmethyl ester;

3-Benzyl-1-methyl-2,4-dioxo-1,2,3,4-tetrahydro-thieno[2,3-d]pyrimidine-6-carboxylic acid 1-methyl-1H-indol-5-ylmethyl ester;

3-Benzyl-2,4-dioxo-1,2,3,4-tetrahydro-thieno[2,3-d]pyrimidine-6-carboxylic acid thiophen-3-ylmethyl ester;

3-1,3-Benzodioxol-5-ylmethyl-1-methyl-2,4-dioxo-1,2,3,4-tetrahydro-thieno[2,3-d]pyrimidine-6-carboxylic acid benzyl ester;

1-Methyl-2,4-dioxo-3-pyridin-4-ylmethyl-1,2,3,4-tetrahydro-thieno[2,3-d]pyrimidine-6-carboxylic acid benzyl ester;

3-(4-tert-Butyl-benzyl)-1-methyl-2,4-dioxo-1,2,3,4-tetrahydro-thieno[2,3-d]pyrimidine-6-carboxylic acid benzyl ester;

3-(3,4-Dichloro-benzyl)-5-methyl-2,4-dioxo-1,2,3,4-tetrahydro-thieno[2,3-d]pyrimidine-6-carboxylic acid benzyl ester;

1-Methyl-2,4-dioxo-3-(4-trifluoromethoxy-benzyl)-1,2,3,4-tetrahydro-thieno[2,3-d]pyrimidine-6-carboxylic acid benzyl ester;

1-Methyl-3-naphthalen-2-ylmethyl-2,4-dioxo-1,2,3,4-tetrahydro-thieno[2,3-d]pyrimidine-6-carboxylic acid benzyl ester;

3-(4-Cyano-benzyl)-1-methyl-2,4-dioxo-1,2,3,4-tetrahydro-thieno[2,3-d]pyrimidine-6-carboxylic acid benzyl ester;

3-Benzyl-1-methyl-2,4-dioxo-1,2,3,4-tetrahydro-thieno[2,3-d]pyrimidine-6-carboxylic acid benzofuran-5-ylmethyl ester;

3-(3,5-Dimethoxy-benzyl)-1-methyl-2,4-dioxo-1,2,3,4-tetrahydro-thieno[2,3-d]pyrimidine-6-carboxylic acid benzyl ester;

3-Benzyl-1-methyl-2,4-dioxo-1,2,3,4-tetrahydro-thieno[2,3-d]pyrimidine-6-carboxylic acid benzyl ester;

3-(3,5-Dinitro-benzyl)-1-methyl-2,4-dioxo-1,2,3,4-tetrahydro-thieno[2,3-d]pyrimidine-6-carboxylic acid benzyl ester;

3-Benzyl-1-methyl-2,4-dioxo-1,2,3,4-tetrahydro-thieno[2,3-d]pyrimidine-6-carboxylic acid; and

3-(4-Carboxy-benzyl)-1-methyl-2,4-dioxo-1,2,3,4-tetrahydro-thieno[2,3-d]pyrimidine-6-carboxylic acid 2-ethoxy-benzyl ester.

Another invention embodiment is a compound selected from:

3-Benzyl-1-methyl-2,4-dioxo-1,2,3,4-tetrahydro-thieno[2,3-d]pyrimidine-6-carboxylic acid[2-(3,4-dimethoxy-phenyl)-ethyl]-amide;

3-Benzyl-1-methyl-2,4-dioxo-1,2,3,4-tetrahydro-thieno[2,3-d]pyrimidine-6-carboxylic acid 4-amino-benzylamide;

3-Benzyl-1-methyl-2,4-dioxo-1,2,3,4-tetrahydro-thieno[2,3-d]pyrimidine-6-carboxylic acid[2-(4-chloro-phenyl)-ethyl]-amide;

3-Benzyl-1-methyl-2,4-dioxo-1,2,3,4-tetrahydro-thieno[2,3-d]pyrimidine-6-carboxylic acid (biphenyl-2-ylmethyl)-amide;

3-Benzyl-1-methyl-2,4-dioxo-1,2,3,4-tetrahydro-thieno[2,3-d]pyrimidine-6-carboxylic acid 3,4-dimethoxy-benzylamide;

3-Benzyl-1-methyl-2,4-dioxo-1,2,3,4-tetrahydro-thieno[2,3-d]pyrimidine-6-carboxylic acid (2-pyridin-4-yl-ethyl)-amide;

3-Benzyl-1-methyl-2,4-dioxo-1,2,3,4-tetrahydro-thieno[2,3-d]pyrimidine-6-carboxylic acid 2-difluoromethoxy-benzylamide;

3-Benzyl-1-methyl-2,4-dioxo-1,2,3,4-tetrahydro-thieno[2,3-d]pyrimidine-6-carboxylic acid[2-(3-ethoxy-phenyl)-ethyl]-amide;

3-Benzyl-1-methyl-2,4-dioxo-1,2,3,4-tetrahydro-thieno[2,3-d]pyrimidine-6-carboxylic acid 3-chloro-4-fluoro-benzylamide;

3-Benzyl-1-methyl-2,4-dioxo-1,2,3,4-tetrahydro-thieno[2,3-d]pyrimidine-6-carboxylic acid 2,4-dichloro-benzylamide;

3-Benzyl-1-methyl-2,4-dioxo-1,2,3,4-tetrahydro-thieno[2,3-d]pyrimidine-6-carboxylic acid (2-phenyl-propyl)-amide;

3-Benzyl-1-methyl-2,4-dioxo-1,2,3,4-tetrahydro-thieno[2,3-d]pyrimidine-6-carboxylic acid 3,4,5-trimethoxy-benzylamide;

3-Benzyl-1-methyl-2,4-dioxo-1,2,3,4-tetrahydro-thieno[2,3-d]pyrimidine-6-carboxylic acid 3-chloro-benzylamide;

3-Benzyl-1-methyl-2,4-dioxo-1,2,3,4-tetrahydro-thieno[2,3-d]pyrimidine-6-carboxylic acid 3,5-dimethoxy-benzylamide;

3-Benzyl-1-methyl-2,4-dioxo-1,2,3,4-tetrahydro-thieno[2,3-d]pyrimidine-6-carboxylic acid 2,3-dimethoxy-benzylamide;

3-Benzyl-1-methyl-2,4-dioxo-1,2,3,4-tetrahydro-thieno[2,3-d]pyrimidine-6-carboxylic acid 3-trifluoromethyl-benzylamide;

3-Benzyl-1-methyl-2,4-dioxo-1,2,3,4-tetrahydro-thieno[2,3-d]pyrimidine-6-carboxylic acid 2-methoxy-benzylamide;

3-Benzyl-1-methyl-2,4-dioxo-1,2,3,4-tetrahydro-thieno[2,3-d]pyrimidine-6-carboxylic acid 2-methyl-benzylamide;

3-Benzyl-1-methyl-2,4-dioxo-1,2,3,4-tetrahydro-thieno[2,3-d]pyrimidine-6-carboxylic acid (4-phenyl-butyl)-amide;

3-Benzyl-1-methyl-2,4-dioxo-1,2,3,4-tetrahydro-thieno[2,3-d]pyrimidine-6-carboxylic acid (pyridin-3-ylmethyl)-amide;

3-Benzyl-1-methyl-2,4-dioxo-1,2,3,4-tetrahydro-thieno[2,3-d]pyrimidine-6-carboxylic acid 4-methoxy-benzylamide;

3-Benzyl-1-methyl-2,4-dioxo-1,2,3,4-tetrahydro-thieno[2,3-d]pyrimidine-6-carboxylic acid ((S)-2,2-dimethyl-4-phenyl-1,3-dioxinan-5-yl)-amide;

3-Benzyl-1-methyl-2,4-dioxo-1,2,3,4-tetrahydro-thieno[2,3-d]pyrimidine-6-carboxylic acid[2-(3-methoxy-phenyl)-ethyl]-amide;

3-Benzyl-1-methyl-2,4-dioxo-1,2,3,4-tetrahydro-thieno[2,3-d]pyrimidine-6-carboxylic acid 3-methoxy-benzylamide;

3-Benzyl-1-methyl-2,4-dioxo-1,2,3,4-tetrahydro-thieno[2,3-d]pyrimidine-6-carboxylic acid (thiophen-2-ylmethyl)-amide;

3-Benzyl-1-methyl-2,4-dioxo-1,2,3,4-tetrahydro-thieno[2,3-d]pyrimidine-6-carboxylic acid 2-chloro-benzylamide;

3-Benzyl-1-methyl-2,4-dioxo-1,2,3,4-tetrahydro-thieno[2,3-d]pyrimidine-6-carboxylic acid (5-methyl-furan-2-ylmethyl)-amide;

3-Benzyl-1-methyl-2,4-dioxo-1,2,3,4-tetrahydro-thieno[2,3-d]pyrimidine-6-carboxylic acid (2,2-diphenyl-ethyl)-amide;

3-Benzyl-1-methyl-2,4-dioxo-1,2,3,4-tetrahydro-thieno[2,3-d]pyrimidine-6-carboxylic acid[2-(2-methoxy-phenyl)-ethyl]-amide;

3-Benzyl-1-methyl-2,4-dioxo-1,2,3,4-tetrahydro-thieno[2,3-d]pyrimidine-6-carboxylic acid[2-(3-trifluoromethyl-phenyl)-ethyl]-amide;

3-Benzyl-1-methyl-2,4-dioxo-1,2,3,4-tetrahydro-thieno[2,3-d]pyrimidine-6-carboxylic acid 4-bromo-benzylamide;

3-Benzyl-1-methyl-2,4-dioxo-1,2,3,4-tetrahydro-thieno[2,3-d]pyrimidine-6-carboxylic acid[2-(1H-indol-3-yl)-ethyl]-amide;

3-Benzyl-1-methyl-2,4-dioxo-1,2,3,4-tetrahydro-thieno[2,3-d]pyrimidine-6-carboxylic acid 3,5-dichloro-benzylamide;

3-Benzyl-1-methyl-2,4-dioxo-1,2,3,4-tetrahydro-thieno[2,3-d]pyrimidine-6-carboxylic acid indan-1-ylamide;

3-Benzyl-1-methyl-2,4-dioxo-1,2,3,4-tetrahydro-thieno[2,3-d]pyrimidine-6-carboxylic acid (furan-2-ylmethyl)-amide;

3-Benzyl-1-methyl-2,4-dioxo-1,2,3,4-tetrahydro-thieno[2,3-d]pyrimidine-6-carboxylic acid[2-(4-methoxy-phenyl)-ethyl]-amide;

3-Benzyl-1-methyl-2,4-dioxo-1,2,3,4-tetrahydro-thieno[2,3-d]pyrimidine-6-carboxylic acid 2,4-dimethoxy-benzylamide;

3-Benzyl-1-methyl-2,4-dioxo-1,2,3,4-tetrahydro-thieno[2,3-d]pyrimidine-6-carboxylic acid 4-chloro-benzylamide;

3-Benzyl-1-methyl-2,4-dioxo-1,2,3,4-tetrahydro-thieno[2,3-d]pyrimidine-6-carboxylic acid (1-phenyl-ethyl)-amide;

3-Benzyl-1-methyl-2,4-dioxo-1,2,3,4-tetrahydro-thieno[2,3-d]pyrimidine-6-carboxylic acid 3,4-dichloro-benzylamide;

3-Benzyl-1-methyl-2,4-dioxo-1,2,3,4-tetrahydro-thieno[2,3-d]pyrimidine-6-carboxylic acid 4-fluoro-3-trifluoromethyl-benzylamide;

3-Benzyl-1-methyl-2,4-dioxo-1,2,3,4-tetrahydro-thieno[2,3-d]pyrimidine-6-carboxylic acid (2-pyridin-2-yl-ethyl)-amide;

3-Benzyl-1-methyl-2,4-dioxo-1,2,3,4-tetrahydro-thieno[2,3-d]pyrimidine-6-carboxylic acid[2-(2,4-dimethyl-phenyl)-ethyl]-amide;

3-Benzyl-1-methyl-2,4-dioxo-1,2,3,4-tetrahydro-thieno[2,3-d]pyrimidine-6-carboxylic acid[2-(2,4-dichloro-phenyl)-ethyl]-amide;

1,3-Dimethyl-2,4-dioxo-1,2,3,4-tetrahydro-thieno[2,3-d]pyrimidine-6-carboxylic acid 3-methoxy-benzylamide;

3-Cyanomethyl-1-methyl-2,4-dioxo-1,2,3,4-tetrahydro-thieno[2,3-d]pyrimidine-6-carboxylic acid 4-methoxy-benzylamide;

3-Benzyl-1-methyl-2,4-dioxo-1,2,3,4-tetrahydro-thieno[2,3-d]pyrimidine-6-carboxylic acid 3-methoxy-benzylamide;

3-(4-Cyclopropylsulfamoyl-benzyl)-1-methyl-2,4-dioxo-1,2,3,4-tetrahydro-thieno[2,3-d]pyrimidine-6-carboxylic acid 3-methoxy-benzylamide;

1-Methyl-3-(6-nitro-pyridin-3-ylmethyl)-2,4-dioxo-1,2,3,4-tetrahydro-thieno[2,3-d]pyrimidine-6-carboxylic acid 3-methoxy-benzylamide;

1-Methyl-3-(6-nitro-pyridin-3-ylmethyl)-2,4-dioxo-1,2,3,4-tetrahydro-thieno[2,3-d]pyrimidine-6-carboxylic acid 4-methoxy-benzylamide;

1-Methyl-3-(6-nitro-pyridin-3-ylmethyl)-2,4-dioxo-1,2,3,4-tetrahydro-thieno[2,3-d]pyrimidine-6-carboxylic acid (2-methoxy-pyridin-4-ylmethyl)-amide;

3-Cyclohexylmethyl-1-methyl-2,4-dioxo-1,2,3,4-tetrahydro-thieno[2,3-d]pyrimidine-6-carboxylic acid (2-methoxy-pyridin-4-ylmethyl)-amide;

3-{2-[(1H-Benzimidazole-5-carbonyl)-amino]-ethyl}-1-methyl-2,4-dioxo-1,2,3,4-tetrahydro-thieno[2,3-d]pyrimidine-6-carboxylic acid 3-methoxy-benzylamide;

1-Methyl-2,4-dioxo-3-[2-(3-piperidin-1-yl-propionylamino)-ethyl]-1,2,3,4-tetrahydro-thieno[2,3-d]pyrimidine-6-carboxylic acid 3-methoxy-benzylamide;

1-Methyl-2,4-dioxo-3-{2-[(6-pyrazol-1-yl-pyridine-3-carbonyl)-amino]-ethyl}-1,2,3,4-tetrahydro-thieno[2,3-d]pyrimidine-6-carboxylic acid 3-methoxy-benzylamide;

3-[2-(4-Diethylamino-benzoylamino)-ethyl]-1-methyl-2,4-dioxo-1,2,3,4-tetrahydro-thieno[2,3-d]pyrimidine-6-carboxylic acid 3-methoxy-benzylamide;

3-{2-[(6-Chloro-pyridine-3-carbonyl)-amino]-ethyl}-1-methyl-2,4-dioxo-1,2,3,4-tetrahydro-thieno[2,3-d]pyrimidine-6-carboxylic acid 3-methoxy-benzylamide;

1-Methyl-2,4-dioxo-3-{2-[(1H-pyrrole-2-carbonyl)-amino]-ethyl}-1,2,3,4-tetrahydro-thieno[2,3-d]pyrimidine-6-carboxylic acid 3-methoxy-benzylamide;

3-[2-(2-Dimethylamino-acetylamino)-ethyl]-1-methyl-2,4-dioxo-1,2,3,4-tetrahydro-thieno[2,3-d]pyrimidine-6-carboxylic acid 3-methoxy-benzylamide;

1-Methyl-2,4-dioxo-3-{2-[(pyrazine-2-carbonyl)-amino]-ethyl}-1,2,3,4-tetrahydro-thieno[2,3-d]pyrimidine-6-carboxylic acid 3-methoxy-benzylamide;

1-Methyl-3-[2-(2-methyl-2-methylamino-propionylamino)-ethyl]-2,4-dioxo-1,2,3,4-tetrahydro-thieno[2,3-d]pyrimidine-6-carboxylic acid 3-methoxy-benzylamide;

1-Methyl-2,4-dioxo-3-{2-[(pyrrolidine-2-carbonyl)-amino]-ethyl}-1,2,3,4-tetrahydro-thieno[2,3-d]pyrimidine-6-carboxylic acid 3-methoxy-benzylamide;

1-Methyl-2,4-dioxo-3-{2-[3-(5-phenyl-1H-pyrrol-2-yl)-propionylamino]-ethyl}-1,2,3,4-tetrahydro-thieno[2,3-d]pyrimidine-6-carboxylic acid 3-methoxy-benzylamide;

1-Methyl-2,4-dioxo-3-{2-[2-(pyridin-4-ylsulfanyl)-acetylamino]-ethyl}-1,2,3,4-tetrahydro-thieno[2,3-d]pyrimidine-6-carboxylic acid 3-methoxy-benzylamide;

3-(6-Amino-pyridin-3-ylmethyl)-1-methyl-2,4-dioxo-1,2,3,4-tetrahydro-thieno[2,3-d]pyrimidine-6-carboxylic acid 3-methoxy-benzylamide;

1-Methyl-2,4-dioxo-3-(3-phenyl-prop-2-ynyl)-1,2,3,4-tetrahydro-thieno[2,3-d]pyrimidine-6-carboxylic acid 4-methoxy-benzylamide;

3-(6-Amino-pyridin-3-ylmethyl)-1-methyl-2,4-dioxo-1,2,3,4-tetrahydro-thieno[2,3-d]pyrimidine-6-carboxylic acid 4-methoxy-benzylamide;

3-(6-Amino-pyridin-3-ylmethyl)-1-methyl-2,4-dioxo-1,2,3,4-tetrahydro-thieno[2,3-d]pyrimidine-6-carboxylic acid (pyridin-4-ylmethyl)-amide;

1-Methyl-2,4-dioxo-3-[2-(pyridin-2-ylamino)-ethyl]-1,2,3,4-tetrahydro-thieno[2,3-d]pyrimidine-6-carboxylic acid 4-methoxy-benzylamide;

1-Methyl-2,4-dioxo-3-[2-(pyrimidin-2-ylamino)-ethyl]-1,2,3,4-tetrahydro-thieno[2,3-d]pyrimidine-6-carboxylic acid 4-methoxy-benzylamide; and

1-Methyl-2,4-dioxo-3-[2-(pyrimidin-2-ylamino)-ethyl]-1,2,3,4-tetrahydro-thieno[2,3-d]pyrimidine-6-carboxylic acid 3-methoxy-benzylamide.

A further embodiment of this invention is use of a compound of Formula I, or a pharmaceutically acceptable salt thereof, in the manufacture of a medicament for the treatment of a disease mediated by an MMP-13 enzyme.

Another invention embodiment is use of a compound of Formulas II, III, VI, VII, or XI, or a pharmaceutically acceptable salt thereof, in the manufacture of a medicament for the treatment of a disease mediated by an MMP-13 enzyme.

Another invention embodiment is use of a compound of Formula I, or a pharmaceutically acceptable salt thereof, in the manufacture of a medicament for the treatment of cancer.

Another invention embodiment is use of a compound of Formula I, or a pharmaceutically acceptable salt thereof, in the manufacture of a medicament for the treatment of rheumatoid arthritis.

Another invention embodiment is use of a compound of Formula I, or a pharmaceutically acceptable salt thereof, in the manufacture of a medicament for the treatment of osteoarthritis.

A further embodiment of this invention is a pharmaceutical composition, comprising a compound of Formula I, or a pharmaceutically acceptable salt thereof, admixed with a pharmaceutically acceptable carrier, excipient, or diluent.

Another invention embodiment is a pharmaceutical composition, comprising a compound of any one of Formulas II, III, VI, VII, and XI, or a pharmaceutically acceptable salt thereof, admixed with a pharmaceutically acceptable carrier, excipient, or diluent.

Another embodiment of this invention is a method for inhibiting MMP-13, in an animal, comprising administering to the animal an MMP-13 inhibiting amount of a compound of Formula I, or a pharmaceutically acceptable salt thereof.

A further embodiment is a method for treating a disease mediated by MMP-13 enzymes, comprising administering to a patient suffering from such disease a effective amount of a compound of Formula I, or a pharmaceutically acceptable salt thereof.

Another invention embodiment is a method for treating a cancer, comprising administering to a patient suffering from such a disease an anticancer effective amount of a compound of Formula I, or a pharmaceutically acceptable salt thereof.

Another invention embodiment is a method for treating breast carcinoma, comprising administering to a patient suffering from such a disease an anticancer effective amount of a compound of Formula I, or a pharmaceutically acceptable salt thereof.

Another invention embodiment is a method for treating a rheumatoid arthritis, comprising administering to a patient suffering from such a disease an effective amount of a compound of Formula I, or a pharmaceutically acceptable salt thereof.

Another invention embodiment is a method for treating a osteoarthritis, comprising administering to a patient suffering from such a disease an effective amount of a compound of Formula I, or a pharmaceutically acceptable salt thereof.

Another invention embodiment is a method for treating a heart failure, comprising administering to a patient suffering from such a disease an effective amount of a compound of Formula I, or a pharmaceutically acceptable salt thereof.

Another invention embodiment is a method for treating a inflammation, comprising administering to a patient suffering from such a disease an effective amount of a compound of Formula I, or a pharmaceutically acceptable salt thereof.

Another invention embodiment is a method of treating a disease mediated by MMP-13 enzymes, comprising administering to a patient suffering from such disease an effective amount of a compound of any one of Formulas II, III, VI, VII, and XI, or a pharmaceutically acceptable salt thereof.

Another embodiment of the present invention is a process for preparing a compound of Formula I

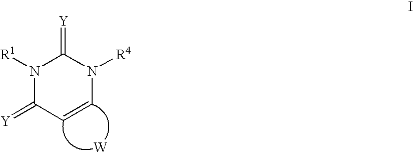

or a pharmaceutically acceptable salt thereof; wherein:

W, together with the carbon atoms to which it is attached, form a 5-membered ring diradical

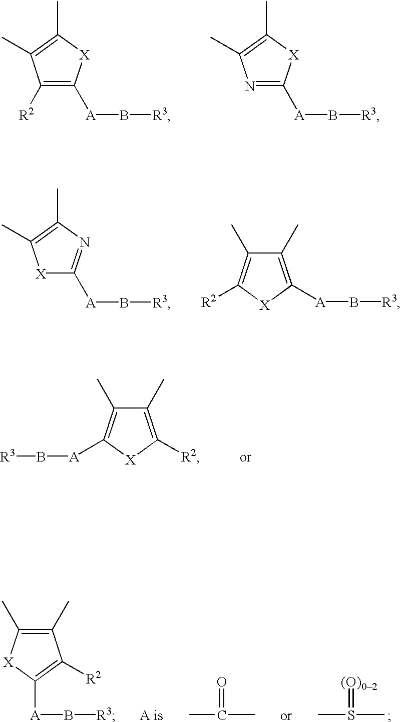

A is

B is O or NR⁵; or

A and B are taken together to form —C≡C—;

X is O, S, SO, SO₂, NR⁵, or CH₂;

each Y independently is O or S;

R¹, R⁴, and R⁵ independently are hydrogen, C₁-C₆ alkyl, C₂-C₆ alkenyl, C₂-C₆ alkynyl, (CH₂)ₙ cycloalkyl, (CH₂)ₙ heterocyclic, C₁-C₆ alkanoyl, (CH₂)ₙ aryl, or (CH₂)ₙ heteroaryl;

R² and R³ independently are hydrogen, C₁-C₆ alkyl, C₂-C₆ alkenyl, C₂-C₆ alkynyl, CN, NO₂, NR⁴R⁵, (CH₂)ₙ cycloalkyl, (CH₂)ₙ aryl, or (CH₂)ₙ heteroaryl;

R² may further be halo;

n is an integer of from 0 to 5; and

R⁴ and R⁵ when taken together with the nitrogen to which they are attached complete a 3- to 8-membered ring containing carbon atoms and optionally containing O, S, or N, and substituted or unsubstituted;

with the proviso that R¹ and R³ are not both selected from: hydrogen and C₁-C₆ alkyl,

the process comprising the step of: contacting a compound of Formula (A)

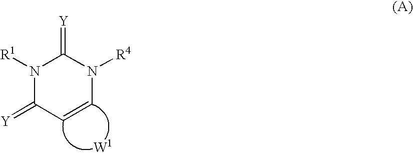

wherein Y, R¹, and R⁴ are as defined above; and

W¹, together with the carbon atoms to which it is attached, form a 5-membered ring diradical

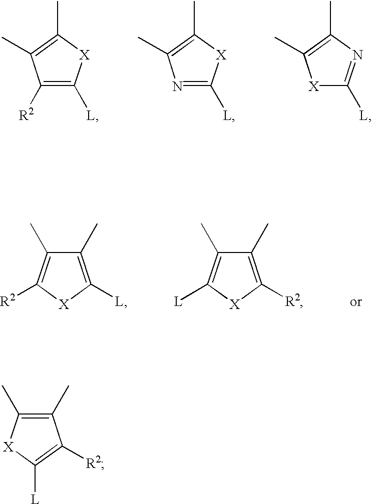

R² and X are as defined above; and

L is a group K or Q, wherein

K is halo, B(OH)₂, Sn(C₁-C₆ alkyl)₃, or OS(O)₂CF₃, and

Q is CO₂H, CO₂M, C(═O)-halo, C(═O)—OR⁷, C(═O)NR⁸R⁹, C(═O)—C(halo)₃, or C≡N,

wherein R⁷ is pentafluorophenyl, C(═O)R⁴, wherein R⁴ is as defined above, or S(O)₂R⁴, wherein R⁴ is as defined above;

R⁸ and R⁹ are taken together with the nitrogen atom to which they are attached to form imidazol-1-yl, phthalimid-1-yl, benzotriazol-1-yl, or tetrazol-1-yl; and

M is an alkali earth metal cation or alkaline earth metal cation; with a solvent and, when L is the group Q, a compound of Formula (B)

D—R³ (B)

wherein R³ is as defined above and D is HO, HN(R⁵), MO, or MN(R⁵);

wherein R⁵ and M are as defined above;

optionally in the presence of from 1 to 3 agents selected from:

a coupling agent, a tertiary organic amine, an acid catalyst, a base catalyst, an acid halide, and an acid anhydride.

Another invention embodiment is a process comprising the step of:

contacting a compound of Formula (A)

as defined above with a solvent and, when

L is the group K, a compound of Formula (C)

G—C≡C—R³ (C)

wherein R³ is as defined above and G is hydrogen or halo;

optionally in the presence of a coupling catalyst.

Another invention embodiment is the invention process wherein Y is O and X is S; or

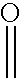

Another invention embodiment is the invention process wherein A is —C—, B is O or NR⁵, Y is O, and X is S.

Another invention embodiment is the invention process wherein A and B are taken together to form —C≡C, Y is O, and X is S.

Another invention embodiment is the invention process wherein R¹ and R³ independently are (CH₂)ₙ aryl, or (CH₂)ₙ heteroaryl, wherein n is an integer of from 0 to 5.

Another invention embodiment is the invention process wherein n is 1.

Another invention embodiment is any one of the above invention process embodiments wherein L is CO₂H, CO₂M, C(═O)-halo, wherein M is an alkali earth metal cation or an alkaline earth metal cation.

Another invention embodiment is any one of the above invention process embodiments wherein L is halo.

Another invention embodiment is any one of the above invention process embodiments wherein G is H.

### DETAILED DESCRIPTION OF THE INVENTION

The compounds provided by this invention are those defined by Formula I. In Formula I, R¹-R⁴ include “C₁-C₆ alkyl” groups. These are straight and branched carbon chains having from 1 to 6 carbon atoms. Examples of such alkyl groups include methyl, ethyl, isopropyl, tert-butyl, neopentyl, and n-hexyl. The alkyl groups can be substituted if desired, for instance, with groups such as aryl-O—, wherein aryl is as defined below, heteroaryl-O—, wherein heteroaryl is as defined below, hydroxy, amino, alkyl, and dialkylamino, halo, trifluoromethyl, carboxy, nitro, and cyano. Typical substituted alkyl groups thus are aminomethyl, 2-nitroethyl, 4-cyanobutyl, 2,3-dichloropentyl, and 3-hydroxy-5-carboxyhexyl.

Examples of NR⁴R⁵ groups include amino, methylamino, di-isopropylamino, acetyl amino, propionyl amino, 3-aminopropyl amino, s 3-ethylaminobutyl amino, 3-di-n-propylamino-propyl amino, 4-diethylaminobutyl amino, and 3-carboxypropionyl amino. R⁴ and R⁵ can be taken together with the nitrogen to which they are attached to form a ring having 3 to 7 carbon atoms and 1, 2, or 3 heteroatoms selected from the group consisting of nitrogen, substituted nitrogen, oxygen, and sulfur. Examples of such cyclic NR⁴R⁵ groups include pyrrolidinyl, piperazinyl, 4-methylpiperazinyl, 4-benzylpiperazinyl, pyridinyl, piperidinyl, pyrazinyl, morpholinyl, and the like.

“Halo” includes fluoro, chloro, bromo, and iodo.

“Alkenyl” means straight and branched hydrocarbon radicals having from 2 to 6 carbon atoms and one double bond and includes ethenyl, 3-buten-1-yl, 2-ethenylbutyl, 3-hexen-1-yl, and the like.

“Alkynyl” means straight and branched hydrocarbon radicals having from 2 to 6 carbon atoms and one triple bond and includes ethynyl, 3-butyn-1-yl, propynyl, 2-butyn-1-yl, 3-pentyn-1-yl, and the like.

“Carbocycle” or “Cycloalkyl” mean a monocyclic or polycyclic hydrocarbyl group such as cyclopropyl, cycloheptyl, cyclooctyl, cyclodecyl, cyclobutyl, adamantyl, norpinanyl, decalinyl, norbornyl, cyclohexyl, and cyclopentyl. Such groups can be substituted with groups such as hydroxy, keto, and the like. Also included are rings in which 1 to 3 heteroatoms replace carbons. Such groups are termed “heterocycle” or “heterocyclic” or “heterocyclyl,” which mean a cycloalkyl group also bearing at least one heteroatom selected from O, S, or NR₂, examples being oxiranyl, pyrrolidinyl, piperidyl, tetrahydropyran, and morpholine.

“Alkoxy” refers to the alkyl groups mentioned above bound through oxygen, examples of which include methoxy, ethoxy, isopropoxy, tert-butoxy, and the like. In addition, alkoxy refers to polyethers such as —O—(CH₂)₂—O—OH₃, and the like.

“Alkanoyl” groups are alkyl linked through a carbonyl, ie, C₁-C₅—C(O)—. Such groups include formyl, acetyl, propionyl, butyryl, and isobutyryl.

“Acyl” means an alkyl or aryl (Ar) group bonded through a carbonyl group, i.e., R—C(O)—. For example, acyl includes a C₁-C₆ alkanoyl, including substituted alkanoyl, wherein the alkyl portion can be substituted by NR⁴R⁵ or a carboxylic or heterocyclic group. Typical acyl groups include acetyl, benzoyl, and the like.

The alkyl, alkenyl, alkoxy, and alkynyl groups described above are optionally substituted, preferably by 1 to 3 groups selected from NR⁴R⁵, phenyl, substituted phenyl, thio C₁-C₆ alkyl, C₁-C₆ alkoxy, hydroxy, carboxy, aryl-O—, wherein aryl is as defined below, heteroaryl-O—, wherein heteroaryl is as defined below, C₁-C₆ alkoxycarbonyl, halo, nitrile, cycloalkyl, and a 5- or 6-membered carbocyclic ring or heterocyclic ring having 1 or 2 heteroatoms selected from nitrogen, substituted nitrogen, oxygen, and sulfur. “Substituted nitrogen” means nitrogen bearing C₁-C₆ alkyl or (CH₂)ₙPh where n is 1, 2, or 3. Perhalo and polyhalo substitution is also embraced. Oxo (═O) substitution of a CH₂ carbon group to provide a carbonyl (C═O) is also embraced.

Examples of substituted alkyl groups include 2-aminoethyl, pentachloroethyl, trifluoromethyl, 2-diethylaminoethyl, 2-dimethylaminopropyl, ethoxycarbonylmethyl, 3-phenylbutyl, methanylsulfanylmethyl, methoxymethyl, 3-hydroxypentyl, 2-carboxybutyl, 4-chlorobutyl, 3-cyclopropylpropyl, pentafluoroethyl, 3-morpholinopropyl, piperazinylmethyl, and 2-(4-methylpiperazinyl)ethyl.

Examples of substituted alkynyl groups include 2-methoxyethynyl, 2-ethylsulfanyethynyl, 4-(1-piperazinyl)-3-(butynyl), 3-phenyl-5-hexynyl, 3-diethylamino-3-butynyl, 4-chloro-3-butynyl, 4-cyclobutyl-4-hexenyl, and the like.

Typical substituted alkoxy groups include aminomethoxy, trifluoromethoxy, 2-diethylaminoethoxy, 2-ethoxycarbonylethoxy, 3-hydroxypropoxy, 6-carboxhexyloxy, and the like.

Further, examples of substituted alkyl, alkenyl, and alkynyl groups include dimethylaminomethyl, carboxymethyl, 4-dimethylamino-3-buten-1-yl, 5-ethylmethylamino-3-pentyn-1-yl, 4-morpholinobutyl, 4-tetrahydropyrinidylbutyl, 3-imidazolidin-1-ylpropyl, 4-tetrahydrothiazol-3-yl-butyl, phenylmethyl, 3-chlorophenylmethyl, and the like.

The terms “Ar” and “aryl” refer to unsubstituted and substituted aromatic groups. Heteroaryl groups have from 4 to 10 ring atoms which are carbon atoms, and from 1 to 4 of which are independently selected from the group consisting of O, S, and N. Preferred heteroaryl groups have 1 or 2 heteroatoms in a 5- or 6-membered aromatic ring. Mono and bicyclic aromatic ring systems are included in the definition of aryl and heteroaryl. Typical aryl and heteroaryl groups include phenyl, 3-chlorophenyl, 2,6-dibromophenyl, pyridyl, 3-methylpyridyl, benzothienyl, 2,4,6-tribromophenyl, 4-ethylbenzothienyl, furanyl, 3,4-diethylfuranyl, naphthyl, 4,7-dichloronaphthyl, morpholinyl, indolyl, benzotriazolyl, indazolyl, pyrrole, pyrazole, imidazole, thiazole, and the like.

Preferred Ar groups are phenyl and phenyl substituted by 1, 2, or 3 groups independently selected from the group consisting of alkyl, alkoxy, thio, thioalkyl, 1H-tetrazol-5-yl, halo, hydroxy, —COOR⁶, trifluoromethyl, nitro, amino of the formula —NR⁴R⁵, and T(CH₂)ₘQR⁴ or T(CH₂)ₘCO₂R⁴ wherein m is 1 to 6, T is O, S, NR^{4,}N(O)R⁴, NR⁴R⁵Y, or CR⁴R⁵, Q is O, S, NR⁵, N(O)R⁵, or NR⁴R⁵Y wherein R⁴ and R⁵ are as described above, and R⁶ is hydrogen, alkyl, or substituted alkyl, for example, methyl, trichloroethyl, diphenylmethyl, and the like. The alkyl and alkoxy groups can be substituted as defined above. For example, typical groups are carboxyalkyl, alkoxycarbonylalkyl, hydroxyalkyl, hydroxyalkoxy, and alkoxyalkyl. Typical substituted aryl groups include 2,6-dichlorophenyl, 3-methoxyphenyl, 4-trifluoromethylphenyl, 4-styrylphenyl, 3-amino-4-nitrophenyl, 3,5-dihydroxyphenyl, and the like.

Most preferred aryl is phenyl, 4- or 3-methoxy-phenyl, 4-fluorophenyl, and 3-fluorophenyl, and each of 3,4-disubstituted phenyls wherein the substituents are methoxy and fluoro.

Most preferred heteroaryl is pyridin-4-yl or 2-methoxypyridin-4-yl.

The phrase “tertiary organic amine” means a trisubstituted nitrogen group wherein the 3 substituents are independently selected from C₁-C₁₂ alkyl, C₃-C₁₂ cycloalkyl, benzyl, or wherein two of the substituents are taken together with the nitrogen atom to which they are attached to form a 5- or 6-membered, monocyclic heterocycle containing one nitrogen atom and carbon atoms, and the third substituent is selected from C₁-C₁₂ alkyl and benzyl, or wherein the three substituents are taken together with the nitrogen atom to which they are attached to form a 7- to 12-membered bicyclic heterocycle containing 1 or 2 nitrogen atoms and carbon atoms, and optionally a C═N double bond when 2 nitrogen atoms are present. Illustrative examples of tertiary organic amine include triethylamine, diisopropylethylamine, benzyl diethylamino, dicyclohexylmethyl-amine, 1,8-diazabicycle[5.4.0]undec-7-ene (“DBU”), 1,4-diazabicyclo[2.2.2]-octane (“TED”), and 1,5-diazabicycle[4.3.0]non-5-ene.

The term “coupling agent” includes any reagent, or any combination of two, three, or four reagents, conventionally used to promote coupling of a carboxylic acid, or a pharmaceutically acceptable salt thereof, with an alcohol or an amine to yield a carboxylic ester or carboxylic amide, respectively. The coupling agents are described in Reagents for Organic Synthesis by Fieser and Fieser, New York: John Wiley & Sons, Inc., 2000; Comprehensive Organic Transformations by Richard C. Larock, New York: VCH Publishers, Inc., 1989; the series Compendium of Organic Synthetic Methods by Wiley-Interscience, 1989; and the text Advanced Organic Chemistry, 5^{th} edition, by Jerry March, New York: Wiley-Interscience, 2001. Illustrative examples of coupling agents include N,N′-carbonyldiimidazole (“CDI”), N,N′-dicyclohexylcarbodiimide (“DCC”), triphenylphosphine with diethylazodicarboxylate, bis(2-oxo-3-oxazolidinyl)phosphinic chloride (“BOP-Cl”), POCl₃, Ti(Cl)₄, and 1-(3-dimethylaminopropyl)-3-ethylcarbodiimide hydrochloride (“EDAC”).

The phrase “acid catalyst” means any protic or Lewis acid that is conventionally used to catalyze coupling of a carboxylic acid, or a pharmaceutically acceptable salt thereof, a nitrile, carboxylic ester, carboxylic amide, carboxylic acid halide, or carboxylic acid anhydride with an alcohol or an amine to yield a carboxylic ester or carboxylic amide, respectively. The acid catalysts are described in Fieser and Fieser, supra., 2000; Larock R C, supra., 1989; Wiley-Interscience, supra., 1989; and March J, supra., 2001. Illustrative examples include anhydrous hydrogen chloride, hydrochloric acid, hydrogen bromide in acetic acid, zinc chloride, titanium tetrachloride, acetic acid, trifluoroacetic acid, phenol, sulfuric acid, methanesulfonic acid, magnesium sulfate, Amberlyst-15 resin, silica gel, and the like.

It should be appreciated that a nitrile may be contacted with an alcohol or an amine in the presence of an acid catalyst, and the resulting intermediate imidate or amidine, respectfully, may be contacted with water to yield the carboxylic ester or carboxylic amide, respectively.

The phrase “base catalyst” means any base that is conventionally used to catalyze coupling of a carboxylic acid, or a pharmaceutically acceptable salt thereof, carboxylic ester, carboxylic amide, carboxylic acid halide, or carboxylic acid anhydride with an alcohol or an amine to yield a carboxylic ester or carboxylic amide, respectively. The base catalysts are described in Fieser and Fieser, supra., 2000;. Larock R C, supra., 1989; Wiley-Interscience, supra., 1989; and March J, supra., 2001. Illustrative examples include sodium hydroxide, sodium hydride, potassium tert-butoxide, a tertiary organic amine, titanium tetraisopropoxide, sodium methoxide, sodium acetate, sodium bicarbonate, potassium carbonate, basic alumina, and the like.

The phrase “acid halide” means any carboxylic acid halide or sulfonic acid halide that is conventionally used to catalyze coupling of a carboxylic acid, or a pharmaceutically acceptable salt thereof, with an alcohol or an amine to yield a carboxylic ester or carboxylic amide, respectively. The acid halides are described in Fieser and Fieser, supra., 2000; Larock R C, supra., 1989; Wiley-Interscience, supra., 1989; and March J, supra., 2001. Illustrative examples include acetyl chloride, trifluoromethanesulfonyl chloride, 2,2-dimethylacetyl bromide, para-toluenesulfonyl chloride, pentafluoro-benzoyl chloride, and the like.

The phrase “acid anhydride” means any carboxylic acid anhydride or sulfonic acid anhydride that is conventionally used to catalyze coupling of a carboxylic acid, or a pharmaceutically acceptable salt thereof, with an alcohol or an amine to yield a carboxylic ester or carboxylic amide, respectively. The acid anhydrides are described in Fieser and Fieser, supra., 2000; Larock R C, supra., 1989; Wiley-Interscience, supra., 1989; and March J, supra., 2001. Illustrative examples include acetic anhydride, trifluoroacetic anhydride, trifluoromethanesulfonic acid anhydride, pentafluoro-benzoic anhydride, mixed anhydrides like trifluoroacetyloxycarbonylmethyl, and the like.

The term “halide” includes fluoride, chloride, bromide, and iodide.

The phrase “coupling catalyst” means any metal catalyst, preferably a transition metal catalyst, that is conventionally used to catalyze coupling of an aryl halide, aryl trifluoromethanesulfonate, heteroaryl halide, or heteroaryl trifluoromethanesulfonate, or activated derivatives thereof, including arylboronic acids, heteroarylboronic acids, aryl stannanes, heteroarylstannanes, aryl magnesium halides, heteroaryl magnesium halides, aryl lithiums, or heteroaryl lithiums, with an terminal alkyne to yield an arylalkyne or heteroarylalkyne. The coupling catalysts are described in Fieser and Fieser, supra., 2000; Larock R C, supra., 1989; Wiley-Interscience, supra., 1989; and March J, supra., 2001. Illustrative examples of coupling catalysts include tetrakis(triphenylphosphine)-palladium (0), palladium (II) chloride, palladium (II) acetate, iron (III) chloride, Heck reaction catalysts, Suzuki reaction catalysts, Stille reaction catalysts, and the like.

The group

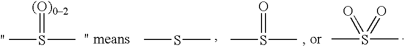

The phrase “pharmaceutical composition” means a composition suitable for administration in medical or veterinary use.

The term “admixed” and the phrase “in admixture” are synonymous and mean in a state of being in a homogeneous or heterogeneous mixture. Preferred is a homogeneous mixture.

The term “patient” means a mammal. Preferred patients are humans, cats, dogs, cows, horses, pigs, and sheep.

The term “animal” means a mammal, as defined above. Preferred animals include humans, cats, dogs, horses, pigs, sheep, cows, monkeys, rats, mice, guinea pigs, and rabbits.

The phrase “anticancer effective amount” means an amount of invention compound, or a pharmaceutically acceptable salt thereof, sufficient to inhibit, halt, or cause regression of the cancer being treated in a particular patient or patient population. For example in a human or other mammal, an anticancer effective amount can be determined experimentally in a laboratory or clinical setting, or may be the amount required by the guidelines of the United States Food and Drug Administration, or equivalent foreign agency, for the particular cancer and patient being treated.

The phrase “antiarthritic effective amount” means an amount of invention compound, or a pharmaceutically acceptable salt thereof, sufficient to inhibit, halt, or cause regression of the arthritis being treated in a particular patient or patient population. For example in a human or other mammal, an antiarthritic effective amount can be determined experimentally in a laboratory or clinical setting, or may be the amount required by the guidelines of the United States Food and Drug Administration, or equivalent foreign agency, for the particular arthritis and patient being treated.

The phrase “MMP-13 inhibiting amount” means an amount of invention compound, or a pharmaceutically acceptable salt thereof, sufficient to inhibit an enzyme matrix metalloproteinase-13, including a truncated form thereof, including a catalytic domain thereof, in a particular animal or animal population. For example in a human or other mammal, an MMP-13 inhibiting amount can be determined experimentally in a laboratory or clinical setting, or may be the amount required by the guidelines of the United States Food and Drug Administration, or equivalent foreign agency, for the particular MMP-13 enzyme and patient being treated.

It should be appreciated that determination of proper dosage forms, dosage amounts, and routes of administration, is within the level of ordinary skill in the pharmaceutical and medical arts, and is described below.

The phrases “effective amount” and “therapeutically effective amount” are synonymous and mean an amount of a compound of the present invention, a pharmaceutically acceptable salt thereof, or a solvate thereof, sufficient to effect an improvement of the condition being treated when administered to a patient suffering from a disease that is mediated by MMP-13 and optionally from 0 to 12 additional MMP enzymes.

The term “IC₅₀” means the concentration of test compound required to inhibit activity of a biological target, such as a receptor or enzyme, by 50%.

It should be appreciated that the matrix metalloproteinases include the following enzymes:

MMP-1, also known as interstitial collagenase, collagenase-1, or fibroblast-type collagenase;

MMP-2, also known as gelatinase A or 72 kDa Type IV collagenase;

MMP-3, also known as stromelysin or stromelysin-1;

MMP-7, also known as matrilysin or PUMP-1;

MMP-8, also known as collagenase-2, neutrophil collagenase, or polymorphonuclear-type (“PMN-type”) collagenase;

MMP-9, also known as gelatinase B or 92 kDa Type IV collagenase;

MMP-10, also known as stromelysin-2;

MMP-11, also known as stromelysin-3;

MMP-12, also known as metalloelastase;

MMP-13, also known as collagenase-3;

MMP-14, also known as membrane-type (“MT”) 1-MMP or MT1-MMP;

MMP-15, also known as MT2-MMP;

MMP-16, also known as MT3-MMP;

MMP-17, also known as MT4-MMP;

MMP-18; and

MMP-19.

Other MMPs include MMP-26, also known as matrilysin-2.

One aspect of the present invention is novel compounds that are selective inhibitors of the enzyme MMP-13. A selective inhibitor of MMP-13, as used in the present invention, is a compound that is ≧5 times more potent in vitro versus MMP-13 than versus at least one other matrix metalloproteinase enzyme such as, for example, MMP-1, MMP-2, MMP-3, MMP-7, MMP-8, MMP-9, or MMP-14, or versus tumor necrosis factor alpha convertase (“TACE”). A preferred aspect of the present invention is novel compounds that are selective inhibitors of MMP-13 versus MMP-1. Other aspects of the present invention are compounds that are ≧10, ≧20, ≧50, ≧100, or ≧1000 times more potent in vitro versus MMP-13 than versus at least one other MMP enzyme or TACE.

Still other aspects of the present invention are compounds of Formula I, or a pharmaceutically acceptable salt thereof, that are selective inhibitors of MMP-13 versus 2, 3, 4, 5, 6, or 7 other MMP enzymes, or versus TACE and 1, 2, 3, 4, 5, 6, or 7 other MMP enzymes.

Some of the compounds in the present invention may exist as stereoisomers, including enantiomers, diastereomers, and geometric isomers. Geometric isomers include compounds of the present invention that have alkenyl groups, which may exist as entgegen or zusammen conformations, in which case all geometric forms thereof, both entgegen and zusammen, cis and trans, and mixtures thereof, are within the scope of the present invention. Some compounds of the present invention have cycloalkyl groups, which may be substituted at more than one carbon atom, in which case all geometric forms thereof, both cis and trans, and mixtures thereof, are within the scope of the present invention. All of these forms, including (R), (S), epimers, diastereomers, cis, trans, syn, anti, (E), (Z), and mixtures thereof, are contemplated in the invention compounds of Formulas I to XI.

The compounds to be used in the present invention can exist in unsolvated forms as well as solvated forms, including hydrated forms. In general, the solvated forms, including hydrated forms, are equivalent to unsolvated forms and are intended to be encompassed within the scope of the present invention.

The compounds of Formulas I through XI are capable of further forming both pharmaceutically acceptable salts, including but not limited to acid addition and/or base salts. This invention also provides pharmaceutical compositions comprising a compound of Formula I together with a pharmaceutically acceptable carrier, diluent, or excipient therefor. All of these forms can be used in the method of the present invention.

Pharmaceutically acceptable acid addition salts of the compounds of Formula I include salts derived form inorganic acids such as hydrochloric, nitric, phosphoric, sulfuric, hydrobromic, hydroiodic, phosphorus, and the like, as well as the salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are the salts of amino acids such as arginate, gluconate, galacturonate, and the like; see, for example, Berge et al., “Pharmaceutical Salts,” J. of Pharmaceutical Science, 1977;66:1-19.

The acid addition salts of the basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metal hydroxides, or of organic amines. Examples of metals used as cations are sodium, potassium, magnesium, calcium, and the like. Examples of suitable amines are N,N′-dibenzylethylenediamine, chloroprocaine, choline, diethanolamine, ethylenediamine, N-methylglucamine, and procaine; see, for example, Berge et al., supra., 1977.

The base addition salts of acidic compounds are prepared by contacting the free acid form with a sufficient amount of the desired base to produce the salt in the conventional manner. The free acid form may be regenerated by contacting the salt form with an acid and isolating the free acid in a conventional manner. The free acid forms differ from their respective salt forms somewhat in certain physical properties such as solubility in polar solvents, but otherwise the salts are equivalent to their respective free acid for purposes of the present invention.

The compounds of the present invention can be formulated and administered in a wide variety of oral and parenteral dosage forms, including transdermal and rectal administration. All that is required is that an MMP inhibitor be administered to a mammal suffering from a disease in an effective amount, which is that amount required to cause an improvement in the disease and/or the symptoms associated with such disease. It will be recognized to those skilled in the art that the following dosage forms may comprise as the active component, either a compound of Formula I or a corresponding pharmaceutically acceptable salt or solvate of a compound of Formula I.

A compound of Formula I, or a pharmaceutically acceptable salt thereof, may be prepared by one of ordinary skill in the art of organic chemistry by procedures found in the chemical literature such as, for example, Fieser and Fieser, supra., 2000; Larock R C, supra., 1989; Wiley-Interscience, supra., 1989; March J, supra., 2001; or the Handbook of Heterocyclic Chemistry by Alan R. Katritzky, London: Pergamon Press Ltd., 1985, to name a few. Alternatively, a skilled artisan may find methods useful for preparing the invention compounds in the chemical literature by searching widely available databases such as, for example, those available from the Chemical Abstracts Service, Columbus, Ohio, or MDL Information Systems GmbH (formerly Beilstein Information Systems GmbH), Frankfurt, Germany.

Preparations of the compounds of the present invention may use starting materials, reagents, solvents, and catalysts that may be purchased from commercial sources or they may be readily prepared by adapting procedures in the references or resources cited above. Commercial sources of starting materials, reagents, solvents, and catalysts useful in preparing invention compounds include, for example, The Aldrich Chemical Company, and other subsidiaries of Sigma-Aldrich Corporation, St. Louis, Mo., BACHEM, BACHEM A. G., Switzerland, or Lancaster Synthesis Ltd., United Kingdom.

Fieser and Fieser, supra., 2000; Larock R C, supra., 1989; Wiley-Interscience, supra., 1989; March J, supra., 2001; and Katritzky A R, supra., 1985, are hereby incorporated by reference.

The invention compounds are prepared by methods well known to those skilled in the art of organic chemistry. The compounds of Formula I are prepared utilizing commercially available starting materials, or reactants that are readily prepared by standard organic synthetic techniques. A typical synthesis of the invention compounds of Formula I is shown in Scheme 1 below. The first step in Scheme 1 comprises reacting a chlorouracil analog with 2-mercapto acetate ester;. The reaction generally is carried out in a solvent such as an alkanol, for example ethanol, and in the presence of a base such as sodium carbonate. The reaction is usually substantially complete after about 2 to 6 hours when carried out at an elevated temperature of about 40° C. to about 80° C. The product, an alkylthio substituted tetrahydro pyrimidine, can be isolated and purified if desired, or can be used directly in the next step. The next step is a cyclization reaction (Vilsmeier reaction). The alkylthio substituted tetrahydro pyrimidine is reacted with POCl₃ in a polar solvent such as dimethylformamide or dimethylsulfoxide to effect cyclization to the corresponding tetrahydro-thieno[2,3-d]pyrimidine-2,4-dione. The thienopyrimidinone can be further modified by standard procedures, for example alkylation at the 1-position by reaction with an alkylating agent R⁴L, where L is a leaving group such as chloro or bromo, and R⁴ is as defined above. Ester groups can be hydrolyzed by reaction with a base such as sodium hydroxide, and carboxylic groups can be esterified by standard procedures such as reaction with an alcohol R³OH in the presence of an acid such as hydrochloric acid, or in the presence of a coupling reagent such as DCC (dicyclohexylcarbodiimide) and CMC (1-cyclohexyl-3-(2-morpholinoethyl)carbodiimide metho-p-toluenesulfonate. Carboxylic acid groups can be converted to amides by standard methods, for example by first reaction with oxalyl chloride to form an acid chloride, and then reaction of the acid chloride with an amine of the formula HNR⁴R⁵.

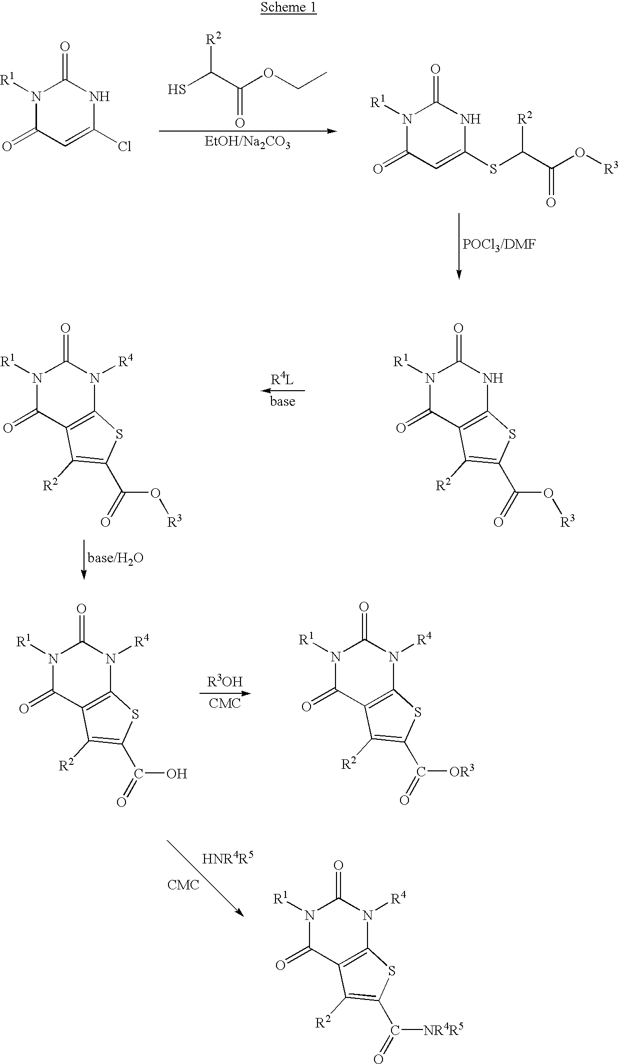

Scheme 2 illustrates the synthesis of compounds of Formula 1 starting from a benzyl alkanoylacetate, which reacts with a cyanoacetic acid ester; in the presence of powdered sulfur (when X is S) and a base such as morpholine to give an amino substituted heterocycle. This condensation typically is carried out by combining the reactants in a solvent such as methanol or ethanol, and generally is complete within about 2 to 10 hours when carried out at an elevated temperature of about 40° C. to 60° C. The 5-benzyloxycarbonyl-2-amino-substituted heterocycle (e.g., thiophene when X is S, furan when X is O, and pyrrole when X is NH) is next reacted with an isocyanate (R¹NCO) to effect cyclization to form the pyrimidinone ring. This cyclization reaction is carried out by mixing the reactants in a solvent such as dioxane in the presence of a strong base such as sodium hydride. The cyclization is generally complete within about 8 to 24 hours when carried out at a temperature of about 24° C. to 60° C. The product, a compound of Formula I wherein R⁴ is H, can be alkylated or arylated by reaction with an alkyl or aryl halide (R⁴L, where L is a leaving group such as chloro or bromo). The invention compound can be further modified by standard methods, for instance by hydrolyzing the ester; forming group R³ to give the corresponding acid (where R³═H), and then re-esterifying or amidating by reaction with an amine in the presence of a coupling agent such as DCC or CMC.

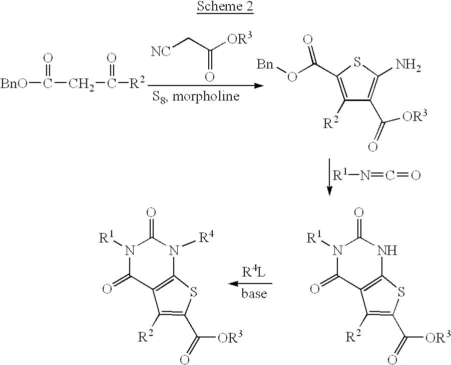

Scheme 3 illustrates reaction of a 4-alkoxycarbonyl-5-amino thiazole (where X is S) with an isocyanate in the presence of a strong base such as sodium hydride to form the 6-member pyrimidinone ring. The unsubstituted ring nitrogen can be alkylated or arylated by standard reactions, for example by reactions with a alkylating agent R⁴L, where L is a leaving group such as halo.

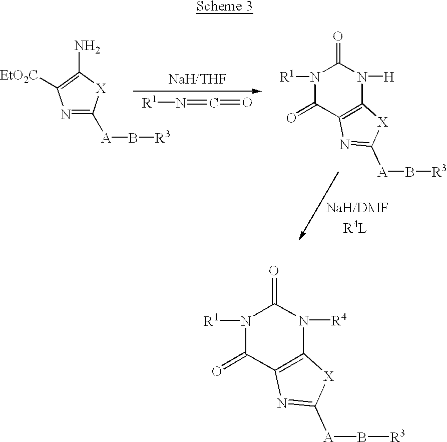

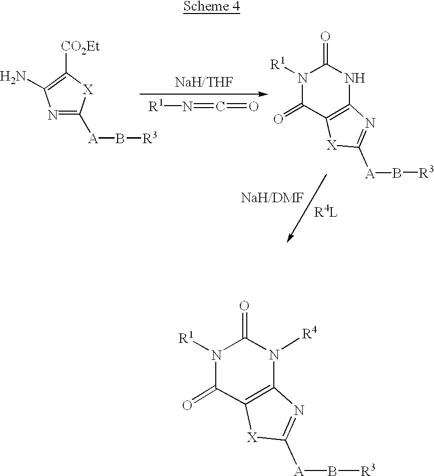

The corresponding sulfoxide and sulfone analogs can be prepared in the same fashion.

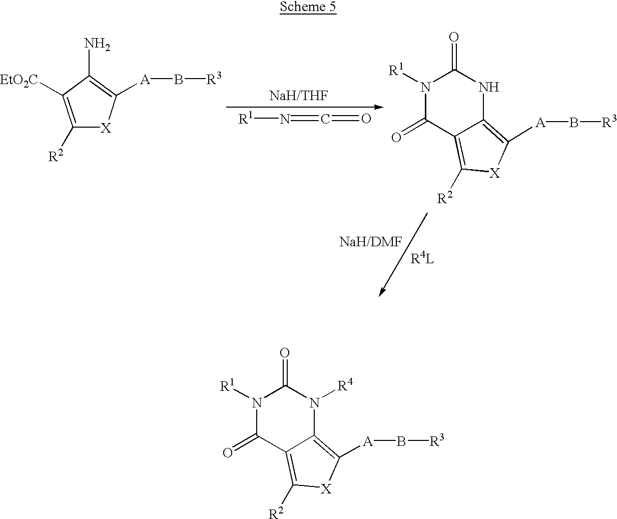

The corresponding ester and amide analogs can be prepared in the same fashion.

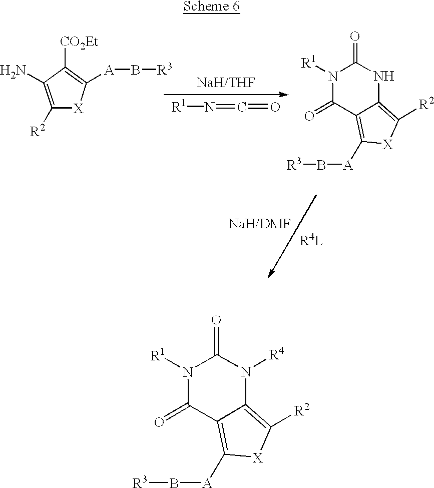

The corresponding ester and amide analogs can be prepared in the same fashion.

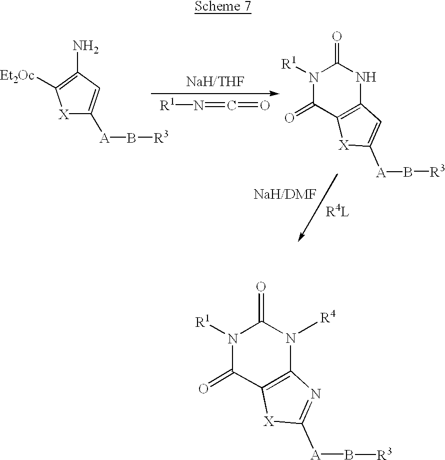

The corresponding ester and amide analogs can be prepared in the same fashion.

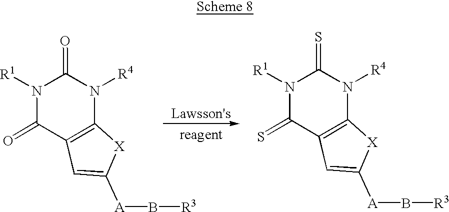

The corresponding ester and amide analogs can be prepared in the same fashion.

The alkynes can be prepared in a conventional manner as illustrated in Scheme 9. In Scheme 9, an aryl iodide (or, optionally, an aryl bromide, aryl chloride, or aryl trifluoromethanesulfonate) is coupled to a terminal alkyne in the presence of a palladium catalyst, cuprous (I) iodide, and a base such as a tertiary amine base.

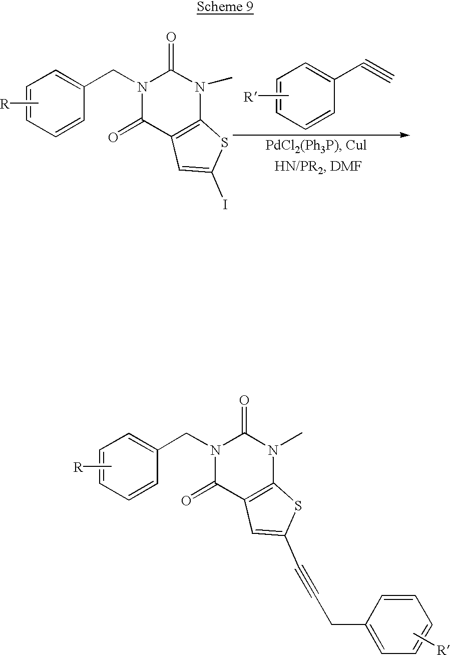

wherein R and R′ independently are hydrogen or from 1 to 3 substituents as defined above for substituted phenyl.

The following detailed examples further illustrate the synthesis of typical invention compounds of Formula I. The examples are representative only, and are not to be construed as limiting the invention in any respect.

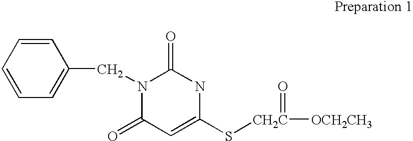

### (1-Benzyl-2,6-dioxo-1,2,3,6-tetrahydro-pyrimidin-4-ylsulfanyl)-acetic acid ethyl ester

To 250 mL of ethanol in a round bottom flask was added 3-benzyl-6-chloro-1H-pyrimidine-2,4-dione (11.55 g, 48.94 mmol), sodium carbonate (5.19 g, 48.94 mmol), and mercapto-acetic acid ethyl ester (6.47 g, 53.83 mmol). The mixture is stirred at reflux for 5 hours. The reaction solution is filtered, and the filtrate is chromatographed on a silica gel column, eluting with 4:1 Hexane/Ethyl Acetate (400 mL) followed by 1000 mL of 4:1 Dichloromethane/Ethyl Acetate. Removing the solvents by vacuum yielded 10.5 g of white powder identified as the titled product (67%). ¹H NMR (DMSO), δ1.16 (t, J=7.1 Hz, 3H), 4.06 (s, 2H), 4.12 (q, J=7.1 Hz, 2H), 4.88 (s, 2H), 5.54 (s, 1H), 7.22-7.30 (m, 5H), 11.71 (broad s, 1H). MS (APCI−), m/z 321 (M⁺).

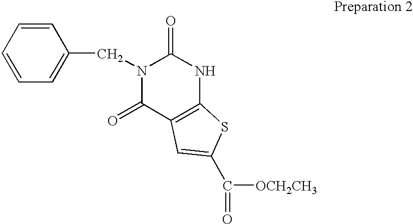

### 3-Benzyl-2,4-dioxo-1,2,3,4-tetrahydro-thieno[2,3-d]pyrimidine-6-carboxylic acid ethyl ester

To a solution of (1-benzyl-2,6-dioxo-1,2,3,6-tetrahydro-pyrimidin-4-ylsulfanyl)-acetic acid ethyl ester from Preparation 1 (6.37 g, 19.8 mmol) in anhydrous DMF (60 mL) was added POCl₃ (9.11 g, 59.5 mmol) dropwise. The reaction is then stirred at room temperature overnight, and then heated to 70° C. for 30 minutes. The reaction is cooled to room temperature and poured into 600 mL of stirring ice water. The product is filtered and washed with water to yield 6.2 g (95%) very light yellow powder as the titled compound. ¹H NMR (DMSO), δ1.27 (t, J=7.1 Hz, 3H), 4.26 (q, J=7.1 Hz, 2H), 5.00 (s, 2H), 7.19-7.29 (m, 5H), 7.76 (s, 1H), 12.6 (broad s, 1H). MS (APCI−), m/z 331 (M⁺).

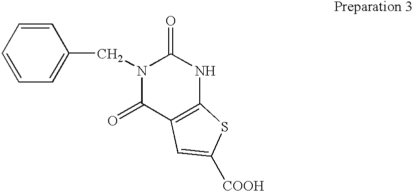

### 3-Benzyl-2,4-dioxo-1,2,3,4-tetrahydro-thieno[2,3-d]pyrimidine-6-carboxylic acid

To a solution of 3-benzyl-2,4-dioxo-1,2,3,4-tetrahydro-thieno[2,3-d]pyrimidine-6-carboxylic acid ethyl ester from Preparation 2 (2.9 g, 8.79 mmol) in a solution of 90% THF: 10% water (v/v) was added lithium hydroxide (3.69 g, 87.9 mmol). The solution is refluxed for 2 hours. The solvent was removed by vacuum, and the residual was diluted with water (100 mL). HCl was added until the solution has a pH of 1. The solution was extracted with ethyl acetate (3×100 mL). The combined organic layer was concentrated to yield 2.62 g of white powder as product (96%). ¹H NMR (DMSO), δ4.99 (s, 2H), 7.19-7.29 (m, 5H), 7.68 (s, 1H). MS (APCI−), m/z 331 (M⁺).

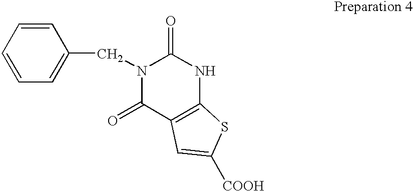

### 3-Benzyl-2,4-dioxo-1,2,3,4-tetrahydro-thieno[3,2-d]pyrimidine-6-carboxylic acid

Step (1): Thiophene-2,5-dicarboxylic acid diethyl ester

To a solution of 2,5-thiophene dicarboxylic acid (5.0 g, 29 mmol) in methanol (100 mL) was added sulfuric acid (1.0 mL), and the reaction was refluxed for 72 hours. The mixture was then concentrated under reduced pressure. The crude residue was diluted with ethyl acetate (250 mL), and the mixture was washed with water (3×100 mL). The organic phase was dried (MgSO₄) and concentrated to yield 6.52 g (98%) of thiophene-2,5-dicarboxylic acid diethyl ester as an orange oil.

Step (2): 3-Nitro-thiophene-2,5-dicarboxylic acid diethyl ester

To a chloroform solution (40 mL) of the product of Step (1) (6.52 g, 28.6 mmol) and trifluoroacetic anhydride (20 mL) was slowly added copper (II) nitrate hemipentahydrate (7.31 g, 31.5 mmol), and the reaction mixture was heated to 60° C. over 4 hours. The reaction mixture was poured into ice (200 g), and was extracted with chloroform (2×150 mL). The chloroform layers were combined, dried (MgSO₄), filtered, and concentrated. The resulting orange oil residue was purified by flash chromatography on silica gel (eluting with cyclohexane:ethyl acetate, 4:1) to yield 4.61 g (60%) of 3-nitro-thiophene-2,5-dicarboxylic acid diethyl ester as a yellow solid.

Step (3): 3-Amino-thiophene-2,5-dicarboxylic acid diethyl ester

A solution of the product of Step (2) (4.61 g, 16.9 mmol) in ethanol (20 mL) was hydrogenated over 10% Pd-C (460 mg, 10 wt %) in a Parr shaker at 200 psi over 48 hours at 60° C. The catalyst was then filtered off, and the filtrate was concentrated under vacuum, and the resulting residue was purified by flash chromatography on silica gel (cyclohexane:ethyl acetate, 4:1) to yield 3.20 g (78%) of 3-amino-thiophene-2,5-dicarboxylic acid diethyl ester as a white solid.

Step (4): 3-(3-Benzyl-ureido)-thiophene-2,5-dicarboxylic acid diethyl ester

A pyridine solution (10 mL) of the product of Step (3) (2.06 mmol, 500 mg), benzylisocyanate (2.06 mmol, 255 mL), and 4-(dimethylamino)pyridine (0.41 mmol, 50 mg) was heated to 90° C. for 48 hours. The reaction mixture was then concentrated under reduced pressure, and the residue was purified by flash chromatography on silica gel (4:1:5 cyclohexane:ethyl acetate:toluene) to yield 599 mg (77%) of 3-(3-benzyl-ureido)-thiophene-2,5-dicarboxylic acid diethyl ester as a white solid.

Step (5): 3-Benzyl-2,4-dioxo-1,2,3,4-tetrahydro-thieno[3,2-d]pyrimidine-6-carboxylic acid

To a solution of the product of Step (4) (1.57 mmol, 590 mg) in ethanol (15 mL) was added sodium ethoxide (3.14 mmol, 214 mg), and resulting solution was refluxed for 4 hours. The reaction mixture was then allowed to reach room temperature, and lithium hydroxide (3.9 mmol, 94 mg) was added. The reaction mixture was stirred for 17 hours and concentrated under reduced pressure to afford a crude product. The crude product was dissolved in 1.0M hydrochloric acid (10 mL). The resulting white precipitate was collected, washed with water (3×10 mL), cold acetonitrile (3×5 mL), and dried under vacuum to yield 336 mg (71%) of 3-benzyl-2,4-dioxo-1,2,3,4-tetrahydro-thieno[3,2-d]pyrimidine-6-carboxylic acid as a white solid.

### EXAMPLE 1

### 3-Benzyl-2,4-dioxo-1,2,3,4-tetrahydro-thieno[2,3-d]pyrimidine-6-carboxylic acid benzyl ester

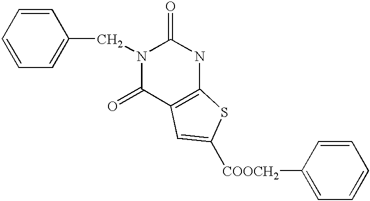

A dichloromethane (30 mL) solution of 3-benzyl-2,4-dioxo-1,2,3,4-tetrahydro-thieno[2,3-d]pyrimidine-6-carboxylic acid (0.8 g, 2.65 mmol) from Preparation 3, 1-cyclohexyl-3-(2-morpholinoethyl)carbodiimide metho-p-toluenesulfonate (CMC, 1.35 g, 3.18 mmol), and benzyl alcohol (0.32 g, 2.91 mmol) is refluxed for 3 hours. The solution is then diluted with dichloromethane (100 mL) and washed with water (3×100 mL). The organic layer is concentrated and purified by chromatography over a silica gel column using 2:1 Hexane:Ethyl Acetate to yield 120 mg of white solid as product (12%). MP: 195-197° C.; ¹H NMR (CDCl₃), δ5.18 (s, 2H), 5.33 (s, 2H), 7.26-7.49 (m, 10H), 8.03 (s, 1H), 10.84 (s, 1H). MS (APCI−), m/z 303 (M⁺).

Calcd for C₂₁H₁₆N₂O₄S₁: C, 64.27; H, 4.11; N, 7.14. Found: C, 64.24; H, 3.80; N, 7.04.

### EXAMPLE 2

### 3-Benzyl-2,4-dioxo-1,2,3,4-tetrahydro-thieno[2,3-d]pyrimidine-6-carboxylic acid pyridin-4-ylmethyl ester

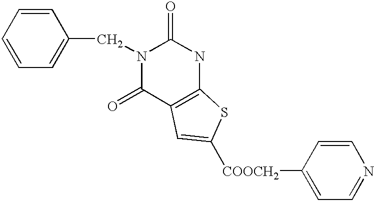

The procedure of Example 1 was repeated, except that benzyl alcohol is replaced with 4-pyridyl methyl alcohol to provide 3-benzyl-2,4-dioxo-1,2,3,4-tetrahydro-thieno[2,3-d]pyrimidine-6-carboxylic acid pyridin-4-ylmethyl ester as a white powder. (32%). MP: 248-250° C.; ¹H NMR (DMSO), δ5.00 (s, 2H), 5.36 (s, 2H), 7.22-7.34 (m, 5H), 7.41 (d, J=5.7 Hz, 2H), 7.91 (s, 1H), 8.57 (d, J=5.7 Hz, 2H), 12.62 (broad s,1H). MS (APCI−), m/z 394 (M⁺).

### EXAMPLE 3

### 3-Benzyl-2,4-dioxo-1,2,3,4-tetrahydro-thieno[2,3-d]pyrimidine-6-carboxylic acid benzyl amide

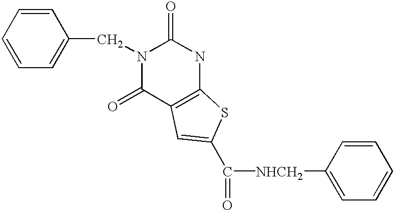

The procedure of Example 1 was repeated, except that benzyl alcohol is replaced with benzylamine, to provide 3-benzyl-2,4-dioxo-1,2,3,4-tetrahydro-thieno[2,3-d]pyrimidine-6-carboxylic acid benzyl amide as a white solid (20%). MP:>255° C.; ¹H NMR (CDCl₃), δ4.53 (s, 2H), 4.90 (s, 1H), 5.17 (s, 2H), 7.16-7.41 (m, 10H), 7.77 (s, 1H). MS (APCI−), m/z 392 (M⁺).

### EXAMPLE 4

### 3-Benzy-2,4,dioxo-1,2,3,4-tetrahydro-thieno[2,3-d]pyrimidine-6 carboxylic acid 4-((E)-styryl-benzyl ester

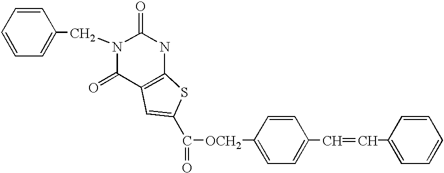

The procedure of Example 1 was repeated, except that benzyl alcohol is replaced with [4-((E)-styryl-phenyl]-methanol, to give 3-benzy-2,4,dioxo-1,2,3,4-tetrahydro-thieno[2,3-d]pyrimidine-6-carboxylic acid 4-((E)-styryl-benzyl ester as a white solid. MP: 247-249° C.; ¹H NMR (d₈-THF), δ10.85 (bd s, 1H), 7.91 (s, 1H), 7.58-7.19 (m, 16H), 5.31 (s, 2H), 5.08 (s, 2H). MS m/z 495.3 (m+1), m/z 493.3 (m−1).

### EXAMPLE 5

### 5-Methyl-2,4-dioxo-3-phenylethyl-1,2,3,4-tetrahydro-thieno[2,3-d]pyrimidine-6-carboxylic acid benzyl ester

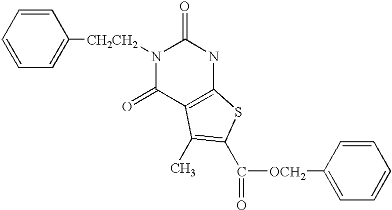

To a stirred solution of the 5-amino-3-methyl-thiophene-2,4-dicarboxylic acid 2-benzyl ester 4-ethyl ester in dioxane (0.5 g, 1.57 mmol) was added NaH (0.042 g, 1.72 mmol). The mixture was further stirred until no more hydrogen gas was evolved and then 2-isocyanato-ethyl-benzene (0.23 g, 1.57 mmol) was added slowly. The resulting mixture was refluxed under nitrogen until the reaction was complete by MS and TLC. The dioxane was removed by rotary evaporation. The reaction mixture was then purified by flash column chromatography eluting with 4:1 (Hex:EtOAc), 2:1 (Hex:EtOAc), and 1:1 (Hex:EtOAc) sequentially. The fractions containing the product were collected and concentrated to yield a white solid which was triturated with 4:1 (Hex:EtOAc). 5-Methyl-2,4-dioxo-3-phenylethyl-1,2,3,4-tetrahydro-thieno[2,3-d]pyrimidine-6-carboxylic acid benzyl ester was collected by filtration and dried in a vacuum oven overnight.

Calcd for C₂₃H₂₀N₂O₄: C, 65.70; H, 4.79; N, 6.66. Found: C, 64.90; H, 4.82; N, 6.42.

MP: 207-209° C.; ¹H NMR (d₁-CDCl₃), δ9.35 (s, 1H), 7.52-7.21 (m, 10H), 5.33 (s, 2H), 4.19 (t, 2H, J=8 Hz), 2.95 (t, 2H, J=8z Hz), 1.55 (s, 3H). MS m/z 467.3 (m+1), m/z 465.2 (m−1).

### EXAMPLE 6

### 3-(4-Acetyl-phenyl)-5-methyl-2,4 dioxo-1,2,3,4-tetrahydro-thieno[2,3-d]-pyrimidine-6-carboxylic acid benzyl ester

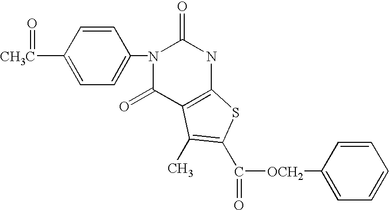

The procedure of Example 5 was repeated, except that 2-isocyanato-ethyl-benzene is replaced with 4-isocyanatoacetophenone to give 3-(4-acetyl-phenyl)-5-methyl-2,4-dioxo-1,2,3,4-tetrahydro-thieno[2,3-d]pyrimidine-6-carboxylic acid benzyl ester as an off white solid. ¹H NMR (d₁-CDCl₃), δ8.84 (s, 1H), 8.11 (d, 2H, J=9 Hz) 7.41-7.25 (m, 7H), 5.34 (s, 2H), 2.84 (s, 3H), 2.64 (s, 3H). MS m/z 435.2 (m+1), m/z 434.2 (m−1).

### EXAMPLE 7

### 5-Methyl-2,4-dioxo-3-p-tolyl-1,2,3,4-tetrahydro-thieno[2,3-d]-6-carboxylic acid benzyl ester

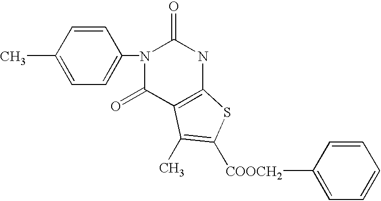

When in the procedure of Example 5,2-isocyanato-ethyl-benzene is replaced with 4-tolyl isocyanate, 5-methyl-2,4-dioxo-3-tolyl-1,2,3,4-tetrahydro-thieno[2,3-d]-6-carboxylic acid benzyl ester is obtained as a white solid. MP 267-269° C.; ¹H NMR (d₁-CDCl₃), δ8.79 (s, 1H), 7.41-6.99 (m, 9H), 5.33 (s, 2H), 2.84 (s, 3H), 2.40 (s, 3H). MS m/z 407.2 (m+1), m/z 405.3 (m−1).

### EXAMPLE 8

### 5-Methyl-3-(4-nitro-phenyl)-2,4-dioxo-1,2,3,4-tetrahydro-thieno[2,3-d]-6-carboxylic acid benzyl ester

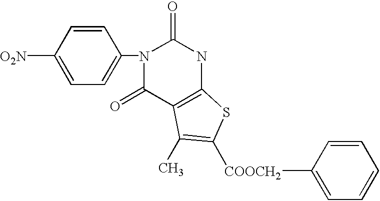

The procedure of Example 5 was repeated, except that 2-isocyanato-ethyl-benzene is replaced with 4-nitrophenyl isocyanate to give 5-methyl-3-(4-nitro-phenyl)-2,4-dioxo-1,2,3,4-tetrahydro-thieno[2,3-d]-6-carboxylic acid benzyl ester as a yellow solid. 1H NMR (d₆-DMSO), δ9.64 (s, 1H), 8.31 (d, 2H, J=9 Hz), 7.62 (d, 2H, J=9 Hz), 7.44-7.35 (m, 5H), 5.30 (s, 2H), 2.70 (s, 3H). MS m/z 436.1 (m−1).

### EXAMPLE 9

### 3-Benzyl-1-methyl-2,4-dioxo-1,2,3,4-tetrahydro-thieno[2,3-d]pyrimidine-6-carboxylic acid benzyl ester

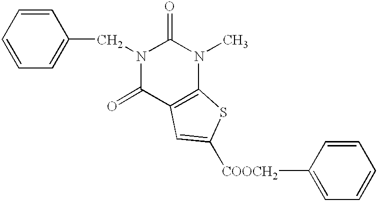

To a solution of 3-benzyl-2,4-dioxo-1,2,3,4-tetrahydro-thieno[2,3-d]-pyrimidine-6-carboxylic acid benzyl ester (300 mg, 0.765 mmol) in DMF was added NaH (46 mg, 1.5 mmol). After 5 minutes, MeI (0.15 mL, 2.3 mmol) was added, and the reaction mixture was stirred at room temperature for 30 minutes. After removal of all volatiles, the residue was purified using flash chromatography to give the desired product as a white solid (204 mg, 66%). R_{f}=0.51

(2:1 hexane/EtOAc). MP: 143-145° C.

Calcd for C₂₂H₁₈N₂O₄S₁: C, 65.01; H, 4.46; N, 6.89. Found: C, 64.61; H4.31; N, 6.74.

### EXAMPLE 10

### 3-Benzyl-2,4-dioxo-1,2,3,4-tetrahydro-thieno[2,3-d]pyrimidine-6-carboxylic acid 1,3-benzodioxol-5-ylmethyl ester

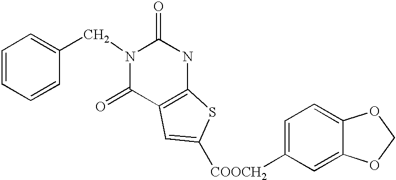

The procedure of Example 1 was repeated, except that benzyl alcohol is replaced with benzo[1,3]dioxol-5-yl-methanol to give 3-benzyl-2,4-dioxo-1,2,3,4-tetrahydro-thieno[2,3-d]pyrimidine-6-carboxylic acid 1,3-benzodioxol-5-ylmethyl ester as a white solid. 1H NMR (d₈-THF), δ10.86 (s, 1H), 7.89 (s, 1H), 6.80-7.49 (m, 8H), 5.96 (s, 2H), 5.21 (s, 2H), 5.09 (s, 2H). MS (APCI−), m/z 393.2 (M⁺+1).

### EXAMPLE 11

### 3-Benzyl-1-methyl-2,4-dioxo-1,2,3,4-tetrahydro-thieno[2,3-d]pyrimidine-6-carboxylic acid benzyl amide

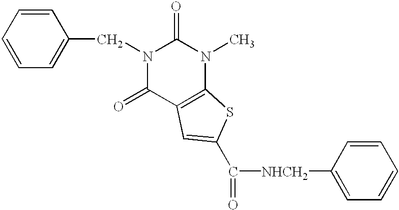

A dichloromethane (30 mL) solution of 3-benzyl-1-methyl-2,4-dioxo-1,2,3,4-tetrahydro-thieno[2,3-d]pyrimidine-6-carboxylic acid (367 mg, 1.16 mmol), CMC (392 g, 0.92 mmol), and benzylamine (149 mg, 1.39 mmol) is refluxed for 3 hours. The solution is then diluted with dichloromethane (100 mL) and washed with water (3×100 mL). The organic layer is concentrated and purified by chromatography over a silica gel column using 1:1 Hexane:Ethyl Acetate to yield 200 mg of white solid as product. ¹H NMR (d₈-THF), δ9.23 (t, 1H), 8.11 (s, 1H), 7.20-7.38 (m, 10H), 5.04 (s, 2H), 4.43 (s, 2H), 3.46 (s, 3H). MS (APCI−), m/z 406.1 (M⁺+1).

### EXAMPLES 12-14

By following the general procedures of Examples 1 through 11, the following invention compounds were prepared:

3-Benzyl-2,4-dioxo-1,2,3,4-tetrahydro-thieno[2,3-d]pyrimidine-6-carboxylic acid 2-phenylethyl ester;

3-Benzyl-2,4-dioxo-1,2,3,4-tetrahydro-thieno[2,3-d]pyrimidine-6-carboxylic acid furan-3-ylmethyl ester; and

3-Benzyl-2,4-dioxo-1,2,3,4-tetrahydro-thieno[2,3-d]pyrimidine-6-carboxylic acid furfuryl-(5-carboxaldelhyde)ester (also known as 3-benzyl-2,4-dioxo-1,2,3,4-tetrahydro-thieno[2,3-d]pyrimidine-6-carboxylic acid 5-formyl-furan-2-ylmethyl ester).

### EXAMPLE 15

### 3-(3-Methoxycarbonyl-benzyl)-1-methyl-2,4-dioxo-1,2,3,4-tetrahydro-thieno[2,3-d]pyrimidine-6-carboxylic acid benzyl ester

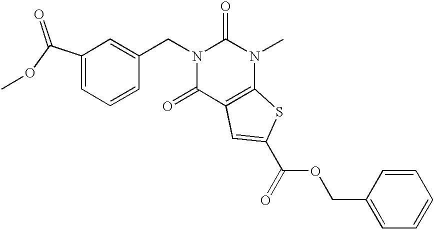

To a solution of 1-methoyl-2,4-dioxo-1,2,3,4-tetrahydro-thieno[2,3-d]pyrimidine-6-carboxylic acid benzyl ester (0.2 g, 0.63 mmol) in anhydrous DMF was added cesium carbonate (0.31 g, 0.945 mmol) and 3-bromomethyl-benzoic acid methyl ester (0.145 g, 0.63 mmol). The reaction was stirred at room temperature for overnight. Poured into water (150 mL) and extracted with EtOAc. The organic layer washed with water and brine, dried over MgSO₄ and then filtered. The filtrate was concentrated in vacuo. Triturating the residue with 4:1 Hexane/EtOAc yielded a white solid as the desired product (40%). MS (APCI+), m/z 465.1(M+)

### EXAMPLE 16

### 3-(3-Methoxycarbonyl-benzyl)-1-methyl-2,4-dioxo-1,2,3,4-tetrahydro-thieno[2,3-d]pyrimidine-6-carboxylic acid benzyl ester

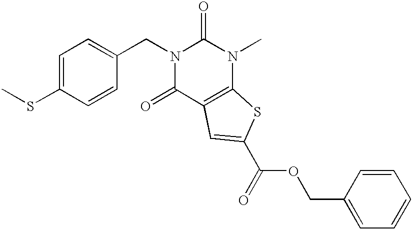

The procedure of Example 15 was repeated, except 3-bromomethyl-benzoic acid methyl ester is replaced by 1-chloromethyl-4-methylsulfanyl-benzene, to give 3-(3-methoxycarbonyl-benzyl)-1-methyl-2,4-dioxo-1,2,3,4-tetrahydro-thieno[2,3-d]pyrimidine-6-carboxylic acid benzyl ester as an off white solid (50%). MS (APCI+), m/z 453 (M+).

### EXAMPLE 17

### 3-Benzofuran-5-ylmethyl-1-methyl-2,4-dioxo-1,2,3,4-tetrahydro-thieno[2,3-d]pyrimidine-6-carboxylic acid benzyl ester

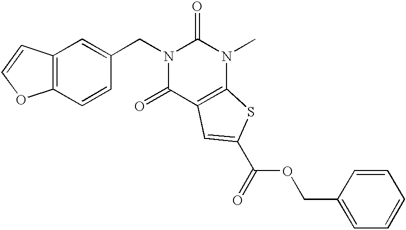

The procedure of Example 15 was repeated, except 3-bromomethyl-benzoic acid methyl ester is replaced by 5-bromomethyl-benzofuran. Instead of trituration, the crude product was chromatographed using 8:1 Hexane/EtOAc to 4:1 Hexane/EtOAc to give 3-benzofuran-5-ylmethyl-1-methyl-2,4-dioxo-1,2,3,4-tetrahydro-thieno[2,3-d]pyrimidine-6-carboxylic acid benzyl ester as a white solid (57%). MS (APCI+), m/z 447 (M+).

### EXAMPLE 18

### 1-Methyl-3-(4-methyl-benzyl)-2,4-dioxo-1,2,3,4-tetrahydro-thieno[2,3-d]pyrimidine-6-carboxylic acid benzyl ester

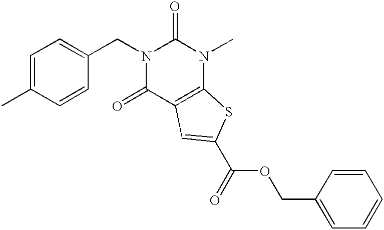

The procedure of Example 15 was repeated, except 3-bromomethyl-benzoic acid methyl ester is replaced by 1-ethyl-4-methyl-benzene, to give 1-methyl-3-(4-methyl-benzyl)-2,4-dioxo-1,2,3,4-tetrahydro-thieno[2,3-d]pyrimidine-6-carboxylic acid benzyl ester as an off white solid (50%). MS (APCI+), m/z 421 (M+).

### EXAMPLE 19

### 3-(4-Acetylamino-benzyl)-1-methyl-2,4-dioxo-1,2,3,4-tetrahydro-thieno[2,3-d]pyrimidine-6-carboxylic acid benzyl ester

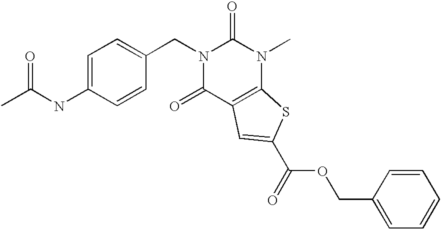

The procedure of Example 15 was repeated, except 3-bromomethyl-benzoic acid methyl ester is replaced by N-(4-chloromethyl-phenyl)-acetamide, to give 3-(4-Acetylamino-benzyl)-1-methyl-2,4-dioxo-1,2,3,4-tetrahydro-thieno[2,3-d]pyrimidine-6-carboxylic acid benzyl ester as an off white solid (57%). MS (APCI+), m/z 464 (M+).

### EXAMPLE 20

### 1-Methyl-2,4-dioxo-3-(4-vinyl-benzyl)-1,2,3,4-tetrahydro-thieno[2,3-d]pyrimidine-6-carboxylic acid benzyl ester

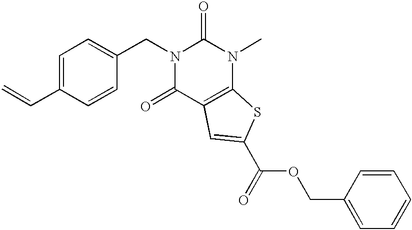

The procedure of Example 15 was repeated, except 3-bromomethyl-benzoic acid methyl ester is replaced by 1 ethyl-4-vinyl-benzene. Instead of trituration, the crude product was chromatographed using 8:1 Hexane/EtOAc to 4:1 Hexane/EtOAc to give 1-methyl-2,4-dioxo-3-(4-vinyl-benzyl)-1,2,3,4-tetrahydro-thieno[2,3-d]pyrimidine-6-carboxylic acid benzyl ester as a white solid (73%). MS (APCI+), m/z 433 (M+).

### EXAMPLE 21

### 1-Methyl-2,4-dioxo-3-(4-sulfamoyl-benzyl)-1,2,3,4-tetrahydro-thieno[2,3-d]pyrimidine-6-carboxylic acid benzyl ester

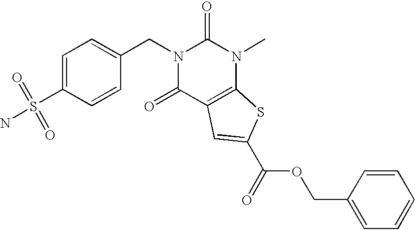

The procedure of Example 15 was repeated, except 3-bromomethyl-benzoic acid methyl ester is replaced by 4-bromomethyl-benzenesulfonamide. Instead of trituration, the crude product was chromatographed using 2:1 EtOAc/EtOAc to 100% EtOAc to give 1-methyl-2,4-dioxo-3-(4-sulfamoyl-benzyl)-1,2,3,4-tetrahydro-thieno[2,3-d]pyrimidine-6-carboxylic acid benzyl ester as a white solid (60%). MS (APCI+), m/z 486 (M+).

### EXAMPLE 22

### 3-(4-Bromo-benzyl)-2,4-dioxo-1,2,3,4-tetrahydro-thieno[2,3-d]pyrimidine-6-carboxylic acid pyridin-4-ylmethyl ester

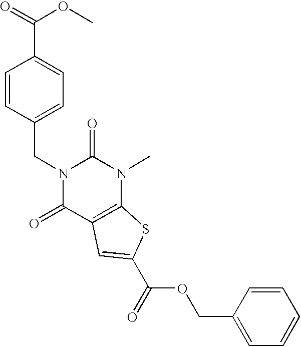

The procedure of Example 15 was repeated, except 3-bromomethyl-benzoic acid methyl ester is replaced by 4-ethylbenzoic acid methyl ester, to give 3-(4-bromo-benzyl)-2,4-dioxo-1,2,3,4-tetrahydro-thieno[2,3-d]pyrimidine-6-carboxylic acid pyridin-4-ylmethyl ester as an off white solid (82%). MS (APCI+), m/z 465 (M+).

### EXAMPLE 23

### 1-Methyl-2,4-dioxo-3-phenethyl-1,2,3,4-tetrahydro-thieno[2,3-d]pyrimidine-6-carboxylic acid benzyl ester

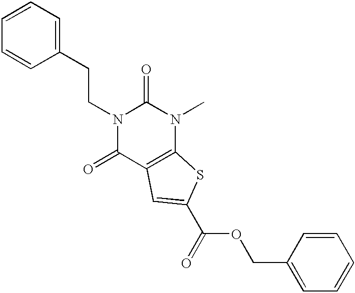

The procedure of Example 15 was repeated, except 3-bromomethyl-benzoic acid methyl ester is replaced by (2-bromo-ethyl)-benzene, to give 1-methyl-2,4-dioxo-3-phenethyl-1,2,3,4-tetrahydro-thieno[2,3-d]pyrimidine-6-carboxylic acid benzyl ester as an off white solid (50%). MS (APCI+), m/z 421 (M+).

### EXAMPLE 24

### 1-Methyl-2,4-dioxo-3-[4-(2H-tetrazol-5-yl)-benzyl]-1,2,3,4-tetrahydro-thieno[2,3-d]pyrimidine-6-carboxylic acid benzyl ester

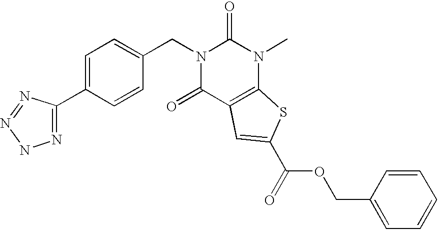

To a solution of 3-(4-cyano-benzyl)-1-methyl-2,4-dioxo-1,2,3,4-tetrahydro-thieno[2,3-d]pyrimidine-6-carboxylic acid (0.615 g, 1.42 mmol) in 10 mL of dioxane was added tributyltin azide (0.71 g, 2.14 mmol). The reaction solution was refluxed overnight. After removing the solvent in vacuo, the residue was dissolved in ether and HCl gas was bubbled in for 1 hour. The precipitant was filtered, dissolved in chloroform and chromatographed using EtOAc and THF. The fractions were collected and concentrated. The residue was triturated with 4:1 Hexane/EtOAc, to yield the 1-methyl-2,4-dioxo-3-[4-(2H-tetrazol-5-yl)-benzyl]-1,2,3,4-tetrahydro-thieno[2,3-d]pyrimidine-6-carboxylic acid benzyl ester as a white solid (20%). MS (APCI), m/z 473 (M−).

### EXAMPLE 25

### 3-(4-Fluoro-benzyl)-2,4-dioxo-1,2,3,4-tetrahydro-thieno[2,3-d]pyrimidine-6-carboxylic acid pyridin-4-ylmethyl ester

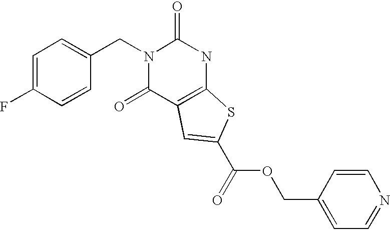

The procedure of Example 2 was repeated, except the 3-benzyl-2,4-dioxo-1,2,3,4-tetrahydro-thieno[2,3-d]pyrimidine-6-carboxylic acid was replaced by 3-(4-fluoro-benzyl)-2,4-dioxo-1,2,3,4-tetrahydro-thieno[2,3-d]pyrimidine-6-carboxylic acid made using 4-flurobenzyl in place of benzyl during the synthesis outlined in preparation 1-3, to give 3-(4-fluoro-benzyl)-2,4-dioxo-1,2,3,4-tetrahydro-thieno[2,3-d]pyrimidine-6-carboxylic acid pyridin-4-ylmethyl ester as a pink solid. MS (APCI+), m/z 412 (M+).

### EXAMPLE 26

### 3-(4-tert-butyoxycarbonyl-benzyl)-1-methyl-2,4-dioxo-1,2,3,4-tetrahydro-thieno[2,3-d]pyrimidine-6-carboxylic acid benzyl ester

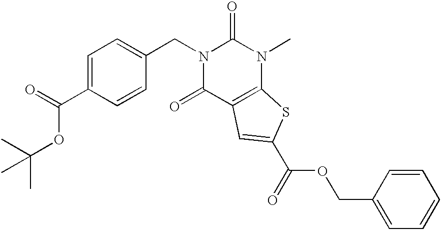

The procedure of Example 15 was repeated, except 3-bromomethyl-benzoic acid methyl ester is replaced by 4-bromomethyl-benzoic acid tert-butyl ester, to 3-(4-tert-butyoxycarbonyl-benzyl)-1-methyl-2,4-dioxo-1,2,3,4-tetrahydro-thieno[2,3-d]pyrimidine-6-carboxylic acid benzyl ester as a white solid (70%). MS (APCI+), m/z 493 (M+).

### EXAMPLE 27

### 3-(4-tert-butyoxycarbonyl-benzyl)-1-methyl-2,4-dioxo-1,2,3,4-tetrahydro-thieno[2,3-d]pyrimidine-6-carboxylic acid

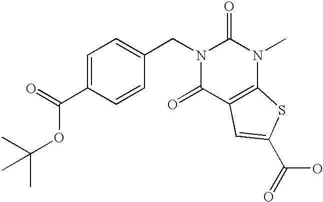

To a solution of 3-(4-tert-butyoxycarbonyl-benzyl)-1-methyl-2,4-dioxo-1,2,3,4-tetrahydro-thieno[2,3-d]pyrimidine-6-carboxylic acid benzyl ester (0.5 g, 0.98 mmol) in 40 mL of 10:1 THF/water, was added 0.24 g of LiOH. Reaction was stirred at room temperature for 5 hours. THF was removed in reduced pressure, and 50 mL of water was added along with 150 mL of EtOAc. The solution is then acidified by HCl and shaken. The organic layer was washed by water and brine, and concentrated to yield 3-(4-tert-butyoxycarbonyl-benzyl)-1-methyl-2,4-dioxo-1,2,3,4-tetrahydro-thieno[2,3-d]pyrimidine-6-carboxylic acid as an off white solid (66%). MS (APCI+), m/z 417 (M+).

### EXAMPLE 28

### 4-[6-(4-Fluoro-benzylcarbamoyl)-1-methyl-2,4-dioxo-1,4-dihydro-2H-thieno[2,3-d]pyrimidin-3-ylmethyl]-benzoic acid

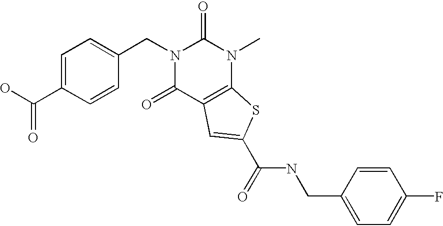

To a solution of 3-(4-tert-butyoxycarbonyl-benzyl)-1-methyl-2,4-dioxo-1,2,3,4-tetrahydro-thieno[2,3-d]pyrimidine-6-carboxylic acid (0.2 g, 0.48 mmol) and Mukiyama reagent (0.147 g, 0.57 mmol) in 6 mL of CH₂Cl₂ was added Et₃N (0.116 g, 1.14 mmol) and 4-fluorobenzyl amine (0.065 g, 0.52 mmol). The reaction solution was stirred at room temperature for overnight. The reaction solution was then chromatographed using 4:1 Hexane/EtOAc. The isolated product was then concentrated and dissolved in 5 mL of TFA. After stirring at room temperature for 30 minutes, the solution was concentrated and triturated using 4:1 Hexane/EtOAc to yield 4-[6-(4-fluoro-benzylcarbamoyl)-1-methyl-2,4-dioxo-1,4-dihydro-2H-thieno[2,3-d]pyrimidin-3-ylmethyl]-benzoic acid as a white solid (77%). MS (APCI+), m/z 468 (M+).

### EXAMPLE 29

### 4-[6-(4-Dimethylamino-benzylcarbamoyl)-1-methyl-2,4-dioxo-1,4-dihydro-2H-thieno[2,3-d]pyrimidin-3-ylmethyl]-benzoic acid; compound with trifluoro-acetic acid

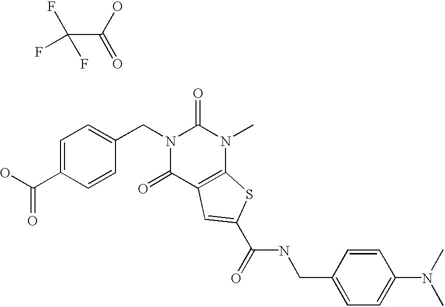

The procedure of Example 28 was repeated, 4-fluorobenzyl amine is replaced by (4-ethyl-phenyl)-dimethyl-amine, to give 4-[6-(4-dimethylamino-benzylcarbamoyl)-1-methyl-2,4-dioxo-1,4-dihydro-2H-thieno[2,3-d]pyrimidin-3-ylmethyl]-benzoic acid; compound with trifluoro-acetic acid as an off white solid (15%). MS (APCI+), m/z 549 (M+).

### EXAMPLE 30

### 4-[6-(2-Ethoxy-benzylcarbamoyl)-1-methyl-2,4-dioxo-1,2,3,4-tetrahydro-thieno[2,3-d]pyrimidine-3-ylmethyl]-benzoic acid

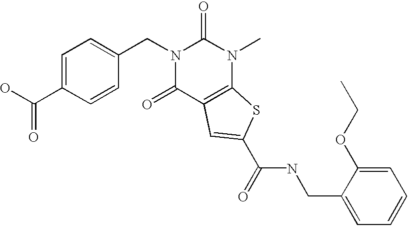

The procedure of Example 28 was repeated, 4-fluorobenzyl amine is replaced by 1-ethoxy-2-ethyl-benzene, to give 4-[6-(2-ethoxy-benzylcarbamoyl)-1-methyl-2,4-dioxo-1,2,3,4-tetrahydro-thieno[2,3-d]pyrimidine-3-ylmethyl]-benzoic acid as an off white solid (20%). MS (APCI+), m/z 494 (M+).

### EXAMPLE 31

### 1-Methyl-2,4-dioxo-1,2,3,4-tetrahydro-thieno[2,3-d]pyrimidine-6-carboxylic acid

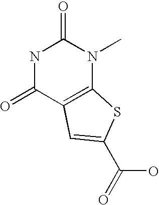

1-Methyl-2,4-dioxo-1,2,3,4-tetrahydro-thieno[2,3-d]pyrimidine-6-carboxylic acid benzyl ester (4.45 g, 14.1 mmol) was put in 100 mL of HBr in Acetic acid. The solution was stirred at room temperature for overnight. The precipitant was filtered and washed with excess water to yield 1-methyl-2,4-dioxo-1,2,3,4-tetrahydro-thieno[2,3-d]pyrimidine-6-carboxylic acid as a white solid (2.89 g). MS (APCI+), m/z 227 (M+).

### EXAMPLE 32

### 1-Methyl-2,4-dioxo-1,2,3,4-tetrahydro-thieno[2,3-d]pyrimidine-6-carboxylic acid 3-methoxy-benzylamide

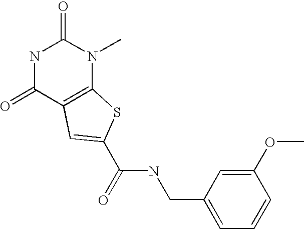

To a suspension of 1-methyl-2,4-dioxo-1,2,3,4-tetrahydro-thieno[2,3-d]pyrimidine-6-carboxylic acid (1.25 g, 5.53 mmol) in 50 mL of 2:1 CH₂Cl₂/THF was added HOBT (0.821 g, 6.08 mmol), 4-methyl morpholine (2.79 g, 27.6 mmol), 4-methoxy benzyl amine (0.91 g, 6.63 mmol) and EDAC (1.27 g, 6.631 mmol) in that order. The reaction is stirred at room temperature for overnight, and then was acidified by 5% HCl. The reaction was diluted with 100 mL of CH₂Cl₂ and was shaken. The precipitant was filtered and washed with 100 mL of 5% HCl and 100 mL of 5% NaHCO₃ to yield 1-methyl-2,4-dioxo-1,2,3,4-tetrahydro-thieno[2,3-d]pyrimidine-6-carboxylic acid 3-methoxy-benzylamide as a white solid (79%). MS (APCI+), m/z 346 (M+).

### EXAMPLE 33

### 1-Methyl-2,4-dioxo-3-[4-(1H-tetrazol-5-yl)-benzyl]-1,2,3,4-tetrahydro-thieno[2,3-d]pyrimidine-6-carboxylic acid 3-methoxy-benzylamide

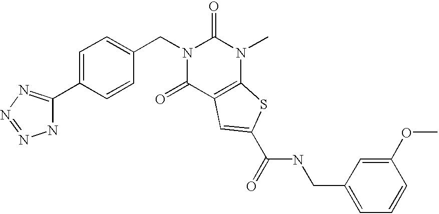

To a solution of 1-methyl-2,4-dioxo-1,2,3,4-tetrahydro-thieno[2,3-d]pyrimidine-6-carboxylic acid (0.58 g, 2.07 mmol) in DMF was added cesium carbonate (0.68 g, 2.08 mmol) and 5-(4-bromomethyl-phenyl)-2-phenyl-2H-tetrazole (1.0 g, 2.08 mmol). The solution was stirred overnight at room temperature. 170 mL of water was then added, causing precipitation. The precipitant was filtered and then stirred in excess TFA at room temperature for overnight, concentrated and washed with Hexane and ether to give 3-(3-methoxycarbonyl-benzyl)-1-methyl-2,4-dioxo-1,2,3,4-tetrahydro-thieno[2,3-d]pyrimidine-6-carboxylic acid benzyl ester as an off white solid (61%). MS (APCI+), m/z 504 (M+).

### EXAMPLE 34

### 1-Methyl-3-[4-(morpholine-4-sulfonyl)-benzyl]-2,4-dioxo-1,2,3,4-tetrahydro-thieno[2,3-d]pyrimidine-6-carboxylic acid 3-methoxy-benzylamide

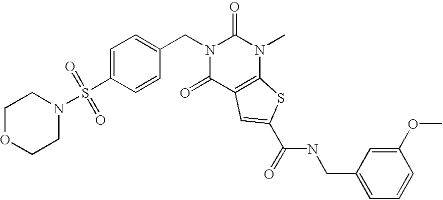

To a solution of 1-methyl-2,4-dioxo-1,2,3,4-tetrahydro-thieno[2,3-d]pyrimidine-6-carboxylic acid 3-methoxy-benzylamide (0.3 g, 0.87 mmol) in 50 mL of DMF was added cesium carbonate (0.283 g, 0.87 mmol) and 4-(4-bromomethyl-benzenesulfonyl)-morpholine (0.287 g, 0.87 mmol). The reaction was then stirred at room temperature overnight. The solution was then poured into 500 mL of water and extracted with EtOAc. The organic layer was washed with water and brine, dried over MgSO₄ and concentrated. The residue was triturated with 4:1 Hexane/EtOAc to yield 1-methyl-3-[4-(morpholine-4-sulfonyl)-benzyl]-2,4-dioxo-1,2,3,4-tetrahydro-thieno[2,3-d]pyrimidine-6-carboxylic acid 3-methoxy-benzylamide as a white solid (66%). MS (APCI+), m/z 585 (M+).

### EXAMPLE 35

### 1-Methyl-3-[4-(morpholine-4-carbonyl)-benzyl]-2,4-dioxo-1,2,3,4-tetrahydro-thieno[2,3-d]pyrimidine-6-carboxylic acid 3-methoxy-benzylamide

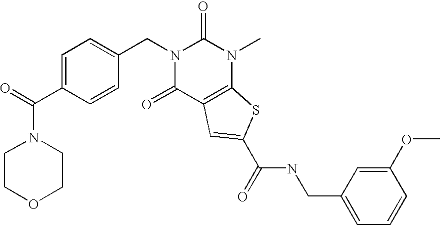

The procedure of Example 34 was repeated, except 4-(4-bromomethyl-benzenesulfonyl)-morpholine is replaced by 1-(4-bromomethyl-phenyl)-1-morpholin-4-yl-methanone, to give 1-methyl-3-[4-(morpholine-4-carbonyl)-benzyl]-2,4-dioxo-1,2,3,4-tetrahydro-thieno[2,3-d]pyrimidine-6-carboxylic acid 3-methoxy-benzylamide as an off white solid (25%). MS (APCI+), m/z 459 (M+).

### EXAMPLE 36

### 3-But-2-ynyl-1-methyl-2,4-dioxo-1,2,3,4-tetrahydro-thieno[2,3-d]pyrimidine-6-carboxylic acid 3-methoxy-benzylamide

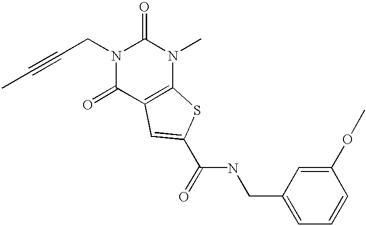

The procedure of Example 34 was repeated, except 4-(4-bromomethyl-benzenesulfonyl)-morpholine is replaced by 1-bromo-but-2-yne, to give 3-but-2-ynyl-1-methyl-2,4-dioxo-1,2,3,4-tetrahydro-thieno[2,3-d]pyrimidine-6-carboxylic acid 3-methoxy-benzylamide as an off white solid (97%). MS (APCI+), m/z 398 (M+).

### EXAMPLE 37

### 1-Methyl-2,4-dioxo-3-[3-(1H-tetrazol-5-yl)-benzyl]-1,2,3,4-tetrahydro-thieno[2,3-d]pyrimidine-6-carboxylic acid 3-methoxy-benzylamide

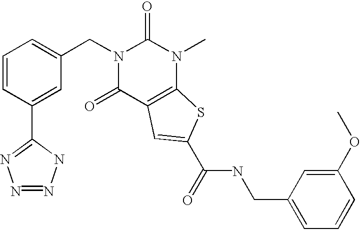

The procedure of Example 33 was repeated, except 5-(4-bromomethyl-phenyl)-2-phenyl-2H-tetrazole is replaced by except 5-(3-bromomethyl-phenyl)-2-phenyl-2H-tetrazole, to give 1-methyl-2,4-dioxo-3-[3-(1H-tetrazol-5-yl)-benzyl]-1,2,3,4-tetrahydro-thieno[2,3-d]pyrimidine-6-carboxylic acid 3-methoxy-benzylamide as an off white solid (70%). MS (APCI+), m/z 504 (M+).

### EXAMPLE 38

### 3-(4-Cyano-benzyl)-1-methyl-2,4-dioxo-1,2,3,4-tetrahydro-thieno[2,3-d]pyrimidine-6-carboxylic acid 3-methoxy-benzylamide

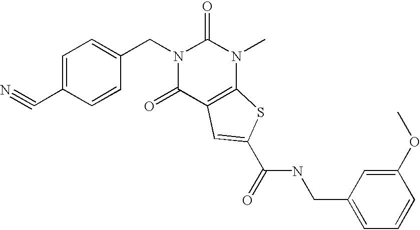

The procedure of Example 34 was repeated, except 4-(4-bromomethyl-benzenesulfonyl)-morpholine is replaced by 4-bromomethyl benzonitrile, to give 3-(4-cyano-benzyl)-1-methyl-2,4-dioxo-1,2,3,4-tetrahydro-thieno[2,3-d]pyrimidine-6-carboxylic acid 3-methoxy-benzylamide as an off white solid (70%). MS (APCI+), m/z 431 (M−).

### EXAMPLE 39

### {4-[6-(3-Methoxy-benzylcarbamoyl)-1-methyl-2,4-dioxo-1,4-dihydro-2H-thieno[2,3-d]pyrimidin-3-ylmethyl]-phenyl}-acetic acid

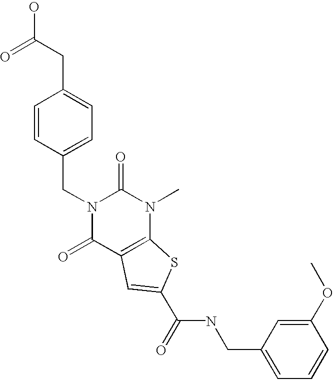

The procedure of Example 33 was repeated, except 5-(4-bromomethyl-phenyl)-2-phenyl-2H-tetrazole is replaced by (4-bromomethyl-phenyl)-acetic acid tert-butyl ester, to give {4-[6-(3-methoxy-benzylcarbamoyl)-1-methyl-2,4-dioxo-1,4-dihydro-2H-thieno[2,3-d]pyrimidin-3-ylmethyl]-phenyl}-acetic acid as a white solid (70%). MS (APCI+), m/z 494 (M+).

### EXAMPLE 40

### 3-[2-(2,4-Dichloro-benzenesulfonyl)-ethyl]-1-methyl-2,4-dioxo-1,2,3,4-tetrahydro-thieno[2,3-d]pyrimidine-6-carboxylic acid 3-methoxy-benzylamide

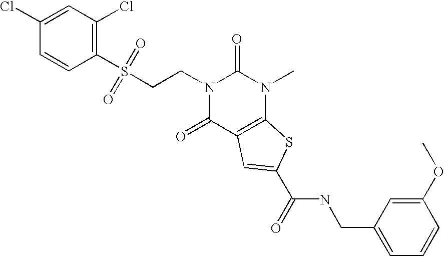

The procedure of Example 34 was repeated, except 4-(4-bromomethyl-benzenesulfonyl)-morpholine is replaced by 2,4-dichloro-1-(2-chloro-ethanesulfonyl)-benzene, to 3-[2-(2,4-dichloro-benzenesulfonyl)-ethyl]-1-methyl-2,4-dioxo-1,2,3,4-tetrahydro-thieno[2,3-d]pyrimidine-6-carboxylic acid 3-methoxy-benzylamide as a white solid. MS (APCI+), m/z 582 (M+)

### EXAMPLE 41

### 3-(4-Methanesulfonyl-benzyl)-1-methyl-2,4-dioxo-1,2,3,4-tetrahydro-thieno[2,3-d]pyrimidine-6-carboxylic acid 3-methoxy-benzylamide

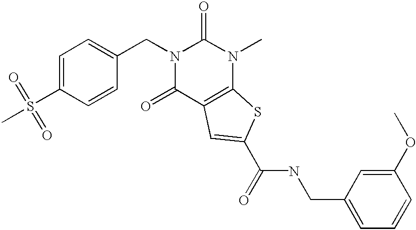

The procedure of Example 34 was repeated, except 4-(4-bromomethyl-benzenesulfonyl)-morpholine is replaced by 1-chloromethyl-4-methanesulfonyl-benzene, to give 3-(4-methanesulfonyl-benzyl)-1-methyl-2,4-dioxo-1,2,3,4-tetrahydro-thieno[2,3-d]pyrimidine-6-carboxylic acid 3-methoxy-benzylamide as a white solid. MS (APCI+), m/z 514 (M+)

### EXAMPLE 42

### 1-Methyl-2,4-dioxo-3-(4-sulfamoyl-benzyl)-1,2,3,4-tetrahydro-thieno[2,3-d]pyrimidine-6-carboxylic acid 3-methoxy-benzylamide

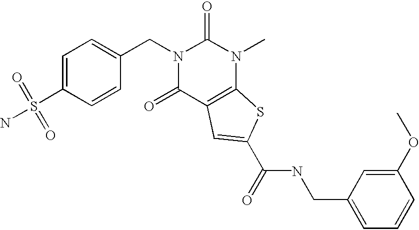

The procedure of Example 34 was repeated, except 4-(4-bromomethyl-benzenesulfonyl)-morpholine is replaced by 4-bromomethyl-benzenesulfonamide, to give 1-methyl-2,4-dioxo-3-(4-sulfamoyl-benzyl)-1,2,3,4-tetrahydro-thieno[2,3-d]pyrimidine-6-carboxylic acid 3-methoxy-benzylamide as a white solid. MS (APCI+), m/z 515 (M+)

### EXAMPLE 43

### 3-Benzyl-1-methyl-2,4-dioxo-1,2,3,4-tetrahydro-thieno[2,3-d]pyrimidine-6-carboxylic acid (2-methoxy-pyridin-4-ylmethyl)-amide

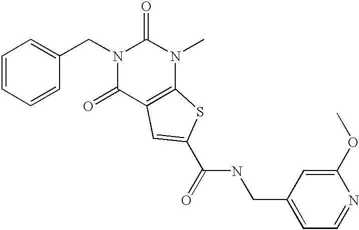

The procedure of Example 34 was repeated, except 4-(4-bromomethyl-benzenesulfonyl)-morpholine is replaced by benzyl bromide, and the amide starting material was a 2-methoxy-pyridine-4-yl methyl amide, to give 3-benzyl-1-methyl-2,4-dioxo-1,2,3,4-tetrahydro-thieno[2,3-d]pyrimidine-6-carboxylic acid (2-methoxy-pyridin-4-ylmethyl)-amide as a white solid (30%). ¹H NMR (DMSO), δ3.47 (s, 3H), 3.81 (s, 3H), 4.41 (d, J=7.0 Hz, 2H), 5.03 (s, 2H), 6.66 (s, 1H), 6.88 (d, J=4.9 Hz, 1H), 7.21-7.36 (m, 5H), 8.08 (d, J=7.0 Hz, 2H), 8.14 (s, 1H), 9.27 (t, J=7.0 Hz, 1H).

### EXAMPLE 44

### 1-Methyl-3-(4-methylsulfamoyl-benzyl)-2,4-dioxo-1,2,3,4-tetrahydro-thieno[2,3-d]pyrimidine-6-carboxylic acid 3-methoxy-benzylamide

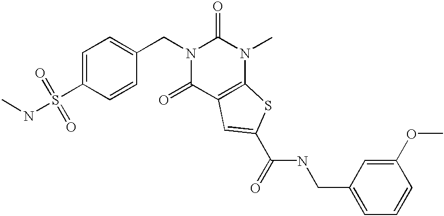

The procedure of Example 34 was repeated, except 4-(4-bromomethyl-benzenesulfonyl)-morpholine is replaced by 4-bromomethyl-N-methyl-benzenesulfonamide, to give 1-methyl-3-(4-methylsulfamoyl-benzyl)-2,4-dioxo-1,2,3,4-tetrahydro-thieno[2,3-d]pyrimidine-6-carboxylic acid 3-methoxy-benzylamide as a white solid. ¹H NMR (DMSO), δ2.37 (d, J=5.0 Hz, 3H), 3.47 (s, 3H), 3.72 (s, 3H), 4.41 (d, J=5.9 Hz, 2H), 5.12 (s, 2H), 6.80-6.87 (m, 3H), 7.24 (t, J=8.0 Hz, 1H), 7.40-7.51 (m, 3H), 7.69 (d, J=8.0 Hz, 2H), 8.13 (s, 1H), 9.21 (t,J=5.7 Hz, 1H).

### EXAMPLE 45

### 3-(4-Isopropylsulfamoyl-benzyl)-1-methyl-2,4-dioxo-1,2,3,4-tetrahydro-thieno[2,3-d]pyrimidine-6-carboxylic acid 3-methoxy-benzylamide

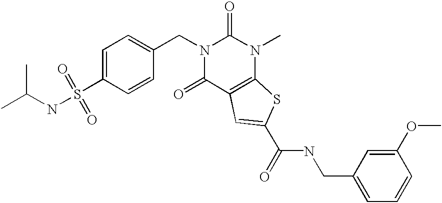

The procedure of Example 34 was repeated, except 4-(4-bromomethyl-benzenesulfonyl)-morpholine is replaced by 4-bromomethyl-N-isopropyl-benzenesulfonamide, to give 3-(4-isopropylsulfamoyl-benzyl)-1-methyl-2,4-dioxo-1,2,3,4-tetrahydro-thieno[2,3-d]pyrimidine-6-carboxylic acid 3-methoxy-benzylamide as a white solid. ¹H NMR (DMSO), δ0.92 (d, J=6.6 Hz, 6H), 3.48 (s, 3H), 3.72 (s, 3H), 4.41 (d, J=5.8 Hz, 2H), 5.12 (s, 2H), 6.80-6.87 (m, 3H), 7.24 (t, J=8.1 Hz, 1H), 7.46-7.55 (m, 3H), 7.71 (d, J=8.0 Hz, 2H), 8.13 (s, 1H), 9.21 (t, J=5.7 Hz, 1H).

### EXAMPLE 46

### 1-Methyl-2,4-dioxo-3-[4-(pyrrolidine-1-sulfonyl)-benzyl]-1,2,3,4-tetrahydro-thieno[2,3-d]pyrimidine-6-carboxylic acid 3-methoxy-benzylamide

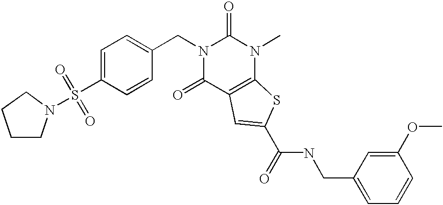

The procedure of Example 34 was repeated, except 4-(4-bromomethyl-benzenesulfonyl)-morpholine is replaced by 1-(4-bromomethyl-benezensulfonyl)-pyrrolidine, to give 1-methyl-2,4-dioxo-3-[4-(pyrrolidine-1-sulfonyl)-benzyl]-1,2,3,4-tetrahydro-thieno[2,3-d]pyrimidine-6-carboxylic acid 3-methoxy-benzylamide as a white solid. ¹H NMR (DMSO), δ1.64 (m, 4H), 3.11 (m, 4H), 3.49 (s, 3H), 3.73 (s, 3H), 4.42 (d, J=5.8 Hz, 2H), 5.14 (s, 2H), 6.83-6.88 (m, 3H), 7.24 (m, 1H), 7.53 (d, J=8.1, 2H), 7.73 (d, J=8.0 Hz, 2H), 8.13 (s, 1H), 9.21 (t, J=5.7 Hz, 1H).

### EXAMPLE 47

### 1-Methyl-3-[4-(4-methyl-piperidine-1-sulfonyl)-benzyl]-2,4-dioxo-1,2,3,4-tetrahydro-thieno[2,3-d]pyrimidine-6-carboxylic acid 3-methoxy-benzylamide

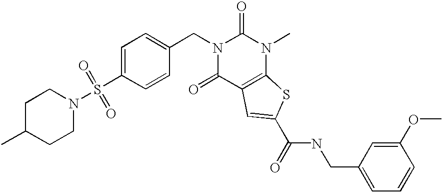

The procedure of Example 34 was repeated, except 4-(4-bromomethyl-benzenesulfonyl)-morpholine is replaced by 1-(4-bromomethyl-benzenesulfonyl)-4-methyl-piperidine, to give 1-methyl-3-[4-(4-methyl-piperidine-1-sulfonyl)-benzyl]-2,4-dioxo-1,2,3,4-tetrahydro-thieno[2,3-d]pyrimidine-6-carboxylic acid 3-methoxy-benzylamide as a white solid. ¹H NMR (DMSO), δ0.83 (d, J=5.4 Hz, 3H), 1.15 (m, 2H), 1.28 (s, 1H), 1.62 (d, J=12.7 Hz, 2H), 2.16 (t, J=12.3 Hz, 2H), 3.49 (s, 3H), 3.58 (d, J=10.5 Hz, 2H), 3.73 (s, 3H), 4.43 (d, J=5.1 Hz, 2H), 5.15 (s, 2H), 6.81-6.87 (m, 3H), 7.24 (m, 1H), 7.53 (d, J=7.1, 2H), 7.66 (d, J=6.8 Hz, 2H), 8.14 (s, 1H), 9.23 (t, J=5.7 Hz, 1H).

### EXAMPLE 48

### 3-Benzyl-1-methyl-2,4-dioxo-1,2,3,4-tetrahydro-thieno[2,3-d]pyrimidine-6-carboxylic acid benzofuran-2-ylmethyl ester

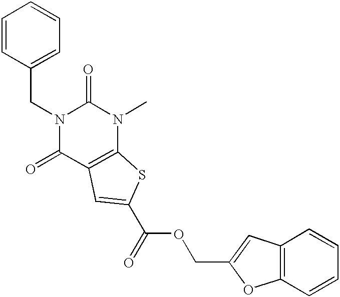

To a solution of 3-benzyl-1-methyl-2,4-dioxo-1,2,3,4-tetrahydro-thieno[2,3-d]pyrimidine-6-carboxylic acid (0.2 g, 0.63 mmol) and Mukiyama reagent (0.194 g, 0.76 mmol) in 6 mL of CH₂Cl₂ was added Et₃N (0.154 g, 1.52 mmol) and benzofuran-2-yl-methanol (0.103 g, 0.696 mmol). The reaction solution was stirred at room temperature for overnight. The reaction solution was then chromatographed using 4:1 Hexane/EtOAc, to yield 3-benzyl-1-methyl-2,4-dioxo-1,2,3,4-tetrahydro-thieno[2,3-d]pyrimidine-6-carboxylic acid benzofuran-2-ylmethyl ester as a white solid (135 mg, 48%). MS (APCI+), m/z 447 (M+).

### EXAMPLE 49

### 3-(4-Bromo-benzyl)-2,4-dioxo-1,2,3,4-tetrahydro-thieno[2,3-d]pyrimidine-6-carboxylic acid pyridin-4-ylmethyl ester

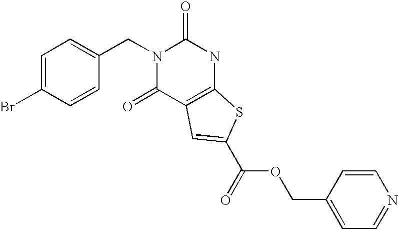

The procedure of Example 2 was repeated, except the 3-benzyl-2,4-dioxo-1,2,3,4-tetrahydro-thieno[2,3-d]pyrimidine-6-carboxylic acid was replaced by 3-(4-bromo-benzyl)-2,4-dioxo-1,2,3,4-tetrahydro-thieno[2,3-d]pyrimidine-6-carboxylic acid made using 4-bromo in place of benzyl during the synthesis outlined in preparation 1-3, to give 3-(4-bromo-benzyl)-2,4-dioxo-1,2,3,4-tetrahydro-thieno[2,3-d]pyrimidine-6-carboxylic acid pyridin-4-ylmethyl ester as an off white solid. MS (APCI+), m/z 472 (M+).

### EXAMPLE 50

### 3-Benzyl-2,4-dioxo-1,2,3,4-tetrahydro-thieno[2,3-d]pyrimidine-6-carboxylic acid 4-methoxy-benzyl ester

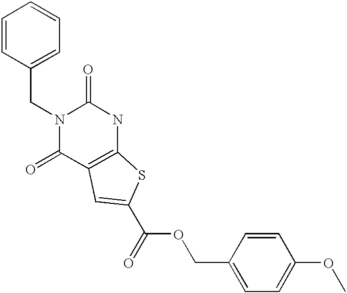

The procedure of Example 1 was repeated, except that benzyl alcohol is replaced by 4-methoxy benzyl alcohol to provide 3-benzyl-2,4-dioxo-1,2,3,4-tetrahydro-thieno[2,3-d]pyrimidine-6-carboxylic acid 4-methoxy-benzyl ester as a white powder (30 mg, 10%). MS (APCI+), m/z 423 (M+).

### EXAMPLE 51

### 4-{1-Methyl-2,4-dioxo-6-[(pyridin-4-ylmethyl)-carbamoyl]-1,4-dihydro-2H-thieno[2,3-d]pyrimidin-3-ylmethyl}-benzoic acid; compound with trifluoro-acetic acid

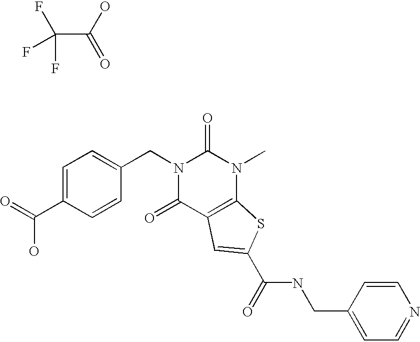

The procedure of Example 28 was repeated, except that 4-fluorobenzyl amine is replaced by (4-methylamino) pyridine, to give 4-{1-methyl-2,4-dioxo-6-[(pyridin-4-ylmethyl)-carbamoyl]-1,4-dihydro-2H-thieno[2,3-d]pyrimidin-3-ylmethyl}-benzoic acid; compound with trifluoro-acetic acid as an off white solid (82%). MS (APCI+), m/z 451 (M+).

### EXAMPLE 52

### 4-[6-(4-Methoxy-benzylcarbamoyl)-1-methyl-2,4-dioxo-1,4-dihydro-2H-thieno[2,3-d]pyrimidin-3-ylmethyl]-benzoic acid

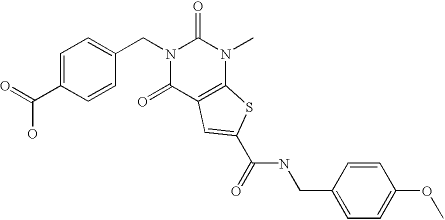

The procedure of Example 28 was repeated, except that 4-fluorobenzyl amine is replaced by 4-methoxy benzyl amine, to give 4-[6-(4-methoxy-benzylcarbamoyl)-1-methyl-2,4-dioxo-1,4-dihydro-2H-thieno[2,3-d]pyrimidin-3-ylmethyl]-benzoic acid as an off white solid (38%). MS (APCI+), m/z 480 (M+).

### EXAMPLE 53

### 4-[6-(3,4-Dimethoxy-benzylcarbamoyl)-1-methyl-2,4-dioxo-1,4-dihydro-2H-thieno[2,3-d]pyrimidin-3-ylmethyl]-benzoic acid tert-butyl ester

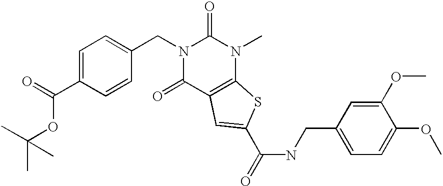

To a solution of 3-(4-tert-butyoxycarbonyl-benzyl)-1-methyl-2,4-dioxo-1,2,3,4-tetrahydro-thieno[2,3-d]pyrimidine-6-carboxylic acid (0.15 g, 0.36 mmol) and Mukiyama reagent (0.11 g, 0.43 mmol) in 6 mL of CH₂Cl₂ was added Et₃N (0.87 g, 87 mmol) and 3,4-dimethoxy benzyl amine (0.067 g, 0.39 mmol). The reaction solution was stirred at room temperature for overnight. The reaction solution was then chromatographed using 4:1 Hexane/EtOAc, to yield 4-[6-(3,4-dimethoxy-benzylcarbamoyl)-1-methyl-2,4-dioxo-1,4-dihydro-2H-thieno[2,3-d]pyrimidin-3-ylmethyl]-benzoic acid tert-butyl ester as a white solid (78%). MS (APCI+), m/z 567 (M+).

### EXAMPLE 54

### 4-[6-(3,4-Dimethoxy-benzylcarbamoyl)-1-methyl-2,4-dioxo-1,4-dihydro-2H-thieno[2,3-d]pyrimidin-3-ylmethyl]-benzoic acid

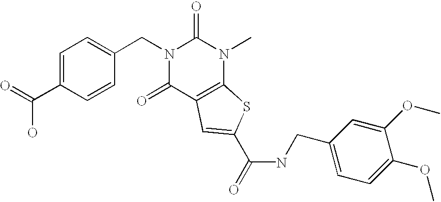

4-[6-(3,4-Dimethoxy-benzylcarbamoyl)-1-methyl-2,4-dioxo-1,4-dihydro-2H-thieno[2,3-d]pyrimidin-3-ylmethyl]-benzoic acid tert-butyl ester (0.1 g, 0.176 mmol) was dissolved in 5 mL of TFA. The solution was stirred at room temperature for 30 minutes then concentrated. The residue was triturated with 4:1 Hexane/EtOAc to yield 4-[6-(3,4-dimethoxy-benzylcarbamoyl)-1-methyl-2,4-dioxo-1,4-dihydro-2H-thieno[2,3-d]pyrimidin-3-ylmethyl]-benzoic acid as a white solid (65 mg, 73%). MS (APCI+), m/z 510 (M+).

### EXAMPLE 55

### 4-[6-(4-Bromo-benzylcarbamoyl)-1-methyl-2,4-dioxo-1,4-dihydro-2H-thieno[2,3-d]pyrimidin-3-ylmethyl]-benzoic acid

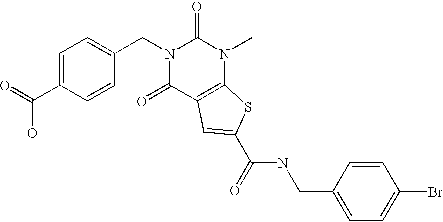

The procedure of Example 28 was repeated, except that 4-fluorobenzyl amine is replaced by 4-bromobenzyl amine, to give 4-[6-(4-bromo-benzylcarbamoyl)-1-methyl-2,4-dioxo-1,4-dihydro-2H-thieno[2,3-d]pyrimidin-3-ylmethyl]-benzoic acid as an off white solid (55%). MS (APCI+), m/z 530 (M+).

### EXAMPLE 56

### 4-[6-(4-Bromo-benzylcarbamoyl)-1-methyl-2,4-dioxo-1,4-dihydro-2H-thieno[2,3-d]pyrimidin-3-ylmethyl]-benzoic acid tert-butyl ester

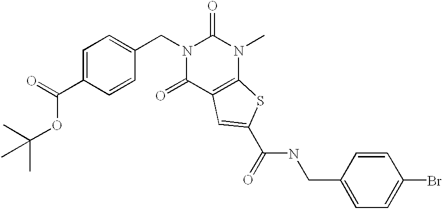

The procedure of Example 53 was repeated, except that 3,4-dimethoxy benzyl amine is replaced by 4-bromobenzyl amine, to give 4-[6-(4-bromo-benzylcarbamoyl)-1-methyl-2,4-dioxo-1,4-dihydro-2H-thieno[2,3-d]pyrimidin-3-ylmethyl]-benzoic acid tert-butyl ester as an off white solid (71%). MS (APCI−), m/z 584 (M−).

### EXAMPLE 57

### 4-[6-(3,5-Bis-trifluoromethyl-benzylcarbamoyl)-1-methyl-2,4-dioxo-1,4-dihydro-2H-thieno[2,3-d]pyrimidin-3-ylmethyl]-benzoic acid

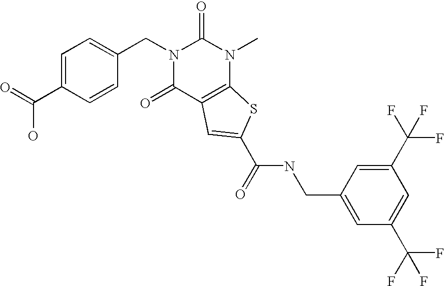

The procedure of Example 28 was repeated, except that 4-fluorobenzyl amine is replaced by 3,5-bis-trifluoromethyl benzyl amine, to give 4-[6-(3,5-bis-trifluoromethyl-benzylcarbamoyl)-1-methyl-2,4-dioxo-1,4-dihydro-2H-thieno[2,3-d]pyrimidin-3-ylmethyl]-benzoic acid as an off white solid (65%). MS (APCI+), m/z 586 (M+).

### EXAMPLE 58

### 4-[6-(4-Chloro-benzylcarbamoyl)-1-methyl-2,4-dioxo-1,4-dihydro-2H-thieno[2,3-d]pyrimidin-3-ylmethyl]-benzoic acid

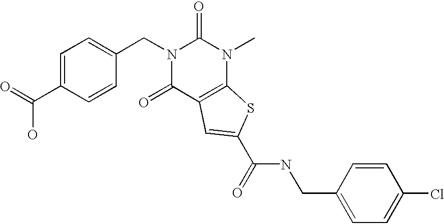

The procedure of Example 28 was repeated, 4-fluorobenzyl amine is replaced by 4-chlorobenzyl amine, to give 4-[6-(4-chloro-benzylcarbamoyl)-1-methyl-2,4-dioxo-1,4-dihydro-2H-thieno[2,3-d]pyrimidin-3-ylmethyl]-benzoicacid as an off white solid (39%). MS (APCI+), m/z 484 (M+).

### EXAMPLE 59

### 4-[1-Methyl-2,4-dioxo-6-(4-sulfamoyl-benzylcarbamoyl)-1,4-dihydro-2H-thieno[2,3-d]pyrimidin-3-ylmethyl]-benzoic acid

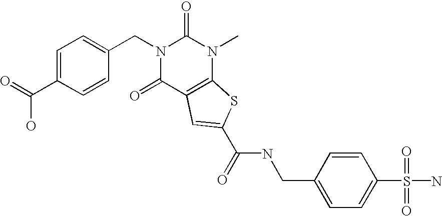

The procedure of Example 28 was repeated, except that 4-fluorobenzyl amine is replaced by 4-aminoethyl-benzenesulfonamide, to give 4-[1-methyl-2,4-dioxo-6-(4-sulfamoyl-benzylcarbamoyl)-1,4-dihydro-2H-thieno[2,3-d]pyrimidin-3-ylmethyl]-benzoic acid as an off white solid (41%). MS (APCI+), m/z 529 (M+).

### EXAMPLE 60

### 3-(4-Fluoro-benzyl)-1-methyl-2,4-dioxo-1,2,3,4-tetrahydro-thieno[2,3-d]pyrimidine-6-carboxylic acid 4-methoxy-benzylamide

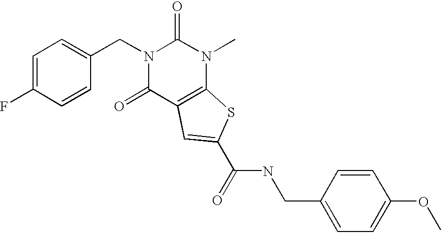

The procedure of Example 34 was repeated, except that 4-(4-bromomethyl-benzenesulfonyl)-morpholine is replaced by 1-bromomethyl-4-fluoro-benzene, and 1-methyl-2,4-dioxo-1,2,3,4-tetrahydro-thieno[2,3-d]pyrimidine-6-carboxylic acid 3-methoxy-benzylamide is replaced by 1-methyl-2,4-dioxo-1,2,3,4-tetrahydro-thieno[2,3-d]pyrimidine-6-carboxylic acid 4-methoxy-benzylamide, to give 3-(4-fluoro-benzyl)-1-methyl-2,4-dioxo-1,2,3,4-tetrahydro-thieno[2,3-d]pyrimidine-6-carboxylic acid 4-methoxy-benzylamide as an off white solid (65%). MS (APCI+), m/z 454 (M+).

### EXAMPLE 61

### 3-(4-Iodo-benzyl)-1-methyl-2,4-dioxo-1,2,3,4-tetrahydro-thieno[2,3-d]pyrimidine-6-carboxylic acid 3-methoxy-benzylamide

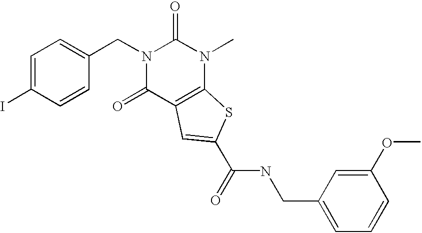

The procedure of Example 34 was repeated, except that 4-(4-bromomethyl-benzenesulfonyl)-morpholine is replaced by 1-bromomethyl-4-iodo-benzene, to give 3-(4-iodo-benzyl)-1-methyl-2,4-dioxo-1,2,3,4-tetrahydro-thieno[2,3-d]pyrimidine-6-carboxylic acid 3-methoxy-benzylamide as an off white solid (27%). MS (APCI+), m/z 562 (M+).

### EXAMPLE 62

### 3-(4-Dimethylsulfamoyl-benzyl)-1-methyl-2,4-dioxo-1,2,3,4-tetrahydro-thieno[2,3-d]pyrimidine-6-carboxylic acid 4-methoxy-benzylamide

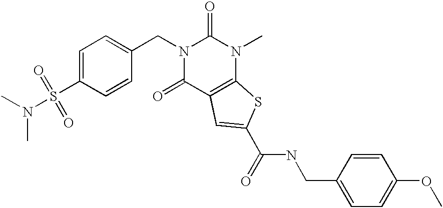

The procedure of Example 34 was repeated, except that 4-(4-bromomethyl-benzenesulfonyl)-morpholine is replaced by 4-bromomethyl-N,N-dimethyl-benzenesulfonamide, and 1-methyl-2,4-dioxo-1,2,3,4-tetrahydro-thieno[2,3-d]pyrimidine-6-carboxylic acid 3-methoxy-benzylamide is replaced by 1-methyl-2,4-dioxo-1,2,3,4-tetrahydro-thieno[2,3-d]pyrimidine-6-carboxylic acid 4-methoxy-benzylamide, to give 3-(4-dimethylsulfamoyl-benzyl)-1-methyl-2,4-dioxo-1,2,3,4-tetrahydro-thieno[2,3-d]pyrimidine-6-carboxylic acid 4-methoxy-benzylamide as an off white solid (67%). MS (APCI+), m/z 543 (M+).

### EXAMPLE 63

### 3-(3-Methoxy-benzyl)-1-methyl-2,4-dioxo-1,2,3,4-tetrahydro-thieno[2,3-d]pyrimidine-6-carboxylic acid 4-methoxy-benzylamide

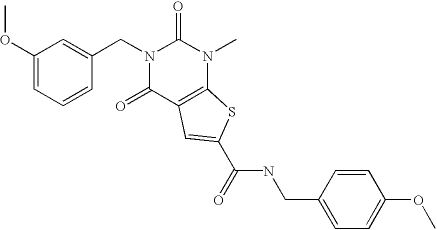

The procedure of Example 34 was repeated, except that 4-(4-bromomethyl-benzenesulfonyl)-morpholine is replaced by 3-methoxy benzyl bromide, and 1-methyl-2,4-dioxo-1,2,3,4-tetrahydro-thieno[2,3-d]pyrimidine-6-carboxylic acid 3-methoxy-benzylamide is replaced by 1-methyl-2,4-dioxo-1,2,3,4-tetrahydro-thieno[2,3-d]pyrimidine-6-carboxylic acid 4-methoxy-benzylamide, to give 3-(3-methoxy-benzyl)-1-methyl-2,4-dioxo-1,2,3,4-tetrahydro-thieno[2,3-d]pyrimidine-6-carboxylic acid 4-methoxy-benzylamide as an off white solid (83%). MS (APCI+), m/z 5466 (M+).

### EXAMPLE 64

### 3-(4-Cyano-benzyl)-1-methyl-2,4-dioxo-1,2,3,4-tetrahydro-thieno[2,3-d]pyrimidine-6-carboxylic acid 4-methoxy-benzylamide

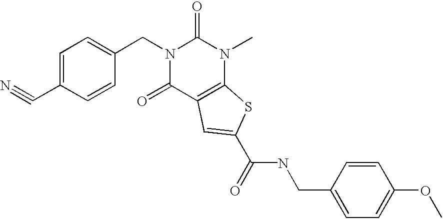

The procedure of Example 34 was repeated, except that 4-(4-bromomethyl-benzenesulfonyl)-morpholine is replaced by 4-cyanobenzyl bromide, and 1-methyl-2,4-dioxo-1,2,3,4-tetrahydro-thieno[2,3-d]pyrimidine-6-carboxylic acid 3-methoxy-benzylamide is replaced by 1-methyl-2,4-dioxo-1,2,3,4-tetrahydro-thieno[2,3-d]pyrimidine-6-carboxylic acid 4-methoxy-benzylamide, to give 3-(4-cyano-benzyl)-1-methyl-2,4-dioxo-1,2,3,4-tetrahydro-thieno[2,3-d]pyrimidine-6-carboxylic acid 4-methoxy-benzylamide as an off white solid (97%). MS (APCI+), m/z 461 (M+).

### EXAMPLE 65

### 3-(4-Acetylamino-benzyl)-1-methyl-2,4-dioxo-1,2,3,4-tetrahydro-thieno[2,3-d]pyrimidine-6-carboxylic acid 3-methoxy-benzylamide

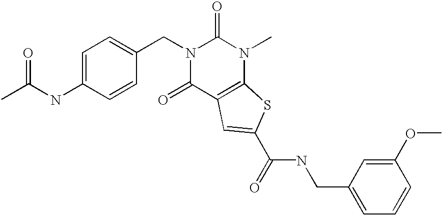

The procedure of Example 34 was repeated, except that 4-(4-bromomethyl-benzenesulfonyl)-morpholine is replaced by N-(4-chloromethyl-phenyl)-acetamide, to give 3-(4-acetylamino-benzyl)-1-methyl-2,4-dioxo-1,2,3,4-tetrahydro-thieno[2,3-d]pyrimidine-6-carboxylic acid 3-methoxy-benzylamide as an off white solid (42%). MS (APCI+), m/z 493 (M+).

### EXAMPLE 65a

### 5-[6-(3-Methoxy-benzylcarbamoyl)-1-methyl-2,4-dioxo-1,4-dihydro-2H-thieno[2,3-d]pyrimidin-3-ylmethyl]-furan-2-carboxylic acid ethyl ester

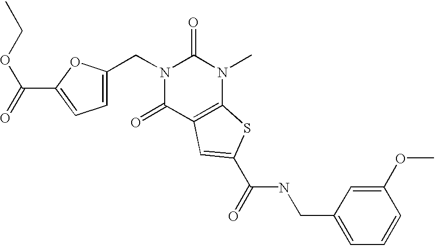

The procedure of Example 34 was repeated, except that 4-(4-bromomethyl-benzenesulfonyl)-morpholine is replaced by 5-chloromethyl-furan-2-carboxylic acid ethyl ester, to give 5-[6-(3-methoxy-benzylcarbamoyl)-1-methyl-2,4-dioxo-1,4-dihydro-2H-thieno[2,3-d]pyrimidin-3-ylmethyl]-furan-2-carboxylic acid ethyl ester as an off which solid (41%). MS (APCI+), m/z 498 (M+).

### EXAMPLE 66

### 3-(4-Cyano-benzyl)-2,4-dioxo-1,2,3,4-tetrahydro-thieno[2,3-d]pyrimidine-6-carboxylic acid 3-methoxy-benzyl ester

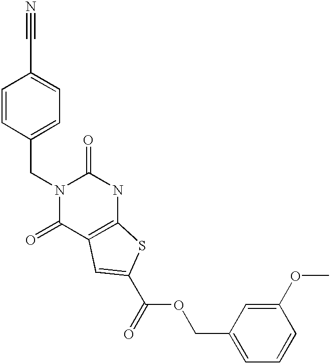

The procedure of preparation 2 was repeated, except that 3-benzyl-2,4-dioxo-1,2,3,4-tetrahydro-thieno[2,3-d]pyrimidine-6-carboxylic acid ethyl ester is replaced by [1-(4-cyano-benyl)-2,6-dioxo-1,2,3,6-tetrahydro-pyrimidine-4-sulfanyl-acetic acid 3-methoxy-benzyl ester, to yield 3-(4-cyano-benzyl)-2,4-dioxo-1,2,3,4-tetrahydro-thieno[2,3-d]pyrimidine-6-carboxylic acid 3-methoxy-benzyl ester as an off white solid (91%). ¹H NMR (DMSO), δ3.74 (s, 3H), 5.07 (s, 2H), 5.28 (s, 2H), 6.91 (d, J=8.2 Hz, 1H), 6.99 (d, J=3.0 Hz, 2H), 7.30 (t, J=8.0 Hz, 1H), 7.47 (d, J=7.7 Hz, 2H), 7.75 (d, J=8.0 Hz, 2H), 7.83 (s, 1H), 12.68 (s, 1H).

### EXAMPLE 67

### 2,4-Dioxo-3-[4-(2H-tetrazol-4-yl)-benzyl]-1,2,3,4-tetrahydro-thieno[2,3-d]pyrimidine-6-carboxylic acid 3-methoxy-benzyl ester

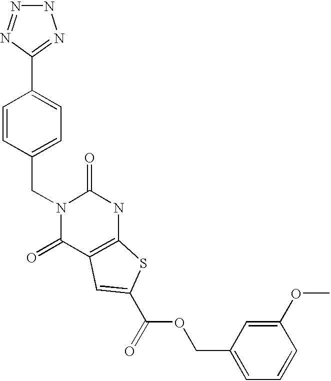

The procedure of Example 24 was repeated, except that 3-(4-cyano-benzyl)-1-methyl-2,4-dioxo-1,2,3,4-tetrahydro[2,3-d]pyrimidine-6-carboxylic acid 3-methoxy-benzyl ester is replaced by 3-(4-cyano-benzyl)-2,4-dioxo-1,2,3,4-tetrahydro[2,3-d]pyrimidine-6-carboxylic acid benzyl ester, to give 2,4-dioxo-3-[4-(2H-tetrazol-5-yl)-benzyl]-1,2,3,4-tetrahydro-thieno[2,3-d]pyrimidine-6-carboxylic acid 3-methoxy-benzyl ester as an off white solid (7%). MS (APCI+), m/z 491 (M+).

### EXAMPLE 68

### 4-[6-(3-Methoxy-benzylcarbamoyl)-1-methyl-2,4-dioxo-1,4-dihydro-2H-thieno[2,3-d]pyrimidin-3-ylmethyl]-benzoic acid 2-dimethylamino-ethyl ester

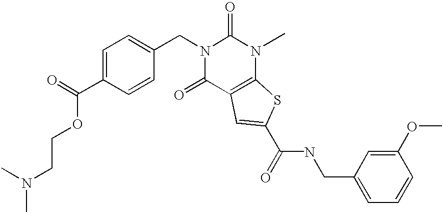

To a solution of 4-[6-(3-methoxy-benzylcarbamoyl)-1-methyl-2,4-dioxo-1,4-dihydro-2H-thieno[2,3-d]pyrimidin-3-ylmethyl]-benzoic acid (100 mg, 0.289 mmol) in 50 mL of 2:1 CH₂Cl₂/THF, was added HOBT (43 mg, 0.32 mmol), 4-methyl morpholine (146 g, 1.44 mmol), 2-dimethylamino-ethanol (28 mg, 0.318 mmol) and EDAC (66.5 mg, 0.347 mmol) in that order. The reaction is stirred at room temperature for overnight, and directly chromatographed with 10:1 CH₂Cl₂/MeOH. The crude product was then triturated with 4:1 Hexane/EtOAc to yield 4-[6-(3-methoxy-benzylcarbamoyl)-1-methyl-2,4-dioxo-1,4-dihydro-2H-thieno[2,3-d]pyrimidin-3-ylmethyl]-benzoic acid 2-dimethylamino-ethyl ester as a white powder (89%). MS (APCI+), m/z 551 (M+).

### EXAMPLE 69

### 3-Cylcohexylmethyl-1-methyl-2,4-dioxo-1,2,3,4-tetrahydro-thieno[2,3-d]pyrimidine-6-carboxylic acid-3methoxy-benzylamide

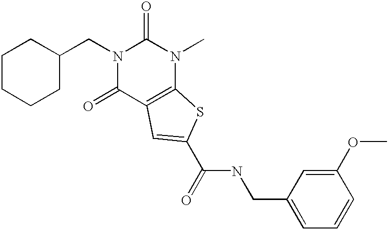

The procedure of Example 34 was repeated, except that 4-(4-bromomethyl-benzenesulfonyl)-morpholine is replaced by bromomethyl-cyclohexane, to give 3-cylcohexylmethyl-1-methyl-2,4-dioxo-1,2,3,4-tetrahydro-thieno[2,3-d]pyrimidine-6-carboxylic acid 3-methoxy-benzylamide as a white solid (62%). MS (APCI+), m/z 442 (M+).

### EXAMPLE 70

### 3-cylcohexylmethyl-1-methyl-2,4-dioxo-1,2,3,4-tetrahydro-thieno[2,3-d]pyrimidine-6-carboxylic acid-4methoxy-benzylamide

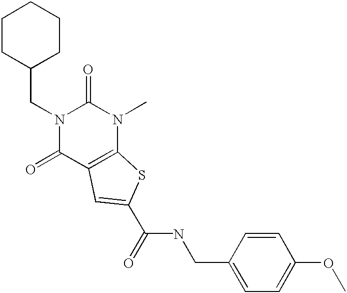

The procedure of Example 69 was repeated, except that 1-methyl-2,4-dioxo-1,2,3,4-tetrahydro-thieno[2,3-d]pyrimidine-6-carboxylic acid 3-methoxy-benzylamide is replaced by 1-methyl-2,4-dioxo-1,2,3,4-tetrahydro-thieno[2,3-d]pyrimidine-6-carboxylic acid 4-methoxy-benzylamide, to give 3-cylcohexylmethyl-1-methyl-2,4-dioxo-1,2,3,4-tetrahydro-thieno[2,3-d]pyrimidine-6-carboxylic acid 4-methoxy-benzylamide as a white solid (80%). MS (APCI+), m/z 442 (M+).

### EXAMPLE 71

### 3-Benzyl-2,4-dioxo-1,2,3,4-tetrahydro-thieno[2,3-d]pyrimidine-6-carboxylic acid furan-3-ylmethyl ester

The title compound was prepared according to the procedure of Example 1; MS (M+1) 383.2.

### EXAMPLE 72

### 3-Benzyl-1-methyl-2,4-dioxo-1,2,3,4-tetrahydro-thieno[2,3-d]pyrimidine-6-carboxylic acid pentafluorophenylmethyl ester

The title compound was prepared according to the procedure of Example 28; MS (M+1) 497.4.

### EXAMPLE 73

### 3-Benzyl-1-ethyl-2,4-dioxo-1,2,3,4-tetrahydro-thieno[2,3-d]pyrimidine-6-carboxylic acid benzyl ester

The title compound was prepared according to the procedure of Example 9; mp 147-148° C.

### EXAMPLE 74

### 3-Benzyl-1-cyclopropylmethyl-2,4-dioxo-1,2,3,4-tetrahydro-thieno[2,3-d]pyrimidine-6-carboxylic acid benzyl ester

The title compound was prepared according to the procedure of Example 9; MS (M+1) 447.1.

### EXAMPLE 75

### 3-Benzyl-1-methyl-2,4-dioxo-1,2,3,4-tetrahydro-thieno[2,3-d]pyrimidine-6-carboxylic acid (pyridin-4-ylmethyl)-amide

The title compound was prepared according to the procedure of Example 28; MS (M+1) 407.1.

### EXAMPLE 76

### 3-Benzyl-1-methyl-2,4-dioxo-1,2,3,4-tetrahydro-thieno[2,3-d]pyrimidine-6-carboxylic acid 3-bromo-benzyl ester

The title compound was prepared according to the procedure of Example 1; MS (M+1) 485.2.

### EXAMPLE 77

### 4-[6-(3-Difluoromethoxy-benzylcarbamoyl)-1-methyl-2,4-dioxo-1,4-dihydro-2H-thieno[2,3-d]pyrimidin-3-ylmethyl]-benzoic acid

The title compound was prepared according to the procedure of Example 28; MS (M+1) 516.1.

### EXAMPLE 78

### 4-[6-(3-Difluoromethoxy-benzylcarbamoyl)-1-methyl-2,4-dioxo-1,4-dihydro-2H-thieno[2,3-d]pyrimidin-3-ylmethyl]-benzoic acid tert-butyl ester

The title compound was prepared according to the procedure of Example 15; MS (M−C₄H₉) 516.1.

### EXAMPLE 79

### 4-[6-(3-Methoxy-benzylcarbamoyl)-1-methyl-2,4-dioxo-1,4-dihydro-2H-thieno[2,3-d]pyrimidin-3-ylmethyl]-benzoic acid

The title compound was prepared according to the procedure of Example 28; MS (M+1) 480.1.

### EXAMPLE 80

### 4-[6-(4-Methanesulfonyl-benzylcarbamoyl)-1-methyl-2,4-dioxo-1,4-dihydro-2H-thieno[2,3-d]pyrimidin-3-ylmethyl]-benzoic acid tert-butyl ester

The title compound was prepared according to the procedure of Example 15; ¹H-NMR (CDCl₃, d): 7.90 (m, 3H), 7.75 (d, 2H), 7.40 (m, 4H), 5.19 (s, 2H), 4.63 (d, 2H), 3.59 (s, 3H), 3.02 (s, 3H), 1.58 (s, 9H).

### EXAMPLE 81

### 4-[6-(4-Methanesulfonyl-benzylcarbamoyl)-1-methyl-2,4-dioxo-1,4-dihydro-2H-thieno[2,3-d]pyrimidin-3-ylmethyl]-benzoic acid

The title compound was prepared according to the procedure of Example 28; MS (M+1) 528.1.

### EXAMPLE 82

### 4-[1-Methyl-2,4-dioxo-6-(2-pyridin-4-yl-ethylcarbamoyl)-1,4-dihydro-2H-thieno[2,3-d]pyrimidin-3-ylmethyl]-benzoic acid

The title compound was prepared according to the procedure of Example 28; MS (M+1) 465.1.

### EXAMPLE 83

### 1-Methyl-2,4-dioxo-3-(4-trifluoromethoxy-benzyl)-1,2,3,4-tetrahydro-thieno[2,3-d]pyrimidine-6-carboxylic acid 3-methoxy-benzylamide

The title compound was prepared according to the procedure of Example 15; MS (M+1) 520.1.

### EXAMPLE 84

### 4-[6-(3-Methoxy-benzylcarbamoyl)-1-methyl-2,4-dioxo-1,4-dihydro-2H-thieno[2,3-d]pyrimidin-3-ylmethyl]-benzoic acid methyl ester

The title compound was prepared according to the procedure of Example 15; MS (M+1) 494.2.

### EXAMPLE 85

### 3-(2,3-Dihydro-benzofuran-6-ylmethyl)-1-methyl-2,4-dioxo-1,2,3,4-tetrahydro-thieno[2,3-d]pyrimidine-6-carboxylic acid 3-methoxy-benzylamide

The title compound was prepared according to the procedure of Example 28; MS (M+1) 478.2.

### EXAMPLE 86

### 1-Methyl-3-(2-methyl-thiazol-5-ylmethyl)-2,4-dioxo-1,2,3,4-tetrahydro-thieno[2,3-d]pyrimidine-6-carboxylic acid 3-methoxy-benzylamide

The title compound was prepared according to the procedure of Example 15; MS (M+1) 457.2.

### EXAMPLE 87

### 1-Methyl-2,4-dioxo-3-[4-(1H-tetrazol-5-yl)-benzyl]-1,2,3,4-tetrahydro-thieno[2,3-d]pyrimidine-6-carboxylic acid 4-fluoro-benzylamide

The title compound was prepared according to the procedure of Example 24; MS (M+1) 492.2.

### EXAMPLE 88

### 3-Benzyl-2-methoxy-4-oxo-3,4-dihydro-thieno[2,3-d]pyrimidine-6-carboxylic acid benzyl ester

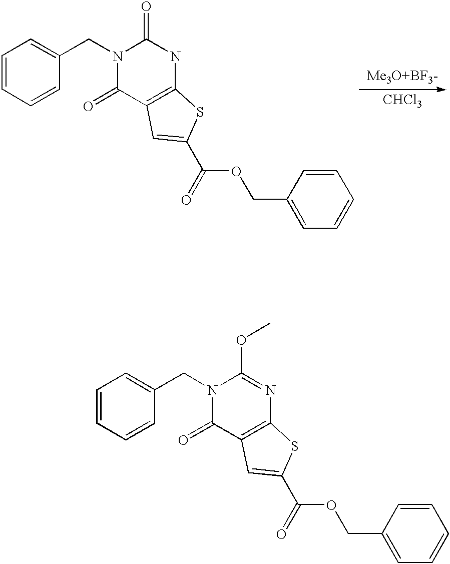

To a solution of 3-benzyl-2,4-dioxo-1,2,3,4-tetrahydro-thieno[2,3-d]pyrimidine-6-carboxylic acid benzyl ester (550 mg, 1.27 mmol) was added Me₃O⁺BF₃⁻ (376 mg, 2.54 mmol). The resulting yellow suspension was stirred at room temperature for 3 days, and MeOH was added to quench the reaction. After removal of volatile solvents in vacuo, the residue was purified using flash chromatography to give the desired product as a brownish oil. M+1 407.2.

### EXAMPLE 89

### 4-[6-(3-Methoxy-benzylcarbamoyl)-1-methyl-2,4-dioxo-1,4-dihydro-2H-thieno[2,3-d]pyrimidin-3-ylmethyl]-benzoic acid 2,2-dimethyl-propionyloxymethyl ester

The title compound was prepared according to the procedure of Example 68; MS (M−CH₃) 479.2.

### EXAMPLE 90

### 4-[6-(3-Methoxy-benzylcarbamoyl)-1-methyl-2,4-dioxo-1,4-dihydro-2H-thieno[2,3-d]pyrimidin-3-ylmethyl]-cyclohexanecarboxylic acid

The title compound was prepared according to the procedure of Example 28; MS (M+1) 486.2.

### EXAMPLE 91

### 4-[6-(3-Methoxy-benzylcarbamoyl)-1-methyl-2,4-dioxo-1,4-dihydro-2H-thieno[2,3-d]pyrimidin-3-ylmethyl]-cyclohexanecarboxylic acid methyl ester

The title compound was prepared according to the procedure of Example 15; MS (M+1) 500.1.

### EXAMPLE 92

### 1-{4-[6-(3-Methoxy-benzylcarbamoyl)-1-methyl-2,4-dioxo-1,4-dihydro-2H-thieno[2,3-d]pyrimidin-3-ylmethyl]-phenyl}-cyclopropanecarboxylic acid methyl ester

The title compound was prepared according to the procedure of Example 15; MS (M+1) 534.2.

### EXAMPLE 93

### 1-{4-[6-(3-Methoxy-benzylcarbamoyl)-1-methyl-2,4-dioxo-1,4-dihydro-2H-thieno[2,3-d]pyrimidin-3-ylmethyl]-phenyl}-cyclopropanecarboxylic acid tert-butyl ester

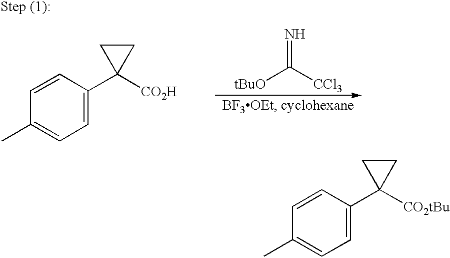

To a 0° C. solution of the starting carboxylic acid (2.63 g, 14.9 mmol) in cyclohexane (100 mL) was added BF₃ etherate (0.18 mL, catalytic). White precipitate was observed. After the reaction was stirred for 1.5 hours, the reaction was then filtered, and the filtrate was purified using a flash chromatography to give the desired ester as a colorless oil. 3.13 g, 90% yield.

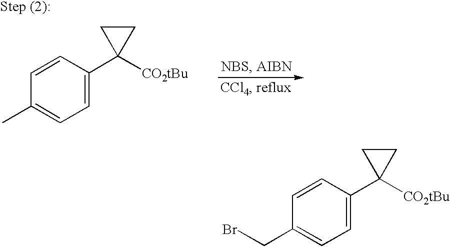

The t-butyl ester (3.13 g, 13.5 mmol), NBS (2.88 g, 16.2 mmol), and a catalytic amount of AIBN (0.2 g) were dissolved in CCl₄ (100 mL). The solution was refluxed for 2 hours and cooled to room temperature. After filtration of the white precipitate, the filtrate was filtered, and the filtrate was purified using a flash chromatography to give the desired ester as an oil. 4.01 g, 96% yield. MS, 267.0, 269.0; ¹H NMR (CDCl₃): δ7.35 (m, 4H), 4.49 (s, 2H), 3.62 (s, 3H), 1.60 (m, 2H), 1.18 (m, 2H), 1.00-1.60 (m, 4H).

Step (3)

The title compound was prepared according to the procedure of Example 28; ¹H-NMR (CDCl₃): δ7.67 (s, 1H), 7.40 (d, 1H), 7.22 (2H), 6.85 (d, 3H), 6.40 (t, 1H), 5.18 (s, 2H), 4.59 (d, 2H), 3.80 (s, 3H), 3.58 (s, 3H), 1.40 (s, 9H).

### EXAMPLE 94

### 1-{4-[6-(3-Methoxy-benzylcarbamoyl)-1-methyl-2,4-dioxo-1,4-dihydro-2H-thieno[2,3-d]pyrimidin-3-ylmethyl]-phenyl}-cyclopropanecarboxylic acid

The title compound was prepared according to the procedure of Example 28; MS (M+1) 520.

### EXAMPLE 95

### 2-{4-[6-(3-Methoxy-benzylcarbamoyl)-1-methyl-2,4-dioxo-1,4-dihydro-2H-thieno[2,3-d]pyrimidin-3-ylmethyl]-phenoxy}-2-methyl-propionic acid tert-butyl ester

The title compound was prepared according to the procedure of Example 15; ¹H-NMR (CDCl₃, d) 7.66 (s, 1H), 7.37 (d, 2H), 7.80 (m, 4H), 6.40 (t, 1H), 5.08 (s, 2H), 4.57 (d, 2H), 3.80 (s, 3H), 1.52 (s, 6H), 1.43 (s, 9H).

### EXAMPLE 96

### 2-{4-[6-(3-Methoxy-benzylcarbamoyl)-1-methyl-2,4-dioxo-1,4-dihydro-2H-thieno[2,3-d]pyrimidin-3-ylmethyl]-phenoxy}-2-methyl-propionic acid

The title compound was prepared according to the procedure of Example 28; MS (M+1) 538.2.

### EXAMPLE 97

### 3-Benzyl-1-methyl-2,4-dioxo-1,2,3,4-tetrahydro-furo[2,3-d]pyrimidine-6-carboxylic acid benzyl ester

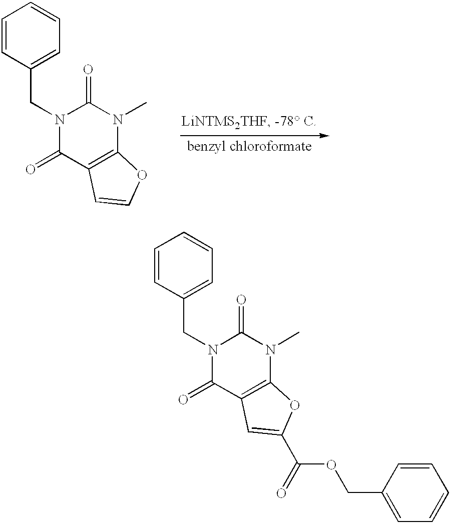

To a solution of the starting furano-pyrimidine-dione (265 mmg, 1.03 mmol) in THF at −78° C. was added LiN(TMS)₂ (1.3 mmol). After 5 minutes, benzyl chloroformate (0.17 mL, 264 mg, 1.55 mmol) was added dropwise, and the reaction was warmed up to room temperature and quenched by aq. NH₄Cl. Then extracted with EtOAc. The organic layer was washed with water and brine, dried over MgSO₄. After removal of volatile solvents in vacuo, the residue was purified using flash chromatography to give the desired product as a brownish oil. M+1 257.1

### EXAMPLE 98

### 3-(3-Methoxy-benzyl)-1-methyl-2,4-dioxo-1,2,3,4-tetrahydro-thieno[2,3-d]pyrimidine-6-carboxylic acid benzyl ester

To a 0.1 M solution of 1-methyl-2,4-dioxo-1,2,3,4-tetrahydro-thieno[2,3-d]pyrimidine-6-carboxylic acid benzyl ester in dimethylformamide was added 60% NaH in mineral oil (1.5 mol equivalents). After stirring for 20 minutes, 1 mL (0.1 mmol) of the reaction mixture was transferred to a 8 mL screw cap vial. To this was added a solution of 1-chloromethyl-3-methoxy-benzene (0.047 g, 0.3 mmol) in dimethylformamide (1 mL). The vial was capped, and the reaction mixture was shaken for 24 hours at room temperature. The reaction mixture was filtered, and the solvent was removed under vacuum. Purification was carried out via reverse-phase HPLC (3% n-propanol in acetonitrile and 3% n-propanol in water as the eluent; C-18 column). 0.023 g (50% yield). MS-APCI: (M+1)=437.486.

The procedure of Example 98 was used to prepare the compounds of Examples 99 to 145.

### EXAMPLE 99

### 3-Benzyl-1-methyl-2,4-dioxo-1,2,3,4-tetrahydro-thieno[2.3-d]pyrimidine-6-carboxylic acid benzyl ester

MS-APCI (M+1) 407.4602 (The compound of Example 99 is the same as the compound of Example 147.)

### EXAMPLE 100

### 3-Biphenyl-4-ylmethyl-1-methyl-2,4-dioxo-1,2,3,4-tetrahydro-thieno[2,3-d]pyrimidine-6-carboxylic acid benzyl ester

MS-APCI (M+1) 483.5578

### EXAMPLE 101

### 3-(4-Methanesulfonyl-benzyl)-1-methyl-2,4-dioxo-1,2,3,4-tetrahydro-thieno[2,3-d]pyrimidine-6-carboxylic acid benzyl ester

MS-APCI (M+1) 485.551

### EXAMPLE 102

### 3-(4-Methanesulfonyl-benzyl)-1-methyl-2,4-dioxo-1,2,3,4-tetrahydro-thieno[2,3-d]pyrimidine-6-carboxylic acid benzyl ester

MS-APCI (M+1) 485.551

### EXAMPLE 103

### 1-Methyl-3-(4-methyl-benzyl)-2,4-dioxo-1,2,3,4-tetrahydro-thieno[2,3-d]pyrimidine-6-carboxylic acid benzyl ester

MS-APCI (M+1) 421.487

### EXAMPLE 104

### 1-Methyl-2,4-dioxo-3-phenethyl-1,2,3,4-tetrahydro-thieno[2,3-d]pyrimidine-6-carboxylic acid benzyl ester

MS-APCI (M+1) 421.487

### EXAMPLE 105

### 3(4-Amino-6-phenylamino-1,3,5-triazin-2-ylmethyl)-1-methyl-2,4-dioxo-1,2,4-tetrahydro-thieno[2,3-d]pyrimidine-6-carboxylic acid benzyl ester

MS-APCI (M+1) 516.552

### EXAMPLE 106

### 1-Methyl-2,4-dioxo-3-(4-trifluoromethyl-benzyl)-1,2,3,4-tetrahydro-thieno[2,3-d]pyrimidine-6-carboxylic acid benzyl ester

MS-APCI (M+1) 475.457

### EXAMPLE 107

### 3-(6-Cyano-hexyl)-1-methyl-2,4-dioxo-1,2,3,4-tetrahydro-thieno[2,3-d]pyrimidine-6-carboxylic acid benzyl ester

MS-APCI (M+1) 426.507

### EXAMPLE 108

### 3-[2-(2,5-Dimethoxy-phenyl)-2-oxo-ethyl]-1-methyl-2,4-dioxo-1,2,3,4-tetrahydro-thieno[2,3-d]pyrimidine-6-carboxylic acid benzyl ester

MS-APCI (M+1) 495.522

### EXAMPLE 109

### 3-(3-Iodo-benzyl)-1-methyl-2,4-dioxo-1,2,3,4-tetrahydro-thieno[2,3-d]pyrimidine-6-carboxylic acid benzyl ester

MS-APCI (M+1) 533.352

### EXAMPLE 110

### 1-Methyl-2,4-dioxo-3-(3-trifluoromethyl-benzyl)-1,2,3,4-tetrahydro-thieno[2,3-d]pyrimidine-6-carboxylic acid benzyl ester

MS-APCI (M+1) 475.457

### EXAMPLE 111

### 3-(2,4-Bis-trifluoromethyl-benzyl)-1-methyl-2,4-dioxo-1.2.3.4-tetrahydro-thieno[2,3-d]pyrimidine-6-carboxylic acid benzyl ester

MS-APCI (M+1) 543.454

### EXAMPLE 112

### 3-[2-(1,3-Dioxo-1,3-dihydro-isoindol-2-yl)-ethyl]-1-methyl-2,4-dioxo-1,2,3,4-tetrahydro-thieno[2,3-d]pyrimidine-6-carboxylic acid benzyl ester

MS-APCI (M+1) 490.506

### EXAMPLE 113

### 3-[2-(1,3-Dioxo-1,3-dihydro-isoindol-2-yl)-ethyl]-1-methyl-2,4-dioxo-1,2,3,4-tetrahydro-thieno[2,3-d]pyrimidine-6-carboxylic acid benzyl ester

MS-APCI (M+1) 490.506

### EXAMPLE 114

### 3-(2-Carboxy-allyl)-1-methyl-2,4-dioxo-1,2,3,4-tetrahydro-thieno]2,3-d]pyrimidine-6-carboxylic acid benzyl ester

MS-APCI (M+1) 401.4094

### EXAMPLE 115

### 3-(2-Carboxy-allyl)-1-methyl-2,4-dioxo-1,2,3,4-tetrahydro-thieno[2,3-d]pyrimidine-6-carboxylic acid benzyl ester

MS-APCI (M+1) 401.4094

### EXAMPLE 116

### 3-(3-Amino-propyl)-1-methyl-2,4-dioxo-1,2,3,4-tetrahydro-thieno[2,3-d]pyrimidine-6-carboxylic acid benzyl ester

MS-APCI (M+1) 374.431

### EXAMPLE 117

### 3-(1,3-Dioxo-1,3-dihydro-isoindol-2-ylmethyl)-1-methyl-2,4-dioxo-1,2,3,4-tetrahydro-thieno[2,3-d]pyrimidine-6-carboxylic acid benzyl ester

MS-APCI (M+1) 476.479

### EXAMPLE 118

### 3-(4-Fluoro-benzyl)-1-methyl-2,4-dioxo-1,2,3,4-tetrahydro-thieno[2,3-d]pyrimidine-6-carboxylic acid benzyl ester

MS-APCI (M+1) 425.45

### EXAMPLE 119

### 1-Methyl-3-oxiranylmethyl-2,4-dioxo-1,2,3,4-tetrahydro-thieno[2,3-d]pyrimidine-6-carboxylic acid benzyl ester

MS-APCI (M+1) 373.399

### EXAMPLE 120

### 1-Methyl-3-(2-methyl-butyl)-2,4-dioxo-1,2,3,4-tetrahydro-thieno[2,3-d]pyrimidine-6-carboxylic acid benzyl ester

MS-APCI (M+1) 387.47

### EXAMPLE 121

### 1-Methyl-2,4-dioxo-3-(4-phenoxy-butyl)-1,2,3,4-tetrahydro-thieno[2,3-d]pyrimidine-6-carboxylic acid benzyl ester

MS-APCI (M+1) 465.54

### EXAMPLE 122

### 3-(2-Cyano-benzyl)-1-methyl-2,4-dioxo-1,2,3,4-tetrahydro-thieno[2,3-d]pyrimidine-6-carboxylic acid benzyl ester

MS-APCI (M+1) 432.47

### EXAMPLE 123

### 1-Methyl-2,4-dioxo-3-(3-phenoxy-propyl)-1,2,3,4-tetrahydro-thieno[2,3-d]pyrimidine-6-carboxylic acid benzyl ester

MS-APCI (M+1) 451.513

### EXAMPLE 124

### 3-Hex-5-enyl-1-methyl-2,4-dioxo-1,2,3,4-tetrahydro-thieno[2,3-d]pyrimidine-6-carboxylic acid benzyl ester

MS-APCI (M+1) 399.481

### EXAMPLE 125

### 1-Methyl-2,4-dioxo-3-pyridin-3-ylmethyl-1,2,3,4-tetrahydro-thieno[2,3-d]pyrimidine-6-carboxylic acid benzyl ester

MS-APCI (M+1) 408.448

### EXAMPLE 126

### 3-[2-Hydroxy-3-(naphthalen-1-yloxy)-propyl]-1-methyl-2,4-dioxo-1,2,3,4-tetrahydro-thieno[2,3-d]pyrimidine-6-carboxylic acid benzyl ester

MS-APCI (M+1) 517.572

### EXAMPLE 127

### 1,3-Dimethyl-2,4-dioxo-1,2,3,4-tetrahydro-thieno[2,3-d]pyrimidine-6-carboxylic acid benzyl ester

MS-APCI (M+1) 331.363

### EXAMPLE 28

### 3-Cyclobutylmethyl-1-methyl-2,4-dioxo-1,2,3,4-tetrahydro-thieno[2,3-d]pyrimidine-6-carboxylic acid benzyl ester

MS-APCI (M+1) 385.454

### EXAMPLE 129

### 3-Allyl-1-methyl-2,4-dioxo-1,2,3,4-tetrahydro-thieno[2,3-d]pyrimidine-6-carboxylic acid benzyl ester

MS-APCI (M+1) 357.4

### EXAMPLE 130

### 1-Methyl-2,4-dioxo-3-prop-2-ynyl-1,2,3,4-tetrahydro-thieno[2,3-d]pyrimidine-6-carboxylic acid benzyl ester

MS-APCI (M+1) 355.385

### EXAMPLE 131

### 3-But-2-ynyl-1-methyl-2,4-dioxo-1,2,3,4-tetrahydro-thieno[2,3-d]pyrimidine-6-carboxylic acid benzyl ester

MS-APCI (M+1) 369.411

### EXAMPLE 132

### 1-Methyl-2,4-dioxo-3-(2-phenoxy-ethyl)-1,2,3,4-tetrahydro-thieno[2,3-d]pyrimidine-6-carboxylic acid benzyl ester

MS-APCI (M+1) 437.486

### EXAMPLE 133

### 1-Methyl-2,4-dioxo-3-(2-phenoxy-ethyl)-1,2,3,4-tetrahydro-thieno[2,3-d]pyrimidine-6-carboxylic acid benzyl ester

MS-APCI (M+1) 437.486

### EXAMPLE 134

### 3-(3-Hydroxy-2-methyl-propyl)-1-methyl-2,4-dioxo-1,2,3,4-tetrahydro-thieno[2,3-d]pyrimidine-6-carboxylic acid benzyl ester

MS-APCI (M+1) 390.442

### EXAMPLE 135

### 3-Isobutyl-1-methyl-2,4-dioxo-1,2,3,4-tetrahydro-thieno[2,3-d]pyrimidine-6-carboxylic acid benzyl ester

MS-APCI (M+1) 373.443

### EXAMPLE 136

### 3-(6-Chloro-pyridin-3-ylmethyl)-1-methyl-2,4-dioxo-1,2,3,4tetrahydro-thieno[2,3-d]pyrimidine-6-carboxylic acid benzyl ester

MS-APCI (M+1) 442.8934

### EXAMPLE 137

### 3-(2-Benzenesulfonylmethyl-benzyl)-1-methyl-2,4-dioxo-1,2,3,4tetrahydro-thieno[2,3-d]pyrimidine-6-carboxylic acid benzyl ester

MS-APCI (M+1) 561.649

### EXAMPLE 138

### 1-Methyl-3-naphthalen-1-ylmethyl-2,4-dioxo-1,2,3,4-tetrahydro-thieno[2,3-d]pyrimidine-6-carboxylic acid benzyl ester

MS-APCI (M+1) 457.52

### EXAMPLE 139

### 1-Methyl-2,4-dioxo-3-(2-trifluoromethyl-benzyl)-1,2,3,4-tetrahydro-thieno[2,3-d]pyrimidine-6-carboxylic acid benzyl ester

MS-APCI (M+1) 475.457

### EXAMPLE 140

### 3-(3-Chloro-benzyl-1-methyl-2,4-dioxo-1,2,3,4-tetrahydro-thieno[2,3-d]pyrimidine-6-carboxylic acid benzyl ester

MS-APCI (M+1) 441.905

### EXAMPLE 141

### 3-(4-Methoxycarboxyl-butyl)-1-methyl-2,4-dioxo-1,2,3,4-tetrahydro-thieno[2,3-d]pyrimidine-6-carboxylic acid benzyl ester

MS-APCI (M+1) 431.479

### EXAMPLE 142

### 3-Ethyl-1-methyl-2,4-dioxo-1,2,3,4-tetrahydro-thieno[2,3-d]pyrimidine-6-carboxylic acid benzyl ester

MS-APCI (M+1) 345.389

### EXAMPLE 143

### 1-Methyl-2,4-dioxo-3-(3-phenyl-propyl)-1,2,3,4-tetrahydro-thieno[2,3-d]pyrimidine-6-carboxylic acid benzyl ester

MS-APCI (M+1) 435.514

### EXAMPLE 144

### 3-[2-(4-Chloro-benzenesulfonyl)-ethyl]-1-methyl-2,4-dioxo-1,2,3,4-tetrahydro-thieno[2,3-d]pyrimidine-6-carboxylic acid benzyl ester

MS-APCI (M+1) 519.996

### EXAMPLE 145

### 3-(2-Acetoxy-ethyl)-1-methyl-2,4-dioxo-1,2,3,4-tetrahydro-thieno[2,3-d]pyrimidine-6-carboxylic acid benzyl ester

MS-APCI (M+1) 403.425

### EXAMPLE 146

### 3-Benzyl-1-methyl-2,4-dioxo-1,2,3,4-tetrahydro-thieno[2,3-d]pyrimidine-6-carboxylic acid 2-phenoxyethyl ester

In a 8 mL screw cap vial was added a mixture of 3-benzyl-1-methyl-2,4-dioxo-1,2,3,4-tetrahydro-thieno[2,3-d]pyrimidine-6-carboxylic acid, (0.032 g, 0.1 mmol), triethylamine (0.024 g, 0.24 mmol), and 2-chloro-1-methylpyridinium iodine (0.031 g, 0.12 mmol) in dichloromethane (2 mL), followed by 2-phenoxy ethanol (0.015 g, 0.11 mmol) in dichloromethane (1 mL). The vial was capped, and the reaction mixture was shaken for 24 hours at room temperature. The solvent was removed under vacuum. Purification was carried out via reverse-phase HPLC (3% n-propanol in acetonitrile and 3% n-propanol in water as the eluent; C-18 column). 0.023 g (50% yield). MS-APCI (M+1)=437.486.

The procedure of Example 146 was used to prepare the compounds of Examples 147 to 215.

### EXAMPLE 147

### 3-Benzyl-1-methyl-2,4-dioxo-1,2,3,4-tetrahydro-thieno[2,3-d]pyrimidine-6-carboxylic acid benzyl ester

MS-APCI (M+1) 407.4602 (The compound of Example 147 is the same as the compound of Example 99).

### EXAMPLE 148

### 3-Benzyl-1-methyl-2,4-dioxo-1,2,3,4-tetrahydro-thieno[2,3-d]pyrimidine-6-carboxylic acid benzyl amide

MS-APCI (M+1) 406.4761

### EXAMPLE 149

### 3-Benzyl-1-methyl-2,4-dioxo-1,2,3,4-tetrahydro-thieno[2,3-d]pyrimidine-6-carboxylic acid 2,6-dichloro-benzyl ester

MS-APCI (M+1) 476.35

### EXAMPLE 150

### 3-Benzyl-1-methyl-2,4-dioxo-1,2,3,4-tetrahydro-thieno[2,3-d]pyrimidine-6-carboxylic acid butyl ester

MS-APCI (M+1) 373.443

### EXAMPLE 151

### 3-Benzyl-1-methyl-2,4-dioxo-1,2,3,4-tetrahydro-thieno[2,3-d]pyrimidine-6-carboxylic acid 2,3-dihydro-1,4-benzodioxin-2-ylmethyl ester

MS-APCI (M+1) 465.496

### EXAMPLE 152

### 3-Benzyl-1-methyl-2,4-dioxo-1,2,3,4-tetrahydro-thieno[2,3-d]pyrimidine-6-carboxylic acid 2-diethylamino-1-methyl-ethyl ester

MS-APCI (M+1) 430.538

### EXAMPLE 153

### 3-Benzyl-1-methyl-2,4-dioxo-1,2,3,4-tetrahydro-thieno[2,3-d]pyrimidine-6-carboxylic acid 4-fluoro-benzyl ester

MS-APCI (M+1) 425.45

### EXAMPLE 154

### 3-Benzyl-1-methyl-2,4-dioxo-1,2,3,4-tetrahydro-thieno[2,3-d]pyrimidine-6-carboxylic acid 4-isopropyl-benzyl ester

MS-APCI (M+1) 449.541

### EXAMPLE 155

### 3-Benzyl-1-methyl-2,4-dioxo-1,2,3,4-tetrahydro-thieno[2,3-d]pyrimidine-6-carboxylic acid 2-p-tolyl-ethyl ester

MS-APCI (M+1) 435.514

### EXAMPLE 156

### 3-Benzyl-1-methyl-2,4-dioxo-1,2,3,4-tetrahydro-thieno[2,3-d]pyrimidine-6-carboxylic acid 4-trifluoromethyl-benzyl ester

MS-APCI (M+1) 475.457

### EXAMPLE 157

### 3-Benzyl-1-methyl-2,4-dioxo-1,2,3,4-tetrahydro-thieno[2,3-d]pyrimidine-6-carboxylic acid cyclobutylmethyl ester

MS-APCI (M+1) 385.454

### EXAMPLE 158

### 3-Benzyl-1-methyl-2,4-dioxo-1,2,3,4-tetrahydro-thieno[2,3-d]pyrimidine-6-carboxylic acid 2,6-difluoro-benzyl ester

MS-APCI (M+1) 443.44

### EXAMPLE 159

### 3-Benzyl-1-methyl-2,4-dioxo-1,2,3,4-tetrahydro-thieno[2,3-d]pyrimidine-6-carboxylic acid 2-(2-hydroxy-phenyl)-ethyl ester

MS-APCI (M+1) 437.486

### EXAMPLE 160

### 3-Benzyl-1-methyl-2,4-dioxo-1,2,3,4-tetrahydro-thieno[2,3-d]pyrimidine-6-carboxylic acid 2-(2-hydroxy-phenyl)-ethyl ester

MS-APCI (M+1) 437.486

### EXAMPLE 161

### 3-Benzyl-1-methyl-2,4-dioxo-1,2,3,4-tetrahydro-thieno[2,3-d]pyrimidine-6-carboxylic acid 1-methyl-piperidin-4-yl ester

MS-APCI (M+1) 414.496

### EXAMPLE 162

### 3-Benzyl-1-methyl-2,4-dioxo-1,2,3,4-tetrahydro-thieno[2,3-d]pyrimidine-6-carboxylic acid 1-methyl-piperidin-4-yl ester

MS-APCI (M+1) 414.496

### EXAMPLE 163

### 3-Benzyl-1-methyl-2,4-dioxo-1,2,3,4-tetrahydro-thieno[2,3-d]pyrimidine-6-carboxylic acid pyridin-3-ylmethyl ester

MS-APCI (M+1) 408.448

### EXAMPLE 164

### 3-Benzyl-1-methyl-2,4-dioxo-1,2,3,4-tetrahydro-thieno[2,3-d]pyrimidine-6-carboxylic acid 3-pyridin-3-yl-propyl ester

MS-APCI (M+1) 436.502

### EXAMPLE 165

### 3-Benzyl-1-methyl-2,4-dioxo-1,2,3,4-tetrahydro-thieno[2,3-d]pyrimidine-6-carboxylic acid 2-dimethylamino-1-methyl-ethyl ester

MS-APCI (M+1) 402.485

### EXAMPLE 166

### 3-Benzyl-1-methyl-2,4-dioxo-1,2,3,4-tetrahydro-thieno[2,3-d]pyrimidine-6-carboxylic acid 4-methoxy-benzyl ester

MS-APCI (M+1) 437.486

### EXAMPLE 167

### 3-Benzyl-1-methyl-2,4-dioxo-1,2,3,4-tetrahydro-thieno[2,3-d]pyrimidine-6-carboxylic acid tetrahydro-pyran-4-yl ester

MS-APCI (M+1) 401.453

### EXAMPLE 168

### 3-Benzyl-1-methyl-2,4-dioxo-1,2,3,4-tetrahydro-thieno[2,3-d]pyrimidine-6-carboxylic acid 2,2,2-trifluoro-1-trifluoromethyl-ethyl ester

MS-APCI (M+1) 487.357

### EXAMPLE 169

### 3-Benzyl-1-methyl-2,4-dioxo-1,2,3,4-tetrahydro-thieno[2,3-d]pyrimidine-6-carboxylic acid 2-trifluoromethyl-benzyl ester

MS-APCI (M+1) 475.457

### EXAMPLE 170

### 3-Benzyl-1-methyl-2,4-dioxo-1,2,3,4-tetrahydro-thieno[2,3-d]pyrimidine-6-carboxylic acid 2-benzyloxy-ethyl ester

MS-APCI (M+1) 451.513

### EXAMPLE 171

### 3-Benzyl-1-methyl-2,4-dioxo-1,2,3,4-tetrahydro-thieno[2,3-d]pyrimidine-6-carboxylic acid 2,2,2-trichloro-ethyl ester

MS-APCI (M+1) 448.725

### EXAMPLE 172

### 3-Benzyl-1-methyl-2,4-dioxo-1,2,3,4-tetrahydro-thieno[2,3-d]pyrimidine-6-carboxylic acid phenethyl ester

MS-APCI (M+1) 421.487

### EXAMPLE 173

### 3-Benzyl-1-methyl-2,4-dioxo-1,2,3,4-tetrahydro-thieno[2,3-d]pyrimidine-6-carboxylic acid 3-ethyl-oxetan-3-ylmethyl ester

MS-APCI (M+1) 415.48

### EXAMPLE 174

### 3-Benzyl-1-methyl-2,4-dioxo-1,2,3,4-tetrahydro-thieno[2,3-d]pyrimidine-6-carboxylic acid 2-morpholin-4-yl-ethyl ester

MS-APCI (M+1) 430.495

### EXAMPLE 175

### 3-Benzyl-1-methyl-2,4-dioxo-1,2,3,4-tetrahydro-thieno[2,3-d]pyrimidine-6-carboxylic acid 2-pyrrolidin-1-yl-ethyl ester

MS-APCI (M+1) 414.496

### EXAMPLE 176

### 3-Benzyl-1-methyl-2,4-dioxo-1,2,3,4-tetrahydro-thieno[2,3-d]pyrimidin-6-carboxylic acid 2-pyrrolidin-1-yl-ethyl ester

MS-APCI (M+1) 414.96

### EXAMPLE 177

### 3-Benzyl-1-methyl-2,4-dioxo-1,2,3,4-tetrahydro-thieno[2,3-d]pyrimidine-6-carboxylic acid 2-(2-ethoxy-ethoxy)-ethyl ester

MS-APCI (M+1) 433.495

### EXAMPLE 178

### 3-Benzyl-1-methyl-2,4-dioxo-1,2,3,4-tetrahydro-thieno[2,3-d]pyrimidine-6-carboxylic acid tetrahydro-pyran-2-ylmethyl ester

MS-APCI (M+1) 415.48

### EXAMPLE 179

### 3-Benzyl-1-methyl-2,4-dioxo-1,2,3,4-tetrahydro-thieno[2,3-d]pyrimidine-6-carboxylic acid 4-nitro-benzyl ester

MS-APCI (M+1) 452.457

### EXAMPLE 180

### 3-Benzyl-1-methyl-2,4-dioxo-1,2,3,4-tetrahydro-thieno[2,3-d]pyrimidine-6-carboxylic acid pentyl ester

MS-APCI (M+1) 387.47

### EXAMPLE 181

### 3-Benzyl-1-methyl-2,4-dioxo-1,2,3,4-tetrahydro-thieno[2,3-d]pyrimidine-6-carboxylic acid 3-phenyl-propyl ester

MS-APCI (M+1) 435.514

### EXAMPLE 182

### 3-Benzyl-1-methyl-2,4-dioxo-1,2,3,4-tetrahydro-thieno[2,3-d]pyrimidine-6-carboxylic acid 3-phenoxy-benzyl ester

MS-APCI (M+1) 499.557

### EXAMPLE 183

### 3-Benzyl-1-methyl-2,4-dioxo-1,2,3,4-tetrahydro-thieno[2,3-d]pyrimidine-6-carboxylic acid 3,5-dimethoxy-benzyl ester

MS-APCI (M+1) 467.512

### EXAMPLE 184

### 3-Benzyl-1-methyl-2,4-dioxo-1,2,3,4-tetrahydro-thieno[2,2-d]pyrimidine-6-caboxylic acid 3-methyl-butyl ester

MS-APCI (M+1) 387.47

### EXAMPLE 185

### 3-Benzyl-1-methyl-2,4-dioxo-1,2,3,4-tetrahydro-thieno[2,3-d]pyrimidine-6-carboxylic acid 4-chloro-benzyl ester

MS-APCI (M+1) 441.905

### EXAMPLE 186

### 3-Benzyl-1-methyl-2,4-dioxo-1.2.3.4-tetrahydro-thieno[2,3-d]pyrimidine-6-carboxylic acid 1-ethyl-piperidin-3-yl ester

MS-APCI (M+1) 428.522

### EXAMPLE 187

### 3-Benzyl-1-methyl-2,4-dioxo-1,2,3,4-tetrahydro-thieno[2,3-d]pyrimidine-6-carboxylic acid 4-benzyloxy-benzyl ester

MS-APCI (M+1) 513.584

### EXAMPLE 188

### 3-Benzyl-1-methyl-2,4-dioxo-1,2,3,4-tetrahydro-thieno[2,3-d]pyrimidine-6-carboxylic acid isobutyl ester

MS-APCI (M+1) 373.443

### EXAMPLE 189

### 3-Benzyl-1-methyl-2,4-dioxo-1,2,3,4-tetrahydro-thieno[2,3-d]pyrimidine-6-carboxylic acid 3-(4-methoxy-phenyl)-propyl ester

MS-APCI (M+1) 465.54

### EXAMPLE 190

### 3-Benzyl-1-methyl-2,4-dioxo-1,2,3,4-tetrahydro-thieno[2,3-d]pyrimidine-6-carboxylic acid 2-chloro-6-fluoro-benzyl ester

MS-APCI (M+1) 459.895

### EXAMPLE 191

### 3-Benzyl-1-methyl-2,4-dioxo-1,2,3,4-tetrahydro-thieno[2,3-d]pyrimidine-6-carboxylic acid tetrahydro-furan-3-yl ester

MS-APCI (M+1) 387.426

### EXAMPLE 192

### 3-Benzyl-1-methyl-2,4-dioxo-1,2,3,4-tetrahydro-thieno[2,3-d]pyrimidine-6-carboxylic acid 3-methoxy-benzyl ester

MS-APCI (M+1) 437.486

### EXAMPLE 193

### 3-Benzyl-1-methyl-2,4-dioxo-1,2,3,4-tetrahydro-thieno[2,3-d]pyrimidine-6-carboxylic acid 3-methoxy-benzyl ester

MS-APCI (M+1) 437.486

### EXAMPLE 194

### 3-Benzyl-1-methyl-2,4-dioxo-1,2,3,4-tetrahydro-thieno[2,3-d]pyrimidine-6-carboxylic acid 3-pyridin-2-yl-propyl ester

MS-APCI (M+1) 436.502

### EXAMPLE 195

### 3-Benzyl-1-methyl-2,4-dioxo-1,2,3,4-tetrahydro-thieno[2,3-d]pyrimidine-6-carboxylic acid 2-piperidin-2-yl-ethyl ester

MS-APCI (M+1) 428.522

### EXAMPLE 196

### 3-Benzyl-1-methyl-2,4-dioxo-1,2,3,4-tetrahydro-thieno[2,3-d]pyrimidine-6-carboxylic acid 5-bromo-2-methoxy-benzyl ester

MS-APCI (M+1) 516.382

### EXAMPLE 197

### 3-Benzyl-1-methyl-2,4-dioxo-1,2,3,4-tetrahydro-thieno[2,3-d]pyrimidine-6-carboxylic acid cycloheptylmethyl ester

MS-APCI (M+1) 427.534

### EXAMPLE 198

### 3-Benzyl-1-methyl-2,4-dioxo-1,2,3,4-tetrahydro-thieno[2,3-d]pyrimidine-6-carboxylic acid 1,2,3,4-tetrahydro-naphthalen-1-yl ester

MS-APCI (M+1) 447.525

### EXAMPLE 199

### 3-Benzyl-1-methyl-2,4-dioxo-1,2,3,4-tetrahydro-thieno[2,3-d]pyrimidine-6-carboxylic acid pyrrolidin-2-ylmethyl ester

MS-APCI (M+1) 400.469

### EXAMPLE 200

### 3-Benzyl-1-methyl-2,4-dioxo-1,2,3,4-tetrahydro-thieno[2,3-d]pyrimidine-6-carboxylic acid 3-chloro-benzyl ester

MS-APCI (M+1) 441.905

### EXAMPLE 201

### 3-Benzyl-1-methyl-2,4-dioxo-1,2,3,4-tetrahydro-thieno[2,3-d]pyrimidine-6-carboxylic acid 1,3-benzodioxol-5-ylmethyl ester

MS-APCI (M+1) 451.469

### EXAMPLE 202

### 3-Benzyl-1-methyl-2,4-dioxo-1,2,3,4-tetrahydro-thieno[2,3-d]pyrimidine-6-carboxylic acid 4-methylsulfanyl-benzyl ester

MS-APCI (M+1) 453.553

### EXAMPLE 203

### 3-Benzyl-1-methyl-2,4-dioxo-1,2,3,4-tetrahydro-thieno[2,3-d]pyrimidine-6-carboxylic acid 4-methylsulfanyl-benzyl ester

MS-APCI (M+1) 453.553

### EXAMPLE 204

### 3-Benzyl-1-methyl-2,4-dioxo-1,2,3,4-tetrahydro-thieno[2,3-d]pyrimidine-6-carboxylic acid 3,4-dichloro-benzyl ester

MS-APCI (M+1) 476.35

### EXAMPLE 205

### 3-Benzyl-1-methyl-2,4-dioxo-1,2,3,4-tetrahydro-thieno[2,3-d]pyrimidine-6-carboxylic acid 3,3-diphenyl-propyl ester

MS-APCI (M+1) 511.611

### EXAMPLE 206

### 3-Benzyl-1-methyl-2,4-dioxo-1,2,3,4-tetrahydro-thieno[2,3-d]pyrimidine-6-carboxylic acid 2-pyridin-2-yl-ethyl ester

MS-APCI (M+1) 422.475

### EXAMPLE 207

### 3-Benzyl-1-methyl-2,4-dioxo-1,2,3,4-tetrahydro-thieno[2,3-d]pyrimidine-6-carboxylic acid furan-3-ylmethyl ester

MS-APCI (M+1) 397.421

### EXAMPLE 208

### 3-Benzyl-1-methyl-2,4-dioxo-1,2,3,4-tetrahydro-thieno[2,3-d]pyrimidine-6-carboxylic acid but-3-enyl ester

MS-APCI (M+1) 371.427

### EXAMPLE 209

### 3-Benzyl-1-methyl-2,4-dioxo-1,2,3,4-tetrahydro-thieno[2,3-d]pyrimidine-6-carboxylic acid 2-cyano-ethyl ester

MS-APCI (M+1) 370.399

### EXAMPLE 210

### 3-Benzyl-1-methyl-2,4-dioxo-1,2,3,4-tetrahydro-thieno[2,3-d]pyrimidine-6-carboxylic acid 2-ethoxy-ethyl ester

MS-APCI (M+1) 389.442

### EXAMPLE 211

### 3-Benzyl-1-methyl-2,4-dioxo-1,2,3,4-tetrahydro-thieno[2,3-d]pyrimidine-6-carboxylic acid cyano-phenyl-methyl ester

MS-APCI (M+1) 432.47

### EXAMPLE 212

### 3-Benzyl-1-methyl-2,4-dioxo-1,2,3,4-tetrahydro-thieno[2,3-d]pyrimidine-6-carboxylic acid 4-trifluoromethyl-benzylamide

MS-APCI (M+1) 474.473

### EXAMPLE 213

### 3-Benzyl-1-methyl-2,4-dioxo-1,2,3,4-tetrahydro-thieno[2,3-d]pyrimidine-6-carboxylic acid 4-methyl-benzylamide

MS-APCI (M+1) 420.503

### EXAMPLE 214

### 3-Benzyl-1-methyl-2,4-dioxo-1,2,3,4-tetrahydro-thieno[2,3-d]pyrimidine-6-carboxylic acid phenethyl-amide

MS-APCI (M+1) 420.503

### EXAMPLE 215

### 3-Benzyl-1-methyl-2,4-dioxo-1,2,3,4-tetrahydro-thieno[2,3-d]pyrimidine-6-carboxylic acid cyclopropylamide

MS-APCI (M+1) 356.416

### EXAMPLE 216

### 1-Methyl-1,2,3,4-tetrahydro-thieno[2,3-d]pyrimidine-6-carboxylic acid 4-methoxy-benzylamide

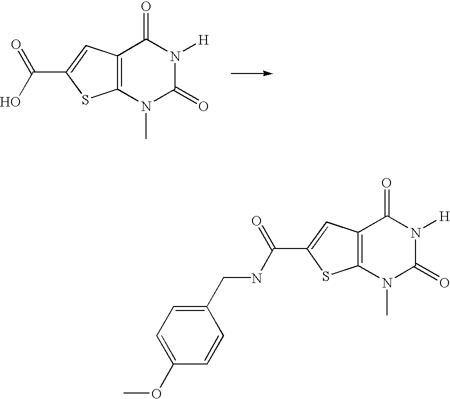

1-Methyl-1,2,3,4-tetrahydro-thieno[2,3-d]pyrimidine-6-carboxylic acid (1.0589 g, 4.681 mmol) was suspended in 50 mL methylene chloride and 50 mL THF. To this solution, 4-methyl morpholine (2,5895 g, 26.59 mmol), 1-hydroxybenzotriazole monohydrate (“HOBT-H₂O”) (0.7685 g, 5,688 mmol), and 4-methoxybenzylamine (0.7848 g, 5.721 mmol) were added. Then 1-(3-dimethylaminopropyl)-3-ethylcarbodiimide hydrochloride (“EDAC-HCl”) (1.0795 g, 5.631 mmol) was added, and the reaction was stirred at room temperature overnight. The solvent was removed under vacuum, and ˜20 mL 5% HCl was added, stirred for approximately 30 minutes, and the product was suction filtered. The filter cake was washed with 5% HCl, then 5% NaHCO₃, water, and dried at 40° C. under vacuum, to yield 1.4877 g white solid; mp 252-254° C.

### EXAMPLE 217

### 1-Methyl-2,4-dioxo-3-(4-sulfamoyl-benzyl)-1,2,3,4-tetrahydro-thieno[2,3-d]pyrimidine-6-carboxylic acid 4-methoxy-benzylamide

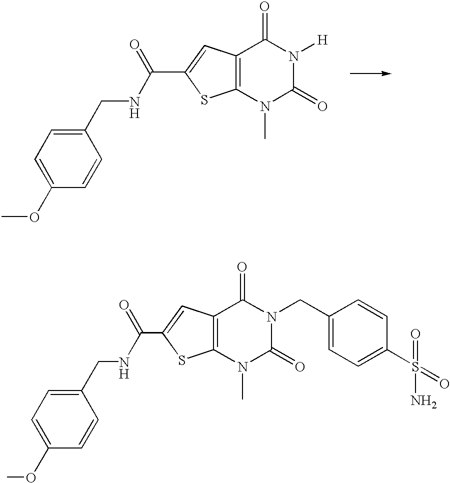

1-Methyl-1,2,3,4-tetrahydro-thieno[2,3-d]pyrimidine-6-carboxylic acid 4-methoxy-benzylamide (0.1484 g, 0.4297 mmol) was dissolved in 8 mL DMF, then cesium carbonate (0.2020 g, 0.62 mmol) was added and stirred at room temperature for 10 minutes. The 4-bromomethyl benzenesulfonamide (0.1084 g, 0.4334 mmol) was added and stirred at room temperature overnight. The solution was added dropwise to 70 mL of water, and the precipitate was suction filtered and dried under vacuum to give a white solid (0.1038 g). NMR (DMSO, ppm) 9.19 (1H, t, J=5.9 Hz), 8.12 (1H, s), 7.75 (2H, d, J=8.3 Hz), 7.47 (2H, d, J=8.5 Hz), 7.32 (2H, br), 7.23 (2H, d, J=8.5 Hz), 6.90 (2H, d, J=8.5 Hz), 5.12 (2H, s), 4.38 (2H, d, J=5.6 Hz), 3.73 (3H, s), 3.48 (3H, s).

### EXAMPLE 218

### 1-Methyl-1,2,3,4-tetrahydro-thieno[2,3-d]pyrimidine-6-carboxylic acid 3-methoxy-benzylamide

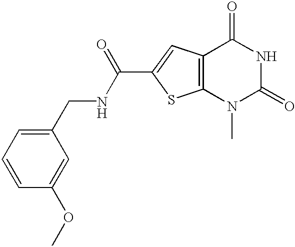

Made by the procedure of Example 216 using 3-methoxybenzyl amine; mp 279-281° C.,

### EXAMPLE 219

### 1-methyl-2,4-dioxo-1,2,3,4-tetrahydro-thieno[2,3-d]pyrimidine-6-carboxylic acid (2-methoxy-pyridin-4-ylmethyl)-amide

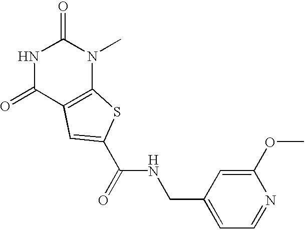

Made by the procedure of Example 216 with 1-methyl-1,2,3,4-tetrahydro-thieno[2,3-d]pyrimidine-6-carboxylic acid and 4-(aminomethyl)-2-methoxy-pyridine. NMR (DMSO) 11.58 (1H, br), 9.25 (1H, t, J=6.1 Hz), 8.15-8.05 (2H, m), 6.9 (1H, m), 6.70 (1H, s), 4.42 (2H, d, J=5.9 Hz), 3.83 (3H, s), 3.41 (3H, s). MS (APCI+)=347.

### EXAMPLE 220

### 3-[4-(N-Hydroxycarbamimidoyl)-benzyl]-1-methyl-2,4-dioxo-1,2,3,4-tetrahydro-thieno[2,3-d]pyrimidine-6-carboxylic acid 4-methoxy-benzylamide

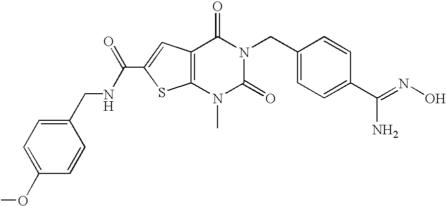

3-(4-Cyano-benzyl)-1-methyl-2,4-dioxo-1,2,3,4-tetrahydro-thieno[2,3-d]pyrimidine-6-carboxylic acid 4-methoxy-benzylamide (3.03 g, 6.59 mmol) was suspended in 50 absolute ethanol, and hydroxylamine hydrochloride (2.17 g, 31.16 mmol) and solid potassium hydroxide (1.92 g, 34.15 mmol) were added. The solution was refluxed for 4 hours, cooled to room temperature, and the solid was filtered off, washed with cold water, and dried under vacuum to give a white solid (2.69 g). mp 226-228° C. (dec.), MS (APCI+)=494.

### EXAMPLE 221

### 1-Methyl-2,4-dioxo-3-[4-(5-oxo-4,5-dihydro-1,2,4-oxadiazol-3-yl)-benzyl]-1,2,3,4-tetrahydro-thieno[2,3-d]pyrimidine-6-carboxylic acid 4-methoxy-benzylamide

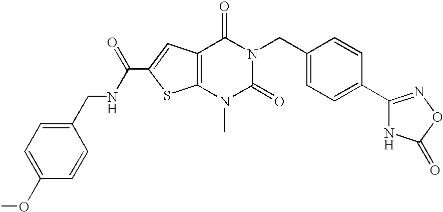

3-[4-(N-Hydroxycarbamimidoyl)-benzyl]-1-methyl-2,4-dioxo-1,2,3,4-tetrahydro-thieno[2,3-d]pyrimidine-6-carboxylic acid 4-methoxy-benzylamide (0.2897 g, 0.5870 mmol) was dissolved in 5 mL DMF and cooled in an ice bath, and pyridine (dissolved in 0.5 mL DMF) was added to this solution. Then ethyl chloroformate (0.0742 g, 0.6837 mmol) dissolved in 0.5 mL DMF was added, and the reaction stirred at 0° C. for 45 minutes. The reaction mixture was poured into 50 mL of water, the solid filtered off, washed with hexane, and suction dried to give a white powder, 1-methyl-2,4-dioxo-3-[4-(5-oxo-4,5-dihydro-1,2,4-oxadiazol-3-yl)-benzyl]-1,2,3,4-tetrahydro-thieno[2,3-d]pyrimidine-6-carboxylic acid 4-methoxy-benzylamide (0.24 g). CHNS calculated: C, 57.80%; H, 4.07%; N, 13.58%; S, 6.17%. Found: C, 57.47%; H, 4.19%; N, 13.19%; S, 6.13%.

### EXAMPLE 222

### 1-Methyl-2,4-dioxo-3-[4-(5-thioxo-4,5-dihydro-1,2,4-oxadiazol-3-yl)-benzyl]-1,2,3,4-tetrahydro-thieno[2,3-d]pyrimidine-6-carboxylic acid 4-methoxy-benzylamide

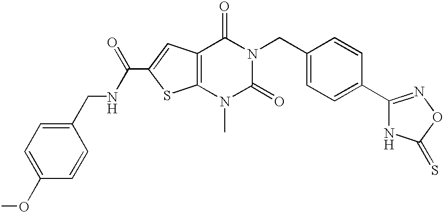

3-[4-(N-Hydroxycarbamimidoyl)-benzyl]-1-methyl-2,4-dioxo-1,2,3,4-tetrahydro-thieno[2,3-d]pyrimidine-6-carboxylic acid 4-methoxy-benzylamide (0.2137 g, 0.4330 mmol) was suspended in 5 mL DMF, then thiocarbonyl diimidazole (0.1225 g, 0.6187 mmol) and 1,8-diazabicyclo[5.4.0]undec-7-ene (“DBU”) (0.3427 g, 2.25 mmol) were added. After stirring at room temperature for 4 hours, the solution was poured into 60 mL water and acidified to pH 5 with 5% citric acid solution. The solid was filtered, resuspended in 5% citric acid solution, and sonicated. The solid was filtered and vacuum dried to give 1-methyl-2,4-dioxo-3-[4-(5-thioxo-4,5-dihydro-1,2,4-oxadiazol-3-yl)-benzyl]-1,2,3,4-tetrahydro-thieno[2,3-d]pyrimidine-6-carboxylic acid 4-methoxy-benzylamide (0.2511 g) as a tan powder. NMR (DMSO): 9.18 (1H, t, J=5.9 Hz), 8.12, (1H, s), 7.81 (2H, d, J=8.3 Hz), 7.48 (2H, d, J=8.3 Hz), 7.23 (2H, d, J=9.12 Hz), 6.90 (2H, d, J=8.8 Hz), 5.13 (2H, s), 4.38 (2H, d, J=5.9 Hz), 3.73 (3H, s), 3.49 (3H, s). MS (APCI+)=536.

### EXAMPLE 223

### 3-Cyanomethyl-1-methyl-2,4-dioxo-1,2,3,4-tetrahydro-thieno[2,3-d]pyrimidine-6-carboxylic acid 4-methoxy-benzylamide

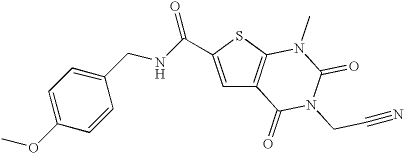

Made by the procedure of Example 217 with chloroacetonitrile; mp 180-183° C.

### EXAMPLE 224

### 3-Cyanomethyl-1-methyl-2,4-dioxo-1,2,3,4-tetrahydro-thieno[2,3-d]pyrimidine-6-carboxylic acid 3-methoxy-benzylamide

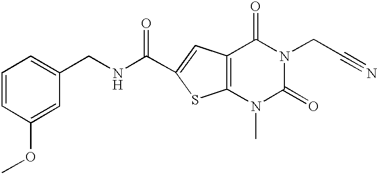

Made by the procedure of Example 217 with chloroacetonitrile; mp 159-163° C.

### EXAMPLE 225

### (E)-4-[6-(4-Methoxy-benzylcarbamoyl)-1-methyl-2,4-dioxo-1,4-dihydro-2H-thieno[2,3-d]pyrimidin-3-yl]-but-2-enoic acid methyl ester

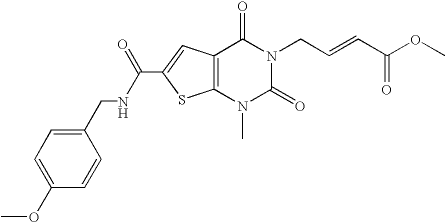

Made by the procedure of Example 217 with methyl 4-bromocrotonate; mp 169-171° C.

### EXAMPLE 226

### (E)-4-[6-(4-Methoxy-benzylcarbamoyl)-1-methyl-2,4-dioxo-1,4-dihydro-2H-thieno[2,3-d]pyrimidin-3-yl]-but-2-enoic acid

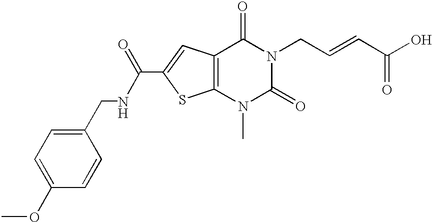

(E)-4-[6-(4-Methoxy-benzylcarbamoyl)-1-methyl-2,4-dioxo-1,4-dihydro-2H-thieno[2,3-d]pyrimidin-3yl]-but-2-enoic acid (0.053 g, 0.1195 mmol) was dissolved in 5 mL methanol and 5 mL water with potassium carbonate (0.0264 g, 0.191 mmol) and heated to reflux for 4 hours. The reaction was cooled to room temperature, and the solution concentrated to approximately 5 mL. The solution was acidified with 5% HCl, and the white precipitate was filtered and dried under vacuum. NMR (DMSO, ppm): 9.17 (1H, t, J=6.1 Hz), 8.11 (1H, s), 7.23 (2H, d, J=8.5 Hz), 6.91 (2H, d), 6.8 (1H, m), 5.76 (1H, m), 4.62 (2H, m), 4.37 (2H, d, J=5.9 Hz), 3.72 (3H, s), 3.48 (3H, s).

### EXAMPLE 227

### 3-(2-Benzenesulfonyl-ethyl)-1-methyl-2,4-dioxo-1,2,3,4-tetrahydro-thieno[2,3-d]pyrimidine-6-carboxylic acid 4-methoxy-benzylamide

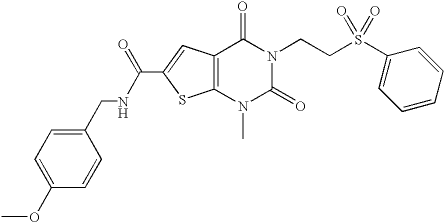

Made by the procedure of Example 217 with (2-chloro-ethanesulfonyl)-benzene; mp 118-121° C.

### EXAMPLE 228

### 2-Methoxy-4-[6-(4-methoxy-benzylcarbamoyl)-1-methyl-2,4-dioxo-1,4-dihydro-2H-thieno[2,3-d]pyrimidin-3-ylmethyl]-benzoic acid methyl ester

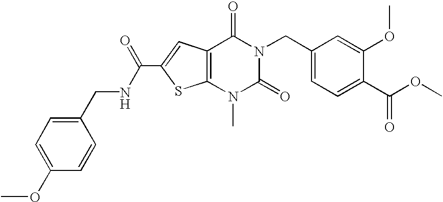

Made by the procedure of Example 217 with methyl 4-bromomethyl-2-methoxybenzoate; mp 201-203° C.

### EXAMPLE 229

### 3-(2-Methoxymethyl-1,1,3-trioxo-2,3-dihydro-1H-1l⁶l -1,2-benzisothiazol-6-ylmethyl)-1-methyl-2,4-dioxo-1,2,3,4-tetrahydro-thieno[2,3-d]pyrimidine-6-carboxylic acid 4-methoxy-benzylamide

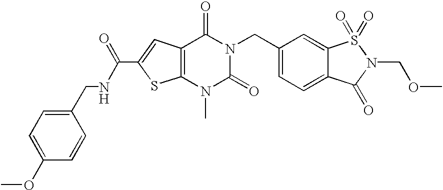

Made by the procedure of Example 217 with 6-bromomethyl-(2-methoxymethyl-1,1,3-trioxo-2,3-dihydro-1H-1l⁶-1,2-benzisothiazole. NMR (DMSO) 9.22 (1H, m), 8.3-7.9 (4H, m), 7.24 (2H, d, J=8.88 Hz), 6.9 (2H, d, J=8.5 Hz), 5.27 (2H, s), 5.15 (2H, s), 4.37 (2H, m), 3.73 (3H, s), 3.51 (3H, s), 3.35 (3H, s). MS (APCI−)=583.

### EXAMPLE 230

### 1-Methyl-3-oct-2-ynyl-2,4-dioxo-1,2,3,4-tetrahydro-thieno[2,3-d]pyrimidine-6-carboxylic acid 4-methoxy-benzylamide

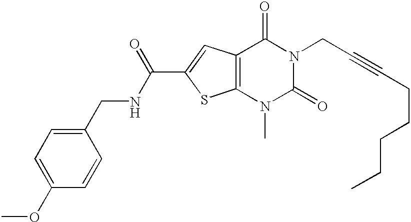

Made by the procedure of Example 217 with 1-bromo-2-octyne. NMR (DMSO): 9.18 (1H, t, J=6.1 Hz), 8.11 (1H, s), 7.24, (2H, d, J=8.5), 6.90 (2H, d, J=8.5), 4.58 (2H, s), 4.38 (2H, d, J=5.9 Hz), 3.73 (3H, s), 3.49 (3H, s), 2.13 (2H, m), 1.45-1.2 (6H, m), 0.83 (3H, m). MS (APCI+)=454.

### EXAMPLE 231

### 3-[2-(4-Chloro-benzenesulfonylamino)-ethyl]-1-methyl-2,4-dioxo-1,2,3,4-tetrahydro-thieno[2,3-d]pyrimidine-6-carboxylic acid 4-methoxy-benzylamide

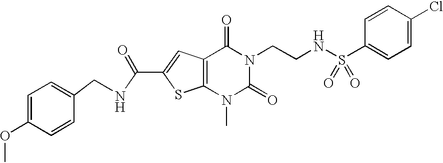

Made by the procedure of Example 217 with 1-chloro-2-(4-chloro-benzenesulfonylamino)-ethane. NMR (DMSO): 9.16 (1H, t, J=5.9 Hz), 8.04 (1H, s), 7.71 (2H, d), 7.60 (2H, d), 7.24 (2H, d), 6.90 (2H, d), 4.37 (2H, d), 3.91 (2H, t), 3.73 (3H, s), 3.44 (s), 3.06 (2H, t). MS (APCI+)=563.

EXAMPLE 231a

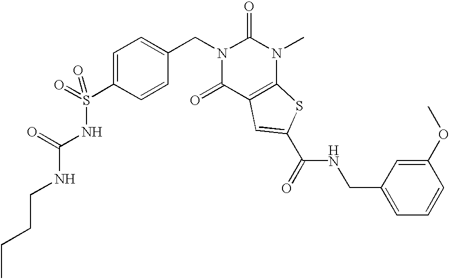

1-Methyl-2,4-dioxo-3-(4-sulfamoyl-benzyl)-1,2,3,4-tetrahydro-thieno[2,3-d]pyrimidine-6-carboxylic acid 3-methoxy-benzylamide (0.1031 g, 0.2002 mmol) was dissolved in 2 mL N,N-dimethylformamide (“DMF”), cooled in an ice bath, and sodium hydride (60% in oil, 0.019 g, 0.8041 mmol) was added, and stirred at 0° for 1 hour. Then n-butyl isocyanate (0.0273 g, 0.2752 mmol) was added and stirred for 2 hours. The reaction mixture was added dropwise to 25 mL of water, and the pH was adjusted to 4 with 5% citric acid solution. The mixture was extracted twice with ethyl acetate, and once with chloroform. The combined organic extracts were washed with water, then brine, and dried over magnesium sulfate. The solution was filtered, and the solvent removed to give a solid that was triturated with ethyl acetate. The solid was collected by filtration and suction dried to give a white solid (0.0691 g) NMR (DMSO): 10.35 (1H, s), 9.24 (1H, t), 8.15 (1H, s), 7.81 (2H, d), 7.50 (2H, d), 7.24 (1H, m), 6.9-6.8 (3H, m), 6.43 (1H, m), 5.14 (2H, s), 4.43 (2H, d), 3.73 (3H, s), 3.49 (3H, s), 2.9 (2H, m), 1.4-1.1 (4H, m), 0.8 (3H, m). CHNS (calculated): C, 54.80%; H, 5.09%; N, 11.41%; S, 10.45%. Found: C, 54.29%; H5.06%; N, 11.40%; S, 10.69%; water 0.64%.

### EXAMPLE 232

### 3-[2-(4-Bromo-phenoxy)-ethyl]-1-methyl-2,4-dioxo-1,2,3,4-tetrahydro-thieno[2,3-d]pyrimidine-6-carboxylic acid 4-methoxy-benzylamide

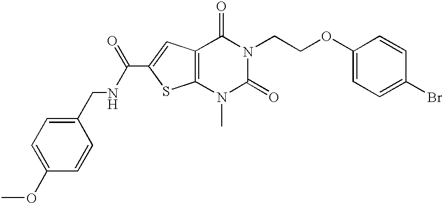

Made by the procedure of Example 217 with 1-methyl-1,2,3,4-tetrahydro-thieno[2,3-d]pyrimidine-6-carboxylic acid 4-methoxy-benzylamide and 1-bromo-2-(2-chloro-ethoxy)benzene; mp 184-185° C.

### EXAMPLE 233

### 3-[2-(4-Bromo-phenoxy)-ethyl]-1-methyl-2,4-dioxo-1,2,3,4-tetrahydro-thieno[2,3-d]pyrimidine-6-carboxylic acid 3-methoxy-benzylamide

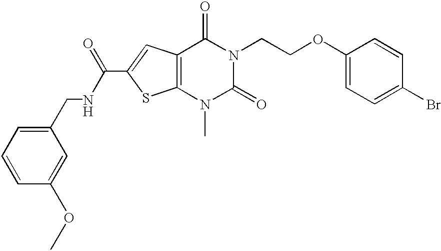

Made by the procedure of Example 217 from 1-methyl-1,2,3,4-tetrahydro-thieno[2,3-d]pyrimidine-6-carboxylic acid 3-methoxy-benzylamide and 1-bromo-4-(2-chloro-ethoxy)benzene; mp 165-167° C.

### EXAMPLE 234

### 3-[2-(4-Fluoro-phenoxy)-ethyl]-1-methyl-2,4-dioxo-1,2,3,4-tetrahydro-thieno[2,3-d]pyrimidine-6-carboxylic acid 4-methoxy-benzylamide

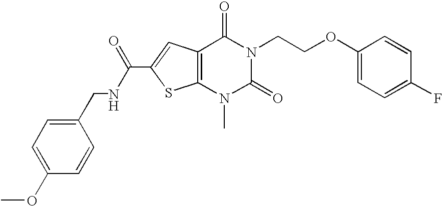

Made by the procedure of Example 217 from 1-methyl-1,2,3,4-tetrahydro-thieno[2,3-d]pyrimidine-6-carboxylic acid 3-methoxy-benzylamide and 1-fluoro-4-(2-chloro-ethoxy)benzene; mp 170-171° C.

### EXAMPLE 235

### 3-(2-aminoethyl)-1-methyl-2,4-dioxo-1,2,3,4-tetrahydro-thieno[2,3-d]pyrimidine-6-carboxylic acid 4-methoxy-benzylamide

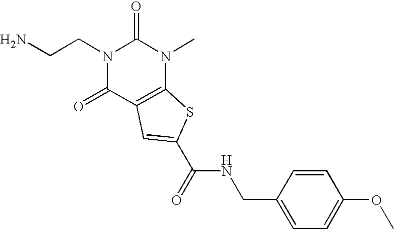

3-Cyanomethyl-1-methyl-2,4-dioxo-1,2,3,4-tetrahydro-thieno[2,3-d]pyrimidine-6-carboxylic acid 4-methoxy-benzylamide (1.95 g) was dissolved in 95 mL tetrahydrofuran (“THF”) with 5 mL triethylamine, and 0.45 Raney Ni catalyst added. The reaction was placed under a hydrogen atmosphere (50 psi) and shaken for 39 hours. The catalyst was filtered off, and the solvent removed to give a white solid. NMR (DMSO): 9.15 (1H, t, J=5.6), 8.09 (1H, s), 7.23 (2H, d, J=8.5 Hz), 6.90 (2H, d, J=8.5 Hz), 4.37 (2H, d, J=5.9 Hz), 3.88 (2H, t, J=6.4 Hz), 3.73 (3H, s), 4.47 (3H, s), 2.7 (2H, t, J=6.8 Hz). MS (APCI+)=389.

### EXAMPLE 236

### 3-[2-(4-fluoro-phenoxy)-ethyl]-1-methyl-2,4-dioxo-1,2,3,4-tetrahydro-thieno[2,3-d]pyrimidine-6-carboxylic acid 3-methoxy-benzylamide

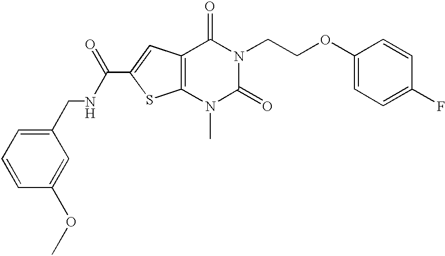

Made by the procedure of Example 217 from 1-methyl-1,2,3,4-tetrahydro-thieno[2,3-d]pyrimidine-6-carboxylic acid 3-methoxy-benzylamide and 1-fluoro-4-(2-chloro-ethoxy)benzene. NMR (DMSO): 9.21 (1H, t, J=5.9 Hz), 8.13 (1H, s), 7.3-6.8 (8H, m), 4.5-4.1 (6H, m), 3.74 (3H, s), 3.49 (3H, s). MS (APCI+)=484. CHNS calculated: C, 59.62%; H, 4.59%; N, 8.69%; S, 6.63%. F, 3.93%. Found: C, 59.71%; H, 4.61%; N, 8.62%; S, 6.69%; F, 4.03%.

### EXAMPLE 237

### 3-[2-(4-chloro-phenoxy)-ethyl]-1-methyl-2,4-dioxo-1,2,3,4-tetrahydro-thieno[2,3-d]pyrimidine-6-carboxylic acid 3-methoxy-benzylamide

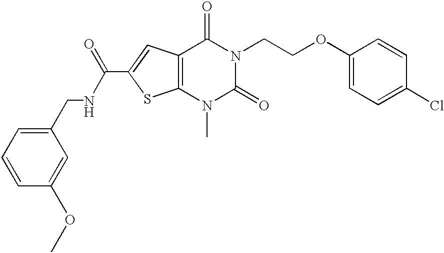

Made by the procedure of Example 217 from 1-methyl-1,2,3,4-tetrahydro-thieno[2,3-d]pyrimidine-6-carboxylic acid 3-methoxy-benzylamide and 4-(2-bromo-ethoxy)-1-chlorobenzene; mp 109-112° C.

### EXAMPLE 238

### 4-[6-(4-Methoxy-benzylcarbamoyl)-1-methyl-2,4-dioxo-1,4-dihydro-2H-thieno[2,3-d]pyrimidin-3-ylmethyl]-2-methyl-benzoic acid methyl ester

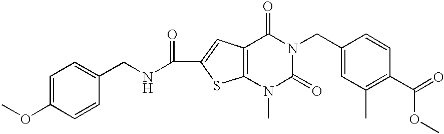

Made by the procedure of Example 217 with 4-bromomethyl-2-methylbenzoic acid methyl ester; mp 179-181° C.

### EXAMPLE 239

### 4-[6-(4-Methoxy-benzylcarbamoyl)-1-methyl-2,4-dioxo-1,4-dihydro-2H-thieno[2,3-d]pyrimidin-3-ylmethyl]-benzoic acid methyl ester

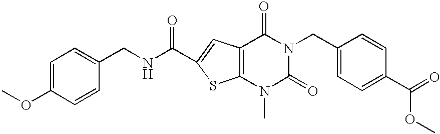

Made by the procedure of Example 217 with 4-bromomethyl benzoic acid methyl ester; mp 235-237° C.

### EXAMPLE 240

### 2-Methoxy-4-[6-(3-methoxy-benzylcarbamoyl)-1-methyl-2,4-dioxo-1,4-dihydro-2H-thieno[2,3-d]pyrimidin-3-ylmethyl]-benzoic acid methyl ester

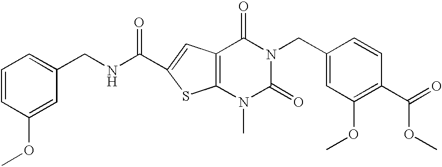

Made by the procedure of Example 217 with 4-bromomethyl-2-methoxy-benzoic acid methyl ester; mp 200-203° C.

### EXAMPLE 241

### 4-[6-(3-Methoxy-benzylcarbamoyl)-1-methyl-2,4-dioxo-1,4-dihydro-2H-thieno[2,3-d]pyrimidin-3-ylmethyl]-2-methyl-benzoic acid methyl ester

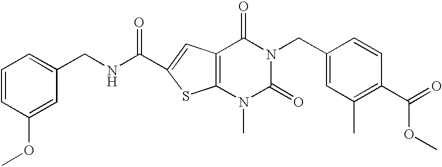

Made by the procedure of Example 217 with 4-bromomethyl-2-methyl-benzoic acid methyl ester; mp 175-177° C.

### EXAMPLE 242

### 1-Methyl-2,4-dioxo-3-(3-oxo-3-phenyl-propyl)-1,2,3,4-tetrahydro-thieno[2,3-d]pyrimidine-6-carboxylic acid 4-methoxy-benzylamide

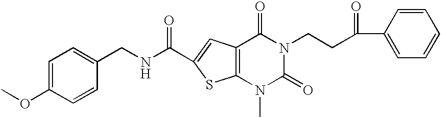

Made by the procedure of Example 217 with 3-chloro-1-phenyl-propan-1-one; mp. 208-211° C.

### EXAMPLE 243

### 1-Methyl-2,4-dioxo-3-(3-oxo-3-phenyl-propyl)-1,2,3,4-tetrahydro-thieno[2,3-d]pyrimidine-6-carboxylic acid 3-methoxy-benzylamide

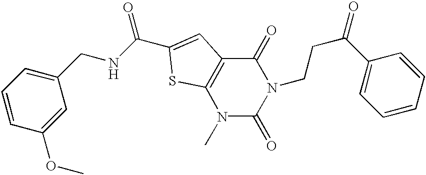

Made by the procedure of Example 217 with 3-chloro-1-phenyl-propan-1-one; mp 188-191° C.

### EXAMPLE 244

### 1-Methyl-2,4-dioxo-3-[2-(3-trifluoromethyl-benzenesulfonyl)-ethyl]-1,2,3,4-tetrahydro-thieno[2,3-d]pyrimidine-6-carboxylic acid 3-methoxy-benzylamide

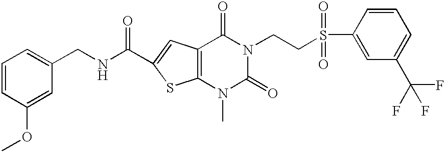

Made by the procedure of Example 217 with (2-chloro-ethanesulfanyl)-3-trifluoromethyl benzene; mp 203-205° C.

### EXAMPLE 245

### 1-Methyl-2,4-dioxo-3-[2-(3-trifluoromethyl-benzenesulfonyl)-ethyl]-1,2,3,4-tetrahydro-thieno[2,3-d]pyrimidine-6-carboxylic acid 4-methoxy-benzylamide

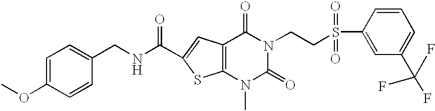

Made by the procedure of Example 217 with (2-chloro-ethanesulfanyl)-3-trifluoromethyl benzene; mp 222-225° C.

### EXAMPLE 246

### 3-[2-(4-Chloro-benzenesulfonyl)-ethyl]-1-methyl-2,4-dioxo-1,2,3,4-tetrahydro-thieno[2,3-d]pyrimidine-6-carboxylic acid 3-methoxy-benzylamide

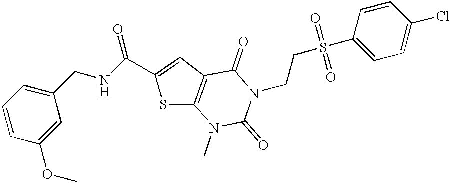

Made by the procedure of Example 217 with 4-(2-chloro-ethanesulfanyl)-chlorobenzene; mp 186-190° C.

### EXAMPLE 247

### 3-[2-(4-Chloro-benzenesulfonyl)-ethyl]-1-methyl-2,4-dioxo-1,2,3,4-tetrahydro-thieno[2,3-d]pyrimidine-6-carboxylic acid 4-methoxy-benzylamide

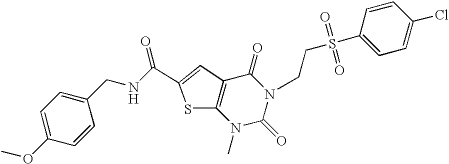

Made by the procedure of Example 217 with 4-(2-chloro-ethanesulfanyl)-chlorobenzene; mp 222-225° C.

### EXAMPLE 248

### 1-Methyl-2,4-dioxo-1,2,3,4-tetrahydro-thieno[2,3-d]pyrimidine-6-carboxylic acid

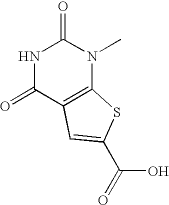

1-Methyl-2,4-dioxo-1,2,3,4-tetrahydro-thieno[2,3-d]pyrimidine-6-carboxylic acid benzyl ester (1.70 g, 5.387 mmol) was stirred with anhydrous HBr/acetic acid for 3 days at room temperature. An equal volume of water was added, and the solid was filtered off and dried under vacuum to give 1.06 g white solid. MS (APCI−) 225.

### EXAMPLE 249

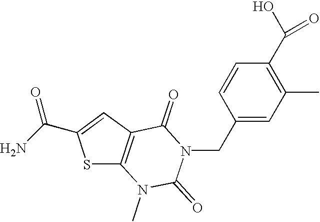

### EXAMPLE 249a

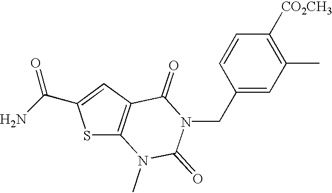

4-[6-(4-Methoxy-benzylcarbamoyl)-1-methyl-2,4-dioxo-1,4-dihydro-2H-thieno[2,3-d]pyrimidin-3-ylmethyl]-2-methyl-benzoic acid methyl ester (0.1543 g, 0.304 mmol) was stirred with 5 mL anhydrous HBr/acetic acid for 2 days at room temperature. The solution was added dropwise to 50 mL water, and the solid filtered off. The solid was purified by column chromatography to give Example 249 [MS (APCI+) 374] and Example 249a [MS (APCI+) 388].

### EXAMPLE 250

### 4-[6-(3-hydroxy-benzylcarbamoyl)-1-methyl-2,4-dioxo-1,4-dihydro-2H-thieno[2,3-d]pyrimidin-3-ylmethyl]-2-methyl-benzoic acid

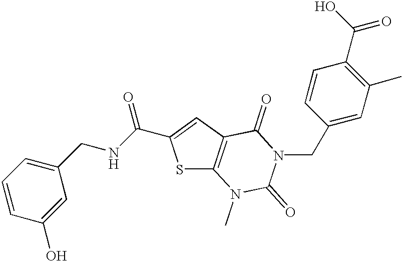

4-[6-(3-Methoxy-benzylcarbamoyl)-1-methyl-2,4-dioxo-1,4-dihydro-2H-thieno[2,3-d]pyrimidin-3-ylmethyl]-2-methyl-benzoic acid methyl ester (0.2489 g, 0.4903 mmol) was stirred with 5 mL anhydrous HBr/acetic acid for 3 days at room temperature. The solution was added dropwise to 50 mL of 5% hydrochloric acid and stirred 1 hour. The solid was filtered off and suction dried. The process was repeated, and the resulting solid was purified by column chromatography to give 4-[6-(3-hydroxy-benzylcarbamoyl)-1-methyl-2,4-dioxo-1,4-dihydro-2H-thieno[2,3-d]pyrimidin-3-ylmethyl]-2-methyl-benzoic acid. MS (APCI+) 480.

### EXAMPLE 251

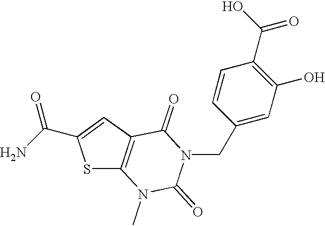

This was made analogously to Example 249. MS (APCI−) 374.

### EXAMPLE 252

### 3-(2-Aminoethyl)-1-methyl-2,4-dioxo-1,2,3,4-tetrahydro-thieno[2,3-d]pyrimidine-6-carboxylic acid 3-methoxy-benzylamide

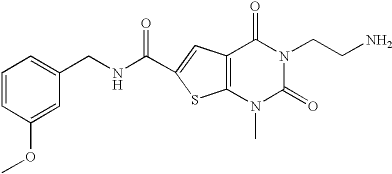

3-Cyanomethyl-1-methyl-2,4-dioxo-1,2,3,4-tetrahydro-thieno[2,3-d]pyrimidine-6-carboxylic acid 3-methoxy-benzylamide (4.3 g, 11.19 mmol) was reduced with Raney Nickel in THF under hydrogen. The catalyst was filtered off, and the solvent removed under vacuum to give the product as a white solid. MS (APCI+) 389.

### EXAMPLE 252a

In a 8 mL screw cap vial was added a solution of the compound of Example 216 (0.034 g, 0.1 mmol) in dimethylformamide (1 mL), a solution of 3-chloro-1-(Z)-phenyl-propan-1-one(0.039 g, 0.23 mmol) in dimethylformamide (575 mL) and anhydrous cesium carbonate(0.075 g, 0.023 mmol). The vial was capped, and the reaction mixture was shaken for 24 hours at room temperature. The reaction mixture was filtered, and the solvent was removed under vacuum. Purification was carried out via reverse-phase HPLC (3% n-propanol in acetonitrile and 3% n-propanol in water as the eluent; C-18 column). 0.012 g 30% yield). MS-APCI (M+1) 478.1.

In a manner similar to the procedure of Example 252a, the following invention compounds were prepared:

| Example | | |
|---|---|---|
| No. | Structure | APCI MS (M + 1) |
| 253 | 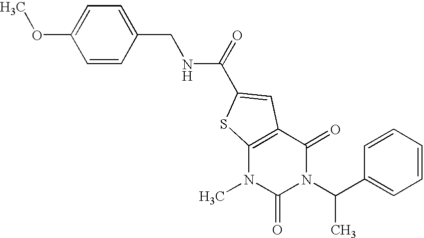 | 450.5287 |
| 254 | 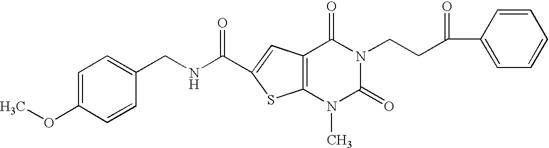 | 478.5387 |
| 255 | 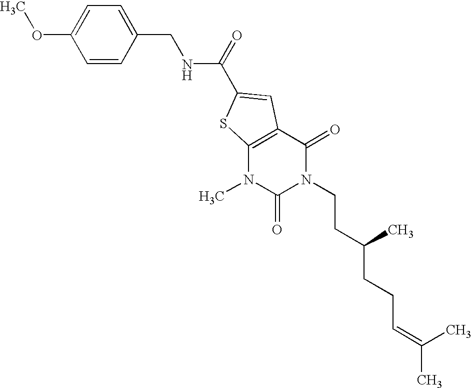 | 484.6297 |
| 256 | 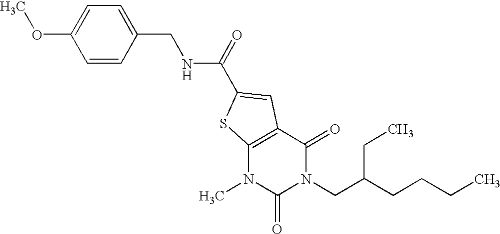 | 458.5919 |
| 257 | 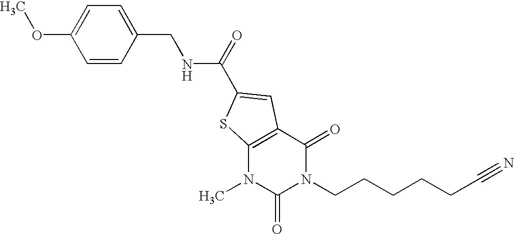 | 441.5216 |
| 258 | 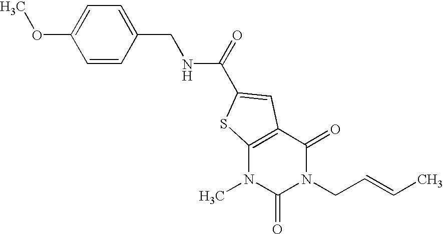 | 400.4689 |
| 259 | 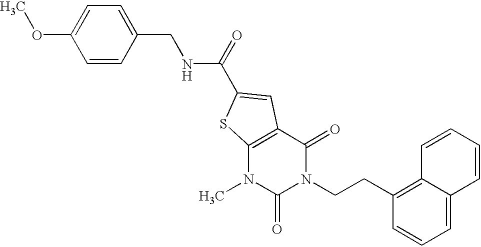 | 500.5885 |
| 260 | 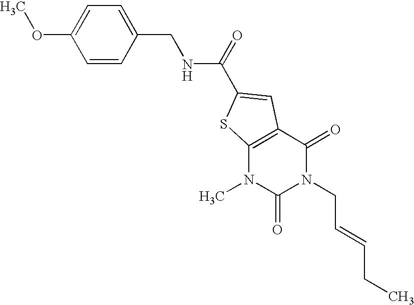 | 414.4957 |
| 261 | 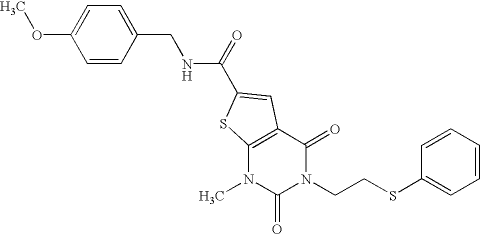 | 482.5947 |
| 262 | 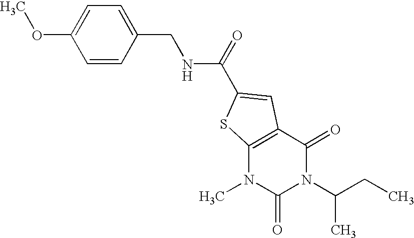 | 402.4847 |
| 263 | 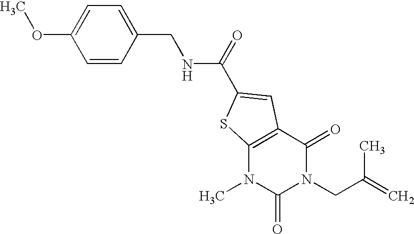 | 400.4689 |
| 264 | 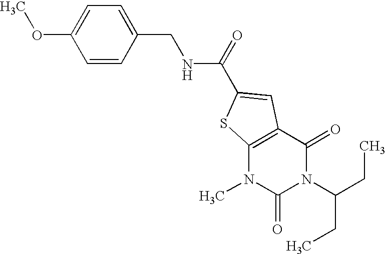 | 416.5115 |
| 265 | 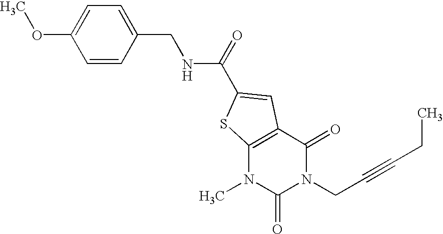 | 412.4799 |
| 266 | 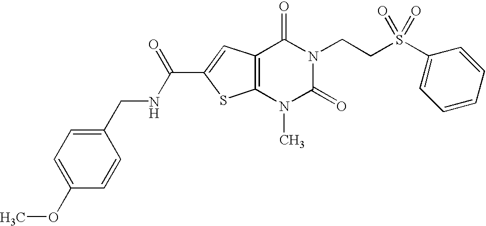 | 514.5927 |
| 267 | 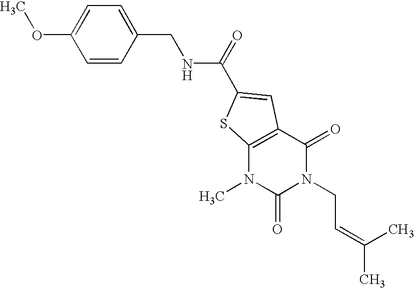 | 414.4957 |
| 268 | 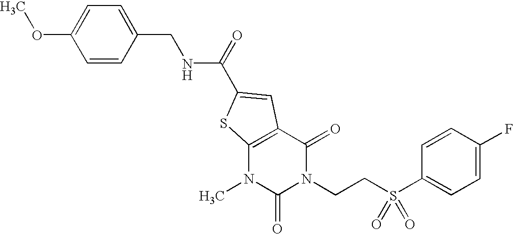 | 532.5828 |
| 269 | 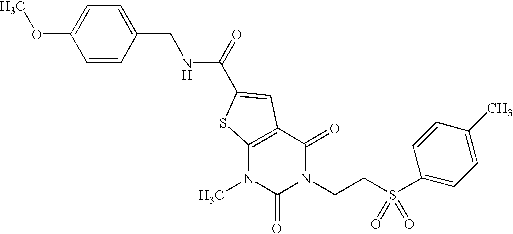 | 528.6195 |
| 270 | 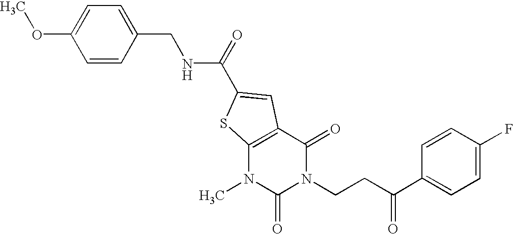 | 496.5288 |
| 271 | 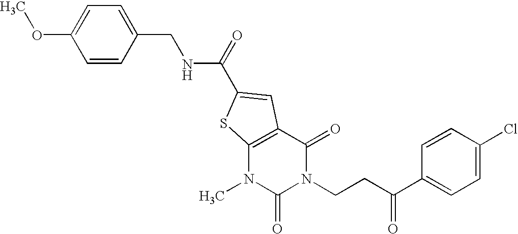 | 512.9838 |
| 272 | 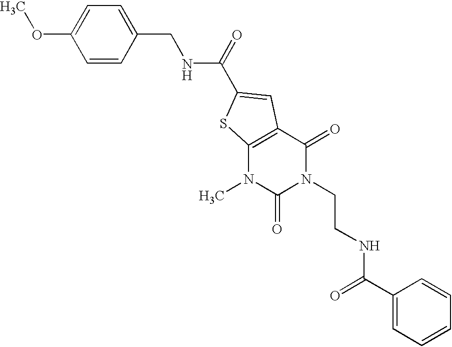 | 493.5536 |
| 273 | 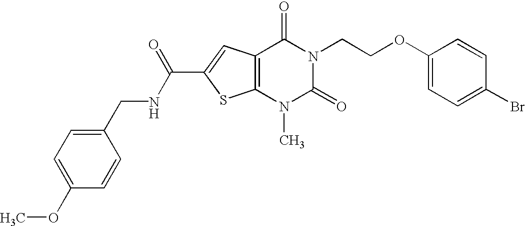 | 545.4238 |
| 274 | 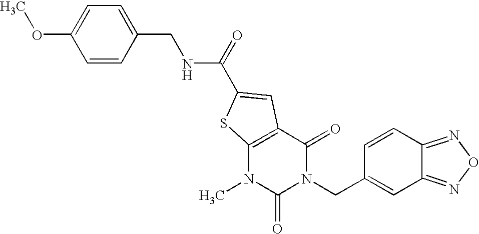 | 478.4991 |
| 275 | 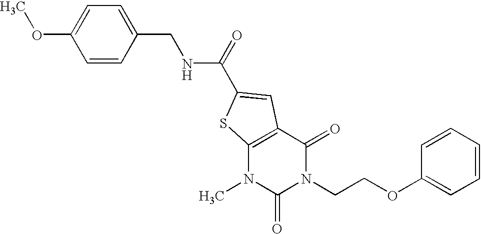 | 466.5277 |
| 276 | 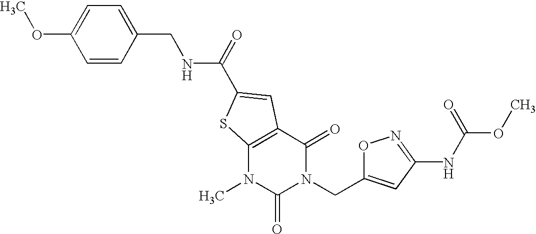 | 500.5019 |
| 277 | 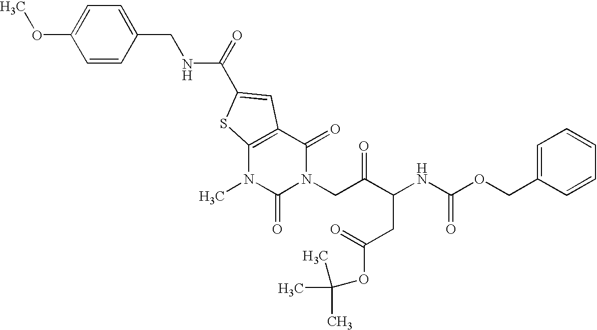 | 665.7324 |
| 278 | 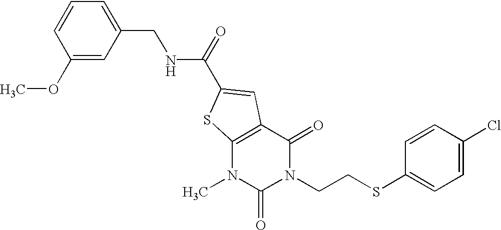 | 517.0398 |
| 279 | 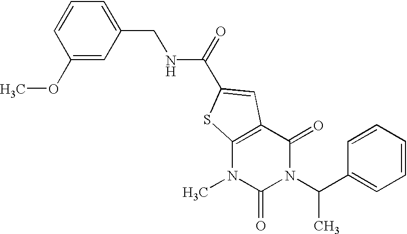 | 450.5287 |
| 280 | 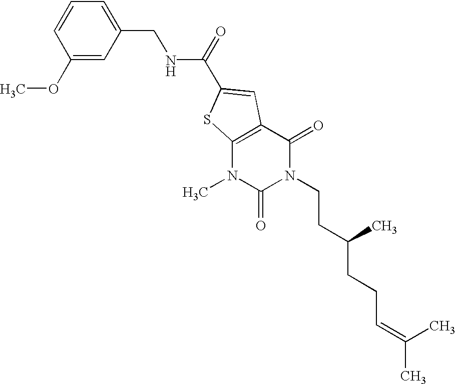 | 484.6297 |
| 281 | 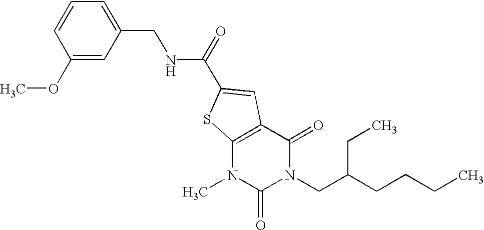 | 458.5919 |
| 282 | 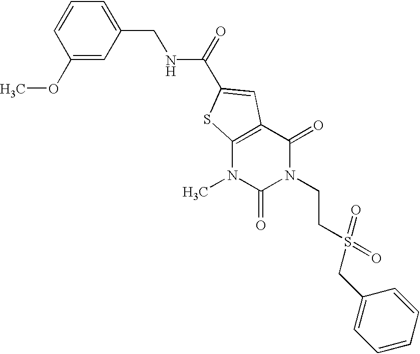 | 528.6195 |
| 283 | 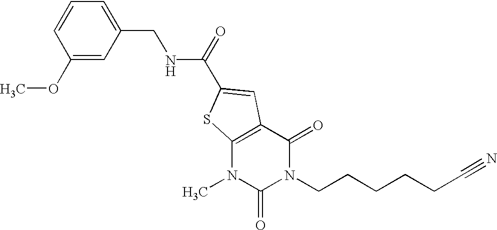 | 441.5216 |
| 284 | 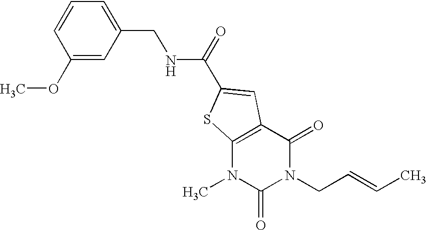 | 400.4689 |
| 285 | 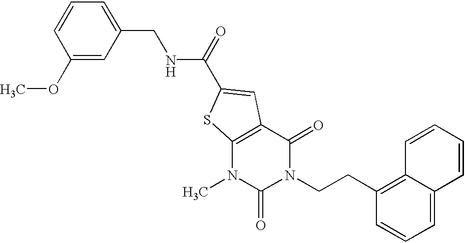 | 500.5885 |
| 286 | 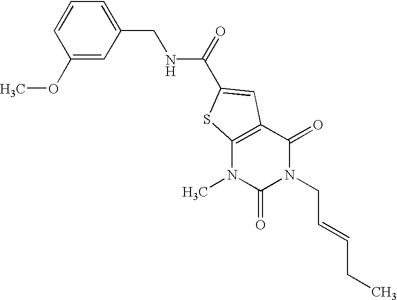 | 414.4957 |
| 287 | 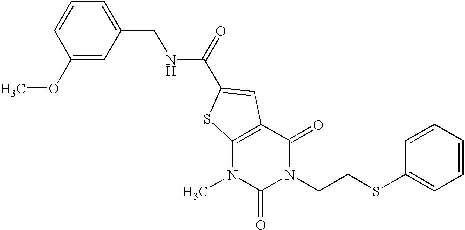 | 482.5947 |
| 288 | 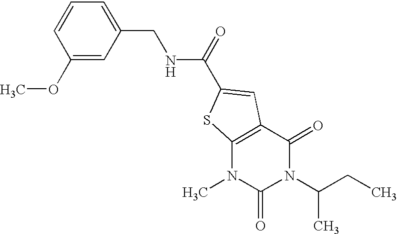 | 402.4847 |
| 289 | 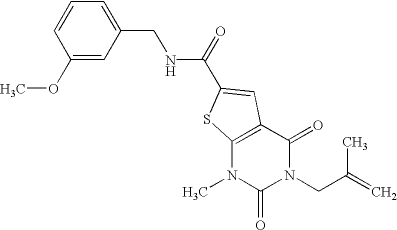 | 400.4689 |
| 290 | 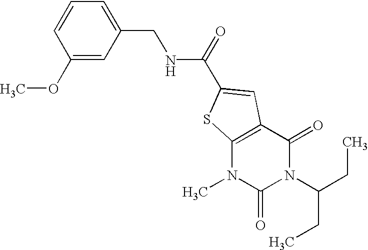 | 416.5115 |
| 291 | 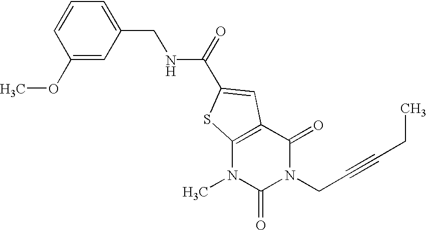 | 412.4799 |
| 292 | 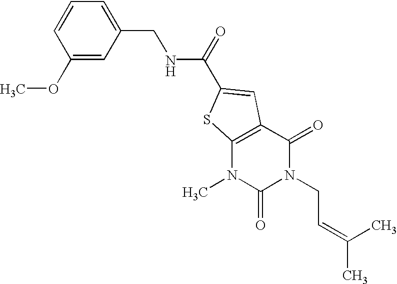 | 414.4957 |
| 293 | 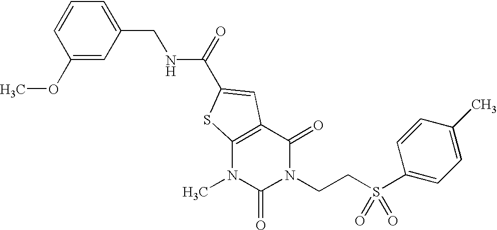 | 528.6195 |
| 294 | 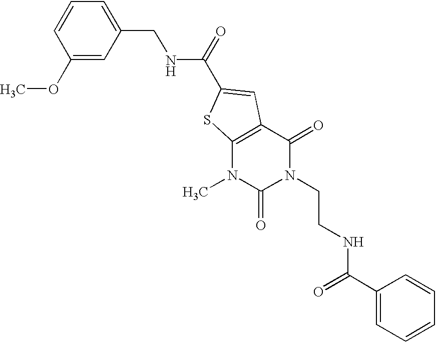 | 493.5536 |
| 295 | 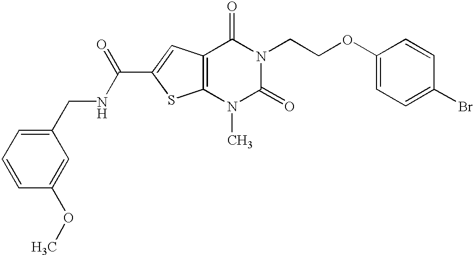 | 545.4238 |
| 296 | 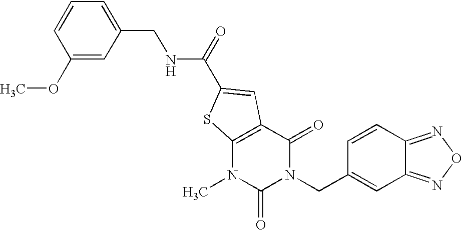 | 478.4991 |
| 297 | 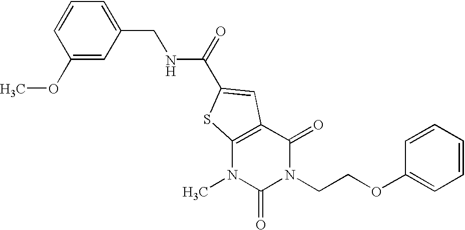 | 466.5277 |
| 298 | 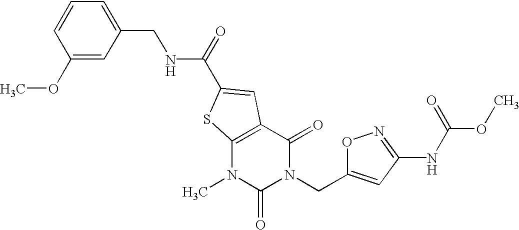 | 500.5019 |
| 299 | 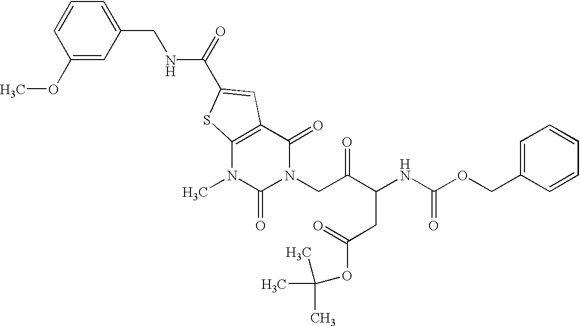 | 665.7324 |

Also prepared by the methods exemplified above are the compounds of 300 to 386.

### EXAMPLE 300

### 3-Benzyl-1-methyl-2,4-dioxo-1,2,3,4-tetrahydro-thieno[2,3-d]pyrimidine-6-carboxylic acid methyl ester made by the procedure of Example 97

MS-APCI (M+1) 331.2.

### EXAMPLE 301

### 3-(4-Bromo-benzyl)-5-methyl-2,4-dioxo-1,2,3,4-tetrahydro-thieno[2,3-d]pyrimidine-6-carboxylic acid benzyl ester

To a solution of 5-amino-3-methyl-thiophene-2,4-dicarboxylic acid 2-benzyl ester 4-ethyl ester (0.5 g, 1.57 mmol) in dioxane (50 mL), was added sodium hydride (42 mg, 1.72 mmol). 1-bromo-4-isocyanatomethyl-benzene (0.332 g, 1.57 mmol) was added 5 minutes later. The reaction mixture was stirred at room temperature for 1 hour. The reaction mixture was chromatographed using 2:1 hexane:ethyl acetate to yield 82 mg of title compound as a white solid (11%); MS-APCI (M+) 487.

### EXAMPLE 302

### 3-(4-Fluoro-benzyl)-5-methyl-2,4-dioxo-1,2,3,4-tetrahydro-thieno[2,3-d]pyrimidine-6-carboxylic acid benzyl ester

The procedure of Example 74 was repeated, except 1-bromo-4-isocyanatomethyl-benzene was replaced by 1-fluoro-4-isocyanatomethyl-benzene to give 3-(4-fluoro-benzyl)-5-methyl-2,4-dioxo-1,2,3,4-tetrahydro-thieno[2,3-d]pyrimidine-6-carboxylic acid benzyl ester as a white solid (13%); MS-APCI (M+) 425.

### EXAMPLE 303

### 3-Benzyl-1-methyl-2,4-dioxo-1,2,3,4-tetrahydro-thieno[2,3-d]pyrimidine-6-carboxylic acid pyridin-4-ylmethyl ester

### EXAMPLE 304

### 3-Benzyl-1-methyl-2,4-dioxo-1,2,3,4-tetrahydro-thieno[2,3-d]pyrimidine-6-carboxylic acid benzo[b]thiophen-2-ylmethyl ester

The procedure of Example 48 was repeated, except and benzofuran-2-yl-methanol was replaced by benzothiophene-2-yl-methanol to give 3-benzyl-1-methyl-2,4-dioxo-1,2,3,4-tetrahydro-thieno[2,3-d]pyrimidine-6-carboxylic acid benzo[b]thiophene-2-ylmethyl ester as a white solid (54%); ¹H NMR (CDCl₃), δ3.50 (S, 3H), 5.19 (s, 2H), 5.58 (s, 2H), 7.21-7.38 (m, 6H), 7.49 (d, J=8.7 Hz, 2H), 7.74-7.82 (m, 2H), 8.11 (s, 1H).

### EXAMPLE 305

### 3-Benzyl-1-methyl-2,4-dioxo-1,2,3,4-tetrahydro-thieno[2,3-d]pyrimidine-6-carboxylic acid 1-methyl-1H-indol-5-ylmethyl ester

The procedure of Example 48 was repeated, except and benzofuran-2-yl-methanol was replaced by (1-methyl-1H-indol-5-yl)-methanol to give 3-benzyl-1-methyl-2,4-dioxo-1,2,3,4-tetrahydro-thieno[2,3-d]pyrimidine-6-carboxylic acid 1-methyl-1H-indo-5-ylmethyl ester as a white solid (59%); ¹H NMR (CDCl₃), δ3.51 (s, 3H), 3.80 (s, 3H), 5.17 (s, 2H), 5.43 (s, 2H), 6.50 (d, J=3.2 Hz, 1H), 7.08 (d, J=3.1 Hz, 1H), 7.22-7.34 (m, 5 H), 7.49 (m, 2H), 7.70 (s, 1H), 8.06 (s, 1H).

### EXAMPLE 306

### 3-Benzyl-2,4-dioxo-1,2,3,4-tetrahydro-thieno[2,3-d]pyrimidine-6-carboxylic acid thiophen-3-ylmethyl ester

The procedure of Example 1 was repeated, except that benzyl alcohol was replaced by thiophene-2-ylmethanol to provide 3-benzyl-2,4-dioxo-1,2,3,4-tetrahydro-thieno[2,3-d]pyrimidine-6-carboxylic acid thiophen-3-ylmethyl ester as a white powder (19%); MS-APCI (M+) 400.

### EXAMPLE 307

### 3-1,3-Benzodioxol-5-ylmethyl-1-methyl-2,4-dioxo-1,2,3,4-tetrahydro-thieno[2,3-d]pyrimidine-6-carboxylic acid benzyl ester

The procedure of Example 15 was repeated, except 3-bromomethyl-benzoic acid methyl ester was replaced by 5-bromoethyl-benzo[1,3]dioxole. The crude product was chromatographed using 2:1 hexane/ethyl acetate to 100% ethyl acetate to give 3-1,3-benzodioxol-5-ylmethyl-1-methyl-2,4-dioxo-1,2,3,4-tetrahydro-thieno[2,3-d]pyrimidine-6-carboxylic acid benzyl ester as a white solid (40%); MS-APCI (M+) 451.

### EXAMPLE 308

### 1-Methyl-2,4-dioxo-3-pyridin-4-ylmethyl-1,2,3,4-tetrahydro-thieno[2,3-d]pyrimidine-6-carboxylic acid benzyl ester

The procedure of Example 15 was repeated, except 3-bromomethyl-benzoic acid methyl ester was replaced by 4-bromomethyl pyridine. The crude product was chromatographed using 2:1 hexane:ethyl acetate to 100% ethyl acetate to give 1-methyl-2,4-dioxo-3-pyridin-4-ylmethyl-1,2,3,4-tetrahydro-thieno[2,3-d]pyrimidine-6-carboxylic acid benzyl ester as a white solid (49%); MS-APCI (M+) 408.

### EXAMPLE 309

### 3-(4-tert-Butyl-benzyl)-1-methyl-2,4-dioxo-1,2,3,4-tetrahydro-thieno[2,3-d]pyrimidine-6-carboxylic acid benzyl ester

The procedure of Example 15 was repeated, except 3-bromomethyl-benzoic acid methyl ester was replaced by 1-bromomethyl-4-tert-butyl-benzene. The crude product was chromatographed using 2:1 hexane/ethyl acetate to 100% ethyl acetate to give 3-(4-tert-butyl-benzyl)-1-methyl-2,4-dioxo-1,2,3,4-tetrahydro-thieno[2,3-d]pyrimidine-6-carboxylic acid benzyl ester as a white solid (63%); MS-APCI (M+) 463.

### EXAMPLE 310

### 3-(3,4-Dichloro-benzyl)-5-methyl-2,4-dioxo-1,2,3,4-tetrahydro-thieno[2,3-d]pyrimidine-6-carboxylic acid benzyl ester

The procedure of Example 74 was repeated, except 1-bromo-4-isocyanatomethyl-benzene was replaced by 1,2-dichloro-4-isocyanatomethyl-benzene to give 3-(3,4-dichloro-benzyl)-5-methyl-2,4-dioxo-1,2,3,4-tetrahydro-thieno[2,3-d]pyrimidine-6-carboxylic acid benzyl ester as a white solid (19%); MS-APCI (M+) 475.

### EXAMPLE 311

### 1-Methyl-2,4-dioxo-3-(4-trifluoromethoxy-benzyl)-1,2,3,4-tetrahydro-thieno[2,3-d]pyrimidine-6-carboxylic acid benzyl ester

The procedure of Example 15 was repeated, except 3-bromomethyl-benzoic acid methyl ester was replaced by 1-bromomethyl-4-trifluoromethoxy-benzene. The crude product was chromatographed using 2:1 hexane/ethyl acetate to 100% ethyl acetate to give 1-methyl-2,4-dioxo-3-(4-trifluoromethoxy-benzyl)-1,2,3,4-tetrahydro-thieno[2,3-d]pyrimidine-6-carboxylic acid benzyl ester as a white solid (50%); MS-APCI (M+) 491.

### EXAMPLE 312

### 1-Methyl-3-naphthalen-2-ylmethyl-2,4-dioxo-1,2,3,4-tetrahydro-thieno[2,3-d]pyrimidine-6-carboxylic acid benzyl ester

The procedure of Example 15 was repeated, except 3-bromomethyl-benzoic acid methyl ester was replaced by 2-bromomethyl naphthalene. The crude product was chromatographed using 2:1 hexane/ethyl acetate to 100% ethyl acetate to give 1-methyl-3-naphthalen-1-ylmethyl-2,4-dioxo-1,2,3,4-tetrahydro-thieno[2,3-d]pyrimidine-6-carboxylic acid benzyl ester as a white solid (72%); MS-APCI (M+) 457.

### EXAMPLE 313

### 3-(4-Cyano-benzyl)-1-methyl-2,4-dioxo-1,2,3,4-tetrahydro-thieno[2,3-d]pyrimidine-6-carboxylic acid benzyl ester

The procedure of Example 15 was repeated, except 3-bromomethyl-benzoic acid methyl ester was replaced by 4-bromomethyl-benzonitrile. The crude product was chromatographed using 2:1 hexane/ethyl acetate to 100% ethyl acetate to give 3-(4-cyano-benzyl)-1-methyl-2,4-dioxo-1,2,3,4-tetrahydro-thieno[2,3-d]pyrimidine-6-carboxylic acid benzyl ester as a white solid (80%); MS-APCI (M+) 432.

### EXAMPLE 314

### 3-Benzyl-1-methyl-2,4-dioxo-1,2,3,4-tetrahydro-thieno[2,3-d]pyrimidine-6-carboxylic acid benzofuran-5-ylmethyl ester

The procedure of Example 1 was repeated, except that benzyl alcohol was replaced by benzofuran-5-yl-methanol to provide 3-benzyl-1-methyl-2,4-dioxo-1,2,3,4-tetrahydro-thieno[2,3-d]pyrimidine-6-carboxylic acid benzofuran-5-ylmethyl ester as a white powder (24%); MS-APCI (M+) 447.

### EXAMPLE 315

### 3-(3,5-Dimethoxy-benzyl)-1-methyl-2,4-dioxo-1,2,3,4-tetrahydro-thieno[2,3-d]pyrimidine-6-carboxylic acid benzyl ester

The procedure of Example 15 was repeated, except 3-bromomethyl-benzoic acid methyl ester was replaced by 1-chloromethyl-3,5-dimethoxy-benzene. The crude product was chromatographed using 2:1 hexane/ethyl acetate to 100% ethyl acetate to give 3-(3,5-dimethoxy-benzyl)-1-methyl-2,4-dioxo-1,2,3,4-tetrahydro-thieno[2,3-d]pyrimidine-6-carboxylic acid benzyl ester as a white solid (52%); MS-APCI (M+) 467.

### EXAMPLE 316

### 3-Benzyl-1-methyl-2,4-dioxo-1,2,3,4-tetrahydro-thieno[2,3-d]pyrimidine-6-carboxylic acid benzyl ester

### EXAMPLE 317

### 3-(3,5-Dinitro-benzyl)-1-methyl-2,4-dioxo-1,2,3,4-tetrahydro-thieno[2,3-d]pyrimidine-6-carboxylic acid benzyl ester

The procedure of Example 15 was repeated, except 3-bromomethyl-benzoic acid methyl ester was replaced by 1-chloromethyl-3,5-dinitro-benzene. The crude product was chromatographed using 2:1 hexane/ethyl acetate to 100% ethyl acetate to give 3-(3,5-dinitro-benzyl)-1-methyl-2,4-dioxo-1,2,3,4-tetrahydro-thieno[2,3-d]pyrimidine-6-carboxylic acid benzyl ester as a white solid (10%); MS-APCI (M+) 501.

### EXAMPLE 318

### 3-Benzyl-1-methyl-2,4-dioxo-1,2,3,4-tetrahydro-thieno[2,3-d]pyrimidine-6-carboxylic acid

To a solution of 3-benzyl-1-methyl-2,4-dioxo-1,2,3,4-tetrahydro-thieno[2,3-d]pyrimidine-6-carboxylic acid ethyl ester (5.0 g, 14.5 mmol) in 90% THF/10% water, was added 5.0 g of LiOH. The solution was stirred at room temperature for 5 hours, then poured into 400 mL of 1:1 ethyl acetate/water, and acidified with hydrochloric acid until the pH is acidic. The organic layer was dried over magnesium sulfate and concentrated. The residue was triturated with 4:1 hexane/ethyl acetate to yield 2.8 g (62%) of the title compound as a white solid; MS-APCI (M+) 317.

### EXAMPLE 319

### 3-(4-Carboxy-benzyl)-1-methyl-2,4-dioxo-1,2,3,4-tetrahydro-thieno[2,3-d]pyrimidine-6-carboxylic acid 2-ethoxy-benzyl ester

The procedure of Examples 53 and 54 were repeated, 3,4-dimethoxy benzyl amine was replaced by 2-ethoxy benzylamine, to give 4-[6-(2-ethoxy-benzylcarbamoyl)-1-methyl-2,4-dioxo-1,2,3,4-tetrahydro-thieno[2,3-d]pyrimidin-3-ylmethyl]-benzoic acid as a white solid (20%); MS-APCI (M+) 494.

### EXAMPLE 320

### 3-Benzyl-1-methyl-2,4-dioxo-1,2,3,4-tetrahydro-thieno[2,3-d]pyrimidine-6-carboxylic acid[2-(3,4-dimethoxy-phenyl)-ethyl]-amide

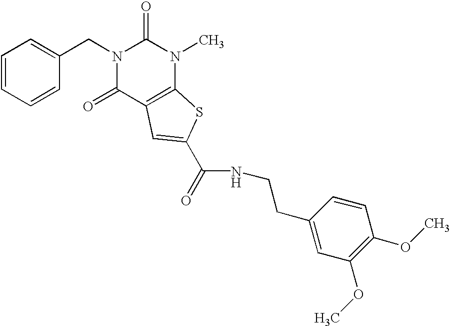

In a 8-mL screw cap vial was added a mixture of the compound of Example 318, namely 3-benzyl-1-methyl-2,4-dioxo-1,2,3,4-tetrahydro-thieno[2,3-d]pyrimidine-6-carboxylic acid, (0.032 g, 0.1 mmol), triethylamine (0.024g, 0.24 mmol), and 2-chloro-1-methylpyridinium iodide (0.031 g, 0.12 mmol) in dichloromethane (2 mL) followed by 3,4-methoxyethyl amine,(0.020 g, 0. 11 mmol) in dichloromethane (1 mL). The vial was capped, and the reaction mixture was shaken for 24 hours at room temperature. The solvent was removed under vacuum. Purification was carried out via reverse-phase HPLC (3% n-propanol in acetonitrile and 3% n-propanol in water as the eluent; C-18 column). 0.023 g (50% yield). MS-APCI (M+1): 480.5.

In a manner similar to the procedure of Example 320, the compounds of Examples 321 to 363 were prepared.

### EXAMPLE 321

### 3-Benzyl-1-methyl-2,4-dioxo-1,2,3,4-tetrahydro-thieno[2,3-d]pyrimidine-6-carboxylic acid 4-amino-benzylamide

MS APCI (M+1): 421.491.

### EXAMPLE 322

### 3-Benzyl-1-methyl-2,4-dioxo-1,2,3,4-tetrahydro-thieno[2,3-d]pyrimidine-6-carboxylic acid[2-(4-chloro-phenyl)-ethyl]-amide

MS APCI (M+1): 454.948.

### EXAMPLE 323

### 3-Benzyl-1-methyl-2,4-dioxo-1,2,3,4-tetrahydro-thieno[2,3-d]pyrimidine-6-carboxylic acid (biphenyl-2-ylmethyl)-amide

MS APCI (M+1): 482.5737.

### EXAMPLE 324

### 3-Benzyl-1-methyl-2,4-dioxo-1,2,3,4-tetrahydro-thieno[2,3-d]pyrimidine-6-carboxylic acid 3,4-dimethoxy-benzylamide

MS APCI (M+1): 466.5277.

### EXAMPLE 325

### 3-Benzyl-1-methyl-2,4-dioxo-1,2,3,4-tetrahydro-thieno[2,3-d]pyrimidine-6-carboxylic acid (2-pyridin-4-yl-ethyl)-amide

MS APCI (M+1): 421.491.

### EXAMPLE 326

### 3-Benzyl-1-methyl-2,4-dioxo-1,2,3,4-tetrahydro-thieno[2,3-d]pyrimidine-6-carboxylic acid 2-difluoromethoxy-benzylamide

MS APCI (M+1): 472.4821.

### EXAMPLE 327

### 3-Benzyl-1-methyl-2,4-dioxo-1,2,3,4-tetrahydro-thieno[2,3-d]pyrimidine-6-carboxylic acid[2-(3-ethoxy-phenyl)-ethyl]-amide

MS APCI (M+1): 464.5555.

### EXAMPLE 328

### 3-Benzyl-1-methyl-2,4-dioxo-1,2,3,4-tetrahydro-thieno[2,3-d]pyrimidine-6-carboxylic acid 3-chloro-4-fluoro-benzylamide

MS APCI (M+1): 458.9113.

### EXAMPLE 329

### 3-Benzyl-1-methyl-2,4-dioxo-1,2,3,4-tetrahydro-thieno[2,3-d]pyrimidine-6-carboxylic acid 2,4-dichloro-benzylamide

MS APCI (M+1): 475.3663.

### EXAMPLE 330

### 3-Benzyl-1-methyl-2,4-dioxo-1,2,3,4-tetrahydro-thieno[2,3-d]pyrimidine-6-carboxylic acid (2-phenyl-propyl)-amide

MS APCI (M+1): 434.5297.

### EXAMPLE 331

### 3-Benzyl-1-methyl-2,4-dioxo-1,2,3,4-tetrahydro-thieno[2,3-d]pyrimidine-6-carboxylic acid 3,4,5-trimethoxy-benzylamide

MS APCI (M+1): 496.5535.

### EXAMPLE 332

### 3-Benzyl-1-methyl-2,4-dioxo-1,2,3,4-tetrahydro-thieno[2,3-d]pyrimidine-6-carboxylic acid 3-chloro-benzylamide

MS APCI (M+1): 440.9212.

### EXAMPLE 333

### 3-Benzyl-1-methyl-2,4-dioxo-1,2,3,4-tetrahydro-thieno[2,3-d]pyrimidine-6-carboxylic acid 3,5-dimethoxy-benzylamide

MS APCI (M+1): 466.5277

### EXAMPLE 334

### 3-Benzyl-1-methyl-2,4-dioxo-1,2,3,4-tetrahydro-thieno[2,3-d]pyrimidine-6-carboxylic acid 2,3-dimethoxy-benzylamide

MS APCI (M+1): 466.5277.

### EXAMPLE 335

### 3-Benzyl-1-methyl-2,4-dioxo-1,2,3,4-tetrahydro-thieno[2,3-d]pyrimidine-6-carboxylic acid 3-trifluoromethyl-benzylamide

MS APCI (M+1): MS APCI (M+1): 474.4732.

### EXAMPLE 336

### 3-Benzyl-1-methyl-2,4-dioxo-1,2,3,4-tetrahydro-thieno[2,3-d]pyrimidine-6-carboxylic acid 2-methoxy-benzylamide

MS APCI (M+1): 436.5019.

### EXAMPLE 337

### 3-Benzyl-1-methyl-2,4-dioxo-1,2,3,4-tetrahydro-thieno[2,3-d]pyrimidine-6-carboxylic acid 2-methyl-benzylamide

MS APCI (M+1): 420.5029.

### EXAMPLE 338

### 3-Benzyl-1-methyl-2,4-dioxo-1,2,3,4-tetrahydro-thieno[2,3-d]pyrimidine-6-carboxylic acid (4-phenyl-butyl)-amide

MS APCI (M+1): 448.5565.

### EXAMPLE 339

### 3-Benzyl-1-methyl-2,4-dioxo-1,2,3,4-tetrahydro-thieno[2,3-d]pyrimidine-6-carboxylic acid (pyridin-3-ylmethyl)-amide

MS APCI (M+1): 407.4642.

### EXAMPLE 340

### 3-Benzyl-1-methyl-2,4-dioxo-1,2,3,4-tetrahydro-thieno[2,3-d]pyrimidine-6-carboxylic acid 4-methoxy-benzylamide

MS APCI (M+1): 436.5019.

### EXAMPLE 341

### 3-Benzyl-1-methyl-2,4-dioxo-1,2,3,4-tetrahydro-thieno[2,3-d]pyrimidine-6-carboxylic acid ((S)-2,2-dimethyl-4-phenyl-1,3-dioxinan-5-yl)-amide

MS APCI (M+1): 405.5923.

### EXAMPLE 342

### 3-Benzyl-1-methyl-2,4-dioxo-1,2,3,4-tetrahydro-thieno[2,3-d]pyrimidine-6-carboxylic acid[2-(3-methoxy-phenyl)-ethyl]-amide

MS APCI (M+1): 450.5287.

### EXAMPLE 343

### 3-Benzyl-1-methyl-2,4-dioxo-1,2,3,4-tetrahydro-thieno[2,3-d]pyrimidine-6-carboxylic acid 3-methoxy-benzylamide

MS APCI (M+1): 436.5019.

### EXAMPLE 344

### 3-Benzyl-1-methyl-2,4-dioxo-1,2,3,4-tetrahydro-thieno[2,3-d]pyrimidine-6-carboxylic acid (thiophen-2-ylmethyl)-amide

MS APCI (M+1): 412.5043.

### EXAMPLE 345

### 3-Benzyl-1-1-methyl-2,4-dioxo-1,2,3,4-tetrahydro-thieno[2,3-d]pyrimidine-6-carboxylic acid 2-chloro-benzylamide

MS APCI (M+1): 440.9212.

### EXAMPLE 346

### 3-Benzyl-1-methyl-2,4-dioxo-1,2,3,4-tetrahydro-thieno[2,3-d]pyrimidine-6-carboxylic acid (5-methyl-furan-2-ylmethyl)-amide

MS APCI (M+1): 410.4641.

### EXAMPLE 347

### 3-Benzyl-1-methyl-2,4-dioxo-1,2,3,4-tetrahydro-thieno[2,3-d]pyrimidine-6-carboxylic acid (2,2-diphenyl-ethyl)-amide

MS APCI (M+1): 496.6005.

### EXAMPLE 348

### 3-Benzyl-1-methyl-2,4-dioxo-1,2,3,4-tetrahydro-thieno[2,3-d]pyrimidine-6-carboxylic acid[2-(2-methoxy-phenyl)-ethyl]-amide

MS APCI (M+1): 450.5287.

### EXAMPLE 349

### 3-Benzyl-1-methyl-2,4-dioxo-1,2,3,4-tetrahydro-thieno[2,3-d]pyrimidine-6-carboxylic acid[2-(3-trifluoromethyl-phenyl)-ethyl]-amide

MS APCI (M+1): 488.5.

### EXAMPLE 350

### 3-Benzyl-1-methyl-2,4-dioxo-1,2,3,4-tetrahydro-thieno[2,3-d]pyrimidine-6-carboxylic acid 4-bromo-benzylamide

MS APCI (M+1): 485.3722.

### EXAMPLE 351

### 3-Benzyl-1-methyl-2,4-dioxo-1,2,3,4-tetrahydro-thieno[2,3-d]pyrimidine-6-carboxylic acid[2-(1H-indol-3-yl)-ethyl]-amide

MS APCI (M+1): 459.5398.

### EXAMPLE 352

### 3-Benzyl-1-methyl-2,4-dioxo-1,2,3,4-tetrahydro-thieno[2,3-d]pyrimidine-6-carboxylic acid 3 ,5-dichloro-benzylamide

MS APCI (M+1): 475.3663.

### EXAMPLE 353

### 3-Benzyl-1-methyl-2,4-dioxo-1,2,3,4-tetrahydro-thieno[2,3-d]pyrimidine-6-carboxylic acid indan-1-ylamide

MS APCI (M+1): 432.5139.

### EXAMPLE 354

### 3-Benzyl-1-methyl-2,4-dioxo-1,2,3,4-tetrahydro-thieno[2,3-d]pyrimidine-6-carboxylic acid (furan-2-ylmethyl)-amide

MS APCI (M+1): 396.4373.

### EXAMPLE 355

### 3-Benzyl-1-methyl-2,4-dioxo-1,2,3,4-tetrahydro-thieno[2,3-d]pyrimidine-6-carboxylic acid[2-(4-methoxy-phenyl)-ethyl]-amide

MS APCI (M+1): 450.5287.

### EXAMPLE 356

### 3-Benzyl-1-methyl-2,4-dioxo-1,2,3,4-tetrahydro-thieno[2,3-d]pyrimidine-6-carboxylic acid 2,4-dimethoxy-benzylamide

MS APCI (M+1): 466.5277.

### EXAMPLE 357

### 3-Benzyl-1-methyl-2,4-dioxo-1,2,3,4-tetrahydro-thieno[2,3-d]pyrimidine-6-carboxylic acid 4-chloro-benzylamide

MS APCI (M+1): 440.9212.

### EXAMPLE 358

### 3-Benzyl-1-methyl-2,4-dioxo-1,2,3,4-tetrahydro-thieno[2,3-d]pyrimidine-6-carboxylic acid (1-phenyl-ethyl)-amide

MS APCI (M+1): 420.5029.

### EXAMPLE 359

### 3-Benzyl-1-methyl-2,4-dioxo-1,2,3,4-tetrahydro-thieno[2,3-d]pyrimidine-6-carboxylic acid 3,4-dichloro-benzylamide

MS APCI (M+1): 475.3663.

### EXAMPLE 360

### 3-Benzyl-1-methyl-2,4-dioxo-1,2,3,4-tetrahydro-thieno[2,3-d]pyrimidine-6-carboxylic acid 4-fluoro-3-trifluoromethyl-benzylamide

MS APCI (M+1): 492.4633.

### EXAMPLE 361

### 3-Benzyl-1-methyl-2,4-dioxo-1,2,3,4-tetrahydro-thieno[2,3-d]pyrimidine-6-carboxylic acid (2-pyridin-2-yl-ethyl)-amide

MS APCI (M+1): 421.491.

### EXAMPLE 362

### 3-Benzyl-1-methyl-2,4-dioxo-1,2,3,4-tetrahydro-thieno[2,3-d]pyrimidine-6-carboxylic acid[2-(2,4-dimethyl-phenyl)-ethyl]-amide

MS APCI (M+1): 448.5565.

### EXAMPLE 363

### 3-Benzyl-1-methyl-2,4-dioxo-1,2,3,4-tetrahydro-thieno[2,3-d]pyrimidine-6-carboxylic acid[2-(2,4-dichloro-phenyl)-ethyl]-amide

MS APCI (M+1): 489.3931.

### EXAMPLE 364

### 1,3-Dimethyl-2,4-dioxo-1,2,3,4-tetrahydro-thieno[2,3-d]pyrimidine-6-carboxylic acid 3-methoxy-benzylamide

The procedure of Example 34 was repeated, except 4-(4-bromomethyl-benzenesulfonyl)-morpholine was replaced by 4-bromomethyl-benzenesulfonic acid methyl ester to give 1,3-dimethyl-2,4-dioxo-1,2,3,4-tetrahydro-thieno[2,3-d]pyrimidine-6-carboxylic acid 3-methoxy-benzylamide as a white solid (15%); MS APCI (M+) 360.

### EXAMPLE 365

### 3-Cyanomethyl-1-methyl-2,4-dioxo-1,2,3,4-tetrahydro-thieno[2,3-d]pyrimidine-6-carboxylic acid 4-methoxy-benzylamide

### EXAMPLE 366

### 3-Benzyl-1-methyl-2,4-dioxo-1,2,3,4-tetrahydro-thieno[2,3-d]pyrimidine-6-carboxylic acid 3-methoxy-benzylamide

The procedure of Example 34 was repeated, except 4-(4-bromomethyl-benzenesulfonyl)-morpholine was replaced by benzyl bromide to give 3-benzyl-1-methyl-2,4-dioxo-1,2,3,4-tetrahydro-thieno[2,3-d]pyrimidine-6-carboxylic acid 3-methoxy-benzylamide as a white solid (86%); MS APCI (M+) 494.

### EXAMPLE 367

### 3-(4-Cyclopropylsulfamoyl-benzyl)-1-methyl-2,4-dioxo-1,2,3,4-tetrahydro-thieno[2,3-d]pyrimidine-6-carboxylic acid 3-methoxy-benzylamide

The procedure of Example 34 was repeated, except 4-(4-bromomethyl-benzenesulfonyl)-morpholine was replaced by 1-bromomethyl-4-cyclopropylmethanesulfonyl-benzene, to 3-(4-cyclopropylsulfamoyl-benzyl)-1-methyl-2,4-dioxo-1,2,3,4-tetrahydro-thieno[2,3-d]pyrimidine-6-carboxylic acid 3-methoxy-benzylamide as a white solid (95%); ¹H NMR (DMSO), δ0.37 (d, J=2.9 Hz, 2H), 0.46 (d, J=4.9 Hz, 2H), 2.04 (m, 1H), 3.49 (s, 3H), 3.73 (s, 3H), 4.42 (d, J=5.9 Hz, 2H), 5.14 (s, 2H), 7.02-6.89 (m, 3H), 7.25 (t, J=8.1 Hz, 1H), 7.52 (d, J=8.1 Hz, 2H), 7.74 (d, J=8.4 Hz, 2H), 7.91 (s, 1H), 8.1 (s, 1H), 9.23 (t, J=5.6 Hz, 1H).

### EXAMPLE 368

### 1-Methyl-3-(6-nitro-pyridin-3-ylmethyl)-2,4-dioxo-1,2,3,4-tetrahydro-thieno[2,3-d]pyrimidine-6-carboxylic acid 3-methoxy-benzylamide

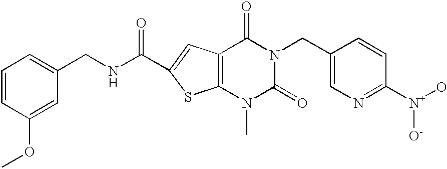

Made by the procedure of Example 217 from 1-methyl-1,2,3,4-tetrahydro-thieno[2,3-d]pyrimidine-6-carboxylic acid 3-methoxy-benzylamide and 5-(bromomethyl)-2-nitropyridine; mp 213-215° C.

### EXAMPLE 369

### 1-Methyl-3-(6-nitro-pyridin-3-ylmethyl)-2,4-dioxo-1,2,3,4-tetrahydro-thieno[2,3-d]pyrimidine-6-carboxylic acid 4-methoxy-benzylamide

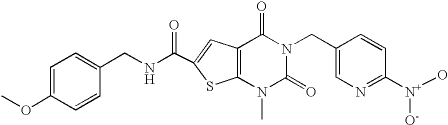

Made by the procedure of Example 217 from 1-methyl-1,2,3,4-tetrahydro-thieno[2,3-d]pyrimidine-6-carboxylic acid 4-methoxy-benzylamide and 5-(bromomethyl)-2-nitropyridine; mp 238-241° C.

### EXAMPLE 370

### 1-Methyl-3-(6-nitro-pyridin-3-ylmethyl)-2,4-dioxo-1,2,3,4-tetrahydro-thieno[2,3-d]pyrimidine-6-carboxylic acid (2-methoxy-pyridin-4-ylmethyl)-amide

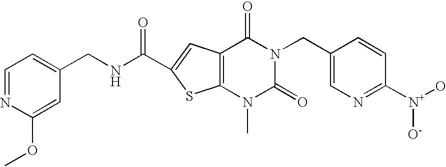

Made by the procedure of Example 217 from 1-methyl-2,4-dioxo-1,2,3,4-tetrahydro-thieno[2,3-d]pyrimidine-6-carboxylic acid (2-methoxy-pyridin-4-ylmethyl)-amide and 5-(bromomethyl)-2-nitropyridine; mp 200-207° C.

### EXAMPLE 371

### 3-Cyclohexylmethyl-1-methyl-2,4-dioxo-1,2,3,4-tetrahydro-thieno[2,3-d]pyrimidine-6-carboxylic acid (2-methoxy-pyridin-4-ylmethyl)-amide

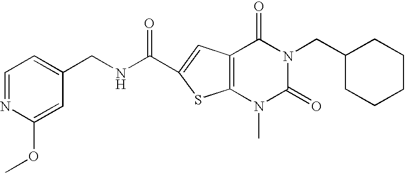

Made by the procedure of Example 217 from 1-methyl-2,4-dioxo-1,2,3,4-tetrahydro-thieno[2,3-d]pyrimidine-6-carboxylic acid (2-methoxy-pyridin-4-ylmethyl)-amide and bromomethyl-cyclohexane; mp 167-210° C.

### EXAMPLE 372

### 3-{2-[(1H-Benzimidazole-5-carbonyl)-amino]-ethyl}-1-methyl-2,4-dioxo-1,2,3,4-tetrahydro-thieno[2,3-d]pyrimidine-6-carboxylic acid 3-methoxy-benzylamide

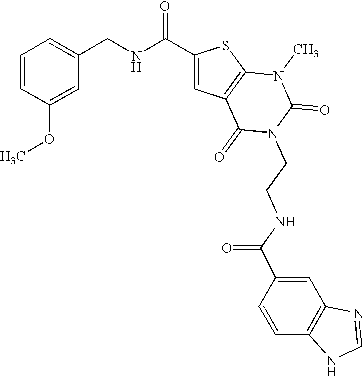

In an 8-mL screw cap vial was added a mixture of the compound of Example 252, namely 3-(2-amino-ethyl)-1-methyl-2,4-dioxo-1,2,3,4-tetrahydro-thieno[2,3-d]pyrimidine-6-carboxylic acid 3-methoxy-benzyl amide, (0.038 g, 0.1 mmol), diisopropylethylamine (0.038 g, 0.30 mmol), O-(benzotriazol-1-yl)-1,1,3,3-tetramethyluronium hexafluorophosphate (0.114 g, 0.30), and 1-hydroxy-7-azabenzotriazole (0.053 g, 0.30 mmol) in dimethylformamide (2 mL) followed by 1 H-benzoimidazole-5-carboxylic acid,(0.045 g, 0.3 mmol) in dimethylformamide (1 mL). The vial was capped and the reaction mixture was shaken for 24 hours at room temperature. The solvent was removed under vacuum. Purification was carried out via reverse-phase HPLC (3% n-propanol in acetonitrile and 3% n-propanol in water as the eluent; C-18 column). 0.023 g (50% yield). MS APCI (M+1): 533.5.

In a manner similar to the procedure of Example 372, the compounds of Examples 373 to 383 were prepared.

### EXAMPLE 373

### 1-Methyl-2,4-dioxo-3-[2-(3-piperidin-1-yl-propionylamino)-ethyl]-1,2,3,4-tetrahydro-thieno[2,3-d]pyrimidine-6-carboxylic acid 3-methoxy-benzylamide

MS APCI (M+1): 528.6427.

### EXAMPLE 374

### 1-Methyl-2,4-dioxo-3-{2-[(6-pyrazol-1-yl-pyridine-3-carbonyl)-amino]-ethyl}-1,2,3,4-tetrahydro-thieno[2,3-d]pyrimidine-6-carboxylic acid 3-methoxy-benzylamide

MS APCI (M+1): 560.6045.

### EXAMPLE 375

### 3-[2-(4-Diethylamino-benzoylamino)-ethyl]-1-methyl-2,4-dioxo-1,2,3,4-tetrahydro-thieno[2,3-d]pyrimidine-6-carboxylic acid 3-methoxy-benzylamide

MS APCI (M+1):564.6757.

### EXAMPLE 376

### 3-{2-[(6-Chloro-pyridine-3-carbonyl)-amino]-ethyl}-1-methyl-2,4-dioxo-1,2,3,4-tetrahydro-thieno[2,3-d]pyrimidine-6-carboxylic acid 3-methoxy-benzylamide

MS APCI (M+1): 528.9868.

### EXAMPLE 377

### 1-Methyl-2,4-dioxo-3-{2-[(1H-pyrrole-2-carbonyl)-amino]-ethyl}-1,2,3,4-tetrahydro-thieno[2,3-d]pyrimidine-6-carboxylic acid 3-methoxy-benzylamide

MS APCI (M+1): 482.5307.

### EXAMPLE 378

### 3-[2-(2-Dimethylamino-acetylamino)-ethyl]-1-methyl-2,4-dioxo-1,2,3,4-tetrahydro-thieno[2,3-d]pyrimidine-6-carboxylic acid 3-methoxy-benzylamide

MS APCI (M+1): 474.5513.

### EXAMPLE 379

### 1-Methyl-2,4-dioxo-3-{2-[(pyrazine-2-carbonyl)-amino]-ethyl}-1,2,3,4-tetrahydro-thieno[2,3-d]pyrimidine-6-carboxylic acid 3-methoxy-benzylamide

MS APCI (M+1): 495.5298.

### EXAMPLE 380

### 1-Methyl-3-[2-(2-methyl-2-methylamino-propionylamino)-ethyl]-2,4-dioxo-1,2,3,4-tetrahydro-thieno[2,3-d]pyrimidine-6-carboxylic acid 3-methoxy-benzylamide

MS APCI (M+1): 488.5781.

### EXAMPLE 381

### 1-Methyl-2,4-dioxo-3-{2-[(pyrrolidine-2-carbonyl)-amino]-ethyl}-1,2,3,4-tetrahydro-thieno[2,3-d]pyrimidine-6-carboxylic acid 3-methoxy-benzylamide

MS APCI (M+1): 486.5623.

### EXAMPLE 382

### 1-Methyl-2,4-dioxo-3-{2-[3-(5-phenyl-1H-pyrrol-2-yl)-propionylamino]-ethyl}-1,2,3,4-tetrahydro-thieno[2,3-d]pyrimidine-6-carboxylic acid 3-methoxy-benzylamide

MS APCI (M+1): 586.6819.

### EXAMPLE 383

### 1-Methyl-2,4-dioxo-3-{2-[2-(pyridin-4-ylsulfanyl)-acetylamino]-ethyl}-1,2,3,4-tetrahydro-thieno[2,3-d]pyrimidine-6-carboxylic acid 3-methoxy-benzylamide

MS APCI (M+1): 540.6345.

### EXAMPLE 384

### 3-(6-Amino-pyridin-3-ylmethyl)-1-methyl-2,4-dioxo-1,2,3,4-tetrahydro-thieno[2,3-d]pyrimidine-6-carboxylic acid 3-methoxy-benzylamide

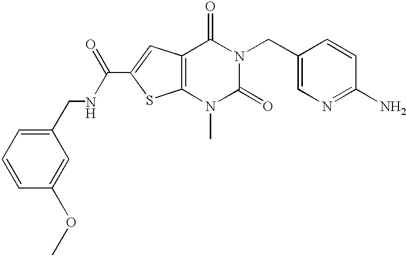

Made by catalytic hydrogenation of the compound of Example 368 with Raney nickel; mp 131-134° C. (dec.).

### EXAMPLE 385

### 1-Methyl-2,4-dioxo-3-(3-phenyl-prop-2-ynyl)-1,2,3,4-tetrahydro-thieno[2,3-d]pyrimidine-6-carboxylic acid 4-methoxy-benzylamide

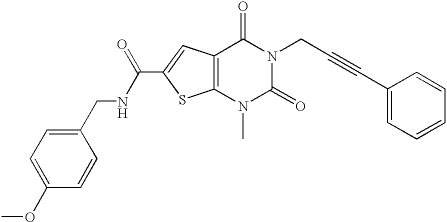

Made by the procedure of Example 217 from 1-methyl-1,2,3,4-tetrahydro-thieno[2,3-d]pyrimidine-6-carboxylic acid 4-methoxy-benzylamide and (3-bromo-prop-1-ynyl)-benzene; mp 168-171° C.

### EXAMPLE 386

### 3-(6-Amino-pyridin-3-ylmethyl)-1-methyl-2,4-dioxo-1,2,3,4-tetrahydro-thieno[2,3-d]pyrimidine-6-carboxylic acid 4-methoxy-benzylamide

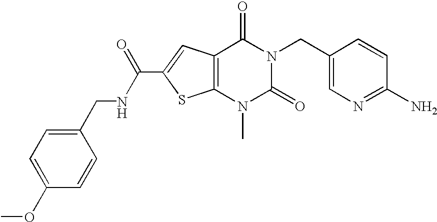

Made by catalytic hydrogenation of the compound of Example 369 with Raney nickel; mp 240-241° C.

### EXAMPLE 387

### 1-Methyl-3-benzyl-2,4-dioxo-1,2,3,4-tetrahydro-thieno[3,2-d]pyrimidine-6-carboxylic acid benzyl ester

Step (1): 3-Benzyl-2,4-dioxo-1,2,3,4-tetrahydro-thieno[3,2-d]pyrimidine-6-carboxylic acid benzyl ester

To a solution of 3-benzyl-2,4-dioxo-1,2,3,4-tetrahydro-thieno[3,2-d]pyrimidine-6-carboxylic acid (obtained by the method of Preparation 4) (0.26 mmol, 80 mg) in anhydrous DMF (4 mL) were added benzyl alcohol (0.29 mmol, 30 μL), diisopropylethylamine (0.58 mmol, 101 μL), and O (7-azabenzotriazol-1-yl)-N,N,N′,N′-tetramethyluronium hexafluorophosphate (“HATU”) (0.29 mmol, 111 mg). The reaction mixture was stirred at room temperature for 17 hours, and then concentrated under reduced pressure to yield an orange oil. The orange oil was dissolved with 20 mL of dichloromethane. The organic phase was washed with water (2×10 mL), dried (MgSO₄), filtered, and concentrated. The resulting orange oil was purified by flash chromatography on silica gel (98:2 dichloromethane/methanol) to yield 53.9 mg (52%) of 3-benzyl-2,4-dioxo-1,2,3,4-tetrahydro-thieno[3,2-d]pyrimidine-6-carboxylic acid benzyl ester as a white solid.

Step (2): 1-Methyl-3-benzyl-2,4-dioxo-1,2,3,4-tetrahydro-thieno[3,2-d]pyrimidine-6-carboxylic acid benzyl ester

To a solution of the product of Step (1) (89.2 μmol, 35 mg) in anhydrous DMF (3 mL) were added iodomethane (267.8 μmol, 17 μL) and potassium carbonate (133.8 μmol, 18.5 mg). The heterogeneous reaction mixture was then stirred at room temperature for 17 hours, filtered, and concentrated under reduced pressure to afford an orange oil. The orange oil was dissolved with diethyl ether (5 mL). The resulting white precipitate was collected, washed with diethyl ether (2×3 mL), and dried under vacuum to yield 28.8 mg (80%) of 1-methyl-3-benzyl-2,4-dioxo-1,2,3,4-tetrahydro-thieno[3,2-d]pyrimidine-6-carboxylic acid benzyl ester as a white solid.

N.M.R: DMSO ¹H δ (ppm): 3.55 (s, 3H), 5.10 (s, 2H), 5.40 (s, 2H), 7.20-7.50 (m, 10 H), 8.25 (s, 1H); Purity (HPLC, Ultraviolet light detector at 214 nm): 98.0%.

### EXAMPLE 388

### 1-Methyl-3-benzyl-2,4-dioxo-1,2,3,4-tetrahydro-thieno[3.2-d]pyrimidine-6-carboxylic acid benzyl amide

Step (1): 3-Benzyl-2,4-dioxo-1,2,3,4-tetrahydro-thieno[3,2-d]pyrimidine-6-carboxylic acid benzyl amide

To a solution of 3-benzyl-2,4-dioxo-1,2,3,4-tetrahydro-thieno[3,2-d]pyrimidine-6-carboxylic acid (obtained by the method of Preparation 4) (0.12 mmol, 35 mg) in anhydrous DMF (3 mL) were added benzylamine (0.115 mmol, 13 μL), diisopropylethylamine (0.253 mmol, 44 μL), and HATU (0.127 mmol, 49 mg). The reaction mixture was stirred at room temperature for 17 hours, and then concentrated under reduced pressure to yield a white solid. The white solid was dissolved with 4 mL of acetonitrile. The resulting precipitate was collected, washed with cold acetonitrile (2×2 mL), and dried under vacuum to yield 40.1 mg (88%) of 3-benzyl-2,4-dioxo-1,2,3,4-tetrahydro-thieno[3,2-d]pyrimidine-6-carboxylic acid benzyl amide as a white solid.

Step (2): 1-Methyl-3-benzyl-2,4-dioxo-1,2,3,4-tetrahydro-thieno[3,2-d]pyrimidine-6-carboxylic acid benzyl amide

To a solution of the product of Step (1) (76.7 μmol, 30 mg) in anhydrous DMF (3 mL) were added iodomethane (230.1 μmol, 15 μL) and potassium carbonate (115.1 μmol, 16 mg). The heterogeneous reaction mixture was then stirred at room temperature for 17 hours, filtered, and concentrated under reduced pressure to yield an orange oil. The orange oil was dissolved with diisopropyl ether (5 mL). The resulting white precipitate was collected, washed with diisopropyl ether (3×5 mL), and dried under vacuum to yield 28.2 mg (90%) of 1-methyl-3-benzyl-2,4-dioxo-1,2,3,4-tetrahydro-thieno[3,2-d]pyrimidine-6-carboxylic acid benzyl amide as a beige solid.

N.M.R: DMSO ¹H δ (ppm): 3.50 (s, 3H), 4.50 (d, 2H), 5.08 (s, 2H), 7.20-7.40 (m, 10H), 8.00 (s, 1H), 9.40 (t, 1H); Purity (HPLC 214 nm): 95.4%.

### EXAMPLE 389

### 1-Methyl-2,4-dioxo-1,2,3,4-tetrahydro-thieno[3,2-d]pyrimidine-6-carboxylic acid benzyl amide

Step (1): 3-Benzyl-1-methyl-2,4-dioxo-1,2,3,4-tetrahydro-thieno[3,2-d]pyrimidine-6-carboxylic acid methyl ester

To a solution of 3-benzyl-2,4-dioxo-1,2,3,4-tetrahydro-thieno[3,2-d]pyrimidine-6-carboxylic acid (obtained by the method of Preparation 4) (0.33 mmol, 100 mg) in anhydrous DMF (4 mL) were added iodomethane (0.99 mmol, 63 μL) and potassium carbonate (0.99 mmol, 138 mg). The heterogeneous reaction mixture was then stirred at room temperature for 17 hours, filtered, and concentrated under reduced pressure to yield an orange solid. The orange oil was dissolved with pentane (5 mL). The resulting precipitate was collected, washed with pentane (2×5 mL), and dried under vacuum to yield 93.2 mg (84%) of 3-benzyl-1-methyl-2,4-dioxo-1,2,3,4-tetrahydro-thieno[3,2-d]pyrimidine-6-carboxylic acid methyl ester as a yellow solid.

Step (2): 1-Methyl-2,4-dioxo-1,2,3,4-tetrahydro-thieno[3,2-d]pyrimidine-6-carboxylic acid methyl ester

To a solution of the product of Step (1) (0.328 mmol, 108.3 mg) in benzene (12 mL) was added aluminum chloride (1.97 mmol, 262 mg), and the reaction mixture was allowed to warm to 45° C. for 7 hours. The reaction mixture was then diluted with ethyl acetate (20 mL), and the organic phase was washed with water (3×10 mL), dried (MgSO₄), and concentrated under reduced pressure to provide a brown solid. The brown solid was dissolved with diethyl ether. The resulting precipitate was collected, washed with diethyl ether (3×5 mL), and dried under vacuum to yield 42.3 mg (53%) of 1-methyl-2,4-dioxo-1,2,3,4-tetrahydro-thieno[3,2-d]pyrimidine-6-carboxylic acid methyl ester as a mauve-colored solid.

Step (3): 1-Methyl-2,4-dioxo-1,2,3,4-tetrahydro-thieno[3,2-d]pyrimidine-6-carboxylic acid

To a solution of the product of Step (2) (0.175 mmol, 42 mg) in methanol (2 mL) was added a solution of lithium hydroxide (0.437 mmol, 11 mg) in water (1 mL), and the resulting reaction mixture was stirred at room temperature for 17 hours. After concentration under reduced pressure, the crude product was dissolved in 1.0 M hydrochloric acid (10 mL) and extracted with ethyl acetate (2×15 mL). The combined organic phases were washed with water (2×15 mL), dried (MgSO₄), filtered, and concentrated under reduced pressure to yield 34.8 mg (88%) of 1-methyl-2,4-dioxo-1,2,3,4-tetrahydro-thieno[3,2-d]pyrimidine-6-carboxylic acid as a white solid.

Step (4): 1-Methyl-2,4-dioxo-1,2,3,4-tetrahydro-thieno[3,2-d]pyrimidine-6-carboxylic acid benzyl amide

To a solution of the product of Step (3) (0.152 mmol, 34.5 mg) in anhydrous DMF (3 mL) were added benzylamine (0.17 mmol, 19 μL), diisopropylethylamine (0.34 mmol, 59 μL), and HATU (0.17 mmol, 64 mg). The reaction mixture was then stirred at room temperature for 17 hours and evaporated under reduced pressure to yield an orange oil. The orange oil was dissolved with 5 mL of ethanol. The resulting precipitate was collected, washed with cold ethanol (2×3 mL), pentane (2×3 mL), and dried under vacuum to yield 33.8 mg (70%) of 1-methyl-2,4-dioxo-1,2,3,4-tetrahydro-thieno[3,2-d]pyrimidine-6-carboxylic acid benzyl amide as a white solid.

N.M.R: DMSO ¹H δ (ppm): 3.42 (s, 3H), 4.50 (d, 2H), 7.25-7.40 (m, 5H), 7.95 (s, 1H), 9.35 (t, 1H); Purity (HPLC 214 nm): 96.5%.

The invention compounds of Formula I have been evaluated in standard assays for their ability to inhibit the catalytic activity of various MMP enzymes. The assays used to evaluate the biological activity of the invention compounds are well known and routinely used by those skilled in the study of MMP inhibitors and their use to treat clinical conditions.

The assays measure the amount by which a test compound reduces the hydrolysis of a thiopeptolide substrate caused by a matrix metalloproteinase enzyme. Such assays are described in detail by Ye et al., in Biochemistry, 1992;31(45):11231-11235, which is incorporated herein by reference.

Thiopeptolide substrates show virtually no decomposition or hydrolysis at or below neutral pH in the absence of a matrix metalloproteinase enzyme. A typical thiopeptolide substrate commonly utilized for assays is Ac-Pro-Leu-Gly-thioester; -Leu-Leu-Gly-OEt. A 100 μL assay mixture will contain 50 mM of N-2-hydroxyethylpiperazine-N′-2-ethanesulfonic acid buffer (“HEPES,” pH 7.0) 10 mM CaCl₂, 100 μM thiopeptolide substrate, and 1 mM 5,5′-dithio-bis-(2-nitro-benzoic acid) (DTNB). The thiopeptolide substrate concentration may be varied from, for example, 10 to 800 μM to obtain Km and Kcat values. The change in absorbance at 405 nm is monitored on a Thermo Max microplate reader (Molecular Devices, Menlo Park, Calif.) at room temperature (22° C.). The calculation of the amount of hydrolysis of the thiopeptolide substrate is based on E₄₁₂=13600 M⁻¹ cm⁻¹ for the DTNB-derived product 3-carboxy-4-nitrothiophenoxide. Assays are carried out with and without matrix metalloproteinase inhibitor compounds, and the amount of hydrolysis is compared for a determination of inhibitory activity of the test compounds.

Several representative compounds have been evaluated for their ability to inhibit various matrix metalloproteinase enzymes. The invention compounds are uniquely active in inhibiting MMP-13. Table I below presents inhibitory activity for compounds from various classes. In the table, MMP-13CD refers to the catalytic domain of collagenase-3. Test compounds were evaluated at various concentrations in order to determine their respective IC₅₀ values, the micromolar concentration of compound required to cause a 50% inhibition of the hydrolytic activity of the respective enzyme.

TABLE I

| | MMP-13CD |
|---|---|
| Compound of Example No. | IC₅₀, (μM) |
| 1 | 0.74 |
| 2 | 0.31 |
| 3 | 30.0 |
| 4 | 16.0 |
| 5 | 51.0 |
| 6 | >100.0 |
| 7 | 10.0 |
| 8 | 15.0 |
| 9 | 0.007 |
| 10 | 0.068 |
| 11 | 0.47 |
| 12 | >100.0 |
| 13 | 18.0 |
| 14 | 7.5 |
| 48 | 1.45 |
| 49 | 0.26 |
| 50 | 0.0875 |
| 51 | 0.0205 |
| 52 | 0.00395 |
| 53 | 30 |
| 54 | 4.5 |
| 55 | 0.011 |
| 56 | 30 |
| 57 | 5.6 |
| 58 | 0.0115 |
| 59 | 2 |
| 60 | 0.16 |
| 61 | 0.045 |
| 62 | 0.0535 |
| 63 | 0.11 |
| 64 | 0.062 |
| 65 | 0.0535 |
| 65a | 1.05 |
| 66 | 0.0275 |
| 67 | 0.00168 |
| 68 | 0.0635 |
| 69 | 0.057 |
| 70 | 0.1185 |
| 71 | 12.96 |
| 72 | >100 |
| 73 | >100 |
| 74 | 71.5 |
| 75 | 0.345 |
| 76 | |
| 77 | 0.00655 |
| 78 | 0.900 |
| 79 | 0.00205 |
| 80 | 25 |
| 81 | 3.899 |
| 82 | 3.700 |
| 83 | 0.140 |
| 84 | 0.02050 |
| 85 | 0.04750 |
| 86 | 1.3999 |
| 87 | 0.0185 |
| 88 | 3.149 |
| 89 | 0.1135 |
| 90 | 0.00543 |
| 91 | 0.0496 |
| 92 | 0.0109 |
| 93 | 0.111 |
| 94 | 0.005349 |
| 95 | 0.10349 |
| 96 | 0.018499 |
| 97 | >100 |
| 98 | 0.063 |
| 99 | 0.16 |
| 100 | 0.61 |
| 101 | 0.034 |
| 102 | 0.034 |
| 103 | 0.03 |
| 104 | 1.1 |
| 105 | 0.52 |
| 106 | 0.59 |
| 107 | 2.4 |
| 108 | 1.7 |
| 109 | 0.94 |
| 110 | 0.42 |
| 111 | 3.2 |
| 112 | 2.9 |
| 113 | 2.9 |
| 114 | 0.33 |
| 115 | 0.33 |
| 116 | 13 |
| 117 | 0.036 |
| 118 | 0.015 |
| 119 | 0.51 |
| 120 | 0.13 |
| 121 | 0.25 |
| 122 | 4.5 |
| 123 | 7.8 |
| 124 | 0.11 |
| 125 | 0.09 |
| 126 | 13 |
| 127 | 3.9 |
| 128 | 0.19 |
| 129 | 0.16 |
| 130 | 0.097 |
| 131 | 0.019 |
| 132 | 0.074 |
| 133 | 0.074 |
| 134 | 1.5 |
| 135 | 0.086 |
| 136 | 0.051 |
| 137 | 8.3 |
| 138 | 0.66 |
| 139 | 0.25 |
| 140 | 0.017 |
| 141 | 0.15 |
| 142 | 0.39 |
| 143 | 0.28 |
| 144 | 0.003 |
| 145 | 1.3 |
| 146 | 47 |
| 147 | 0.16 |
| 148 | 0.54 |
| 149 | 15 |
| 150 | 13 |
| 151 | >100 |
| 152 | 9.9 |
| 153 | 0.004 |
| 154 | 32 |
| 155 | 62 |
| 156 | 0.18 |
| 157 | >100 |
| 158 | 16 |
| 159 | 30 |
| 160 | 30 |
| 161 | 11 |
| 162 | 11 |
| 163 | 0.016 |
| 164 | 69 |
| 165 | 20 |
| 166 | 0.92 |
| 167 | 26 |
| 168 | 25 |
| 169 | 30 |
| 170 | 0.72 |
| 171 | 14 |
| 172 | 32 |
| 173 | >100 |
| 174 | >100 |
| 175 | >100 |
| 176 | >100 |
| 177 | >100 |
| 178 | >100 |
| 179 | 0.25 |
| 180 | 30 |
| 181 | >100 |
| 182 | 8.6 |
| 183 | 30 |
| 184 | >100 |
| 185 | 0.014 |
| 186 | 4.5 |
| 187 | >100 |
| 188 | 19 |
| 189 | 4.9 |
| 190 | >100 |
| 191 | 2.3 |
| 192 | 0.0034 |
| 193 | 0.0034 |
| 194 | 88 |
| 195 | >100 |
| 196 | 30 |
| 197 | >100 |
| 198 | >100 |
| 199 | 17 |
| 200 | 0.067 |
| 201 | 0.3 |
| 202 | 0.36 |
| 203 | 0.36 |
| 204 | 0.072 |
| 205 | 19 |
| 206 | 15 |
| 207 | 0.2 |
| 208 | 0.1 |
| 209 | >100 |
| 210 | 1.2 |
| 211 | 2.1 |
| 212 | 0.67 |
| 213 | 1.7 |
| 214 | 20 |
| 215 | 24 |
| 216 | 18 |
| 217 | 0.0785 |
| 218 | 18 |
| 219 | 17 |
| 220 | 0.061 |
| 221 | 0.0046 |
| 222 | 0.0042 |
| 223 | N/A^{a} |
| 224 | 0.783 |
| 225 | 0.225 |
| 226 | 4.9 |
| 227 | 3.8 |
| 228 | 0.435 |
| 229 | 0.68 |
| 230 | 0.077 |
| 231 | 2.9 |
| 231a | 0.00895 |
| 232 | 0.175 |
| 233 | 0.069 |
| 234 | 0.15 |
| 235 | 18 |
| 236 | 0.0495 |
| 237 | 0.0925 |
| 238 | 0.0555 |
| 239 | 0.0585 |
| 240 | 0.18 |
| 241 | 0.0195 |
| 242 | 3 |
| 243 | 1.4 |
| 244 | 1.25 |
| 245 | 30 |
| 246 | 5.65 |
| 247 | 7.2 |
| 248 | N/A |
| 249 | 7.8 |
| 249a | 0.64 |
| 250 | 0.00765 |
| 251 | 0.655 |
| 252 | 24 |
| 252a | N/A |
| 253 | 0.81 |
| 254 | 1.5 |
| 255 | 14 |
| 256 | 27.5 |
| 257 | 1.5 |
| 258 | 0.27 |
| 259 | 30 |
| 260 | 0.063 |
| 261 | 0.58 |
| 262 | 3.4 |
| 263 | 2.15 |
| 264 | 7.4 |
| 265 | 0.038 |
| 266 | 4 |
| 267 | 1.1 |
| 268 | 3.6 |
| 269 | 26 |
| 270 | 1.8 |
| 271 | 5.9 |
| 272 | 30 |
| 273 | 0.059 |
| 274 | 0.018 |
| 275 | 0.036 |
| 276 | 0.23 |
| 277 | 20 |
| 278 | 7.6 |
| 279 | 3.5 |
| 280 | 17 |
| 281 | 8.9 |
| 282 | 10 |
| 283 | 1.7 |
| 284 | 1.5 |
| 285 | 30 |
| 286 | 0.27 |
| 287 | 1.9 |
| 288 | 4.2 |
| 289 | 2.7 |
| 290 | 15 |
| 291 | 0.12 |
| 292 | 10 |
| 293 | >100 |
| 294 | >100 |
| 295 | 0.23 |
| 296 | 0.0505 |
| 297 | 0.057 |
| 298 | 0.49 |
| 299 | 30 |
| 300 | >100 |
| 301 | >100 |
| 302 | 30 |
| 303 | 0.0036 |
| 304 | 3.1 |
| 305 | 46.6666 |
| 306 | 30 |
| 307 | 0.0052 |
| 308 | 0.00715 |
| 309 | 0.056 |
| 310 | 30 |
| 311 | 0.0845 |
| 312 | 0.0275 |
| 313 | 0.00645 |
| 314 | 0.0185 |
| 315 | 0.0205 |
| 316 | NA |
| 317 | NA |
| 318 | NA |
| 319 | 8 |
| 320 | >100 |
| 321 | >100 |
| 322 | >100 |
| 323 | >100 |
| 324 | >100 |
| 325 | >100 |
| 326 | −9999 |
| 327 | 2.8 |
| 328 | 2.7 |
| 329 | 30 |
| 330 | >100 |
| 331 | >100 |
| 332 | 1 |
| 333 | >100 |
| 334 | >100 |
| 335 | 0.25 |
| 336 | 30 |
| 337 | >100 |
| 338 | >100 |
| 339 | 0.38 |
| 340 | 0.12 |
| 341 | 14 |
| 342 | >100 |
| 343 | 0.044 |
| 344 | 3.6 |
| 345 | 30 |
| 346 | 9.9 |
| 347 | 16 |
| 348 | 30 |
| 349 | >100 |
| 350 | 0.93 |
| 351 | 2 |
| 352 | >100 |
| 353 | >100 |
| 354 | 30 |
| 355 | >100 |
| 356 | 10 |
| 357 | 0.32 |
| 358 | >100 |
| 359 | 1 |
| 360 | 0.27 |
| 361 | 7.7 |
| 362 | >100 |
| 363 | 30 |
| 364 | NA |
| 365 | 1.55 |
| 366 | NA |
| 367 | 0.00825 |
| 368 | 0.735 |
| 369 | 1.04 |
| 370 | 1.17 |
| 371 | 0.22 |
| 372 | NA |
| 373 | NA |
| 374 | NA |
| 375 | NA |
| 376 | NA |
| 377 | NA |
| 378 | NA |
| 379 | NA |
| 380 | NA |
| 381 | NA |
| 382 | NA |
| 383 | NA |
| 384 | 0.66 |
| 385 | 0.007 |
| 386 | NA |

^{a}NA means data not available

Selectivity of the invention compounds for inhibiting MMP-13 over certain other MMP enzymes is illustrated by a few randomly selected examples in Table II below.

In Table II, MMP-1FL refers to full-length interstitial collagenase; MMP-2FL refers to full length Gelatinase A; MMP-3CD refers to the catalytic domain of stromelysin-1; MMP-7FL refers to full-length matrilysin; MMP-9FL refers to full-length Gelatinase B; MMP-13CD refers to the catalytic domain of collagenase-3; and MMP-14CD refers to the catalytic domain of MMP-14. Test compounds were evaluated at various concentrations in order to determine their respective IC₅₀ values, the micromolar concentration of compound required to cause a 50% inhibition of the hydrolytic activity of the respective enzyme.

It should be appreciated that the assay buffer used with MMP-3CD is 50 mM N-morpholinoethanesulfonate (“MES”) at pH 6.0 rather than the HEPES buffer at pH 7.0 described above.

TABLE II
IC₅₀ With Certain MMP Enzymes

| | MMP- | MMP- | MMP- | MMP- | MMP- | MMP- | MMP- | MMP- |
|---|---|---|---|---|---|---|---|---|
| Ex. No. | 1FL | 2FL | 3CD | 7FL | 9FL | 12CD | 13CD | 14CD |
| 1 | 100 | 100 | 18 | 100 | 100 | 100 | 0.61 | 100 |
| 303 | 100 | 100 | 6 | 32 | 30 | 100 | 0.0036 | 100 |
| 20 | 100 | 100 | 100 | 100 | 100 | 100 | 0.0065 | 100 |
| 49 | 100 | 100 | 23 | 100 | 100 | 100 | 0.26 | 100 |
| 217 | 100 | 100 | 27 | 100 | 30 | 30 | 0.0785 | 100 |
| 63 | 30 | 30 | 18 | 30 | 30 | 30 | 0.11 | 30 |
| 228 | 30 | 30 | 15 | 10 | 30 | 30 | 0.435 | 30 |
| 39 | 100 | 100 | 16 | 100 | 100 | 10 | 0.0038 | 100 |
| 246 | 100 | 30 | 23 | 48 | 30 | 30 | 5.65 | 36 |
| 231a | 100 | 50 | 13 | 46 | 91 | 30 | 0.0090 | 30 |
| 232 | 30 | 30 | 17 | 30 | 30 | 30 | 0.18 | 30 |
| 47 | 30 | 30 | 30 | 14 | 30 | 30 | 0.038 | 10 |
| 249a | 100 | 30 | 100 | 30 | 30 | 100 | 0.64 | 30 |
| 369 | 10 | 30 | 10 | 30 | 30 | 30 | 1.04 | 30 |

The foregoing data in Tables I and II establish that the invention compounds of Formula I are potent inhibitors of MMP enzymes, and are especially useful due to their selective inhibition of MMP-13. Because of this potent and selective inhibitory activity, the invention compounds are especially useful to treat diseases mediated by the MMP enzymes, and particularly those mediated by MMP-13.

Administration of a compound of Formula 1, or a pharmaceutically acceptable salt thereof, to a mammal to treat the diseases mediated by MMP enzymes is preferably, although not necessarily, accomplished by administering the compound, or the salt thereof, in a pharmaceutical dosage form.

The compounds of the present invention can be prepared and administered in a wide variety of oral and parenteral dosage forms. Thus, the compounds of the present invention can be administered by injection, that is, intravenously, intramuscularly, intracutaneously, subcutaneously, intraduodenally, or intraperitoneally. Also, the compounds of the present invention can be administered by inhalation, for example, intranasally. Additionally, the compounds of the present invention can be administered transdermally. It will be obvious to those skilled in the art that the following dosage forms may comprise as the active component, either a compound of Formula I or a corresponding pharmaceutically acceptable salt of a compound of Formula I. The active compound generally is present in a concentration of about 5% to about 95% by weight of the formulation.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding properties in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component, with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogenous mixture is then poured into convenient sized molds, allowed to cool, and thereby to solidify.

Liquid form preparations include solutions, suspensions, and emulsions, for example, water or water propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizing, and thickening agents as desired.

Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 1 mg to 1000 mg, preferably 10 mg to 100 mg according to the particular application and the potency of the active component. The composition can, if desired, also contain other compatible therapeutic agents.

In therapeutic use as agents to inhibit a matrix metalloproteinase enzyme for the treatment of atherosclerotic plaque rupture, aortic aneurysm, heart failure, restenosis, periodontal disease, corneal ulceration, cancer metastasis, tumor angiogenesis, arthritis, or other autoimmune or inflammatory disorders dependent upon breakdown of connective tissue, the compounds utilized in the pharmaceutical method of this invention are administered at a dose that is effective to inhibit the hydrolytic activity of one or more matrix metalloproteinase enzymes. The initial dosage of about 1 mg to about 100 mg per kilogram daily will be effective. A daily dose range of about 25 mg to about 75 mg per kilogram is preferred. The dosages, however, may be varied depending upon the requirements of the patient, the severity of the condition being treated, and the compound being employed. Determination of the proper dosage for a particular situation is within the skill of the art. Generally, treatment is initiated with smaller dosages which are less than the optimum dose of the compound. Thereafter, the dosage is increased by small increments until the optimum effect under the circumstance is reached. For convenience, the total daily dosage may be divided and administered in portions during the day if desired. Typical dosages will be from about 0.1 to about 500 mg/kg, and ideally about 25 to about 250 mg/kg, such that it will be an amount which is effective to treat the particular disease being prevented or controlled.

The following examples illustrate typical formulations provided by the invention.

### Formulation Example 1

Tablet Formulation

| Ingredient | Amount (mg) |
|---|---|
| Compound of Example 1 | 25 |
| Lactose | 50 |
| Corn starch (for mix) | 10 |
| Corn starch (paste) | 10 |
| Magnesium stearate (1%) | 5 |
| Total | 100 |

The fused pyrimidinone of Example 1, lactose, and corn starch (for mix) are blended to uniformity. The corn starch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for treatment of atherosclerosis and arthritis.

### Formulation Example 2

Preparation for Oral Solution

| Ingredient | Amount | |
|---|---|---|
| Compound of Example 210 | 400 | mg |
| Sorbitol solution (70% N.F.) | 40 | mL |
| Sodium benzoate | 20 | mg |
| Saccharin | 5 | mg |
| Red dye | 10 | mg |
| Cherry flavor | 20 | mg |
| Distilled water q.s. | 100 | mL |

The sorbitol solution is added to 40 mL of distilled water, and the fused pyrimidinone of Example 210 is dissolved therein. The saccharin, sodium benzoate, flavor, and dye are added and dissolved. The volume is adjusted to 100 mL with distilled water. Each milliliter of syrup contains 4 mg of invention compound.

### Formulation Example 3

Parenteral Solution

In a solution of 700 mL of propylene glycol and 200 mL of water for injection is suspended 20 g of the compound of Example 14. After suspension is complete, the pH is adjusted to 6.5 with 1N sodium hydroxide, and the volume is made up to 1000 mL with water for injection. The formulation is sterilized, filled into 5.0 mL ampoules each containing 2.0 mL, and sealed under nitrogen.

As matrix metalloproteinase inhibitors, the compounds of Formula I are useful as agents for the treatment of multiple sclerosis. They are also useful as agents for the treatment of atherosclerotic plaque rupture, restenosis, periodontal disease, corneal ulceration, treatment of bums, decubital ulcers, wound repair, heart failure, cancer metastasis, tumor angiogenesis, arthritis, and other inflammatory disorders dependent upon tissue invasion by leukocytes.

It should be appreciated that in all invention embodiments described above or in the claims below, whenever an R group such as, for example, R¹, R², R³, R⁴, R⁵, or R⁶, is used more than once to define an invention compound, each use of the R group is independent of any other use of that same R group or, for that matter, any other R group, unless otherwise specified.

What is claimed is:

1. A compound of Formula I 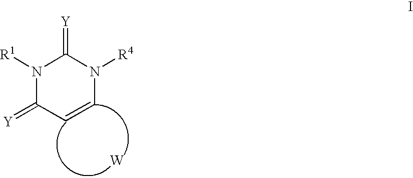
or a pharmaceutically acceptable salt thereof; wherein:
W, together with the carbon atoms to which it is attached, form a 5-membered ring diradical
A is 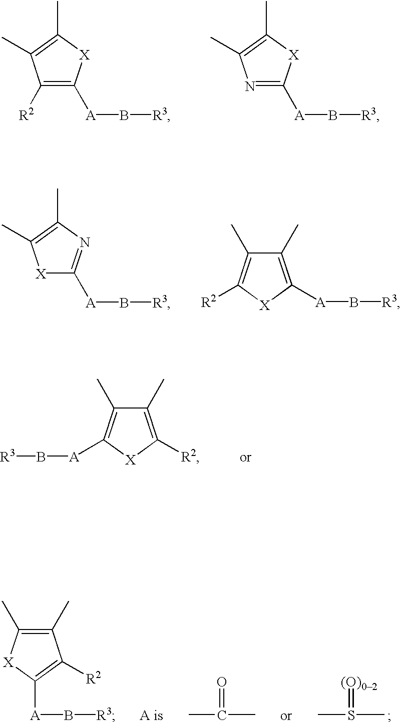
B is O or NR⁵; or
A and B are taken together to form —C≡C—;
X is O, S, SO, SO₂, NR⁵, or CH₂;
each Y independently is O or S;
R¹, R⁴, and R⁵ independently are hydrogen, C₁-C₆ alkyl, C₂-C₆ alkenyl, C₂-C₆ alkynyl, (CH₂)ₙ cycloalkyl, (CH₂)ₙ heterocyclic, C₁-C₆ alkanoyl, (CH₂)ₙ aryl, or (CH₂)ₙ heteroaryl;
R² and R³ independently are hydrogen, C₁-C₆ alkyl, C₂-C₆ alkenyl, C₂-C₆ alkynyl, CN, NO₂, NR⁴R⁵, (CH₂)ₙ cycloalkyl, (CH₂)ₙ aryl, or (CH₂)ₙ heteroaryl;
R² may further be halo;
n is an integer of from 0 to 5; and
R⁴ and R⁵ when taken together with a nitrogen to which they are both attached complete a 3- to 8-membered ring containing carbon atoms and optionally containing O, S, or N, and substituted or unsubstituted;
with the proviso that R¹ and R³ are not both selected from: hydrogen and C₁-C₆ alkyl.

2. A compound of Formula XI 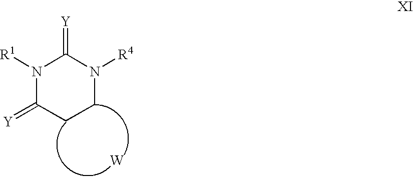
or a pharmaceutically salt thereof, wherein:
W, together with the carbon atoms to which it is attached, form a 5-membered ring diradical 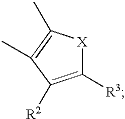
each Y independently is O or S;
X is S, O, or NR⁵;
R¹, R⁴, and R⁵ independently are hydrogen, C₁-C₆ alkyl, C₂-C₆ alkenyl, C₂-C₆ alkynyl, (CH₂)ₙ cycloalkyl, (CH₂)ₙ heterocyclic, C₁-C₆ alkanoyl, (CH₂)ₙ aryl, or (CH₂)ₙ heteroaryl;
R² is hydrogen, halo, C₁-C₆ alkyl, C₂-C₆ alkenyl, C₂-C₆ alkynyl, CN, NO₂, NR⁴R⁵, (CH₂)ₙ cycloalkyl, (CH₂)ₙ aryl, or (CH₂)ₙ heteroaryl;
R³ is hydrogen, halo, C₁-C₆ alkyl, C₂-C₆ alkenyl, CN, NO₂, NR⁴R⁵, (CH₂)_{q} cycloalkyl, (CH₂)_{q} aryl, or (CH₂)_{q} heteroaryl;
n is 0, 1, or 2;
q is 2, 3, or 4; and
R⁴ and R⁵ when taken together with a nitrogen to which they are both attached complete a 3- to 8-membered ring containing carbon atoms and optionally containing O, S, or N, and substituted or unsubstituted;
with the proviso that R¹ and R³ are not both selected from hydrogen and C₁-C₆ alkyl.

3. The compound according to claim 2, or a pharmaceutically acceptable salt thereof, wherein Y is O, and X is S.

4. The compound according to claim 2, or a pharmaceutically acceptable salt thereof, wherein Y is O, and X is O.

5. The compound according to claim 2, or a pharmaceutically acceptable salt thereof, wherein Y is O, and X is NR⁵, wherein R⁵ is hydrogen, C₁-C₆ alkyl, C₂-C₆ alkenyl, C₂-C₆ alkynyl, (CH₂)ₙ cycloalkyl, (CH₂)ₙ heterocyclic, C₁-C₆ alkanoyl, (CH₂)ₙ aryl, or (CH₂)ₙ heteroaryl.

6. A compound of Formula II 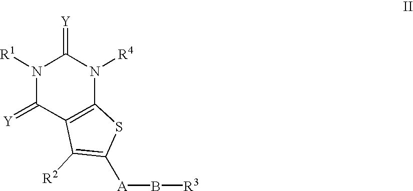
or a pharmaceutically acceptable salt thereof, wherein:
A is 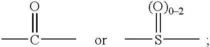
B is O or NR⁵; or
A and B are taken together to form —C≡C—;
each Y independently is O or S;
R¹, R⁴, and R⁵ independently are hydrogen, C₁-C₆ alkyl, C₂-C₆ alkenyl, C₂-C₆ alkynyl, (CH₂)ₙ cycloalkyl, (CH₂)ₙ heterocyclic, C₁-C₆ alkanoyl, (CH₂)ₙ aryl, or (CH₂)ₙ heteroaryl;
R² and R³ independently are hydrogen, C₁-C₆ alkyl, C₂-C₆ alkenyl, C₂-C₆ alkynyl, CN, NO₂, NR⁴R⁵, (CH₂)ₙ cycloalkyl, (CH₂)ₙ aryl, or (CH₂)ₙ heteroaryl;
R² may further be halo;
n is an integer of from 0 to 5; and
R⁴ and R⁵ when taken together with a nitrogen to which they are both attached complete a 3- to 8-membered ring containing carbon atoms and optionally containing O, S, or N, and substituted or unsubstituted;
with the proviso that R¹ and R³ are not both selected from: hydrogen and C₁-C₆ alkyl.

7. The compound according to claim 6, or a pharmaceutically acceptable salt thereof, wherein —A—B— is —C≡C—.

8. The compound according to claim 6, or a pharmaceutically acceptable salt thereof, wherein A is 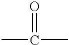
and B is NR⁵, wherein R⁵ is hydrogen, C₁-C₆ alkyl, C₂-C₆ alkenyl, C₂-C₆ alkynyl, (CH₂)ₙ cycloalkyl, (CH₂)ₙ heterocyclic, C₁-C₆ alkanoyl, (CH₂)ₙ aryl, or (CH₂)ₙ heteroaryl.

9. The compound according to claim 6, or a pharmaceutically acceptable salt thereof, wherein A is 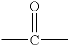
and B is O.

10. A compound of Formula III 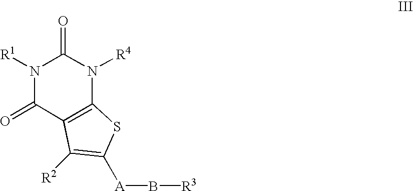
or a pharmaceutically acceptable salt thereof, wherein:
A is 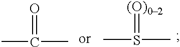
B is O or NR⁵; or
A and B are taken together to form —C≡C—;
R¹, R⁴, and R⁵ independently are hydrogen, C₁-C₆ alkyl, C₂-C₆ alkenyl, C₂-C₆ alkynyl, (CH₂)ₙ cycloalkyl, (CH₂)ₙ heterocyclic, C₁-C₆ alkanoyl, (CH₂)ₙ aryl, or (CH₂)ₙ heteroaryl;
R² and R³ independently are hydrogen, C₁-C₆ alkyl, C₂-C₆ alkenyl, C₂-C₆ alkynyl, CN, NO₂, NR⁴R⁵, (CH₂)ₙ cycloalkyl, (CH₂)ₙ aryl, or (CH₂)ₙ heteroaryl;
R² may further be halo;
n is an integer of from 0 to 5; and
R⁴ and R⁵ when taken together with a nitrogen to which they are both attached complete a 3- to 8-membered ring containing carbon atoms and optionally containing O, S, or N, and substituted or unsubstituted;
with the proviso that R¹ and R³ are not both selected from hydrogen and C₁-C₆ alkyl.

11. A compound of Formula IV 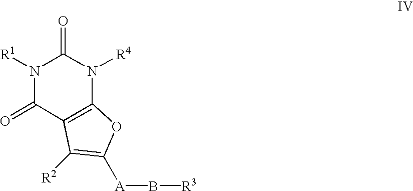
or a pharmaceutically acceptable salt thereof, wherein:
A is 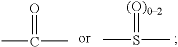
B is O or NR⁵; or
A and B are taken together to form —C≡C—;
R¹, R⁴, and R⁵ independently are hydrogen, C₁-C₆ alkyl, C₂-C₆ alkenyl, C₂-C₆ alkynyl, (CH₂)ₙ cycloalkyl, (CH₂)ₙ heterocyclic, C₁-C₆ alkanoyl, (CH₂)ₙ aryl, or (CH₂)ₙ heteroaryl;
R² and R³ independently are hydrogen, C₁-C₆ alkyl, C₂-C₆ alkenyl, C₂-C₆ alkynyl, CH, NO₂, NR⁴R⁵, (CH₂)ₙ cycloalkyl, (CH₂)ₙ aryl, or (CH₂)ₙ heteroaryl;
R² may further be halo;
n is an integer of from 0 to 5; and
R⁴ and R⁵ when taken together with a nitrogen to which they are both attached complete a 3- to 8-membered ring containing carbon atoms and optionally containing O, S, or N, and substituted or unsubstituted;
with the proviso that R¹ and R³ are not both selected from hydrogen and C₁-C₆ alkyl.

12. A compound of Formula V 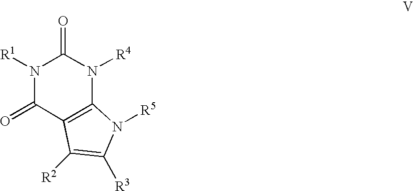
or a pharmaceutically acceptable salt thereof, wherein:
A is 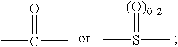
B is O or NR⁵; or
A and B are taken together to form —C≡C—;
R¹, R⁴, and R⁵ independently are hydrogen, C₁-C₆ alkyl, C₂-C₆ alkenyl, C₂-C₆ alkynyl, (CH₂)ₙ cycloalkyl, (CH₂)ₙ heterocyclic, C₁-C₆ alkanoyl, (CH₂)ₙ aryl, or (CH₂)ₙ heteroaryl;
R² and R³ independently are hydrogen, C₁-C₆ alkyl, C₂-C₆ alkenyl, C₂-C₆ alkynyl, CH, NO₂, NR⁴R⁵, (CH₂)ₙ cycloalkyl, (CH₂)ₙ aryl, or (CH₂)ₙ heteroaryl;
R² may further be halo;
n is an integer of from 0 to 5; and
R⁴ and R⁵ when taken together with a nitrogen to which they are both attached complete a 3- to 8-membered ring containing carbon atoms and optionally containing O, S, or N, and substituted or unsubstituted;
with the proviso that R¹ and R³ are not both selected from hydrogen and C₁-C₆ alkyl.

13. The compound according to claim 1, selected from:
4-{6-[3-(4-Methoxy-phenyl)-prop-1-ynyl]-1-methyl-2,4-dioxo-1,4-dihydro-2H-thieno[2,3-d]pyrimidine-3-ylmethyl}-benzoic acid;
3-(4-Methanesulfonyl-benzyl)-6-[3-(4-methoxy-phenyl)-prop-1-ynyl]-1-methyl-1H-thieno[2,3-d]pyrimidine-2,4-dione;
4-{6-[3-(3-Methoxy-phenyl)-prop-1-ynyl]-1-methyl-2,4-dioxo-1,4-dihydro-2H-thieno[2,3-d]pyrimidine-3-ylmethyl}-benzoic acid;
3-(4-Methanesulfonyl-benzyl)-6-[3-(3-methoxy-phenyl)-prop-1-ynyl]-1-methyl-1H-thieno[2,3-d]pyrimidine-2,4-dione;
4-[1-Methyl-2,4-dioxo-6-(3-pyridine-4-yl-prop-1-ynyl)-1,4-dihydro-2H-thieno[2,3-d]pyrimidine-3-ylmethyl]-benzoic acid;
3-(4-Methanesulfonyl-benzyl)-1-6-(3-pyridin-4-yl-prop-1-ynyl)-1H-thieno[2,3-d]pyrimidine-2,4-dione;
4-[1-Methyl-2,4-dioxo-6-(3-pyridine-3-yl-prop-1-ynyl)-1,4-dihydro-2H-thieno[2,3-d]pyrimidine-3-ylmethyl]-benzoic acid;
3-(4-Methanesulfonyl-benzyl)-1-6-(3-pyridin-3-yl-prop-1-ynyl)-1H-thieno[2,3-d]pyrimidine-2,4-dione;
4-{6-[3-(4-Fluoro-phenyl)-prop-1-ynyl]-1-methyl-2,4-dioxo-1,4-dihydro-2H-thieno[2,3-d]pyrimidine-3-ylmethyl}-benzoic acid;
6-[3-(4-Fluoro-phenyl)-prop-1-ynyl]-3-(4-methanesulfonyl-benzyl)-1-methyl-1H-thieno[2,3-d]pyrimidine-2,4-dione;
4-{6-[3-(3-Fluoro-phenyl)-prop-1-ynyl]-1-methyl-2,4-dioxo-1,4-dihydro-2H-thieno[2,3-d]pyrimidine-3-ylmethyl}-benzoic acid;
6-[3-(3-Fluoro-phenyl)-prop-1-ynyl]-3-(4-methanesulfonyl-benzyl)-1-methyl-1H-thieno[2,3-d]pyrimidine-2,4-dione;
4-{6-[3-(4-Chloro-phenyl)-prop-1-ynyl]-1-methyl-2,4-dioxo-1,4-dihydro-2H-thieno[2,3-d]pyrimidine-3-ylmethyl}-benzoic acid;
6-[3-(4-Chloro-phenyl)-prop-1-ynyl]-3-(4-methanesulfonyl-benzyl)-1-methyl-1H-thieno[2,3-d]pyrimidine-2,4-dione;
4-{6-[3-(3-Chloro-phenyl)-prop-1-ynyl]-1-methyl-2,4-dioxo-1,4-dihydro-2H-thieno[2,3-d]pyrimidine-3-ylmethyl}-benzoic acid;
6-[3-(3-Chloro-phenyl)-prop-1-ynyl]-3-(4-methanesulfonyl-benzyl)-1-methyl-1H-thieno[2,3-d]pyrimidine-2,4-dione;
4-{6-[3-(4-Bromo-phenyl)-prop-1-ynyl]-1-methyl-2,4-dioxo-1,4-dihydro-2H-thieno[2,3-d]pyrimidine-3-ylmethyl}-benzoic acid;
6-[3-(4-Bromo-phenyl)-prop-1-ynyl]-3-(4-methanesulfonyl-benzyl)-1-methyl-1H-thieno[2,3-d]pyrimidine-2,4-dione;
4-{6-[3-(3-Bromo-phenyl)-prop-1-ynyl]-1-methyl-2,4-dioxo-1,4-dihydro-2H-thieno[2,3-d]pyrimidine-3-ylmethyl}-benzoic acid;
6-[3-(3-Bromo-phenyl)-prop-1-ynyl]-3-(4-methanesulfonyl-benzyl)-1-methyl-1H-thieno[2,3-d]pyrimidine-2,4-dione;
4-{1-Methyl-6-[3-(4-nitro-phenyl)-prop-1-ynyl]-2,4-dioxo-1,4-dihydro-2H-thieno[2,3-d]pyrimidine-3-ylmethyl}-benzoic acid;
3-(4-Methanesulfonyl-benzyl)-1-methyl-6-[3-(4-nitro-phenyl)-prop-1-ynyl)-1H-thieno[2,3-d]pyrimidine-2,4-dione;
4-{6-[3-(2-Methoxy-pyridin-4-yl)-prop-1-ynyl]-1-methyl-2,4-dioxo-1,4-dihydro-2H-thieno[2,3-d]pyrimidine-3-ylmethyl}-benzoic acid;
3-(4-Methanesulfonyl-benzyl)-6-[3-(2-methoxy-pyridin-4-yl)-prop-1-ynyl]-1-methyl-1H-thieno[2,3-d]pyrimidine-2,4-dione;
4-{1-Methyl-6-[3-(4-methylsulfanyl-phenyl)-prop-1-ynyl]-2,4-dioxo-1,4-dihydro-2H-thieno[2,3-d]pyrimidine-3-ylmethyl}-benzoic acid;
3-(4-Methanesulfonyl-benzyl)-1-methyl-6-[3-(4-methylsulfanyl-phenyl)-prop-1-ynyl]-1H-thieno[2,3-d]pyrimidine-2,4-dione;
4-{1-Methyl-6-[3-(3-methylsulfanyl-phenyl)-prop-1-ynyl]-2,4-dioxo-1,4-dihydro-2H-thieno[2,3-d]pyrimidine-3-ylmethyl}-benzoic acid;
3-(4-Methanesulfonyl-benzyl)-1-methyl-6-[3-(3-methylsulfanyl-phenyl)-prop-1-ynyl]-1H-thieno[2,3-d]pyrimidine-2,4-dione;
4-[1-Methyl-2,4-dioxo-6-(3-p-tolyl-prop-1-ynyl)-1,4-dihydro-2H-thieno[2,3-d]pyrimidin-3-ylmethyl]benzoic acid;
3-(4-Methanesulfonyl-benzyl)-1-methyl-6-(3-p-tolyl-prop-1-ynyl)-1H-thieno[2,3-d]pyrimidine-2,4-dione;
4-[1-Methyl-2,4-dioxo-6-(3-m-tolyl-prop-1-ynyl)-1,4-dihydro-2H-thieno[2,3-d]pyrimidin-3-ylmethyl]benzoic acid;
3-(4-Methanesulfonyl-benzyl)-1-methyl-6-(3-m-tolyl-prop-1-ynyl)-1H-thieno[2,3-d]pyrimidine-2,4-dione;
3-Benzyl-6-[3-(4-methoxy-phenyl)-prop-1-ynyl]-1-methyl-1H-thieno[2,3-d]pyrimidine-2,4-dione;
3-Benzyl-6-[3-(3-methoxy-phenyl)-prop-1-ynyl]-1-methyl-1H-thieno[2,3-d]pyrimidine-2,4-dione;
3-Benzyl-1-methyl-6-(3-pyridin-4-yl-prop-1-ynyl)-1H-thieno[2,3-d]pyrimidine-2,4-dione;
3-Benzyl-1-methyl-6-(3-pyridin-3-yl-prop-1-ynyl)-1H-thieno[2,3-d]pyrimidine-2,4-dione;
3-Benzyl-6-[3-(4-fluoro-phenyl)-prop-1-ynyl]-1-methyl-1H-thieno[2,3-d]pyrimidine-2,4-dione;
3-Benzyl-6-[3-(3-fluoro-phenyl)-prop-1-ynyl]-1-methyl-1H-thieno[2,3-d]pyrimidine-2,4-dione;
3-Benzyl-6-[3-(4-chloro-phenyl)-prop-1-ynyl]-1-methyl-1H-thieno[2,3-d]pyrimidine-2,4-dione;
3-Benzyl-6-[3-(3-chloro-phenyl)-prop-1-ynyl]-1-methyl-1H-thieno[2,3-d]pyrimidine-2,4-dione;
3-Benzyl-6-[3-(4-bromo-phenyl)-prop-1-ynyl]-1-methyl-1H-thieno[2,3-d]pyrimidine-2,4-dione;
3-Benzyl-6-[3-(3-bromo-phenyl)-prop-1-ynyl]-1-methyl-1H-thieno[2,3-d]pyrimidine-2,4-dione;
3-Benzyl-6-[3-(2-methoxy-pyridin-4-yl)-prop-1-ynyl]-1-methyl-1H-thieno[2,3-d]pyrimidine-2,4-dione;
3-Benzyl-1-methyl-6-[3-(4-methylsulfanyl-phenyl)-prop-1-ynyl]-1H-thieno[2,3-d]pyrimidine-2,4-dione;
3-Benzyl-1-methyl-6-[3-(3-methylsulfanyl-phenyl)-prop-1-ynyl]-1H-thieno[2,3-d]pyrimidine-2,4-dione;
3-Benzyl-1-methyl-6-(3-p-tolyl-prop-1-ynyl)-1H-thieno[2,3-d]pyrimidine-2,4-dione;
3-Benzyl-1-methyl-6-(3-m-tolyl-prop-1-ynyl)-1H-thieno[2,3-d]pyrimidine-2,4-dione;
3-(3-Fluoro-benzyl)-6-[3-(4-methoxy-phenyl)-prop-1-ynyl]-1-methyl-1H-thieno[2,3-d]pyrimidine-2,4-dione;
3-(3-Fluoro-benzyl)-6-[3-(3-methoxy-phenyl)-prop-1-ynyl]-1-methyl-1H-thieno[2,3-d]pyrimidine-2,4-dione;
3-(3-Fluoro-benzyl)-1-methyl-6-(3-pyridine-4-yl-prop-1-ynyl)-1H-thieno[2,3-d]pyrimidine-2,4-dione;
3-(3-Fluoro-benzyl)-1-methyl-6-(3-pyridine-3-yl-prop-1-ynyl)-1H-thieno[2,3-d]pyrimidine-2,4-dione;
3-(3-Fluoro-benzyl)-6-[3-(4-fluoro-phenyl)-prop-1-ynyl]-1-methyl-1H-thieno[2,3-d]pyrimidine-2,4-dione;
3-(3-Fluoro-benzyl)-6-[3-(3-fluoro-phenyl)-prop-1-ynyl]-1-methyl-1H-thieno[2,3-d]pyrimidine-2,4-dione;
6-[3-(4-Chloro-phenyl)-prop-1-ynyl]-3-(3-fluoro-benzyl)-1-methyl-1H-thieno[2,3-d]pyrimidine-2,4-dione;
6-[3-(3-Chloro-phenyl)-prop-1-ynyl]-3-(3-fluoro-benzyl)-1-methyl-1H-thieno[2,3-d]pyrimidine-2,4-dione;
6-[3-(4-Bromo-phenyl)-prop-1-ynyl]-3-(3-fluoro-benzyl)-1-methyl-1H-thieno[2,3-d]pyrimidine-2,4-dione;
6-[3-(3-Bromo-phenyl)-prop-1-ynyl]-3-(3-fluoro-benzyl)-1-methyl-1H-thieno[2,3-d]pyrimidine-2,4-dione;
3-(3-Fluoro-benzyl)-6-[3-(2-methoxy-pyridin-4-yl)-prop-1-ynyl]-1-methyl-1H-thieno[2,3-d]pyrimidine-2,4-dione;
3-(3-Fluoro-benzyl)-1-methyl-6-[3-(4-methylsulfanyl-phenyl)-prop-1-ynyl]-1H-thieno[2,3-d]pyrimidine-2,4-dione;
3-(3-Fluoro-benzyl)-1-methyl-6-[3-(3-methylsulfanyl-phenyl)-prop-1-ynyl]-1H-thieno[2,3-d]pyrimidine-2,4-dione;
3-(3-Fluoro-benzyl)-1-methyl-6-(3-p-tolyl-prop-1-ynyl)-1H-thieno[2,3-d]pyrimidine-2,4-dione; and
3-(3-Fluoro-benzyl)-1-methyl-6-(3-m-tolyl-prop-1-ynyl)-1H-thieno[2,3-d]pyrimidine-2,4-dione.

14. The compound according to claim 1, selected from:
3-(3-Methoxycarbonyl-benzyl)-1-methyl-2,4-dioxo-1,2,3,4-tetrahydro-thieno[2,3-d]pyrimidine-6-carboxylic acid benzyl ester;
3-(3-Methoxycarbonyl-benzyl)-1-methyl-2,4-dioxo-1,2,3,4-tetrahydro-thieno[2,3-d]pyrimidine-6-carboxylic acid benzyl ester;
3-Benzofuran-5-ylmethyl-1-methyl-2,4-dioxo-1,2,3,4-tetrahydro-thieno[2,3-d]pyrimidine-6-carboxylic acid benzyl ester;
1-Methyl-3-(4-methyl-benzyl)-2,4-dioxo-1,2,3,4-tetrahydro-thieno[2,3-d]pyrimidine-6-carboxylic acid benzyl ester;
3-(4-Acetylamino-benzyl)-1-methyl-2,4-dioxo-1,2,3,4-tetrahydro-thieno[2,3-d]pyrimidine-6-carboxylic acid benzyl ester;
1-Methyl-2,4-dioxo-3-(4-vinyl-benzyl)-1,2,3,4-tetrahydro-thieno[2,3-d]pyrimidine-6-carboxylic acid benzyl ester;
1-Methyl-2,4-dioxo-3-(4-sulfamoyl-benzyl)-1,2,3,4-tetrahydro-thieno[2,3-d]pyrimidine-6-carboxylic acid benzyl ester;
3-(4-Bromo-benzyl)-2,4-dioxo-1,2,3,4-tetrahydro-thieno[2,3-d]pyrimidine-6-carboxylic acid pyridin-4-ylmethyl ester;
1-Methyl-2,4-dioxo-3-phenethyl-1,2,3,4-tetrahydro-thieno[2,3-d]pyrimidine-6-carboxylic acid benzyl ester;
1-Methyl-2,4-dioxo-3-[4-(2H-tetrazol-5-yl)-benzyl]-1,2,3,4-tetrahydro-thieno[2,3-d]pyrimidine-6-carboxylic acid benzyl ester;
3-(4-Fluoro-benzyl)-2,4-dioxo-1,2,3,4-tetrahydro-thieno[2,3-d]pyrimidine-6-carboxylic acid pyridin-4-ylmethyl ester;
3-(4-tert-Butyoxycarbonyl-benzyl)-1-methyl-2,4-dioxo-1,2,3,4-tetrahydro-thieno[2,3-d]pyrimidine-6-carboxylic acid benzyl ester;
3-(4-tert-Butyoxycarbonyl-benzyl)-1-methyl-2,4-dioxo-1,2,3,4-tetrahydro-thieno[2,3-d]pyrimidine-6-carboxylic acid;
4-[6-(4-Fluoro-benzylcarbamoyl)-1-methyl-2,4-dioxo-1,4-dihydro-2H-thieno[2,3-d]pyrimidin-3-ylmethyl]-benzoic acid;
4-[6-(4-Dimethylamino-benzylcarbamoyl)-1-methyl-2,4-dioxo-1,4-dihydro-2H-thieno[2,3-d]pyrimidin-3-ylmethyl]-benzoic acid, compound with trifluoro-acetic acid;
4-[6-(2-Ethoxy-benzylcarbamoyl)-1-methyl-2,4-dioxo-1,2,3,4-tetrahydro-thieno[2,3-d]pyrimidine-3-ylmethyl]-benzoic acid;
1-Methyl-2,4-dioxo-1,2,3,4-tetrahydro-thieno[2,3-d]pyrimidine-6-carboxylic acid;
1-Methyl-2,4-dioxo-1,2,3,4-tetrahydro-thieno[2,3-d]pyrimidine-6-carboxylic acid 3-methoxy-benzylamide;
1-Methyl-2,4-dioxo-3-[4-(1H-tetrazol-5-yl)-benzyl]-1,2,3,4-tetrahydro-thieno[2,3-d]pyrimidine-6-carboxylic acid 3-methoxy-benzylamide;
1-Methyl-3-[4-(morpholine-4-sulfonyl)-benzyl]-2,4-dioxo-1,2,3,4-tetrahydro-thieno[2,3-d]pyrimidine-6-carboxylic acid 3-methoxy-benzylamide;
1-Methyl-3-[4-(morpholine-4-carbonyl)-benzyl]-2,4-dioxo-1,2,3,4-tetrahydro-thieno[2,3-d]pyrimidine-6-carboxylic acid 3-methoxy-benzylamide;
3-But-2-ynyl-1-methyl-2,4-dioxo-1,2,3,4-tetrahydro-thieno[2,3-d]pyrimidine-6-carboxylic acid 3-methoxy-benzylamide;
1-Methyl-2,4-dioxo-3-[3-(1H-tetrazol-5-yl)-benzyl]-1,2,3,4-tetrahydro-thieno[2,3-d]pyrimidine-6-carboxylic acid 3-methoxy-benzylamide;
3-(4-Cyano-benzyl)-1-methyl-2,4-dioxo-1,2,3,4-tetrahydro-thieno[2,3-d]pyrimidine-6-carboxylic acid 3-methoxy-benzylamide;
{4-[6-(3-Methoxy-benzylcarbamoyl)-1-methyl-2,4-dioxo-1,4-dihydro-2H-thieno[2,3-d]pyrimidin-3-ylmethyl]-phenyl}-acetic acid;
3-[2-(2,4-Dichloro-benzenesulfonyl)-ethyl]-1-methyl-2,4-dioxo-1,2,3,4-tetrahydro-thieno[2,3-d]pyrimidine-6-carboxylic acid 3-methoxy-benzylamide;
3-(4-Methanesulfonyl-benzyl)-1-methyl-2,4-dioxo-1,2,3,4-tetrahydro-thieno[2,3-d]pyrimidine-6-carboxylic acid 3-methoxy-benzylamide;
1-Methyl-2,4-dioxo-3-(4-sulfamoyl-benzyl)-1,2,3,4-tetrahydro-thieno[2,3-d]pyrimidine-6-carboxylic acid 3-methoxy-benzylamide;
3-Benzyl-1-methyl-2,4-dioxo-1,2,3,4-tetrahydro-thieno[2,3-d]pyrimidine-6-carboxylic acid (2-methoxy-pyridin-4-ylmethyl)-amide;
1-Methyl-3-(4-methylsulfamoyl-benzyl)-2,4-dioxo-1,2,3,4-tetrahydro-thieno[2,3-d]pyrimidine-6-carboxylic acid 3-methoxy-benzylamide;
3-(4-Isopropylsulfamoyl-benzyl)-1-methyl-2,4-dioxo-1,2,3,4-tetrahydro-thieno[2,3-d]pyrimidine-6-carboxylic acid 3-methoxy-benzylamide;
1-Methyl-2,4-dioxo-3-[4-(pyrrolidine-1-sulfonyl)-benzyl]-1,2,3,4-tetrahydro-thieno[2,3-d]pyrimidine-6-carboxylic acid 3-methoxy-benzylamide; and
1-Methyl-3-[4-(4-methyl-piperidine-1-sulfonyl)-benzyl]-2,4-dioxo-1,2,3,4-tetrahydro-thieno[2,3-d]pyrimidine-6-carboxylic acid 3-methoxy-benzylamide.

15. The compound according to claim 1, selected from:
3-Benzyl-1-methyl-2,4-dioxo-1,2,3,4-tetrahydro-thieno[2,3-d]pyrimidine-6-carboxylic acid benzofuran-2-ylmethyl ester;
3-(4-Bromo-benzyl)-2,4-dioxo-1,2,3,4-tetrahydro-thieno[2,3-d]pyrimidine-6-carboxylic acid pyridin-4-ylmethyl ester;
3-Benzyl-2,4-dioxo-1,2,3,4-tetrahydro-thieno[2,3-d]pyrimidine-6-carboxylic acid 4-methoxy-benzyl ester;
4-{1-Methyl-2,4-dioxo-6-[(pyridin-4-ylmethyl)-carbamoyl]-1,4-dihydro-2H-thieno[2,3-d]pyrimidin-3-ylmethyl}-benzoic acid, compound with trifluoro-acetic acid;
4-[6-(4-Methoxy-benzylcarbamoyl)-1-methyl-2,4-dioxo-1,4-dihydro-2H-thieno[2,3-d]pyrimidin-3-ylmethyl]-benzoic acid;
4-[6-(3,4-Dimethoxy-benzylcarbamoyl)-1-methyl-2,4-dioxo-1,4-dihydro-2H-thieno[2,3-d]pyrimidin-3-ylmethyl]-benzoic acid tert-butyl ester;
4-[6-(3,4-Dimethoxy-benzylcarbamoyl)-1-methyl-2,4-dioxo-1,4-dihydro-2H-thieno[2,3-d]pyrimidin-3-ylmethyl]-benzoic acid;
4-[6-(4-Bromo-benzylcarbamoyl)-1-methyl-2,4-dioxo-1,4-dihydro-2H-thieno[2,3-d]pyrimidin-3-ylmethyl]-benzoic acid;
4-[6-(4-Bromo-benzylcarbamoyl)-1-methyl-2,4-dioxo-1,4-dihydro-2H-thieno[2,3-d]pyrimidin-3-ylmethyl]-benzoic acid tert-butyl ester;
4-[6-(3,5-Bis-trifluoromethyl-benzylcarbamoyl)-1-methyl-2,4-dioxo-1,4-dihydro-2H-thieno[2,3-d]pyrimidin-3-ylmethyl]-benzoic acid;
4-[6-(4-Chloro-benzylcarbamoyl)-1-methyl-2,4-dioxo-1,4-dihydro-2H-thieno[2,3-d]pyrimidin-3-ylmethyl]-benzoic acid;
4-[1-Methyl-2,4-dioxo-6-(4-sulfamoyl-benzylcarbamoyl)-1,4-dihydro-2H-thieno[2,3-d]pyrimidin-3-ylmethyl]-benzoic acid;
3-(4-Fluoro-benzyl)-1-methyl-2,4-dioxo-1,2,3,4-tetrahydro-thieno[2,3-d]pyrimidine-6-carboxylic acid 4-methoxy-benzylamide;
3-(4-Iodo-benzyl)-1-methyl-2,4-dioxo-1,2,3,4-tetrahydro-thieno[2,3-d]pyrimidine-6-carboxylic acid 3-methoxy-benzylamide;
3-(4-Dimethylsulfamoyl-benzyl)-1-methyl-2,4-dioxo-1,2,3,4-tetrahydro-thieno[2,3-d]pyrimidine-6-carboxylic acid 4-methoxy-benzylamide;
3-(3-Methoxy-benzyl)-1-methyl-2,4-dioxo-1,2,3,4-tetrahydro-thieno[2,3-d]pyrimidine-6-carboxylic acid 4-methoxy-benzylamide;
3-(4-Cyano-benzyl)-1-methyl-2,4-dioxo-1,2,3,4-tetrahydro-thieno[2,3-d]pyrimidine-6-carboxylic acid 4-methoxy-benzylamide;
3-(4-Acetylamino-benzyl)-1-methyl-2,4-dioxo-1,2,3,4-tetrahydro-thieno[2,3-d]pyrimidine-6-carboxylic acid 3-methoxy-benzylamide;
5-[6-(3-Methoxy-benzylcarbamoyl)-1-methyl-2,4-dioxo-1,4-dihydro-2H-thieno[2,3-d]pyrimidin-3-ylmethyl]-furan-2-carboxylic acid ethyl ester;
3-(4-Cyano-benzyl)-2,4-dioxo-1,2,3,4-tetrahydro-thieno[2,3-d]pyrimidine-6-carboxylic acid 3-methoxy-benzyl ester;
1-Methyl-2,4-dioxo-3-[4-(5-thioxo-4,5-dihydro-1,2,4-oxadiazol-3-yl)-benzyl]-1,2,3,4-tetrahydro-thieno[2,3-d]pyrimidine-6-carboxylic acid 4-methoxy-benzylamide;
4-[6-(3-Methoxy-benzylcarbamoyl)-1-methyl-2,4-dioxo-1,4-dihydro-2H-thieno[2,3-d]pyrimidin-3-ylmethyl]-benzoic acid 2-dimethylamino-ethyl ester;
3-Cyclohexylmethyl-1-methyl-2,4-dioxo-1,2,3,4-tetrahydro-thieno[2,3-d]pyrimidine-6-carboxylic acid 4-methoxy-benzylamide;
3-Cyclohexylmethyl-1-methyl-2,4-dioxo-1,2,3,4-tetrahydro-thieno[2,3-d]pyrimidine-6-carboxylic acid 4-methoxy-benzylamide;
3-Benzyl-2,4-dioxo-1,2,3,4-tetrahydro-thieno[2,3-d]pyrimidine-6-carboxylic acid furan-3-ylmethyl ester;
3-Benzyl-1-methyl-2,4-dioxo-1,2,3,4-tetrahydro-thieno[2,3-d]pyrimidine-6-carboxylic acid pentafluorophenylmethyl ester;
3-Benzyl-1-ethyl-2,4-dioxo-1,2,3,4-tetrahydro-thieno[2,3-d]pyrimidine-6-carboxylic acid benzyl ester;
3-Benzyl-1-cyclopropylmethyl-2,4-dioxo-1,2,3,4-tetrahydro-thieno[2,3-d]pyrimidine-6-carboxylic acid benzyl ester;
3-Benzyl-1-methyl-2,4-dioxo-1,2,3,4-tetrahydro-thieno[2,3-d]pyrimidine-6-carboxylic acid (pyridin-4-ylmethyl)-amide
3-Benzyl-1-methyl-2,4-dioxo-1,2,3,4-tetrahydro-thieno[2,3-d]pyrimidine-6-carboxylic acid 3-bromo-benzyl ester;
4-[6-(3-Difluoromethoxy-benzylcarbamoyl)-1-methyl-2,4-dioxo-1,4-dihydro-2H-thieno[2,3-d]pyrimidin-3-ylmethyl]-benzoic acid;
4-[6-(3-Difluoromethoxy-benzylcarbamoyl)-1-methyl-2,4-dioxo-1,4-dihydro-2H-thieno[2,3-d]pyrimidin-3-ylmethyl]-benzoic acid tert-butyl ester;
4-[6-(3-Methoxy-benzylcarbamoyl)-1-methyl-2,4-dioxo-1,4-dihydro-2H-thieno[2,3-d]pyrimidin-3-ylmethyl]-benzoic acid;
4-[6-(4-Methanesulfonyl-benzylcarbamoyl)-1-methyl-2,4-dioxo-1,4-dihydro-2H-thieno[2,3-d]pyrimidin-3-ylmethyl]-benzoic acid tert-butyl ester;
4-[6-(4-Methanesulfonyl-benzylcarbamoyl)-1-methyl-2,4-dioxo-1,4-dihydro-2H-thieno[2,3-d]pyrimidin-3-ylmethyl]-benzoic acid;
4-[1-Methyl-2,4-dioxo-6-(2-pyridin-4-yl-ethylcarbamoyl)-1,4-dihydro-2H-thieno[2,3-d]pyrimidin-3-ylmethyl]-benzoic acid;
1-Methyl-2,4-dioxo-3-(4-trifluoromethoxy-benzyl)-1,2,3,4-tetrahydro-thieno[2,3-d]pyrimidine-6-carboxylic acid 3-methoxy-benzylamide
4-[6-(3-Methoxy-benzylcarbamoyl)-1-methyl-2,4-dioxo-1,4-dihydro-2H-thieno[2,3-d]pyrimidin-3-ylmethyl]-benzoic acid methyl ester;
3-(2,3-Dihydro-benzofuran-6-ylmethyl)-1-methyl-2,4-dioxo-1,2,3,4-tetrahydro-thieno[2,3-d]pyrimidine-6-carboxylic acid 3-methoxy-benzylamide
1-Methyl-3-(2-methyl-thiazol-5-ylmethyl)-2,4-dioxo-1,2,3,4-tetrahydro-thieno[2,3-d]pyrimidine-6-carboxylic acid 3-methoxy-benzylamide
1-Methyl-2,4-dioxo-3-[4-(1H-tetrazol-5-yl)-benzyl]-1,2,3,4-tetrahydro-thieno[2,3-d]pyrimidine-6-carboxylic acid 4-fluoro-benzylamide;
3-Benzyl-2-methoxy-4-oxo-3,4-dihydro-thieno[2,3-d]pyrimidine-6-carboxylic acid benzyl ester;
4-[6-(3-Methoxy-benzylcarbamoyl)-1-methyl-2,4-dioxo-1,4-dihydro-2H-thieno[2,3-d]pyrimidin-3-ylmethyl]-benzoic acid 2,2-dimethyl-propionyloxymethyl ester;
4-[6-(3-Methoxy-benzylcarbamoyl)-1-methyl-2,4-dioxo-1,4-dihydro-2H-thieno[2,3-d]pyrimidin-3-ylmethyl]-cyclohexanecarboxylic acid;
4-[6-(3-Methoxy-benzylcarbamoyl)-1-methyl-2,4-dioxo-1,4-dihydro-2H-thieno[2,3-d]pyrimidin-3-ylmethyl]-cyclohexanecarboxylic acid methyl ester;
1-{4-[6-(3-Methoxy-benzylcarbamoyl)-1-methyl-2,4-dioxo-1,4-dihydro-2H-thieno[2,3-d]pyrimidin-3-ylmethyl]-phenyl}-cyclopropanecarboxylic acid methyl ester;
1-{4-[6-(3-Methoxy-benzylcarbamoyl)-1-methyl-2,4-dioxo-1,4-dihydro-2H-thieno[2,3-d]pyrimidin-3-ylmethyl]-phenyl}-cyclopropanecarboxylic acid tert-butyl ester;
1-{4-[6-(3-Methoxy-benzylcarbamoyl)-1-methyl-2,4-dioxo-1,4-dihydro-2H-thieno[2,3-d]pyrimidin-3-ylmethyl]-phenyl}-cyclopropanecarboxylic acid;
2-{4-[6-(3-Methoxy-benzylcarbamoyl)-1-methyl-2,4-dioxo-1,4-dihydro-2H-thieno[2,3-d]pyrimidin-3-ylmethyl]-phenoxy}-2-methyl-propionic acid tert-butyl ester;
2-{4-[6-(3-Methoxy-benzylcarbamoyl)-1-methyl-2,4-dioxo-1,4-dihydro-2H-thieno[2,3-d]pyrimidin-3-ylmethyl]-phenoxy}-2-methyl-propionic acid;
3-Benzyl-1-methyl-2,4-dioxo-1,2,3,4-tetrahydro-furo[2,3-d]pyrimidine-6-carboxylic acid benzyl ester;
3-(3-Methoxy-benzyl)-1-methyl-2,4-dioxo-1,2,3,4-tetrahydro-thieno[2,3-d]pyrimidine-6-carboxylic acid benzyl ester;
3-Benzyl-1-methyl-2,4-dioxo-1,2,3,4-tetrahydro-thieno[2,3-d]pyrimidine-6-carboxylic acid benzyl ester;
3-Biphenyl-4-ylmethyl-1-methyl-2,4-dioxo-1,2,3,4-tetrahydro-thieno[2,3-d]pyrimidine-6-carboxylic acid benzyl ester;
3-(4-Methanesulfonyl-benzyl)-1-methyl-2,4-dioxo-1,2,3,4-tetrahydro-thieno[2,3-d]pyrimidine-6-carboxylic acid benzyl ester;
3-(4-Methanesulfonyl-benzyl)-1-methyl-2,4-dioxo-1,2,3,4-tetrahydro-thieno[2,3-d]pyrimidine-6-carboxylic acid benzyl ester;
1-Methyl-3-(4-methyl-benzyl)-2,4-dioxo-1,2,3,4-tetrahydro-thieno[2,3-d]pyrimidine-6-carboxylic acid benzyl ester;
1-Methyl-2,4-dioxo-3-phenethyl-1,2,3,4-tetrahydro-thieno[2,3-d]pyrimidine-6-carboxylic acid benzyl ester;
3-(4-Amino-6-phenylamino-1,3,5-triazin-2-ylmethyl)-1-methyl-2,4-dioxo-1,2,3,4-tetrahydro-thieno[2,3-d]pyrimidine-6-carboxylic acid benzyl ester;
1-Methyl-2,4-dioxo-3-(4-trifluoromethyl-benzyl)-1,2,3,4-tetrahydro-thieno[2,3-d]pyrimidine-6-carboxylic acid benzyl ester;
3-(6-Cyano-hexyl)-1-methyl-2,4-dioxo-1,2,3,4-tetrahydro-thieno[2,3-d]pyrimidine-6-carboxylic acid benzyl ester;
3-[2-(2,5-Dimethoxy-phenyl)-2-oxo-ethyl]-1-methyl-2,4-dioxo-1,2,3,4-tetrahydro-thieno[2,3-d]pyrimidine-6-carboxylic acid benzyl ester;
3-(3-Iodo-benzyl)-1-methyl-2,4-dioxo-1,2,3,4-tetrahydro-thieno[2,3-d]pyrimidine-6-carboxylic acid benzyl ester;
1-Methyl-2,4-dioxo-3-(3-trifluoromethyl-benzyl)-1,2,3,4-tetrahydro-thieno[2,3-d]pyrimidine-6-carboxylic acid benzyl ester;
3-(2,4-Bis-trifluoromethyl-benzyl)-1-methyl-2,4-dioxo-1,2,3,4-tetrahydro-thieno[2,3-d]pyrimidine-6-carboxylic acid benzyl ester;
3-[2-(1,3-Dioxo-1,3-dihydro-isoindol-2-yl)-ethyl]-1-methyl-2,4-dioxo-1,2,3,4-tetrahydro-thieno[2,3-d]pyrimidine-6-carboxylic acid benzyl ester;
3-[2-(1,3-Dioxo-1,3-dihydro-isoindol-2-yl)-ethyl]-1-methyl-2,4-dioxo-1,2,3,4-tetrahydro-thieno[2,3-d]pyrimidine-6-carboxylic acid benzyl ester;
3-(2-Carboxy-allyl)-1-methyl-2,4-dioxo-1,2,3,4-tetrahydro-thieno[2,3-d]pyrimidine-6-carboxylic acid benzyl ester;
3-(2-Carboxy-allyl)-1-methyl-2,4-dioxo-1,2,3,4-tetrahydro-thieno[2,3-d]pyrimidine-6-carboxylic acid benzyl ester;
3-(3-Amino-propyl)-1-methyl-2,4-dioxo-1,2,3,4-tetrahydro-thieno[2,3-d]pyrimidine-6-carboxylic acid benzyl ester;
3-(1,3-Dioxo-1,3-dihydro-isoindol-2-ylmethyl)-1-methyl-2,4-dioxo-1,2,3,4-tetrahydro-thieno[2,3-d]pyrimidine-6-carboxylic acid benzyl ester;
3-(4-Fluoro-benzyl)-1-methyl-2,4-dioxo-1,2,3,4-tetrahydro-thieno[2,3-d]pyrimidine-6-carboxylic acid benzyl ester;
1-Methyl-3-oxiranylmethyl-2,4-dioxo-1,2,3,4-tetrahydro-thieno[2,3-d]pyrimidine-6-carboxylic acid benzyl ester;
1-Methyl-3-((S)-2-methyl-butyl)-2,4-dioxo-1,2,3,4-tetrahydro-thieno[2,3-d]pyrimidine-6-carboxylic acid benzyl ester;
1-Methyl-2,4-dioxo-3-(4-phenoxy-butyl)-1,2,3,4-tetrahydro-thieno[2,3-d]pyrimidine-6-carboxylic acid benzyl ester;
3-(2-Cyano-benzyl)-1-methyl-2,4-dioxo-1,2,3,4-tetrahydro-thieno[2,3-d]pyrimidine-6-carboxylic acid benzyl ester;
1-Methyl-2,4-dioxo-3-(3-phenoxy-propyl)-1,2,3,4-tetrahydro-thieno[2,3-d]pyrimidine-6-carboxylic acid benzyl ester;
3-Hex-5-enyl-1-methyl-2,4-dioxo-1,2,3,4-tetrahydro-thieno[2,3-d]pyrimidine-6-carboxylic acid benzyl ester;
1-Methyl-2,4-dioxo-3-pyridin-3-ylmethyl-1,2,3,4-tetrahydro-thieno[2,3-d]pyrimidine-6-carboxylic acid benzyl ester;
3-[2-Hydroxy-3-(naphthalen-1-yloxy)-propyl]-1-methyl-2,4-dioxo-1,2,3,4-tetrahydro-thieno[2,3-d]pyrimidine-6-carboxylic acid benzyl ester;
1,3-Dimethyl-2,4-dioxo-1,2,3,4-tetrahydro-thieno[2,3-d]pyrimidine-6-carboxylic acid benzyl ester;
3-Cyclobutylmethyl-1-methyl-2,4-dioxo-1,2,3,4-tetrahydro-thieno[2,3-d]pyrimidine-6-carboxylic acid benzyl ester;
3-Allyl-1-methyl-2,4-dioxo-1,2,3,4-tetrahydro-thieno[2,3-d]pyrimidine-6-carboxylic acid benzyl ester;
1-Methyl-2,4-dioxo-3-prop-2-ynyl-1,2,3,4-tetrahydro-thieno[2,3-d]pyrimidine-6-carboxylic acid benzyl ester;
3-But-2-ynyl-1-methyl-2,4-dioxo-1,2,3,4-tetrahydro-thieno[2,3-d]pyrimidine-6-carboxylic acid benzyl ester;
1-Methyl-2,4-dioxo-3-(2-phenoxy-ethyl)-1,2,3,4-tetrahydro-thieno[2,3-d]pyrimidine-6-carboxylic acid benzyl ester;
1-Methyl-2,4-dioxo-3-(2-phenoxy-ethyl)-1,2,3,4-tetrahydro-thieno[2,3-d]pyrimidine-6-carboxylic acid benzyl ester;
3-((R)-3-Hydroxy-2-methyl-propyl)-1-methyl-2,4-dioxo-1,2,3,4-tetrahydro-thieno[2,3-d]pyrimidine-6-carboxylic acid benzyl ester;
3-Isobutyl-1-methyl-2,4-dioxo-1,2,3,4-tetrahydro-thieno[2,3-d]pyrimidine-6-carboxylic acid benzyl ester;
3-(6-Chloro-pyridin-3-ylmethyl)-1-methyl-2,4-dioxo-1,2,3,4-tetrahydro-thieno[2,3-d]pyrimidine-6-carboxylic acid benzyl ester;
3-(2-Benzenesulfonylmethyl-benzyl)-1-methyl-2,4-dioxo-1,2,3,4-tetrahydro-thieno[2,3-d]pyrimidine-6-carboxylic acid benzyl ester;
1-Methyl-3-naphthalen-1-ylmethyl-2,4-dioxo-1,2,3,4-tetrahydro-thieno[2,3-d]pyrimidine-6-carboxylic acid benzyl ester;
1-Methyl-2,4-dioxo-3-(2-trifluoromethyl-benzyl)-1,2,3,4-tetrahydro-thieno[2,3-d]pyrimidine-6-carboxylic acid benzyl ester;
3-(3-Chloro-benzyl)-1-methyl-2,4-dioxo-1,2,3,4-tetrahydro-thieno[2,3-d]pyrimidine-6-carboxylic acid benzyl ester;
3-(4-Methoxycarbonyl-butyl)-1-methyl-2,4-dioxo-1,2,3,4-tetrahydro-thieno[2,3-d]pyrimidine-6-carboxylic acid benzyl ester;
3-Ethyl-1-methyl-2,4-dioxo-1,2,3,4-tetrahydro-thieno[2,3-d]pyrimidine-6-carboxylic acid benzyl ester;
3-Benzyl-1-methyl-2,4-dioxo-1,2,3,4-tetrahydro-thieno[2,3-d]pyrimidine-6-carboxylic acid 4-fluoro-benzyl ester;
3-[2-(4-Chloro-benzenesulfonyl)-ethyl]-1-methyl-2,4-dioxo-1,2,3,4-tetrahydro-thieno[2,3-d]pyrimidine-6-carboxylic acid benzyl ester;
3-(2-Acetoxy-ethyl)-1-methyl-2,4-dioxo-1,2,3,4-tetrahydro-thieno[2,3-d]pyrimidine-6-carboxylic acid benzyl ester;
3-Benzyl-1-methyl-2,4-dioxo-1,2,3,4-tetrahydro-thieno[2,3-d]pyrimidine-6-carboxylic acid 2-phenoxy-ethyl ester;
3-Benzyl-1-methyl-2,4-dioxo-1,2,3,4-tetrahydro-thieno[2,3-d]pyrimidine-6-carboxylic acid benzyl ester;
3-Benzyl-1-methyl-2,4-dioxo-1,2,3,4-tetrahydro-thieno[2,3-d]pyrimidine-6-carboxylic acid benzylamide;
3-Benzyl-1-methyl-2,4-dioxo-1,2,3,4-tetrahydro-thieno[2,3-d]pyrimidine-6-carboxylic acid 2,6-dichloro-benzyl ester;
3-Benzyl-1-methyl-2,4-dioxo-1,2,3,4-tetrahydro-thieno[2,3-d]pyrimidine-6-carboxylic acid butyl ester;
3-Benzyl-1-methyl-2,4-dioxo-1,2,3,4-tetrahydro-thieno[2,3-d]pyrimidine-6-carboxylic acid 2,3-dihydro-1,4-benzodioxin-2-ylmethyl ester;
3-Benzyl-1-methyl-2,4-dioxo-1,2,3,4-tetrahydro-thieno[2,3-d]pyrimidine-6-carboxylic acid 2-diethylamino-1-methyl-ethyl ester;
3-Benzyl-1-methyl-2,4-dioxo-1,2,3,4-tetrahydro-thieno[2,3-d]pyrimidine-6-carboxylic acid 4-fluoro-benzyl ester;
3-Benzyl-1-methyl-2,4-dioxo-1,2,3,4-tetrahydro-thieno[2,3-d]pyrimidine-6-carboxylic acid 4-isopropyl-benzyl ester;
3-Benzyl-1-methyl-2,4-dioxo-1,2,3,4-tetrahydro-thieno[2,3-d]pyrimidine-6-carboxylic acid 2-p-tolyl-ethyl ester;
3-Benzyl-1-methyl-2,4-dioxo-1,2,3,4-tetrahydro-thieno[2,3-d]pyrimidine-6-carboxylic acid 4-trifluoromethyl-benzyl ester;
3-Benzyl-1-methyl-2,4-dioxo-1,2,3,4-tetrahydro-thieno[2,3-d]pyrimidine-6-carboxylic acid cyclobutylmethyl ester;
3-Benzyl-1-methyl-2,4-dioxo-1,2,3,4-tetrahydro-thieno[2,3-d]pyrimidine-6-carboxylic acid 2,6-difluoro-benzyl ester;
3-Benzyl-1-methyl-2,4-dioxo-1,2,3,4-tetrahydro-thieno[2,3-d]pyrimidine-6-carboxylic acid 2-(2-hydroxy-phenyl)-ethyl ester;
3-Benzyl-1-methyl-2,4-dioxo-1,2,3,4-tetrahydro-thieno[2,3-d]pyrimidine-6-carboxylic acid 2-(2-hydroxy-phenyl)-ethyl ester;
3-Benzyl-1-methyl-2,4-dioxo-1,2,3,4-tetrahydro-thieno[2,3-d]pyrimidine-6-carboxylic acid 1-methyl-piperidin-4-yl ester;
3-Benzyl-1-methyl-2,4-dioxo-1,2,3,4-tetrahydro-thieno[2,3-d]pyrimidine-6-carboxylic acid 1-methyl-piperidin-4-yl ester;
3-Benzyl-1-methyl-2,4-dioxo-1,2,3,4-tetrahydro-thieno[2,3-d]pyrimidine-6-carboxylic acid pyridin-3-ylmethyl ester;
3-Benzyl-1-methyl-2,4-dioxo-1,2,3,4-tetrahydro-thieno[2,3-d]pyrimidine-6-carboxylic acid 3-pyridin-3-yl-propyl ester;
3-Benzyl-1-methyl-2,4-dioxo-1,2,3,4-tetrahydro-thieno[2,3-d]pyrimidine-6-carboxylic acid 2-dimethylamino-1-methyl-ethyl ester;
3-Benzyl-1-methyl-2,4-dioxo-1,2,3,4-tetrahydro-thieno[2,3-d]pyrimidine-6-carboxylic acid 4-methoxy-benzyl ester;
3-Benzyl-1-methyl-2,4-dioxo-1,2,3,4-tetrahydro-thieno[2,3-d]pyrimidine-6-carboxylic acid tetrahydro-pyran-4-yl ester;
3-Benzyl-1-methyl-2,4-dioxo-1,2,3,4-tetrahydro-thieno[2,3-d]pyrimidine-6-carboxylic acid 2,2,2-trifluoro-1-trifluoromethyl-ethyl ester;
3-Benzyl-1-methyl-2,4-dioxo-1,2,3,4-tetrahydro-thieno[2,3-d]pyrimidine-6-carboxylic acid 2-trifluoromethyl-benzyl ester;
3-Benzyl-1-methyl-2,4-dioxo-1,2,3,4-tetrahydro-thieno[2,3-d]pyrimidine-6-carboxylic acid 2-benzyloxy-ethyl ester;
3-Benzyl-1-methyl-2,4-dioxo-1,2,3,4-tetrahydro-thieno[2,3-d]pyrimidine-6-carboxylic acid 2,2,2-trichloro-ethyl ester;
3-Benzyl-1-methyl-2,4-dioxo-1,2,3,4-tetrahydro-thieno[2,3-d]pyrimidine-6-carboxylic acid phenethyl ester;
3-Benzyl-1-methyl-2,4-dioxo-1,2,3,4-tetrahydro-thieno[2,3-d]pyrimidine-6-carboxylic acid 3-ethyl-oxetan-3-ylmethyl ester;
3-Benzyl-1-methyl-2,4-dioxo-1,2,3,4-tetrahydro-thieno[2,3-d]pyrimidine-6-carboxylic acid 2-morpholin-4-yl-ethyl ester;
3-Benzyl-1-methyl-2,4-dioxo-1,2,3,4-tetrahydro-thieno[2,3-d]pyrimidine-6-carboxylic acid 2-pyrrolidin-1-yl-ethyl ester;
3-Benzyl-1-methyl-2,4-dioxo-1,2,3,4-tetrahydro-thieno[2,3-d]pyrimidine-6-carboxylic acid 2-pyrrolidin-1-yl-ethyl ester;
3-Benzyl-1-methyl-2,4-dioxo-1,2,3,4-tetrahydro-thieno[2,3-d]pyrimidine-6-carboxylic acid 2-(2-ethoxy-ethoxy)-ethyl ester;
3-Benzyl-1-methyl-2,4-dioxo-1,2,3,4-tetrahydro-thieno[2,3-d]pyrimidine-6-carboxylic acid tetrahydro-pyran-2-ylmethyl ester;
3-Benzyl-1-methyl-2,4-dioxo-1,2,3,4-tetrahydro-thieno[2,3-d]pyrimidine-6-carboxylic acid 4-nitro-benzyl ester;
3-Benzyl-1-methyl-2,4-dioxo-1,2,3,4-tetrahydro-thieno[2,3-d]pyrimidine-6-carboxylic acid pentyl ester;
3-Benzyl-1-methyl-2,4-dioxo-1,2,3,4-tetrahydro-thieno[2,3-d]pyrimidine-6-carboxylic acid 3-phenyl-propyl ester;
3-Benzyl-1-methyl-2,4-dioxo-1,2,3,4-tetrahydro-thieno[2,3-d]pyrimidine-6-carboxylic acid 3-phenoxy-benzyl ester;
3-Benzyl-1-methyl-2,4-dioxo-1,2,3,4-tetrahydro-thieno[2,3-d]pyrimidine-6-carboxylic acid 3,5-dimethoxy-benzyl ester;
3-Benzyl-1-methyl-2,4-dioxo-1,2,3,4-tetrahydro-thieno[2,3-d]pyrimidine-6-carboxylic acid 3-methyl-butyl ester;
3-Benzyl-1-methyl-2,4-dioxo-1,2,3,4-tetrahydro-thieno[2,3-d]pyrimidine-6-carboxylic acid 4-chloro-benzyl ester;
3-Benzyl-1-methyl-2,4-dioxo-1,2,3,4-tetrahydro-thieno[2,3-d]pyrimidine-6-carboxylic acid 1-ethyl-piperidin-3-yl ester;
3-Benzyl-1-methyl-2,4-dioxo-1,2,3,4-tetrahydro-thieno[2,3-d]pyrimidine-6-carboxylic acid 4-benzyloxy-benzyl ester;
3-Benzyl-1-methyl-2,4-dioxo-1,2,3,4-tetrahydro-thieno[2,3-d]pyrimidine-6-carboxylic acid isobutyl ester;
3-Benzyl-1-methyl-2,4-dioxo-1,2,3,4-tetrahydro-thieno[2,3-d]pyrimidine-6-carboxylic acid 3-(4-methoxy-phenyl)-propyl ester;
3-Benzyl-1-methyl-2,4-dioxo-1,2,3,4-tetrahydro-thieno[2,3-d]pyrimidine-6-carboxylic acid 2-chloro-6-fluoro-benzyl ester;
3-Benzyl-1-methyl-2,4-dioxo-1,2,3,4-tetrahydro-thieno[2,3-d]pyrimidine-6-carboxylic acid (S)-(tetrahydro-furan-3-yl)ester;
3-Benzyl-1-methyl-2,4-dioxo-1,2,3,4-tetrahydro-thieno[2,3-d]pyrimidine-6-carboxylic acid 3-methoxy-benzyl ester;
3-Benzyl-1-methyl-2,4-dioxo-1,2,3,4-tetrahydro-thieno[2,3-d]pyrimidine-6-carboxylic acid 3-methoxy-benzyl ester;
3-Benzyl-1-methyl-2,4-dioxo-1,2,3,4-tetrahydro-thieno[2,3-d]pyrimidine-6-carboxylic acid 3-pyridin-2-yl-propyl ester;
3-Benzyl-1-methyl-2,4-dioxo-1,2,3,4-tetrahydro-thieno[2,3-d]pyrimidine-6-carboxylic acid 2-piperidin-2-yl-ethyl ester;
3-Benzyl-1-methyl-2,4-dioxo-1,2,3,4-tetrahydro-thieno[2,3-d]pyrimidine-6-carboxylic acid 5-bromo-2-methoxy-benzyl ester;
3-Benzyl-1-methyl-2,4-dioxo-1,2,3,4-tetrahydro-thieno[2,3-d]pyrimidine-6-carboxylic acid cycloheptylmethyl ester;
3-Benzyl-1-methyl-2,4-dioxo-1,2,3,4-tetrahydro-thieno[2,3-d]pyrimidine-6-carboxylic acid 1,2,3,4-tetrahydro-naphthalen-1-yl ester;
3-Benzyl-1-methyl-2,4-dioxo-1,2,3,4-tetrahydro-thieno[2,3-d]pyrimidine-6-carboxylic acid (S)-1-pyrrolidin-2-ylmethyl ester;
3-Benzyl-1-methyl-2,4-dioxo-1,2,3,4-tetrahydro-thieno[2,3-d]pyrimidine-6-carboxylic acid 3-chloro-benzyl ester;
3-Benzyl-1-methyl-2,4-dioxo-1,2,3,4-tetrahydro-thieno[2,3-d]pyrimidine-6-carboxylic acid 1,3-benzodioxol-5-ylmethyl ester;
3-Benzyl-1-methyl-2,4-dioxo-1,2,3,4-tetrahydro-thieno[2,3-d]pyrimidine-6-carboxylic acid 4-methylsulfanyl-benzyl ester;
3-Benzyl-1-methyl-2,4-dioxo-1,2,3,4-tetrahydro-thieno[2,3-d]pyrimidine-6-carboxylic acid 4-methylsulfanyl-benzyl ester;
3-Benzyl-1-methyl-2,4-dioxo-1,2,3,4-tetrahydro-thieno[2,3-d]pyrimidine-6-carboxylic acid 3,4-dichloro-benzyl ester;
3-Benzyl-1-methyl-2,4-dioxo-1,2,3,4-tetrahydro-thieno[2,3-d]pyrimidine-6-carboxylic acid 3,3-diphenyl-propyl ester;
3-Benzyl-1-methyl-2,4-dioxo-1,2,3,4-tetrahydro-thieno[2,3-d]pyrimidine-6-carboxylic acid 2-pyridin-2-yl-ethyl ester;
3-Benzyl-1-methyl-2,4-dioxo-1,2,3,4-tetrahydro-thieno[2,3-d]pyrimidine-6-carboxylic acid furan-3-ylmethyl ester;
3-Benzyl-1-methyl-2,4-dioxo-1,2,3,4-tetrahydro-thieno[2,3-d]pyrimidine-6-carboxylic acid but-3-enyl ester;
3-Benzyl-1-methyl-2,4-dioxo-1,2,3,4-tetrahydro-thieno[2,3-d]pyrimidine-6-carboxylic acid 2-cyano-ethyl ester;
3-Benzyl-1-methyl-2,4-dioxo-1,2,3,4-tetrahydro-thieno[2,3-d]pyrimidine-6-carboxylic acid 2-ethoxy-ethyl ester;
3-Benzyl-1-methyl-2,4-dioxo-1,2,3,4-tetrahydro-thieno[2,3-d]pyrimidine-6-carboxylic acid cyano-phenyl-methyl ester;
3-Benzyl-1-methyl-2,4-dioxo-1,2,3,4-tetrahydro-thieno[2,3-d]pyrimidine-6-carboxylic acid 4-trifluoromethyl-benzylamide;
3-Benzyl-1-methyl-2,4-dioxo-1,2,3,4-tetrahydro-thieno[2,3-d]pyrimidine-6-carboxylic acid 4-methyl-benzylamide;
3-Benzyl-1-methyl-2,4-dioxo-1,2,3,4-tetrahydro-thieno[2,3-d]pyrimidine-6-carboxylic acid phenethyl-amide;
3-Benzyl-1-methyl-2,4-dioxo-1,2,3,4-tetrahydro-thieno[2,3-d]pyrimidine-6-carboxylic acid cyclopropylamide;
1-Methyl-1,2,3,4-tetrahydro-thieno[2,3-d]pyrimidine-6-carboxylic acid 4-methoxy-benzylamide;
1-Methyl-2,4-dioxo-3-(4-sulfamoyl-benzyl)-1,2,3,4-tetrahydro-thieno[2,3-d]pyrimidine-6-carboxylic acid 4-methoxy-benzylamide;
1-Methyl-2,4-dioxo-1,2,3,4-tetrahydro-thieno[2,3-d]pyrimidine-6-carboxylic acid 3-methoxy-benzylamide;
1-Methyl-2,4-dioxo-3-(3-oxo-3-phenyl-propyl)-1,2,3,4-tetrahydro-thieno[2,3-d]pyrimidine-6-carboxylic acid 3-methoxy-benzylamide;
3-[4-(N-Hydroxycarbamimidoyl)-benzyl]-1-methyl-2,4-dioxo-1,2,3,4-tetrahydro-thieno[2,3-d]pyrimidine-6-carboxylic acid 4-methoxy-benzylamide;
1-Methyl-2,4-dioxo-3-[4-(5-oxo-4,5-dihydro-1,2,4-oxadiazol-3-yl)-benzyl]-1,2,3,4-tetrahydro-thieno[2,3-d]pyrimidine-6-carboxylic acid 4-methoxy-benzylamide;
1-Methyl-2,4-dioxo-3-[4-(5-thioxo-4,5-dihydro-1,2,4-oxadiazol-3-yl)-benzyl]-1,2,3,4-tetrahydro-thieno[2,3-d]pyrimidine-6-carboxylic acid 4-methoxy-benzylamide;
4-(5-Isopropyl-2H-pyrazol-3-yl)-pyridine;
3-Cyanomethyl-1-methyl-2,4-dioxo-1,2,3,4-tetrahydro-thieno[2,3-d]pyrimidine-6-carboxylic acid 3-methoxy-benzylamide;
(E)-4-[6-(4-Methoxy-benzylcarbamoyl)-1-methyl-2,4-dioxo-1,4-dihydro-2H-thieno[2,3-d]pyrimidin-3-yl]-but-2-enoic acid methyl ester;
(E)-4-[6-(4-Methoxy-benzylcarbamoyl)-1-methyl-2,4-dioxo-1,4-dihydro-2H-thieno[2,3-d]pyrimidin-3-yl]-but-2-enoic acid;
3-(2-Benzenesulfonyl-ethyl)-1-methyl-2,4-dioxo-1,2,3,4-tetrahydro-thieno[2,3-d]pyrimidine-6-carboxylic acid 4-methoxy-benzylamide;
2-Methoxy-4-[6-(4-methoxy-benzylcarbamoyl)-1-methyl-2,4-dioxo-1,4-dihydro-2H-thieno[2,3-d]pyrimidin-3-ylmethyl]-benzoic acid methyl ester;
3-(2-Methoxymethyl-1,1,3-trioxo-2,3-dihydro-1H-1l⁶-1,2-benzisothiazol-6-ylmethyl)-1-methyl-2,4-dioxo-1,2,3,4-tetrahydro-thieno[2,3-d]pyrimidine-6-carboxylic acid 4-methoxy-benzylamide;
1-Methyl-3-oct-2-ynyl-2,4-dioxo-1,2,3,4-tetrahydro-thieno[2,3-d]pyrimidine-6-carboxylic acid 4-methoxy-benzylamide;
3-[2-(4-Chloro-benzenesulfonylamino)-ethyl]-1-methyl-2,4-dioxo-1,2,3,4-tetrahydro-thieno[2,3-d]pyrimidine-6-carboxylic acid 4-methoxy-benzylamide;
3-[2-(4-Bromo-phenoxy)-ethyl]-1-methyl-2,4-dioxo-1,2,3,4-tetrahydro-thieno[2,3-d]pyrimidine-6-carboxylic acid 4-methoxy-benzylamide;
3-[2-(4-Bromo-phenoxy)-ethyl]-1-methyl-2,4-dioxo-1,2,3,4-tetrahydro-thieno[2,3-d]pyrimidine-6-carboxylic acid 3-methoxy-benzylamide;
3-[2-(4-Fluoro-phenoxy)-ethyl]-1-methyl-2,4-dioxo-1,2,3,4-tetrahydro-thieno[2,3-d]pyrimidine-6-carboxylic acid 4-methoxy-benzylamide;
3-[2-(4-Chloro-benzenesulfonyl)-ethyl]-1-methyl-2,4-dioxo-1,2,3,4-tetrahydro-thieno[2,3-d]pyrimidine-6-carboxylic acid 4-methoxy-benzylamide;
3-[2-(4-Fluoro-phenoxy)-ethyl]-1-methyl-2,4-dioxo-1,2,3,4-tetrahydro-thieno[2,3-d]pyrimidine-6-carboxylic acid 3-methoxy-benzylamide;
1-Methyl-2,4-dioxo-1,2,3,4-tetrahydro-thieno[2,3-d]pyrimidine-6-carboxylic acid (2-methoxy-pyridin-4-ylmethyl)-amide;
3-Cyclohexylmethyl-1-methyl-2,4-dioxo-1,2,3,4-tetrahydro-thieno[2,3-d]pyrimidine-6-carboxylic acid 3-methoxy-benzylamide;
4-[6-(4-Methoxy-benzylcarbamoyl)-1-methyl-2,4-dioxo-1,4-dihydro-2H-thieno[2,3-d]pyrimidin-3-ylmethyl]-2-methyl-benzoic acid methyl ester;
4-[6-(4-Methoxy-benzylcarbamoyl)-1-methyl-2,4-dioxo-1,4-dihydro-2H-thieno[2,3-d]pyrimidin-3-ylmethyl]-benzoic acid methyl ester;
2-Methoxy-4-[6-(3-methoxy-benzylcarbamoyl)-1-methyl-2,4-dioxo-1,4-dihydro-2H-thieno[2,3-d]pyrimidin-3-ylmethyl]-benzoic acid methyl ester;
4-[6-(3-Methoxy-benzylcarbamoyl)-1-methyl-2,4-dioxo-1,4-dihydro-2H-thieno[2,3-d]pyrimidin-3-ylmethyl]-2-methyl-benzoic acid methyl ester;
1-Methyl-2,4-dioxo-3-(3-oxo-3-phenyl-propyl)-1,2,3,4-tetrahydro-thieno[2,3-d]pyrimidine-6-carboxylic acid 4-methoxy-benzylamide;
3-[2-(4-Chloro-phenoxy)-ethyl]-1-methyl-2,4-dioxo-1,2,3,4-tetrahydro-thieno[2,3-d]pyrimidine-6-carboxylic acid 3-methoxy-benzylamide;
1-Methyl-2,4-dioxo-3-[2-(3-trifluoromethyl-benzenesulfonyl)-ethyl]-1,2,3,4-tetrahydro-thieno[2,3-d]pyrimidine-6-carboxylic acid 3-methoxy-benzylamide;
1-Methyl-2,4-dioxo-3-[2-(3-trifluoromethyl-benzenesulfonyl)-ethyl]-1,2,3,4-tetrahydro-thieno[2,3-d]pyrimidine-6-carboxylic acid 4-methoxy-benzylamide;
3-[2-(4-Chloro-benzenesulfonyl)-ethyl]-1-methyl-2,4-dioxo-1,2,3,4-tetrahydro-thieno[2,3-d]pyrimidine-6-carboxylic acid 3-methoxy-benzylamide; and
3-(2-Amino-ethyl)-1-methyl-2,4-dioxo-1,2,3,4-tetrahydro-thieno[2,3-d]pyrimidine-6-carboxylic acid 4-methoxy-benzylamide.

16. The compound according to claim 1, selected from:
1-Methyl-2,4-dioxo-1,2,3,4-tetrahydro-thieno[2,3-d]pyrimidine-6-carboxylic acid;
4-(6-Carbamoyl-1-methyl-2,4-dioxo-1,4-dihydro-2H-thieno[2,3-d]pyrimidin-3-ylmethyl)-2-methyl-benzoic acid;
4-(6-Carbamoyl-1-methyl-2,4-dioxo-1,4-dihydro-2H-thieno[2,3-d]pyrimidin-3-ylmethyl)-2-methyl-benzoic acid methyl ester;
4-[6-(3-Hydroxy-benzylcarbamoyl)-1-methyl-2,4-dioxo-1,4-dihydro-2H-thieno[2,3-d]pyrimidin-3-ylmethyl]-2-methyl-benzoic acid;
4-(6-Carbamoyl-1-methyl-2,4-dioxo-1,4-dihydro-2H-thieno[2,3-d]pyrimidin-3-ylmethyl)-2-hydroxy-benzoic acid;
3-(2-Amino-ethyl)-1-methyl-2,4-dioxo-1,2,3,4-tetrahydro-thieno[2,3-d]pyrimidine-6-carboxylic acid 3-methoxy-benzylamide; and
4-(2,5-Di-pyridin-4-yl-thiophen-3-yl)-benzaldehyde.

17. The compound according to claim 1, selected from:
1-Methyl-2,4-dioxo-3-(1-phenyl-ethyl)-1,2,3,4-tetrahydro-thieno[2,3-d]pyrimidine-6-carboxylic acid 4-methoxy-benzylamide;
1-Methyl-2,4-dioxo-3-(3-oxo-3-phenyl-propyl)-1,2,3,4-tetrahydro-thieno[2,3-d]pyrimidine-6-carboxylic acid 4-methoxy-benzylamide;
3-((S)-3,7-Dimethyl-oct-6-enyl)-1-methyl-2,4-dioxo-1,2,3,4-tetrahydro-thieno[2,3-d]pyrimidine-6-carboxylic acid 4-methoxy-benzylamide;
3-(2-Ethyl-hexyl)-1-methyl-2,4-dioxo-1,2,3,4-tetrahydro-thieno[2,3-d]pyrimidine-6-carboxylic acid 4-methoxy-benzylamide;
3-(5-Cyano-pentyl)-1-methyl-2,4-dioxo-1,2,3,4-tetrahydro-thieno[2,3-d]pyrimidine-6-carboxylic acid 4-methoxy-benzylamide;
3-(E)-But-2-enyl-1-methyl-2,4-dioxo-1,2,3,4-tetrahydro-thieno[2,3-d]pyrimidine-6-carboxylic acid 4-methoxy-benzylamide;
1-Methyl-3-(2-naphthalen-1-yl-ethyl)-2,4-dioxo-1,2,3,4-tetrahydro-thieno[2,3-d]pyrimidine-6-carboxylic acid 4-methoxy-benzylamide;
1-Methyl-2,4-dioxo-3-(E)-pent-2-enyl-1,2,3,4-tetrahydro-thieno[2,3-d]pyrimidine-6-carboxylic acid 4-methoxy-benzylamide;
1-Methyl-2,4-dioxo-3-(2-phenylsulfanyl-ethyl)-1,2,3,4-tetrahydro-thieno[2,3-d]pyrimidine-6-carboxylic acid 4-methoxy-benzylamide;
3-sec-Butyl-1-methyl-2,4-dioxo-1,2,3,4-tetrahydro-thieno[2,3-d]pyrimidine-6-carboxylic acid 4-methoxy-benzylamide;
1-Methyl-3-(2-methyl-allyl)-2,4-dioxo-1,2,3,4-tetrahydro-thieno[2,3-d]pyrimidine-6-carboxylic acid 4-methoxy-benzylamide;
3-(1-Ethyl-propyl)-1-methyl-2,4-dioxo-1,2,3,4-tetrahydro-thieno[2,3-d]pyrimidine-6-carboxylic acid 4-methoxy-benzylamide;
1-Methyl-2,4-dioxo-3-pent-2-ynyl-1,2,3,4-tetrahydro-thieno[2,3-d]pyrimidine-6-carboxylic acid 4-methoxy-benzylamide;
3-(2-Benzenesulfonyl-ethyl)-1-methyl-2,4-dioxo-1,2,3,4-tetrahydro-thieno[2,3-d]pyrimidine-6-carboxylic acid 4-methoxy-benzylamide;
1-Methyl-3-(3-methyl-but-2-enyl)-2,4-dioxo-1,2,3,4-tetrahydro-thieno[2,3-d]pyrimidine-6-carboxylic acid 4-methoxy-benzylamide;
3-[2-(4-Fluoro-benzenesulfonyl)-ethyl]-1-methyl-2,4-dioxo-1,2,3,4-tetrahydro-thieno[2,3-d]pyrimidine-6-carboxylic acid 4-methoxy-benzylamide;
1-Methyl-2,4-dioxo-3-[2-(toluene-4-sulfonyl)-ethyl]-1,2,3,4-tetrahydro-thieno[2,3-d]pyrimidine-6-carboxylic acid 4-methoxy-benzylamide;
3-[3-(4-Fluoro-phenyl)-3-oxo-propyl]-1-methyl-2,4-dioxo-1,2,3,4-tetrahydro-thieno[2,3-d]pyrimidine-6-carboxylic acid 4-methoxy-benzylamide;
3-[3-(4-Chloro-phenyl)-3-oxo-propyl]-1-methyl-2,4-dioxo-1,2,3,4-tetrahydro-thieno[2,3-d]pyrimidine-6-carboxylic acid 4-methoxy-benzylamide;
3-(2-Benzoylamino-ethyl)-1-methyl-2,4-dioxo-1,2,3,4-tetrahydro-thieno[2,3-d]pyrimidine-6-carboxylic acid 4-methoxy-benzylamide;
3-[2-(4-Bromo-phenoxy)-ethyl]-1-methyl-2,4-dioxo-1,2,3,4-tetrahydro-thieno[2,3-d]pyrimidine-6-carboxylic acid 4-methoxy-benzylamide;
3-Benzofurazan-5-ylmethyl-1-methyl-2,4-dioxo-1,2,3,4-tetrahydro-thieno[2,3-d]pyrimidine-6-carboxylic acid 4-methoxy-benzylamide;
1-Methyl-2,4-dioxo-3-(2-phenoxy-ethyl)-1,2,3,4-tetrahydro-thieno[2,3-d]pyrimidine-6-carboxylic acid 4-methoxy-benzylamide;
{5-[6-(4-Methoxy-benzylcarbamoyl)-1-methyl-2,4-dioxo-1,4-dihydro-2H-thieno[2,3-d]pyrimidin-3-ylmethyl]-isoxazol-3-yl}-carbamic acid methyl ester;
3-Benzyloxycarbonylamino-5-[6-(4-methoxy-benzylcarbamoyl)-1-methyl-2,4-dioxo-1,4-dihydro-2H-thieno[2,3-d]pyrimidin-3-yl]-4-oxo-pentanoic acid tert-butyl ester;
3-[2-(4-Chloro-phenylsulfanyl)-ethyl]-1-methyl-2,4-dioxo-1,2,3,4-tetrahydro-thieno[2,3-d]pyrimidine-6-carboxylic acid 3-methoxy-benzylamide;
1-Methyl-2,4-dioxo-3-(1-phenyl-ethyl)-1,2,3,4-tetrahydro-thieno[2,3-d]pyrimidine-6-carboxylic acid 3-methoxy-benzylamide;
1-Methyl-2,4-dioxo-3-(E)-pent-2-enyl-1,2,3,4-tetrahydro-thieno[2,3-d]pyrimdine-6-carboxylic acid 3-methoxy-benzylamide;
3-(2-Ethyl-hexyl)-1-methyl-2,4-dioxo-1,2,3,4-tetrahydro-thieno[2,3-d]pyrimidine-6-carboxylic acid 3-methoxy-benzylamide;
1-Methyl-2,4-dioxo-3-(2-phenylmethanesulfonyl-ethyl)-1,2,3,4-tetrahydro-thieno[2,3-d]pyrimidine-6-carboxylic acid 3-methoxy-benzylamide;
3-(5-Cyano-pentyl)-1-methyl-2,4-dioxo-1,2,3,4-tetrahydro-thieno[2,3-d]pyrimidine-6-carboxylic acid 3-methoxy-benzylamide;
3-(E)-But-2-enyl-1-methyl-2,4-dioxo-1,2,3,4-tetrahydro-thieno[2,3-d]pyrimidine-6-carboxylic acid 3-methoxy-benzylamide;
1-Methyl-3-(2-naphthalen-1-yl-ethyl)-2,4-dioxo-1,2,3,4-tetrahydro-thieno[2,3-d]pyrimidine-6-carboxylic acid 3-methoxy-benzylamide;
1-Methyl-2,4-dioxo-3-(E)-pent-2-enyl-1,2,3,4-tetrahydro-thieno[2,3-d]pyrimidine-6-carboxylic acid 3-methoxy-benzylamide;
1-Methyl-2,4-dioxo-3-(2-phenylsulfanyl-ethyl)-1,2,3,4-tetrahydro-thieno[2,3-d]pyrimidine-6-carboxylic acid 3-methoxy-benzylamide;
3-sec-Butyl-1-methyl-2,4-dioxo-1,2,3,4-tetrahydro-thieno[2,3-d]pyrimidine-6-carboxylic acid 3-methoxy-benzylamide;
1-Methyl-3-(2-methyl-allyl)-2,4-dioxo-1,2,3,4-tetrahydro-thieno[2,3-d]pyrimidine-6-carboxylic acid 3-methoxy-benzylamide;
3-(1-Ethyl-propyl)-1-methyl-2,4-dioxo-1,2,3,4-tetrahydro-thieno[2,3-d]pyrimidine-6-carboxylic acid 3-methoxy-benzylamide;
1-Methyl-2,4-dioxo-3-pent-2-ynyl-1,2,3,4-tetrahydro-thieno[2,3-d]pyrimidine-6-carboxylic acid 3-methoxy-benzylamide;
1-Methyl-3-(3-methyl-but-2-enyl)-2,4-dioxo-1,2,3,4-tetrahydro-thieno[2,3-d]pyrimidine-6-carboxylic acid 3-methoxy-benzylamide;
1-Methyl-2,4-dioxo-3-[2-(toluene-4-sulfonyl)-ethyl]-1,2,3,4-tetrahydro-thieno[2,3-d]pyrimidine-6-carboxylic acid 3-methoxy-benzylamide;
3-(2-Benzoylamino-ethyl)-1-methyl-2,4-dioxo-1,2,3,4-tetrahydro-thieno[2,3-d]pyrimidine-6-carboxylic acid 3-methoxy-benzylamide;
3-[2-(4-Bromo-phenoxy)-ethyl]-1-methyl-2,4-dioxo-1,2,3,4-tetrahydro-thieno[2,3-d]pyrimidine-6-carboxylic acid 3-methoxy-benzylamide;
3-Benzofurazan-5-ylmethyl-1-methyl-2,4-dioxo-1,2,3,4-tetrahydro-thieno[2,3-d]pyrimidine-6-carboxylic acid 3-methoxy-benzylamide;
1-Methyl-2,4-dioxo-3-(2-phenoxy-ethyl)-1,2,3,4-tetrahydro-thieno[2,3-d]pyrimidine-6-carboxylic acid 3-methoxy-benzylamide;
{5-[6-(3-Methoxy-benzylcarbamoyl)-1-methyl-2,4-dioxo-1,4-dihydro-2H-thieno[2,3-d]pyrimidin-3-ylmethyl]-isoxazol-3-yl}-carbamic acid methyl ester; and
3-Benzyloxycarbonylamino-5-[6-(3-methoxy-benzylcarbamoyl)-1-methyl-2,4-dioxo-1,4-dihydro-2H-thieno[2,3-d]pyrimidin-3-yl]-4-oxo-pentanoic acid tert-butyl ester.

18. The compound according to claim 1, selected from:
3-Benzyl-2,4-dioxo-1,2,3,4-tetrahydro-thieno[2,3-d]pyrimidine-6-carboxylic acid benzyl ester;
3-Benzyl-1-methyl-2,4-dioxo-1,2,3,4-tetrahydro-thieno[2,3-d]pyrimidine-6-carboxylic acid benzyl ester;
3-(4-Pyridyl)-2,4-dioxo-1,2,3,4-tetrahydro-thieno[2,3-d]pyrimidine-6-carboxylic acid benzyl ester;
3-(4-Pyridyl)-1-methyl-2,4-dioxo-1,2,3,4-tetrahydro-thieno[2,3-d]-pyrimidine-6-carboxylic acid benzyl ester;
3-Benzyl-2,4-dioxo-1,2,3,4-tetrahydro-thieno[2,3-d]pyrimidine-6-carboxylic acid (4-pyridyl)ester;
3-Benzyl-1-methyl-2,4-dioxo-1,2,3,4-tetrahydro-thieno[2,3-d]pyrimidine-6-carboxylic acid (4-pyridyl)ester;
3-(4-Pyridyl)-2,4-dioxo-1,2,3,4-tetrahydro-thieno[2,3-d]pyrimidine-6-carboxylic acid (4-pyridyl)ester;
3-(4-Pyridyl)-1-methyl-2,4-dioxo-1,2,3,4-tetrahydro-thieno[2,3-d]-pyrimidine-6-carboxylic acid (4-pyridyl)ester;
3-Benzyl-2,4-dioxo-1,2,3,4-tetrahydro-thieno[2,3-d]pyrimidine-6-carboxylic acid piperoyl ester;
3-Benzyl-1-methyl-2,4-dioxo-1,2,3,4-tetrahydro-thieno[2,3-d]pyrimidine-6-carboxylic acid piperoyl ester;
3-Piperoyl-2,4-dioxo-1,2,3,4-tetrahydro-thieno[2,3-d]pyrimidine-6-carboxylic acid piperoyl ester;
3-Piperoyl-1-methyl-2,4-dioxo-1,2,3,4-tetrahydro-thieno[2,3-d]-pyrimidine-6-carboxylic acid piperoyl ester;
3-Benzyl-1-methyl-2,4-dioxo-1,2,3,4-tetrahydro-furo[2,3-d]pyrimidine-6-carboxylic acid benzyl ester;
3-Benzyl-1-methyl-2,4-dioxo-1,2,3,4-tetrahydro-1H-pyrrolo[2,3-d]-pyrimidine-6-carboxylic acid benzyl ester;
3-Benzyl-1,7-dimethyl-2,4-dioxo-1,2,3,4-tetrahydro-1H-pyrrolo[2,3-d]-pyrimidine-6-carboxylic acid benzyl ester;
3-Benzyl-1,7-dimethyl-2,4-dioxo-1,2,3,4-tetrahydro-1H-pyrrolo[2,3-d]-pyrimidine-6-carboxylic acid benzofuran-6-ylmethyl ester;
3-Benzyl-1-dimethyl-2,4-dioxo-1,2,3,4-tetrahydro-1H-pyrrolo[2,3-d]-pyrimidine-6-carboxylic acid benzofuran-6-ylmethyl ester;
3-Benzyl-1-methyl-2,4-dioxo-1,2,3,4-tetrahydro-furo[2,3-d]pyrimidine-6-carboxylic acid benzofuran-6-ylmethyl ester;
3-Benzyl-1-methyl-2,4-dioxo-1,2,3,4-tetrahydro-thieno[2,3-d]pyrimidine-6-carboxylic acid benzofuran-6-ylmethyl ester;
3-Benzyl-1,7-dimethyl-2,4-dioxo-1,2,3,4-tetrahydro-1H-pyrrolo[2,3-d]-pyrimidine-6-carboxylic acid benzothiophene-6-ylmethyl ester;
3-Benzyl-1-dimethyl-2,4-dioxo-1,2,3,4-tetrahydro-1H-pyrrolo[2,3-d]-pyrimidine-6-carboxylic acid benzothiophene-6-ylmethyl ester;
3-Benzyl-1-methyl-2,4-dioxo-1,2,3,4-tetrahydro-furo[2,3-d]pyrimidine-6-carboxylic acid benzothiophene-6-ylmethyl ester; and
3-Benzyl-1-methyl-2,4-dioxo-1,2,3,4-tetrahydro-thieno[2,3-d]pyrimidine-6-carboxylic acid benzothiophene-6-ylmethyl ester.

19. A compound according to claim 1, or a pharmaceutically acceptable salt thereof, selected from:
3-(3-Methoxy-benzyl)-1-methyl-2,4-dioxo-1,2,3,4-tetrahydro-thieno[2,3-d]pyrimidine-6-carboxylic acid benzyl ester;
3-(4-Chloro-benzyl)-1-methyl-2,4-dioxo-1,2,3,4-tetrahydro-thieno[2,3-d]pyrimidine-6-carboxylic acid benzyl ester;
3-(4-Cyano-benzyl)-1-methyl-2,4-dioxo-1,2,3,4-tetrahydro-thieno[2,3-d]pyrimidine-6-carboxylic acid benzyl ester;
3-(4-Fluoro-benzyl)-1-methyl-2,4-dioxo-1,2,3,4-tetrahydro-thieno[2,3-d]pyrimidine-6-carboxylic acid benzyl ester;
3-(3-Chloro-benzyl)-1-methyl-2,4-dioxo-1,2,3,4-tetrahydro-thieno[2,3-d]pyrimidine-6-carboxylic acid benzyl ester;
1-Methyl-3-(2-methyl-benzyl)-2,4-dioxo-1,2,3,4-tetrahydro-thieno[2,3-d]pyrimidine-6-carboxylic acid benzyl ester;
1-Methyl-3-(4-methyl-benzyl)-2,4-dioxo-1,2,3,4-tetrahydro-thieno[2,3-d]pyrimidine-6-carboxylic acid benzyl ester;
3-(4-Carboxy-benzyl)-1-methyl-2,4-dioxo-1,2,3,4-tetrahydro-thieno[2,3-d]pyrimidine-6-carboxylic acid benzyl ester;
1-Methyl-2,4-dioxo-3-(3-trifluoromethyl-benzyl)-1,2,3,4-tetrahydro-thieno[2,3-d]pyrimidine-6-carboxylic acid benzyl ester;
3-Biphenyl-4-ylmethyl-1-methyl-2,4-dioxo-1,2,3,4-tetrahydro-thieno[2,3-d]pyrimidine-6-carboxylic acid benzyl ester;
1-Methyl-2,4-dioxo-3-(2-trifluoromethyl-benzyl)-1,2,3,4-tetrahydro-thieno[2,3-d]pyrimidine-6-carboxylic acid benzyl ester;
3-(3-Cyano-benzyl)-1-methyl-2,4-dioxo-1,2,3,4-tetrahydro-thieno[2,3-d]pyrimidine-6-carboxylic acid benzyl ester;
3-(2-Cyano-benzyl)-1-methyl-2,4-dioxo-1,2,3,4-tetrahydro-thieno[2,3-d]pyrimidine-6-carboxylic acid benzyl ester;
1-Methyl-2,4-dioxo-3-(4-trifluoromethyl-benzyl)-1,2,3,4-tetrahydro-thieno[2,3-d]pyrimidine-6-carboxylic acid benzyl ester;
3-[2-Hydroxy-3-(naphthalen-1-yloxy)-propyl]-1-methyl-2,4-dioxo-1,2,3,4-tetrahydro-thieno[2,3-d]pyrimidine-6-carboxylic acid benzyl ester;
3-(3-Chloro-benzyl)-1-methyl-2,4-dioxo-1,2,3,4-tetrahydro-thieno[2,3-d]pyrimidine-6-carboxylic acid benzyl ester;
1-Methyl-3-naphthalen-1-ylmethyl-2,4-dioxo-1,2,3,4-tetrahydro-thieno[2,3-d]pyrimidine-6-carboxylic acid benzyl ester;
3-(6-Chloro-benzo[1,3]dioxol-5-ylmethyl)-1-methyl-2,4-dioxo-1,2,3,4-tetrahydro-thieno[2,3-d]pyrimidine-6-carboxylic acid benzyl ester;
1-Methyl-2,4-dioxo-3-(4-oxo-4-thiophen-2-yl-butyl)-1,2,3,4-tetrahydro-thieno[2,3-d]pyrimidine-6-carboxylic acid benzyl ester;
1-Methyl-2,4-dioxo-3-pyridin-4-ylmethyl-1,2,3,4-tetrahydro-thieno[2,3-d]pyrimidine-6-carboxylic acid benzyl ester;
1-Methyl-2,4-dioxo-3-(4-m-tolyloxy-butyl)-1,2,3,4-tetrahydro-thieno[2,3-d]pyrimidine-6-carboxylic acid benzyl ester;
3-(3,5-Dimethyl-isoxazol-4-ylmethyl)-1-methyl-2,4-dioxo-1,2,3,4-tetrahydro-thieno[2,3-d]pyrimidine-6-carboxylic acid benzyl ester;
3-Dioxo-1,3-dihydro-isoindol-2-ylmethyl)-1-methyl-2,4-dioxo-1,2,3,4-tetrahydro-thieno[2,3-d]pyrimidine-6-carboxylic acid benzyl ester;
3-Dihydro-benzo[1,4]dioxin-2-ylmethyl)-1-methyl-2,4-dioxo-1,2,3,4-tetrahydro-thieno[2,3-d]pyrimidine-6-carboxylic acid benzyl ester;
1-Methyl-2,4-dioxo-3-pyridin-2-ylmethyl-1,2,3,4-tetrahydro-thieno[2,3-d]pyrimidine-6-carboxylic acid benzyl ester;
3-[2-(2,5-Dimethoxy-phenyl)-2-oxo-ethyl]-1-methyl-2,4-dioxo-1,2,3,4-tetrahydro-thieno[2,3-d]pyrimidine-6-carboxylic acid benzyl ester;
3-Benzyloxymethyl-1-methyl-2,4-dioxo-1,2,3,4-tetrahydro-thieno[2,3-d]pyrimidine-6-carboxylic acid benzyl ester;
1-Methyl-2,4-dioxo-3-(4-m-tolyloxy-butyl)-1,2,3,4-tetrahydro-thieno[2,3-d]pyrimidine-6-carboxylic acid benzyl ester;
1-Methyl-2,4-dioxo-3-(2-phenylmethanesulfonyl-ethyl)-1,2,3,4-tetrahydro-thieno[2,3-d]pyrimidine-6-carboxylic acid benzyl ester;
3-(4-Amino-6-phenylamino-[1,3,5]triazin-2-ylmethyl)-1-methyl-2,4-dioxo-1,2,3,4-tetrahydro-thieno[2,3-d]pyrimidine-6-carboxylic acid benzyl ester;
3-[4-(4-Fluoro-phenyl)-4-oxo-butyl]-1-methyl-2,4-dioxo-1,2,3,4-tetrahydro-thieno[2,3-d]pyrimidine-6-carboxylic acid benzyl ester;
3-[4-(1,3-Dioxo-1,3-dihydro-isoindol-2-yl)-butyl]-1-methyl-2,4-dioxo-1,2,3,4-tetrahydro-thieno[2,3-d]pyrimidine-6-carboxylic acid benzyl ester;
1-Methyl-2,4-dioxo-3-(4-phenoxy-butyl)-1,2,3,4-tetrahydro-thieno[2,3-d]pyrimidine-6-carboxylic acid benzyl ester;
1-Methyl-2,4-dioxo-3-(4-oxo-4-phenyl-butyl)-1,2,3,4-tetrahydro-thieno[2,3-d]pyrimidine-6-carboxylic acid benzyl ester;
1-Methyl-2,4-dioxo-3-(2-phenoxy-ethyl)-1,2,3,4-tetrahydro-thieno[2,3-d]pyrimidine-6-carboxylic acid benzyl ester;
3-{3-[4-(3-Chloro-phenyl)-piperazin-1-yl]-propyl}-1-methyl-2,4-dioxo-1,2,3,4-tetrahydro-thieno[2,3-d]pyrimidine-6-carboxylic acid benzyl ester;
3-[1-Bromo-2-(1H-indol-3-yl)-ethyl]-1-methyl-2,4-dioxo-1,2,3,4-tetrahydro-thieno[2,3-d]pyrimidine-6-carboxylic acid benzyl ester;
3-(2-Benzenesulfinyl-ethyl)-1-methyl-2,4-dioxo-1,2,3,4-tetrahydro-thieno[2,3-d]pyrimidine-6-carboxylic acid benzyl ester;
3-[3-(3-Fluoro-phenylcarbamoyl)-propyl]-1-methyl-2,4-dioxo-1,2,3,4-tetrahydro-thieno[2,3-d]pyrimidine-6-carboxylic acid benzyl ester;
1-Methyl-2,4-dioxo-3-[2-(2-trifluoromethyl-phenylcarbamoyl)-ethyl]-1,2,3,4-tetrahydro-thieno[2,3-d]pyrimidine-6-carboxylic acid benzyl ester;
3-[2-(4-Methoxy-phenyl)-ethyl]-1-methyl-2,4-dioxo-1,2,3,4-tetrahydro-thieno[2,3-d]pyrimidine-6-carboxylic acid benzyl ester;
3-[2-(4-Chloro-2-nitro-phenoxy)-ethyl]-1-methyl-2,4-dioxo-1,2,3,4-tetrahydro-thieno[2,3-d]pyrimidine-6-carboxylic acid benzyl ester;
1-Methyl-3-(5-nitro-furan-2-ylmethyl)-2,4-dioxo-1,2,3,4-tetrahydro-thieno[2,3-d]pyrimidine-6-carboxylic acid benzyl ester;
3-(1-Benzyl-1H-imidazol-2-ylmethyl)-1-methyl-2,4-dioxo-1,2,3,4-tetrahydro-thieno[2,3-d]pyrimidine-6-carboxylic acid benzyl ester;
3-[3-(Benzyl-methyl-amino)-propyl]-1-methyl-2,4-dioxo-1,2,3,4-tetrahydro-thieno[2,3-d]pyrimidine-6-carboxylic acid benzyl ester;
3-(Bis-trifluoromethyl-benzyl)-1-methyl-2,4-dioxo-1,2,3,4-tetrahydro-thieno[2,3-d]pyrimidine-6-carboxylic acid benzyl ester;
3-[3-(2-Bromo-4-methyl-phenoxy)-propyl]-1-methyl-2,4-dioxo-1,2,3,4-tetrahydro-thieno[2,3-d]pyrimidine-6-carboxylic acid benzyl ester;
3-Benzenesulfonylmethyl-1-methyl-2,4-dioxo-1,2,3,4-tetrahydro-thieno[2,3-d]pyrimidine-6-carboxylic acid benzyl ester;
3-[2-(4-Chloro-benzenesulfonyl)-ethyl]-1-methyl-2,4-dioxo-1,2,3,4-tetrahydro-thieno[2,3-d]pyrimidine-6-carboxylic acid benzyl ester;
3-Benzo[1,3]dioxol-5-ylmethyl-1-methyl-2,4-dioxo-1,2,3,4-tetrahydro-thieno[2,3-d]pyrimidine-6-carboxylic acid benzyl ester;
3-(3-Iodo-benzyl)-1-methyl-2,4-dioxo-1,2,3,4-tetrahydro-thieno[2,3-d]pyrimidine-6-carboxylic acid benzyl ester;
1-Methyl-2,4-dioxo-3-(4-trifluoromethoxy-benzyl)-1,2,3,4-tetrahydro-thieno[2,3-d]pyrimidine-6-carboxylic acid benzyl ester;
3-(4-Acetoxy-butyl)-1-methyl-2,4-dioxo-1,2,3,4-tetrahydro-thieno[2,3-d]pyrimidine-6-carboxylic acid benzyl ester;
3-(4-Methanesulfonyl-benzyl)-1-methyl-2,4-dioxo-1,2,3,4-tetrahydro-thieno[2,3-d]pyrimidine-6-carboxylic acid benzyl ester;
1-Methyl-2,4-dioxo-3-(4-[1,2,3]thiadiazol-4-yl-benzyl)-1,2,3,4-tetrahydro-thieno[2,3-d]pyrimidine-6-carboxylic acid benzyl ester;
3-(5-Methoxycarbonyl-furan-2-ylmethyl)-1-methyl-2,4-dioxo-1,2,3,4-tetrahydro-thieno[2,3-d]pyrimidine-6-carboxylic acid benzyl ester;
3-(2-Carboxy-ethyl)-1-methyl-2,4-dioxo-1,2,3,4-tetrahydro-thieno[2,3-d]pyrimidine-6-carboxylic acid benzyl ester;
1-Methyl-2,4-dioxo-3-(3-pyrrol-1-yl-propyl)-1,2,3,4-tetrahydro-thieno[2,3-d]pyrimidine-6-carboxylic acid benzyl ester;
3-(3-Carboxy-propyl)-1-methyl-2,4-dioxo-1,2,3,4-tetrahydro-thieno[2,3-d]pyrimidine-6-carboxylic acid benzyl ester;
3-(2-Cyano-ethyl)-1-methyl-2,4-dioxo-1,2,3,4-tetrahydro-thieno[2,3-d]pyrimidine-6-carboxylic acid benzyl ester;
3-(3-Ethoxycarbonyl-furan-2-ylmethyl)-1-methyl-2,4-dioxo-1,2,3,4-tetrahydro-thieno[2,3-d]pyrimidine-6-carboxylic acid benzyl ester;
3-(3-Amino-propyl)-1-methyl-2,4-dioxo-1,2,3,4-tetrahydro-thieno[2,3-d]pyrimidine-6-carboxylic acid benzyl ester;
3-(3-Cyano-propyl)-1-methyl-2,4-dioxo-1,2,3,4-tetrahydro-thieno[2,3-d]pyrimidine-6-carboxylic acid benzyl ester;
3-(2-Hydroxy-ethyl)-1-methyl-2,4-dioxo-1,2,3,4-tetrahydro-thieno[2,3-d]pyrimidine-6-carboxylic acid benzyl ester;
3-(2-Carboxy-hexyl)-1-methyl-2,4-dioxo-1,2,3,4-tetrahydro-thieno[2,3-d]pyrimidine-6-carboxylic acid benzyl ester;
1-Methyl-2,4-dioxo-3-(2,2,2-trifluoro-ethyl)-1,2,3,4-tetrahydro-thieno[2,3-d]pyrimidine-6-carboxylic acid benzyl ester;
1-Methyl-2,4-dioxo-3-(2,2,2-trifluoro-ethyl)-1,2,3,4-tetrahydro-thieno[2,3-d]pyrimidine-6-carboxyl carboxylic acid benzyl ester;
Iodomethyl-1-methyl-2,4-dioxo-1,2,3,4-tetrahydro-thieno[2,3-d]pyrimidine-6-carboxylic acid benzyl ester;
3-(2-Fluoro-ethyl)-1-methyl-2,4-dioxo-1,2,3,4-tetrahydro-thieno[2,3-d]pyrimidine-6-carboxylic acid benzyl ester;
Methyl-2,4-dioxo-3-(tetrahydro-furan-2-ylmethyl)-1,2,3,4-tetrahydro-thieno[2,3-d]pyrimidine-6-carboxylic acid benzyl ester;
3-[1-(4-Carboxy-phenyl-ethyl]-1-methyl-2,4-dioxo-1,2,3,4-tetrahydro-thieno[2,3-d]pyrimidine-6-carboxylic acid benzyl ester;
3-(Hex-5-enyl-1-methyl-2,4-dioxo-1,2,3,4-tetrahydro-thieno[2,3-d]pyrimidine-6-carboxylic acid benzyl ester;
3-(2-Ethyl-butyl)-1-methyl-2,4-dioxo-1,2,3,4-tetrahydro-thieno[2,3-d]pyrimidine-6-carboxylic acid benzyl ester;
1-Methyl-2,4-dioxo-3-(2,2,2-trifluoro-ethyl)-1,2,3,4-tetrahydro-thieno[2,3-d]pyrimidine-6-carboxyl pyrimidine acid benzyl ester;
3-(Diethoxy-phosphorylmethyl)-1-methyl-2,4-dioxo-1,2,3,4-tetrahydro-thieno[2,3-d]pyrimidine-6-carboxylic acid benzyl ester;
3-But-2-ynyl-1-methyl-2,4-dioxo-1,2,3,4-tetrahydro-thieno[2,3-d]pyrimidine-6-carboxylic acid benzyl ester;
Bromo-ethyl)-1-methyl-2,4-dioxo-1,2,3,4-tetrahydro-thieno[2,3-d]pyrimidine-6-carboxylic acid;
1-Methyl-2,4-dioxo-3-[2-(tetrahydro-pyran-2-yloxy)-ethyl]-1,2,3,4-tetrahydro-thieno[2,3-d]pyrimidine-6-carboxylic acid benzyl ester;
1-Methyl-2,4-dioxo-3-propyl-1,2,3,4-tetrahydro-thieno[2,3-d]pyrimidine-6-carboxylic acid benzyl ester;
3-(2-Acetoxy-ethyl)-1-methyl-2,4-dioxo-1,2,3,4-tetrahydro-thieno[2,3-d]pyrimidine-6-carboxylic acid;
3-Butyl-1-methyl-2,4-dioxo-1,2,3,4-tetrahydro-thieno[2,3-d]pyrimidine-6-carboxylic acid benzyl ester;
3-Isobutyl-1-methyl-2,4-dioxo-1,2,3,4-tetrahydro-thieno[2,3-d]pyrimidine-6-carboxylic acid benzyl ester;
3-Ethyl-1-methyl-2,4-dioxo-1,2,3,4-tetrahydro-thieno[2,3-d]pyrimidine-6-carboxylic acid benzyl ester;
3-(3-Bromo-propyl)-1-methyl-2,4-dioxo-1,2,3,4-tetrahydro-thieno[2,3-d]pyrimidine-6-carboxylic acid benzyl ester;
3-Cyclohexylmethyl-1-methyl-2,4-dioxo-1,2,3,4-tetrahydro-thieno[2,3-d]pyrimidine-6-carboxylic acid benzyl ester;
3-(2-Ethylamino-ethyl)-1-methyl-2,4-dioxo-1,2,3,4-tetrahydro-thieno[2,3-d]pyrimidine-6-carboxylic acid benzyl ester;
3-Cyclobutylmethyl-1-methyl-2,4-dioxo-1,2,3,4-tetrahydro-thieno[2,3-d]pyrimidine-6-carboxylic acid benzyl ester;
3-((R)-3-Hydroxy-2-methyl-propyl)-1-methyl-2,4-dioxo-1,2,3,4-tetrahydro-thieno[2,3-d]pyrimidine-6-carboxylic acid benzyl ester;
3-(4-Hydroxy-butyl)-1-methyl-2,4-dioxo-1,2,3,4-tetrahydro-thieno[2,3-d]pyrimidine-6-carboxylic acid benzyl ester;
3-(2-Ethoxy-ethyl)-1-methyl-2,4-dioxo-1,2,3,4-tetrahydro-thieno[2,3-d]pyrimidine-6-carboxylic acid benzyl ester;
3-Isobutyl-1-methyl-2,4-dioxo-1,2,3,4-tetrahydro-thieno[2,3-d]pyrimidine-6-carboxylic acid benzyl ester;
3-(2-Chloro-ethyl)-1-methyl-2,4-dioxo-1,2,3,4-tetrahydro-thieno[2,3-d]pyrimidine-6-carboxylic acid benzyl ester;
1-Methyl-3-(3-methyl-but-2-enyl)-2,4-dioxo-1,2,3,4-tetrahydro-thieno[2,3-d]pyrimidine-6-carboxylic acid benzyl ester;
3-Allyl-1-methyl-2,4-dioxo-1,2,3,4-tetrahydro-thieno[2,3-d]pyrimidine-6-carboxylic acid benzyl ester;
3-(2,2-Dimethoxy-ethyl)-1-methyl-2,4-dioxo-1,2,3,4-tetrahydro-thieno[2,3-d]pyrimidine-6-carboxylic acid benzyl ester;
1-Methyl-3-oxiranylmethyl-2,4-dioxo-1,2,3,4-tetrahydro-thieno[2,3-d]pyrimidine-6-carboxylic acid benzyl ester;
1-Methyl-2,4-dioxo-3-propyl-1,2,3,4-tetrahydro-thieno[2,3-d]pyrimidine-6-carboxylic acid benzyl ester;
3-Benzo[1,2,5]oxadiazol-5-ylmethyl-1-methyl-2,4-dioxo-1,2,3,4-tetrahydro-thieno[2,3-d]pyrimidine-6-carboxylic acid benzyl ester;
3-(3-Hydroxy-2,2-dimethyl-propyl)-1-methyl-2,4-dioxo-1,2,3,4-tetrahydro-thieno[2,3-d]pyrimidine-6-carboxylic acid benzyl ester;
3-(2-Carboxy-ethyl)-1-methyl-2,4-dioxo-1,2,3,4-tetrahydro-thieno[2,3-d]pyrimidine-6-carboxylic acid benzyl ester;
3-Propyl-1-methyl-2,4-dioxo-1,2,3,4-tetrahydro-thieno[2,3-d]pyrimidine-6-carboxylic benzyl ester;
1-Methyl-2,4-dioxo-3-(4-sulfamoyl-benzyl)-1,2,3,4-tetrahydro-thieno[2,3-d]pyrimidine-6-carboxylic acid benzyl ester;
1-Methyl-3-(4-methylsulfamoyl-benzyl)-2,4-dioxo-1,2,3,4-tetrahydro-thieno[2,3-d]pyrimidine-6-carboxylic acid benzyl ester;
3-(4-Dimethylsulfamoyl-benzyl)-1-methyl-2,4-dioxo-1,2,3,4-tetrahydro-thieno[2,3-d]pyrimidine-6-carboxylic acid benzyl ester;
3-(4-Methanesulfonylamino-benzyl)-1-methyl-2,4-dioxo-1,2,3,4-tetrahydro-thieno[2,3-d]pyrimidine-6-carboxylic acid benzyl ester;
3-[4-(Methanesulfonyl-methyl-amino)-benzyl]-1-methyl-2,4-dioxo-1,2,3,4-tetrahydro-thieno[2,3-d]pyrimidine-6-carboxylic acid benzyl ester;
3-(4-Acetylamino-benzyl)-1-methyl-2,4-dioxo-1,2,3,4-tetrahydro-thieno[2,3-d]pyrimidine-6-carboxylic acid benzyl ester;
3-[4-(Acetyl-methyl-amino)-benzyl]-1-methyl-2,4-dioxo-1,2,3,4-tetrahydro-thieno[2,3-d]pyrimidine-6-carboxylic acid benzyl ester;
3-(4-Dimethylamino-benzyl)-1-methyl-2,4-dioxo-1,2,3,4-tetrahydro-thieno[2,3-d]pyrimidine-6-carboxylic acid benzyl ester;
1-Methyl-3-(4-methylamino-benzyl)-2,4-dioxo-1,2,3,4-tetrahydro-thieno[2,3-d]pyrimidine-6-carboxylic acid benzyl ester;
3-(4-Carbamoyl-benzyl)-1-methyl-2,4-dioxo-1,2,3,4-tetrahydro-thieno[2,3-d]pyrimidine-6-carboxylic acid benzyl ester;
3-(4-Dimethylcarbamoyl-benzyl)-1-methyl-2,4-dioxo-1,2,3,4-tetrahydro-thieno[2,3-d]pyrimidine-6-carboxylic acid benzyl ester;
3-(4-Carboxy-benzyl)-1-methyl-2,4-dioxo-1,2,3,4-tetrahydro-thieno[2,3-d]pyrimidine-6-carboxylic acid benzyl ester;
3-(4-Methoxycarbonyl-benzyl)-1-methyl-2,4-dioxo-1,2,3,4-tetrahydro-thieno[2,3-d]pyrimidine-6-carboxylic acid benzyl ester;
3-{4-[Bis-(2-hydroxy-ethyl)-amino]-benzyl}-1-methyl-2,4-dioxo-1,2,3,4-tetrahydro-thieno[2,3-d]pyrimidine-6-carboxylic acid benzyl ester;
3-(3,5-Dimethoxy-benzyl)-1-methyl-2,4-dioxo-1,2,3,4-tetrahydro-thieno[2,3-d]pyrimidine-6-carboxylic acid benzyl ester;
3-(4-tert-Butyl-benzyl)-1-methyl-2,4-dioxo-1,2,3,4-tetrahydro-thieno[2,3-d]pyrimidine-6-carboxylic acid benzyl ester;
1-Methyl-2,4-dioxo-3-(4-trifluoromethoxy-benzyl)-1,2,3,4-tetrahydro-thieno[2,3-d]pyrimidine-6-carboxylic acid benzyl ester;
3-(4-Methanesulfonyl-benzyl)-1-methyl -2,4-dioxo-1,2,3,4-tetrahydro-thieno[2,3-d]pyrimidine-6-carboxylic acid benzyl ester;
2,4-Dioxo-3-[1,3,4]thiadiazol-2-ylmethyl-1,2,3,4-tetrahydro-thieno[2,3-d]pyrimidine-6-carboxylic acid benzyl ester;
3-Isoxazol-3-ylmethyl-2,4-dioxo-1,2,3,4-tetrahydro-thieno[2,3-d]pyrimidine-6-carboxylic acid benzyl ester;
3-Oxazol-2-ylmethyl-2,4-dioxo-1,2,3,4-tetrahydro-thieno[2,3-d]pyrimidine-6-carboxylic acid benzyl ester;
2,4-Dioxo-3-thiazol-2-ylmethyl-1,2,3,4-tetrahydro-thieno[2,3-d]pyrimidine-6-carboxylic acid benzyl ester;
3-(1H-Imidazol-2-ylmethyl)-2,4-dioxo-1,2,3,4-tetrahydro-thieno[2,3-d]pyrimidine-6-carboxylic acid benzyl ester;
3-(1-Methyl-1H-imidazol-2-ylmethyl)-2,4-dioxo-1,2,3,4-tetrahydro-thieno[2,3-d]pyrimidine-6-carboxylic acid benzyl ester;
3-(1-Methyl-1H-pyrrol-2-ylmethyl)-2,4-dioxo-1,2,3,4-tetrahydro-thieno[2,3-d]pyrimidine-6-carboxylic acid benzyl ester;
2,4-Dioxo-3-(1H-pyrrol-2-ylmethyl)-1,2,3,4-tetrahydro-thieno[2,3-d]pyrimidine-6-carboxylic acid benzyl ester;
2,4-Dioxo-3-(1H-pyrrol-2-ylmethyl)-1,2,3,4-tetrahydro-thieno[2,3-d]pyrimidine-6-carboxylic acid benzyl ester;
2,4-Dioxo-3-thiophen-2-ylmethyl-1,2,3,4-tetrahydro-thieno[2,3-d]pyrimidine-6-carboxylic acid benzyl ester;
2,4-Dioxo-3-[1,2,3,4]tetrazin-5-ylmethyl-1,2,3,4-tetrahydro-thieno[2,3-d]pyrimidine-6-carboxylic acid benzyl ester;
2,4-Dioxo-3-[1,2,4,5]tetrazin-3-ylmethyl-1,2,3,4-tetrahydro-thieno[2,3-d]pyrimidine-6-carboxylic acid benzyl ester;
3-(1-Methyl-piperidin-4-ylmethyl)-2,4-dioxo-1,2,3,4-tetrahydro-thieno[2,3-d]pyrimidine-6-carboxylic acid benzyl ester;
2,4-Dioxo-3-pyrimidin-2-ylmethyl-1,2,3,4-tetrahydro-thieno[2,3-d]pyrimidine-6-carboxylic acid benzyl ester;
2,4-Dioxo-3-(2H-pyran-2-ylmethyl)-1,2,3,4-tetrahydro-thieno[2,3-d]pyrimidine-6-carboxylic acid benzyl ester;
3-(1H-Imidazo[4,5-b]pyridin-2-ylmethyl)-2,4-dioxo-1,2,3,4-tetrahydro-thieno[2,3-d]pyrimidine-6-carboxylic acid benzyl ester;
3-(1H-Benzoimidazol-2-ylmethyl)-2,4-dioxo-1,2,3,4-tetrahydro-thieno[2,3-d]pyrimidine-6-carboxylic acid benzyl ester;
3-Benzo[b]thiophen-2-ylmethyl-2,4-dioxo-1,2,3,4-tetrahydro-thieno[2,3-d]pyrimidine-6-carboxylic acid benzyl ester;
2,4-Dioxo-3-quinolin-2-ylmethyl-1,2,3,4-tetrahydro-thieno[2,3-d]pyrimidine-6-carboxylic acid benzyl ester;
3-(2H-Chromen-2-ylmethyl)-2,4-dioxo-1,2,3,4-tetrahydro-thieno[2,3-d]pyrimidine-6-carboxylic acid benzyl ester;
3-(1H-Benzoimidazol-2-ylmethyl)-2,4-dioxo-1,2,3,4-tetrahydro-thieno[2,3-d]pyrimidine-6-carboxylic acid benzyl ester;
3-(1-Methyl-1H-benzoimidazol-2-ylmethyl)-2,4-dioxo-1,2,3,4-tetrahydro-thieno[2,3-d]pyrimidine-6-carboxylic acid benzyl ester;
3-(1H-Indol-2-ylmethyl)-2,4-dioxo-1,2,3,4-tetrahydro-thieno[2,3-d]pyrimidine-6-carboxylic acid benzyl ester;
3-Benzyl-1-methyl-2,4-dioxo-1,2,3,4-tetrahydro-thieno[2,3-d]pyrimidine-6-carboxylic acid furan-3-ylmethyl ester;
3-Benzyl-1-methyl-2,4-dioxo-1,2,3,4-tetrahydro-thieno[2,3-d]pyrimidine-6-carboxylic acid 1-ethyl-propyl ester;
3-Benzyl-1-methyl-2,4-dioxo-1,2,3,4-tetrahydro-thieno[2,3-d]pyrimidine-6-carboxylic acid 1,1-dioxo-tetrahydro-1l⁶-thiophen-3-yl ester;
3-Benzyl-1-methyl-2,4-dioxo-1,2,3,4-tetrahydro-thieno[2,3-d]pyrimidine-6-carboxylic acid 4-hydroxy-benzyl ester;
3-Benzyl-1-methyl-2,4-dioxo-1,2,3,4-tetrahydro-thieno[2,3-d]pyrimidine-6-carboxylic acid 1-oxy-pyridin-4-ylmethyl ester;
3-Benzyl-1-methyl-2,4-dioxo-1,2,3,4-tetrahydro-thieno[2,3-d]pyrimidine-6-carboxylic acid but-3-enyl ester;
3-Benzyl-1-methyl-2,4-dioxo-1,2,3,4-tetrahydro-thieno[2,3-d]pyrimidine-6-carboxylic acid 3-diethylamino-propyl ester;
3-Benzyl-1-methyl-2,4-dioxo-1,2,3,4-tetrahydro-thieno[2,3-d]pyrimidine-6-carboxylic acid 1-cyano-1-phenyl-methyl ester;
3-Benzyl-1-methyl-2,4-dioxo-1,2,3,4-tetrahydro-thieno[2,3-d]pyrimidine-6-carboxylic acid 3-amino-benzyl ester;
3-Benzyl-1-methyl-2,4-dioxo-1,2,3,4-tetrahydro-thieno[2,3-d]pyrimidine-6-carboxylic acid 3-methoxy-benzyl ester;
3-Benzyl-1-methyl-2,4-dioxo-1,2,3,4-tetrahydro-thieno[2,3-d]pyrimidine-6-carboxylic acid 1-oxy-pyridin-3-ylmethyl ester;
3-Benzyl-1-methyl-2,4-dioxo-1,2,3,4-tetrahydro-thieno[2,3-d]pyrimidine-6-carboxylic acid 2-ethoxy-ethyl ester;
3-Benzyl-1-methyl-2,4-dioxo-1,2,3,4-tetrahydro-thieno[2,3-d]pyrimidine-6-carboxylic acid thiophen-2-ylmethyl ester;
3-Benzyl-1-methyl-2,4-dioxo-1,2,3,4-tetrahydro-thieno[2,3-d]pyrimidine-6-carboxylic acid 2,6-dichloro-benzyl ester;
3-Benzyl-1-methyl-2,4-dioxo-1,2,3,4-tetrahydro-thieno[2,3-d]pyrimidine-6-carboxylic acid dimethylamino-methyl-ethyl ester;
3-Benzyl-1-methyl-2,4-dioxo-1,2,3,4-tetrahydro-thieno[2,3-d]pyrimidine-6-carboxylic acid 2,2-diphenyl-ethyl ester;
3-Benzyl-1-methyl-2,4-dioxo-1,2,3,4-tetrahydro-thieno[2,3-d]pyrimidine-6-carboxylic acid 2-pyridin-2-yl-ethyl ester;
3-Benzyl-1-methyl-2,4-dioxo-1,2,3,4-tetrahydro-thieno[2,3-d]pyrimidine-6-carboxylic acid 2-ethanesulfonyl-ethyl ester;
3-Benzyl-1-methyl-2,4-dioxo-1,2,3,4-tetrahydro-thieno[2,3-d]pyrimidine-6-carboxylic acid diethylamino-methyl-ethyl ester;
3-Benzyl-1-methyl-2,4-dioxo-1,2,3,4-tetrahydro-thieno[2,3-d]pyrimidine-6-carboxylic acid dimethylamino-methyl-propyl ester;
3-Benzyl-1-methyl-2,4-dioxo-1,2,3,4-tetrahydro-thieno[2,3-d]pyrimidine-6-carboxylic acid 2-(2-chloro-phenoxy)-ethyl ester;
3-Benzyl-1-methyl-2,4-dioxo-1,2,3,4-tetrahydro-thieno[2,3-d]pyrimidine-6-carboxylic acid 2-(2-ethoxy-ethoxy)-ethyl ester;
3-Benzyl-1-methyl-2,4-dioxo-1,2,3,4-tetrahydro-thieno[2,3-d]pyrimidine-6-carboxylic acid 2-hydroxy-benzyl ester;
1-Methyl-2,4-dioxo-1,2,3,4-tetrahydro-thieno[2,3-d]pyrimidine-6-carboxylic acid 2-morpholin-4-yl-ethyl ester;
3-Benzyl-1-methyl-2,4-dioxo-1,2,3,4-tetrahydro-thieno[2,3-d]pyrimidine-6-carboxylic acid 2-(1,3-dioxo-1,3-dihydro-isoindol-2-yl)-ethyl ester;
3-Benzyl-1-methyl-2,4-dioxo-1,2,3,4-tetrahydro-thieno[2,3-d]pyrimidine-6-carboxylic acid 2,3-dihydro-benzo[1,4]dioxin-2-ylmethyl ester;
3-Benzyl-1-methyl-2,4-dioxo-1,2,3,4-tetrahydro-thieno[2,3-d]pyrimidine-6-carboxylic acid 1-methyl-piperidin-4-yl ester;
3-Benzyl-1-methyl-2,4-dioxo-1,2,3,4-tetrahydro-thieno[2,3-d]pyrimidine-6-carboxylic acid 2-(4-hydroxy-phenyl)-ethyl ester;
3-Benzyl-1-methyl-2,4-dioxo-1,2,3,4-tetrahydro-thieno[2,3-d]pyrimidine-6-carboxylic acid 2-cyano-ethyl ester;
3-Benzyl-1-methyl-2,4-dioxo-1,2,3,4-tetrahydro-thieno[2,3-d]pyrimidine-6-carboxylic acid hexyl ester;
3-Benzyl-1-methyl-2,4-dioxo-1,2,3,4-tetrahydro-thieno[2,3-d]pyrimidine-6-carboxylic acid 4-fluoro-benzyl ester;
3-Benzyl-1-methyl-2,4-dioxo-1,2,3,4-tetrahydro-thieno[2,3-d]pyrimidine-6-carboxylic acid 3-hydroxy-6-methyl-pyridin-2-ylmethyl ester;
3-Benzyl-1-methyl-2,4-dioxo-1,2,3,4-tetrahydro-thieno[2,3-d]pyrimidine-6-carboxylic acid 2-benzyloxy-ethyl ester;
3-Benzyl-1-methyl-2,4-dioxo-1,2,3,4-tetrahydro-thieno[2,3-d]pyrimidine-6-carboxylic acid 2-methoxy-benzyl ester;
3-Benzyl-1-methyl-2,4-dioxo-1,2,3,4-tetrahydro-thieno[2,3-d]pyrimidine-6-carboxylic acid 4-methoxy-benzyl ester;
3-Benzyl-1-methyl-2,4-dioxo-1,2,3,4-tetrahydro-thieno[2,3-d]pyrimidine-6-carboxylic acid 2,2,2-trifluoro-ethyl ester;
3-Benzyl-1-methyl-2,4-dioxo-1,2,3,4-tetrahydro-thieno[2,3-d]pyrimidine-6-carboxylic acid 2,2,2-trichloro-ethyl ester;
3-Benzyl-1-methyl-2,4-dioxo-1,2,3,4-tetrahydro-thieno[2,3-d]pyrimidine-6-carboxylic acid pyridin-3-ylmethyl ester;
3-Benzyl-1-methyl-2,4-dioxo-1,2,3,4-tetrahydro-thieno[2,3-d]pyrimidine-6-carboxylic acid pyridin-4-ylmethyl ester;
3-Benzyl-1-methyl-2,4-dioxo-1,2,3,4-tetrahydro-thieno[2,3-d]pyrimidine-6-carboxylic acid 3-pyridin-3-yl-propyl ester;
3-Benzyl-1-methyl-2,4-dioxo-1,2,3,4-tetrahydro-thieno[2,3-d]pyrimidine-6-carboxylic acid;
3-Benzyl-1-methyl-2,4-dioxo-1,2,3,4-tetrahydro-thieno[2,3-d]pyrimidine-6-carboxylic acid 2-phenoxy-ethyl ester;
3-Benzyl-1-methyl-2,4-dioxo-1,2,3,4-tetrahydro-thieno[2,3-d]pyrimidine-6-carboxylic acid 1,3-dimethyl-butyl ester;
3-Benzyl-1-methyl-2,4-dioxo-1,2,3,4-tetrahydro-thieno[2,3-d]pyrimidine-6-carboxylic acid 2-methyl-benzyl ester;
3-Benzyl-1-methyl-2,4-dioxo-1,2,3,4-tetrahydro-thieno[2,3-d]pyrimidine-6-carboxylic acid 1-phenyl-ethyl ester;
3-Benzyl-1-methyl-2,4-dioxo-1,2,3,4-tetrahydro-thieno[2,3-d]pyrimidine-6-carboxylic acid 1-benzyl-piperidin-4-yl ester;
3-Benzyl-1-methyl-2,4-dioxo-1,2,3,4-tetrahydro-thieno[2,3-d]pyrimidine-6-carboxylic acid propyl ester;
3-Benzyl-1-methyl-2,4-dioxo-1,2,3,4-tetrahydro-thieno[2,3-d]pyrimidine-6-carboxylic acid methyl ester;
3-Benzyl-1-methyl-2,4-dioxo-1,2,3,4-tetrahydro-thieno[2,3-d]pyrimidine-6-carboxylic acid 2-trifluoromethyl-benzyl ester;
3-Benzyl-1-methyl-2,4-dioxo-1,2,3,4-tetrahydro-thieno[2,3-d]pyrimidine-6-carboxylic acid 2-p-tolyl-ethyl ester;
3-Benzyl-1-methyl-2,4-dioxo-1,2,3,4-tetrahydro-thieno[2,3-d]pyrimidine-6-carboxylic acid 4-trifluoromethyl-benzyl ester;
3-Benzyl-1-methyl-2,4-dioxo-1,2,3,4-tetrahydro-thieno[2,3-d]pyrimidine-6-carboxylic acid tetrahydro-furan-2-ylmethyl ester;
3-Benzyl-1-methyl-2,4-dioxo-1,2,3,4-tetrahydro-thieno[2,3-d]pyrimidine-6-carboxylic acid octahydro-inden-5-yl ester;
3-Benzyl-1-methyl-2,4-dioxo-1,2,3,4-tetrahydro-thieno[2,3-d]pyrimidine-6-carboxylic acid 4-amino-benzyl ester;
3-Benzyl-1-methyl-2,4-dioxo-1,2,3,4-tetrahydro-thieno[2,3-d]pyrimidine-6-carboxylic acid 2-aziridin-1-yl-ethyl ester;
3-Benzyl-1-methyl-2,4-dioxo-1,2,3,4-tetrahydro-thieno[2,3-d]pyrimidine-6-carboxylic acid 3-methyl-but-2-enyl ester;
3-Benzyl-1-methyl-2,4-dioxo-1,2,3,4-tetrahydro-thieno[2,3-d]pyrimidine-6-carboxylic acid benzyl ester;
3-Benzyl-1-methyl-2,4-dioxo-1,2,3,4-tetrahydro-thieno[2,3-d]pyrimidine-6-carboxylic acid trifluoro-trifluoromethyl-ethyl ester;
3-Benzyl-1-methyl-2,4-dioxo-1,2,3,4-tetrahydro-thieno[2,3-d]pyrimidine-6-carboxylic acid phenethyl ester;
3-Benzyl-1-methyl-2,4-dioxo-1,2,3,4-tetrahydro-thieno[2,3-d]pyrimidine-6-carboxylic acid 2-methoxy-ethyl ester;
3-Benzyl-1-methyl-2,4-dioxo-1,2,3,4-tetrahydro-thieno[2,3-d]pyrimidine-6-carboxylic acid biphenyl-4-ylmethyl ester;
3-Benzyl-1-methyl-2,4-dioxo-1,2,3,4-tetrahydro-thieno[2,3-d]pyrimidine-6-carboxylic acid 2-chloro-6-fluoro-benzyl ester;
3-Benzyl-1-methyl-2,4-dioxo-1,2,3,4-tetrahydro-thieno[2,3-d]pyrimidine-6-carboxylic acid tetrahydro-pyran-4-yl ester;
3-Benzyl-1-methyl-2,4-dioxo-1,2,3,4-tetrahydro-thieno[2,3-d]pyrimidine-6-carboxylic acid 3-ethyl-oxetan-3-ylmethyl ester;
3-Benzyl-1-methyl-2,4-dioxo-1,2,3,4-tetrahydro-thieno[2,3-d]pyrimidine-6-carboxylic acid butyl ester;
3-Benzyl-1-methyl-2,4-dioxo-1,2,3,4-tetrahydro-thieno[2,3-d]pyrimidine-6-carboxylic acid 2-(2-hydroxy-phenyl)-ethyl ester;
3-Benzyl-1-methyl-2,4-dioxo-1,2,3,4-tetrahydro-thieno[2,3-d]pyrimidine-6-carboxylic acid 2-(4-fluoro-phenyl)-ethyl ester;
3-Benzyl-1-methyl-2,4-dioxo-1,2,3,4-tetrahydro-thieno[2,3-d]pyrimidine-6-carboxylic acid cyclopropylmethyl ester;
3-Benzyl-1-methyl-2,4-dioxo-1,2,3,4-tetrahydro-thieno[2,3-d]pyrimidine-6-carboxylic acid 4-ethyl-benzyl ester;
3-Benzyl-1-methyl-2,4-dioxo-1,2,3,4-tetrahydro-thieno[2,3-d]pyrimidine-6-carboxylic acid (S)-1-phenyl-ethyl ester;
3-Benzyl-1-methyl-2,4-dioxo-1,2,3,4-tetrahydro-thieno[2,3-d]pyrimidine-6-carboxylic acid 2,6-difluoro-benzyl ester;
3-Benzyl-1-methyl-2,4-dioxo-1,2,3,4-tetrahydro-thieno[2,3-d]pyrimidine-6-carboxylic acid cyclobutyl methyl ester;
3-Benzyl-1-methyl-2,4-dioxo-1,2,3,4-tetrahydro-thieno[2,3-d]pyrimidine-6-carboxylic acid 2-pyridin-4-yl-ethyl ester;
3-Benzyl-1-methyl-2,4-dioxo-1,2,3,4-tetrahydro-thieno[2,3-d]pyrimidine-6-carboxylic acid 3-hydroxy-cyclopentyl ester;
3-Benzyl-1-methyl-2,4-dioxo-1,2,3,4-tetrahydro-thieno[2,3-d]pyrimidine-6-carboxylic acid 1-pentafluorophenyl-ethyl ester;
3-Benzyl-1-methyl-2,4-dioxo-1,2,3,4-tetrahydro-thieno[2,3-d]pyrimidine-6-carboxylic acid 2-benzyloxycarbonylamino-ethyl ester;
3-Benzyl-1-methyl-2,4-dioxo-1,2,3,4-tetrahydro-thieno[2,3-d]pyrimidine-6-carboxylic acid ethyl ester;
2-{[1-(3-Benzyl-1-methyl-2,4-dioxo-1,2,3,4-tetrahydro-thieno[2,3-d]pyrimidin-6-yl)-methanoyl]-amino}-3-phenyl-propionic acid methyl ester;
(3-{[1-(3-Benzyl-1-methyl-2,4-dioxo-1,2,3,4-tetrahydro-thieno[2,3-d]pyrimidin-6-yl)-methanoyl]-amino}-propyl)-carbamic acid tert-butyl ester;
3-Benzyl-1-methyl-2,4-dioxo-1,2,3,4-tetrahydro-thieno[2,3-d]pyrimidine-6-carboxylic acid benzofuran-5-ylmethyl ester;
3-Benzyl-1-methyl-2,4-dioxo-1,2,3,4-tetrahydro-thieno[2,3-d]pyrimidine-6-carboxylic acid benzofuran-2-ylmethyl ester;
3-Benzyl-2,4-dioxo-1,2,3,4-tetrahydro-thieno[2,3-d]pyrimidine-6-carboxylic acid thiophen-3-ylmethyl ester;
3-Benzyl-1-methyl-2,4-dioxo-1,2,3,4-tetrahydro-thieno[2,3-d]pyrimidine-6-carboxylic acid 3H-[1,2,3]oxathiazol-5-ylmethyl ester;
3-Benzyl-2,4-dioxo-1,2,3,4-tetrahydro-thieno[2,3-d]pyrimidine-6-carboxylic acid 3H-[1,2,3]oxathiazol-5-ylmethyl ester;
3-Benzyl-2,4-dioxo-1,2,3,4-tetrahydro-thieno[2,3-d]pyrimidine-6-carboxylic acid[1,4,2]dioxazol-3-ylmethyl ester;
3-Benzyl-1-methyl-2,4-dioxo-1,2,3,4-tetrahydro-thieno[2,3-d]pyrimidine-6-carboxylic acid[1,4,2]dioxazol-3-ylmethyl ester;
3-Benzyl-2,4-dioxo-1,2,3,4-tetrahydro-thieno[2,3-d]pyrimidine-6-carboxylic acid furazan-3-ylmethyl ester;
3-Benzyl-1-methyl-2,4-dioxo-1,2,3,4-tetrahydro-thieno[2,3-d]pyrimidine-6-carboxylic acid furazan-3-ylmethyl ester;
3-Benzyl-1-methyl-2,4-dioxo-1,2,3,4-tetrahydro-thieno[2,3-d]pyrimidine-6-carboxylic acid[1,2,4]oxadiazol-5-ylmethyl ester;
3-Benzyl-2,4-dioxo-1,2,3,4-tetrahydro-thieno[2,3-d]pyrimidine-6-carboxylic acid[1,2,4]oxadiazol-5-ylmethyl ester;
3-Benzyl-2,4-dioxo-1,2,3,4-tetrahydro-thieno[2,3-d]pyrimidine-6-carboxylic acid 3H-[1,2,3]triazol-4-ylmethyl ester;
3-Benzyl-1-methyl-2,4-dioxo-1,2,3,4-tetrahydro-thieno[2,3-d]pyrimidine-6-carboxylic acid 3H-[1,2,3]triazol-4-ylmethyl ester;
3-Benzyl-1-methyl-2,4-dioxo-1,2,3,4-tetrahydro-thieno[2,3-d]pyrimidine-6-carboxylic acid 2H-[1,2,4]triazol-3-ylmethyl ester;
3-Benzyl-2,4-dioxo-1,2,3,4-tetrahydro-thieno[2,3-d]pyrimidine-6-carboxylic acid 2H-[1,2,4]triazol-3-ylmethyl ester;
3-Benzyl-2,4-dioxo-1,2,3,4-tetrahydro-thieno[2,3-d]pyrimidine-6-carboxylic acid isoxazol-5-ylmethyl ester;
3-Benzyl-1-methyl-2,4-dioxo-1,2,3,4-tetrahydro-thieno[2,3-d]pyrimidine-6-carboxylic acid isoxazol-5-ylmethyl ester;
3-Benzyl-1-methyl-2,4-dioxo-1,2,3,4-tetrahydro-thieno[2,3-d]pyrimidine-6-carboxylic acid oxazol-2-ylmethyl ester;
3-Benzyl-2,4-dioxo-1,2,3,4-tetrahydro-thieno[2,3-d]pyrimidine-6-carboxylic acid oxazol-2-ylmethyl ester;
3-Benzyl-2,4-dioxo-1,2,3,4-tetrahydro-thieno[2,3-d]pyrimidine-6-carboxylic acid isothiazol-5-ylmethyl ester;
3-Benzyl-1-methyl-2,4-dioxo-1,2,3,4-tetrahydro-thieno[2,3-d]pyrimidine-6-carboxylic acid isothiazol-5-ylmethyl ester;
3-Benzyl-2,4-dioxo-1,2,3,4-tetrahydro-thieno[2,3-d]pyrimidine-6-carboxylic acid thiazol-2-ylmethyl ester;
3-Benzyl-1-methyl-2,4-dioxo-1,2,3,4-tetrahydro-thieno[2,3-d]pyrimidine-6-carboxylic acid thiazol-2-ylmethyl ester;
3-Benzyl-1-methyl-2,4-dioxo-1,2,3,4-tetrahydro-thieno[2,3-d]pyrimidine-6-carboxylic acid 1H-imidazol-2-ylmethyl ester;
3-Benzyl-2,4-dioxo-1,2,3,4-tetrahydro-thieno[2,3-d]pyrimidine-6-carboxylic acid 2H-imidazol-2-ylmethyl ester;
3-Benzyl-2,4-dioxo-1,2,3,4-tetrahydro-thieno[2,3-d]pyrimidine-6-carboxylic acid 1H-pyrazol-3-ylmethyl ester;
3-Benzyl-1-methyl-2,4-dioxo-1,2,3,4-tetrahydro-thieno[2,3-d]pyrimidine-6-carboxylic acid 2H-pyrazol-3-ylmethyl ester;
3-Benzyl-1-methyl-2,4-dioxo-1,2,3,4-tetrahydro-thieno[2,3-d]pyrimidine-6-carboxylic acid 1H-pyrrol-2-ylmethyl ester;
3-Benzyl-2,4-dioxo-1,2,3,4-tetrahydro-thieno[2,3-d]pyrimidine-6-carboxylic acid 2H-pyrrol-2-ylmethyl ester;
3-Furazan-3-ylmethyl-2,4-dioxo-1,2,3,4-tetrahydro-thieno[2,3-d]pyrimidine-6-carboxylic acid benzyl ester;
3-Benzyl-1-methyl-2,4-dioxo-1,2,3,4-tetrahydro-thieno[2,3-d]pyrimidine-6-carboxylic acid 2H-chromen-2-ylmethyl ester;
3-Benzyl-1-methyl-2,4-dioxo-1,2,3,4-tetrahydro-thieno[2,3-d]pyrimidine-6-carboxylic acid 2H-thiochromen-2-ylmethyl ester;
3-Benzyl-2,4-dioxo-1,2,3,4-tetrahydro-thieno[2,3-d]pyrimidine-6-carboxylic acid 2H-thiochromen-2-ylmethyl ester;
3-Benzyl-2,4-dioxo-1,2,3,4-tetrahydro-thieno[2,3-d]pyrimidine-6-carboxylic acid[1,3,4]thiadiazol-2-ylmethyl ester;
3-Benzyl-1-methyl-2,4-dioxo-1,2,3,4-tetrahydro-thieno[2,3-d]pyrimidine-6-carboxylic acid[1,3,4]thiadiazol-2-ylmethyl ester;
3-Benzyl-2,4-dioxo-1,2,3,4-tetrahydro-thieno[2,3-d]pyrimidine-6-carboxylic acid 1H-benzoimidazol-5-ylmethyl ester;
3-Benzyl-1-methyl-2,4-dioxo-1,2,3,4-tetrahydro-thieno[2,3-d]pyrimidine-6-carboxylic acid 1H-benzoimidazol-5-ylmethyl ester;
3-Benzyl-1-methyl-2,4-dioxo-1,2,3,4-tetrahydro-thieno[2,3-d]pyrimidine-6-carboxylic acid 1H-benzoimidazol-2-ylmethyl ester;
3-Benzyl-2,4-dioxo-1,2,3,4-tetrahydro-thieno[2,3-d]pyrimidine-6-carboxylic acid 1H-benzoimidazol-2-ylmethyl ester;
3-Benzyl-2,4-dioxo-1,2,3,4-tetrahydro-thieno[2,3-d]pyrimidine-6-carboxylic acid 1H-indol-2-ylmethyl ester;
3-Benzyl-1-methyl-2,4-dioxo-1,2,3,4-tetrahydro-thieno[2,3-d]pyrimidine-6-carboxylic acid 1H-indol-2-ylmethyl ester;
3-Benzyl-1-methyl-2,4-dioxo-1,2,3,4-tetrahydro-thieno[2,3-d]pyrimidine-6-carboxylic acid 1H-indol-5-ylmethyl ester;
3-Benzyl-2,4-dioxo-1,2,3,4-tetrahydro-thieno[2,3-d]pyrimidine-6-carboxylic acid 1H-indol-5-ylmethyl ester;
3-Benzyl-2,4-dioxo-1,2,3,4-tetrahydro-thieno[2,3-d]pyrimidine-6-carboxylic acid 2,3-dihydro-benzofuran-5-ylmethyl ester; and
3-Benzyl-1-methyl-2,4-dioxo-1,2,3,4-tetrahydro-thieno[2,3-d]pyrimidine-6-carboxylic acid 2,3-dihydro-benzofuran-5-ylmethyl ester.

20. The compound according to claim 1, selected from:
3-Benzyl-1-methyl-2,4-dioxo-1,2,3,4-tetrahydro-thieno[2,3-d]pyrimidine-6-carboxylic acid (2-pyridin-4-yl-ethyl)-amide;
3-Benzyl-1-methyl-2,4-dioxo-1,2,3,4-tetrahydro-thieno[2,3-d]pyrimidine-6-carboxylic acid (2-morpholin-4-yl-ethyl)-amide;
3-Benzyl-1-methyl-2,4-dioxo-1,2,3,4-tetrahydro-thieno[2,3-d]pyrimidine-6-carboxylic acid 4-methyl-benzylamide;
3-Benzyl-1-methyl-2,4-dioxo-1,2,3,4-tetrahydro-thieno[2,3-d]pyrimidine-6-carboxylic acid sec-butylamide;
3-Benzyl-1-methyl-2,4-dioxo-1,2,3,4-tetrahydro-thieno[2,3-d]pyrimidine-6-carboxylic acid cyclopentylamide;
3-Benzyl-1-methyl-2,4-dioxo-1,2,3,4-tetrahydro-thieno[2,3-d]pyrimidine-6-carboxylic acid cyclopropylamide;
3-Benzyl-1-methyl-2,4-dioxo-1,2,3,4-tetrahydro-thieno[2,3-d]pyrimidine-6-carboxylic acid cyanomethyl-amide;
3-Benzyl-1-methyl-2,4-dioxo-1,2,3,4-tetrahydro-thieno[2,3-d]pyrimidine-6-carboxylic acid cyclohexylamide;
3-Benzyl-1-methyl-2,4-dioxo-1,2,3,4-tetrahydro-thieno[2,3-d]pyrimidine-6-carboxylic acid 3-methyl-benzylamide;
3-Benzyl-1-methyl-2,4-dioxo-1,2,3,4-tetrahydro-thieno[2,3-d]pyrimidine-6-carboxylic acid (3-ethoxy-propyl)-amide;
3-Benzyl-1-methyl-2,4-dioxo-1,2,3,4-tetrahydro-thieno[2,3-d]pyrimidine-6-carboxylic acid 2-chloro-benzylamide;
3-Benzyl-1-methyl-2,4-dioxo-1,2,3,4-tetrahydro-thieno[2,3-d]pyrimidine-6-carboxylic acid 2-methyl-benzylamide;
3-Benzyl-1-methyl-2,4-dioxo-1,2,3,4-tetrahydro-thieno[2,3-d]pyrimidine-6-carboxylic acid (2,2-diphenyl-ethyl)-amide;
3-Benzyl-1-methyl-2,4-dioxo-1,2,3,4-tetrahydro-thieno[2,3-d]pyrimidine-6-carboxylic acid (pyridin-3-ylmethyl)-amide;
3-Benzyl-1-methyl-2,4-dioxo-1,2,3,4-tetrahydro-thieno[2,3-d]pyrimidine-6-carboxylic acid cyclopropylmethyl-amide;
3-Benzyl-1-methyl-2,4-dioxo-1,2,3,4-tetrahydro-thieno[2,3-d]pyrimidine-6-carboxylic acid (1-ethyl-pyrrolidin-2-ylmethyl)-amide;
3-Benzyl-1-methyl-2,4-dioxo-1,2,3,4-tetrahydro-thieno[2,3-d]pyrimidine-6-carboxylic acid (pyridin-2-ylmethyl)-amide;
3-Benzyl-1-methyl-2,4-dioxo-1,2,3,4-tetrahydro-thieno[2,3-d]pyrimidine-6-carboxylic acid[2-(3,4-dimethoxy-phenyl)-ethyl]-amide;
3-Benzyl-1-methyl-2,4-dioxo-1,2,3,4-tetrahydro-thieno[2,3-d]pyrimidine-6-carboxylic acid (furan-2-ylmethyl)-amide;
3-Benzyl-1-methyl-2,4-dioxo-1,2,3,4-tetrahydro-thieno[2,3-d]pyrimidine-6-carboxylic acid 2-fluoro-benzylamide;
3-Benzyl-1-methyl-2,4-dioxo-1,2,3,4-tetrahydro-thieno[2,3-d]pyrimidine-6-carboxylic acid (2-bromo-ethyl)-amide;
3-Benzyl-1-methyl-2,4-dioxo-1,2,3,4-tetrahydro-thieno[2,3-d]pyrimidine-6-carboxylic acid 4-sulfamoyl-benzylamide;
3-Benzyl-1-methyl-2,4-dioxo-1,2,3,4-tetrahydro-thieno[2,3-d]pyrimidine-6-carboxylic acid 3-methoxy-benzylamide;
3-Benzyl-1-methyl-2,4-dioxo-1,2,3,4-tetrahydro-thieno[2,3-d]pyrimidine-6-carboxylic acid 4-methoxy-benzylamide;
3-Benzyl-1-methyl-2,4-dioxo-1,2,3,4-tetrahydro-thieno[2,3-d]pyrimidine-6-carboxylic acid phenethyl-amide;
(S)-2-{[1-(3-Benzyl-1-methyl-2,4-dioxo-1,2,3,4-tetrahydro-thieno[2,3-d]pyrimidin-6-yl)-methanoyl]-amino}-propionic acid;
3-Benzyl-1-methyl-2,4-dioxo-1,2,3,4-tetrahydro-thieno[2,3-d]pyrimidine-6-carboxylic acid (1-phenyl-ethyl)-amide;
3-Benzyl-1-methyl-2,4-dioxo-1,2,3,4-tetrahydro-thieno[2,3-d]pyrimidine-6-carboxylic acid 2-methoxy-benzylamide;
3-Benzyl-1-methyl-2,4-dioxo-1,2,3,4-tetrahydro-thieno[2,3-d]pyrimidine-6-carboxylic acid benzylamide;
3-Benzyl-1-methyl-2,4-dioxo-1,2,3,4-tetrahydro-thieno[2,3-d]pyrimidine-6-carboxylic acid 3-bromo-benzylamide;
3-Benzyl-1-methyl-2,4-dioxo-1,2,3,4-tetrahydro-thieno[2,3-d]pyrimidine-6-carboxylic acid[2-(4-sulfamoyl-phenyl)-ethyl]-amide;
3-Benzyl-1-methyl-2,4-dioxo-1,2,3,4-tetrahydro-thieno[2,3-d]pyrimidine-6-carboxylic acid (3-imidazol-1-yl-propyl)-amide;
3-Benzyl-1-methyl-2,4-dioxo-1,2,3,4-tetrahydro-thieno[2,3-d]pyrimidine-6-carboxylic acid[2-(2-methoxy-phenyl)-ethyl]-amide;
3-Benzyl-1-methyl-2,4-dioxo-1,2,3,4-tetrahydro-thieno[2,3-d]pyrimidine-6-carboxylic acid 4-trifluoromethyl-benzylamide;
3-Benzyl-1-methyl-2,4-dioxo-1,2,3,4-tetrahydro-thieno[2,3-d]pyrimidine-6-carboxylic acid 4-amino-benzylamide;
3-Benzyl-1-methyl-2,4-dioxo-1,2,3,4-tetrahydro-thieno[2,3-d]pyrimidine-6-carboxylic acid[2-(4-fluoro-phenyl)-ethyl]-amide; and
3-Benzyl-1-methyl-2,4-dioxo-1,2,3,4-tetrahydro-thieno[2,3-d]pyrimidine-6-carboxylic acid ((R)-2-hydroxy-1-methyl-ethyl)-amide.

21. The compound according to claim 1, or a pharmaceutically acceptable salt thereof, selected from:
3-Benzyl-1-methyl-2,4-dioxo-1,2,3,4-tetrahydro-thieno[2,3-d]pyrimidine-6-carboxylic acid methyl ester;
3-(4-Bromo-benzyl)-5-methyl-2,4-dioxo-1,2,3,4-tetrahydro-thieno[2,3-d]pyrimidine-6-carboxylic acid benzyl ester;
3-(4-Fluoro-benzyl)-5-methyl-2,4-dioxo-1,2,3,4-tetrahydro-thieno[2,3-d]pyrimidine-6-carboxylic acid benzyl ester;
3-Benzyl-1-methyl-2,4-dioxo-1,2,3,4-tetrahydro-thieno[2,3-d]pyrimidine-6-carboxylic acid pyridin-4-ylmethyl ester;
3-Benzyl-1-methyl-2,4-dioxo-1,2,3,4-tetrahydro-thieno[2,3-d]pyrimidine-6-carboxylic acid benzo[b]thiophen-2-ylmethyl ester;
3-Benzyl-1-methyl-2,4-dioxo-1,2,3,4-tetrahydro-thieno[2,3-d]pyrimidine-6-carboxylic acid 1-methyl-1H-indol-5-ylmethyl ester;
3-Benzyl-2,4-dioxo-1,2,3,4-tetrahydro-thieno[2,3-d]pyrimidine-6-carboxylic acid thiophen-3-ylmethyl ester;
3-1,3-Benzodioxol-5-ylmethyl-1-methyl-2,4-dioxo-1,2,3,4-tetrahydro-thieno[2,3-d]pyrimidine-6-carboxylic acid benzyl ester;
1-Methyl-2,4-dioxo-3-pyridin-4-ylmethyl-1,2,3,4-tetrahydro-thieno[2,3-d]pyrimidine-6-carboxylic acid benzyl ester;
3-(4-tert-Butyl-benzyl)-1-methyl-2,4-dioxo-1,2,3,4-tetrahydro-thieno[2,3-d]pyrimidine-6-carboxylic acid benzyl ester;
3-(3,4-Dichloro-benzyl)-5-methyl-2,4-dioxo-1,2,3,4-tetrahydro-thieno[2,3-d]pyrimidine-6-carboxylic acid benzyl ester;
1-Methyl-2,4-dioxo-3-(4-trifluoromethoxy-benzyl)-1,2,3,4-tetrahydro-thieno[2,3-d]pyrimidine-6-carboxylic acid benzyl ester;
1-Methyl-3-naphthalen-2-ylmethyl-2,4-dioxo-1,2,3,4-tetrahydro-thieno[2,3-d]pyrimidine-6-carboxylic acid benzyl ester;
3-(4-Cyano-benzyl)-1-methyl-2,4-dioxo-1,2,3,4-tetrahydro-thieno[2,3-d]pyrimidine-6-carboxylic acid benzyl ester;
3-Benzyl-1-methyl-2,4-dioxo-1,2,3,4-tetrahydro-thieno[2,3-d]pyrimidine-6-carboxylic acid benzofuran-5-ylmethyl ester;
3-(3,5-Dimethoxy-benzyl)-1-methyl-2,4-dioxo-1,2,3,4-tetrahydro-thieno[2,3-d]pyrimidine-6-carboxylic acid benzyl ester;
3-Benzyl-1-methyl-2,4-dioxo-1,2,3,4-tetrahydro-thieno[2,3-d]pyrimidine-6-carboxylic acid benzyl ester;
3-(3,5-Dinitro-benzyl)-1-methyl-2,4-dioxo-1,2,3,4-tetrahydro-thieno[2,3-d]pyrimidine-6-carboxylic acid benzyl ester;
3-Benzyl-1-methyl-2,4-dioxo-1,2,3,4-tetrahydro-thieno[2,3-d]pyrimidine-6-carboxylic acid; and
3-(4-Carboxy-benzyl)-1-methyl-2,4-dioxo-1,2,3,4-tetrahydro-thieno[2,3-d]pyrimidine-6-carboxylic acid 2-ethoxy-benzyl ester.

22. The compound according to claim 1, or a pharmaceutically acceptable salt thereof, selected from:
3-Benzyl-1-methyl-2,4-dioxo-1,2,3,4-tetrahydro-thieno[2,3-d]pyrimidine-6-carboxylic acid[2-(3,4-dimethoxy-phenyl)-ethyl]-amide;
3-Benzyl-1-methyl-2,4-dioxo-1,2,3,4-tetrahydro-thieno[2,3-d]pyrimidine-6-carboxylic acid 4-amino-benzylamide;
3-Benzyl-1-methyl-2,4-dioxo-1,2,3,4-tetrahydro-thieno[2,3-d]pyrimidine-6-carboxylic acid[2-(4-chloro-phenyl)-ethyl]-amide;
3-Benzyl-1-methyl-2,4-dioxo-1,2,3,4-tetrahydro-thieno[2,3-d]pyrimidine-6-carboxylic acid (biphenyl-2-ylmethyl)-amide;
3-Benzyl-1-methyl-2,4-dioxo-1,2,3,4-tetrahydro-thieno[2,3-d]pyrimidine-6-carboxylic acid 3,4-dimethoxy-benzylamide;
3-Benzyl-1-methyl-2,4-dioxo-1,2,3,4-tetrahydro-thieno[2,3-d]pyrimidine-6-carboxylic acid (2-pyridin-4-yl-ethyl)-amide;
3-Benzyl-1-methyl-2,4-dioxo-1,2,3,4-tetrahydro-thieno[2,3-d]pyrimidine-6-carboxylic acid 2-difluoromethoxy-benzylamide;
3-Benzyl-1-methyl-2,4-dioxo-1,2,3,4-tetrahydro-thieno[2,3-d]pyrimidine-6-carboxylic acid[2-(3-ethoxy-phenyl)-ethyl]-amide;
3-Benzyl-1-methyl-2,4-dioxo-1,2,3,4-tetrahydro-thieno[2,3-d]pyrimidine-6-carboxylic acid 3-chloro-4-fluoro-benzylamide;
3-Benzyl-1-methyl-2,4-dioxo-1,2,3,4-tetrahydro-thieno[2,3-d]pyrimidine-6-carboxylic acid 2,4-dichloro-benzylamide;
3-Benzyl-1-methyl-2,4-dioxo-1,2,3,4-tetrahydro-thieno[2,3-d]pyrimidine-6-carboxylic acid (2-phenyl-propyl)-amide;
3-Benzyl-1-methyl-2,4-dioxo-1,2,3,4-tetrahydro-thieno[2,3-d]pyrimidine-6-carboxylic acid 3,4,5-trimethoxy-benzylamide;
3-Benzyl-1-methyl-2,4-dioxo-1,2,3,4-tetrahydro-thieno[2,3-d]pyrimidine-6-carboxylic acid 3-chloro-benzylamide;
3-Benzyl-1-methyl-2,4-dioxo-1,2,3,4-tetrahydro-thieno[2,3-d]pyrimidine-6-carboxylic acid 3,5-dimethoxy-benzylamide;
3-Benzyl-1-methyl-2,4-dioxo-1,2,3,4-tetrahydro-thieno[2,3-d]pyrimidine-6-carboxylic acid 2,3-dimethoxy-benzylamide;
3-Benzyl-1-methyl-2,4-dioxo-1,2,3,4-tetrahydro-thieno[2,3-d]pyrimidine-6-carboxylic acid 3-trifluoromethyl-benzylamide;
3-Benzyl-1-methyl-2,4-dioxo-1,2,3,4-tetrahydro-thieno[2,3-d]pyrimidine-6-carboxylic acid 2-methoxy-benzylamide;
3-Benzyl-1-methyl-2,4-dioxo-1,2,3,4-tetrahydro-thieno[2,3-d]pyrimidine-6-carboxylic acid 2-methyl-benzylamide;
3-Benzyl-1-methyl-2,4-dioxo-1,2,3,4-tetrahydro-thieno[2,3-d]pyrimidine-6-carboxylic acid (4-phenyl-butyl)-amide;
3-Benzyl-1-methyl-2,4-dioxo-1,2,3,4-tetrahydro-thieno[2,3-d]pyrimidine-6-carboxylic acid (pyridin-3-ylmethyl)-amide;
3-Benzyl-1-methyl-2,4-dioxo-1,2,3,4-tetrahydro-thieno[2,3-d]pyrimidine-6-carboxylic acid 4-methoxy-benzylamide;
3-Benzyl-1-methyl-2,4-dioxo-1,2,3,4-tetrahydro-thieno[2,3-d]pyrimidine-6-carboxylic acid ((S)-2,2-dimethyl-4-phenyl-1,3-dioxinan-5-yl)-amide;
3-Benzyl-1-methyl-2,4-dioxo-1,2,3,4-tetrahydro-thieno[2,3-d]pyrimidine-6-carboxylic acid[2-(3-methoxy-phenyl)-ethyl]-amide;
3-Benzyl-1-methyl-2,4-dioxo-1,2,3,4-tetrahydro-thieno[2,3-d]pyrimidine-6-carboxylic acid 3-methoxy-benzylamide;
3-Benzyl-1-methyl-2,4-dioxo-1,2,3,4-tetrahydro-thieno[2,3-d]pyrimidine-6-carboxylic acid (thiophen-2-ylmethyl)-amide;
3-Benzyl-1-methyl-2,4-dioxo-1,2,3,4-tetrahydro-thieno[2,3-d]pyrimidine-6-carboxylic acid 2-chloro-benzylamide;
3-Benzyl-1-methyl-2,4-dioxo-1,2,3,4-tetrahydro-thieno[2,3-d]pyrimidine-6-carboxylic acid (5-methyl-furan-2-ylmethyl)-amide;
3-Benzyl-1-methyl-2,4-dioxo-1,2,3,4-tetrahydro-thieno[2,3-d]pyrimidine-6-carboxylic acid (2,2-diphenyl-ethyl)-amide;
3-Benzyl-1-methyl-2,4-dioxo-1,2,3,4-tetrahydro-thieno[2,3-d]pyrimidine-6-carboxylic acid[2-(2-methoxy-phenyl)-ethyl]-amide;
3-Benzyl-1-methyl-2,4-dioxo-1,2,3,4-tetrahydro-thieno[2,3-d]pyrimidine-6-carboxylic acid[2-(3-trifluoromethyl-phenyl)-ethyl]-amide;
3-Benzyl-1-methyl-2,4-dioxo-1,2,3,4-tetrahydro-thieno[2,3-d]pyrimidine-6-carboxylic acid 4-bromo-benzylamide;
3-Benzyl-1-methyl-2,4-dioxo-1,2,3,4-tetrahydro-thieno[2,3-d]pyrimidine-6-carboxylic acid[2-(1H-indol-3-yl)-ethyl]-amide;
3-Benzyl-1-methyl-2,4-dioxo-1,2,3,4-tetrahydro-thieno[2,3-d]pyrimidine-6-carboxylic acid 3,5-dichloro-benzylamide;
3-Benzyl-1-methyl-2,4-dioxo-1,2,3,4-tetrahydro-thieno[2,3-d]pyrimidine-6-carboxylic acid indan-1-ylamide;
3-Benzyl-1-methyl-2,4-dioxo-1,2,3,4-tetrahydro-thieno[2,3-d]pyrimidine-6-carboxylic acid (furan-2-ylmethyl)-amide;
3-Benzyl-1-methyl-2,4-dioxo-1,2,3,4-tetrahydro-thieno[2,3-d]pyrimidine-6-carboxylic acid[2-(4-methoxy-phenyl)-ethyl]-amide;
3-Benzyl-1-methyl-2,4-dioxo-1,2,3,4-tetrahydro-thieno[2,3-d]pyrimidine-6-carboxylic acid 2,4-dimethoxy-benzylamide;
3-Benzyl-1-methyl-2,4-dioxo-1,2,3,4-tetrahydro-thieno[2,3-d]pyrimidine-6-carboxylic acid 4-chloro-benzylamide;
3-Benzyl-1-methyl-2,4-dioxo-1,2,3,4-tetrahydro-thieno[2,3-d]pyrimidine-6-carboxylic acid (1-phenyl-ethyl)-amide;
3-Benzyl-1-methyl-2,4-dioxo-1,2,3,4-tetrahydro-thieno[2,3-d]pyrimidine-6-carboxylic acid 3,4-dichloro-benzylamide;
3-Benzyl-1-methyl-2,4-dioxo-1,2,3,4-tetrahydro-thieno[2,3-d]pyrimidine-6-carboxylic acid 4-fluoro-3-trifluoromethyl-benzylamide;
3-Benzyl-1-methyl-2,4-dioxo-1,2,3,4-tetrahydro-thieno[2,3-d]pyrimidine-6-carboxylic acid (2-pyridin-2-yl-ethyl)-amide;
3-Benzyl-1-methyl-2,4-dioxo-1,2,3,4-tetrahydro-thieno[2,3-d]pyrimidine-6-carboxylic acid[2-(2,4-dimethyl-phenyl)-ethyl]-amide;
3-Benzyl-1-methyl-2,4-dioxo-1,2,3,4-tetrahydro-thieno[2,3-d]pyrimidine-6-carboxylic acid[2-(2,4-dichloro-phenyl)-ethyl]-amide;
1,3-Dimethyl-2,4-dioxo-1,2,3,4-tetrahydro-thieno[2,3-d]pyrimidine-6-carboxylic acid 3-methoxy-benzylamide;
3-Cyanomethyl-1-methyl-2,4-dioxo-1,2,3,4-tetrahydro-thieno[2,3-d]pyrimidine-6-carboxylic acid 4-methoxy-benzylamide;
3-Benzyl-1-methyl-2,4-dioxo-1,2,3,4-tetrahydro-thieno[2,3-d]pyrimidine-6-carboxylic acid 3-methoxy-benzylamide;
3-(4-Cyclopropylsulfamoyl-benzyl)-1-methyl-2,4-dioxo-1,2,3,4-tetrahydro-thieno[2,3-d]pyrimidine-6-carboxylic acid 3-methoxy-benzylamide;
1-Methyl-3-(6-nitro-pyridin-3-ylmethyl)-2,4-dioxo-1,2,3,4-tetrahydro-thieno[2,3-d]pyrimidine-6-carboxylic acid 3-methoxy-benzylamide;
1-Methyl-3-(6-nitro-pyridin-3-ylmethyl)-2,4-dioxo-1,2,3,4-tetrahydro-thieno[2,3-d]pyrimidine-6-carboxylic acid 4-methoxy-benzylamide;
1-Methyl-3-(6-nitro-pyridin-3-ylmethyl)-2,4-dioxo-1,2,3,4-tetrahydro-thieno[2,3-d]pyrimidine-6-carboxylic acid (2-methoxy-pyridin-4-ylmethyl)-amide;
3-Cyclohexylmethyl-1-methyl-2,4-dioxo-1,2,3,4-tetrahydro-thieno[2,3-d]pyrimidine-6-carboxylic acid (2-methoxy-pyridin-4-ylmethyl)-amide;
3-{2-[(1H-Benzimidazole-5-carbonyl)-amino]-ethyl}-1-methyl-2,4-dioxo-1,2,3,4-tetrahydro-thieno[2,3-d]pyrimidine-6-carboxylic acid 3-methoxy-benzylamide;
1-Methyl-2,4-dioxo-3-[2-(3-piperidin-1-yl-propionylamino)-ethyl]-1,2,3,4-tetrahydro-thieno[2,3-d]pyrimidine-6-carboxylic acid 3-methoxy-benzylamide;
1-Methyl-2,4-dioxo-3-{2-[(6-pyrazol-1-yl-pyridine-3-carbonyl)-amino]-ethyl}-1,2,3,4-tetrahydro-thieno[2,3-d]pyrimidine-6-carboxylic acid 3-methoxy-benzylamide;
3-[2-(4-Diethylamino-benzoylamino)-ethyl]-1-methyl-2,4-dioxo-1,2,3,4-tetrahydro-thieno[2,3-d]pyrimidine-6-carboxylic acid 3-methoxy-benzylamide;
3-{2-[(6-Chloro-pyridine-3-carbonyl)-amino]-ethyl}-1-methyl-2,4-dioxo-1,2,3,4-tetrahydro-thieno[2,3-d]pyrimidine-6-carboxylic acid 3-methoxy-benzylamide;
1-Methyl-2,4-dioxo-3-{2-[(1H-pyrrole-2-carbonyl)-amino]-ethyl}-1,2,3,4-tetrahydro-thieno[2,3-d]pyrimidine-6-carboxylic acid 3-methoxy-benzylamide;
3-[2-(2-Dimethylamino-acetylamino)-ethyl]-1-methyl-2,4-dioxo-1,2,3,4-tetrahydro-thieno[2,3-d]pyrimidine-6-carboxylic acid 3-methoxy-benzylamide;
1-Methyl-2,4-dioxo-3-{2-[(pyrazine-2-carbonyl)-amino]-ethyl}-1,2,3,4-tetrahydro-thieno[2,3-d]pyrimidine-6-carboxylic acid 3-methoxy-benzylamide;
1-Methyl-3-[2-(2-methyl-2-methylamino-propionylamino)-ethyl]-2,4-dioxo-1,2,3,4-tetrahydro-thieno[2,3-d]pyrimidine-6-carboxylic acid 3-methoxy-benzylamide;
1-Methyl-2,4-dioxo-3-{2-[(pyrrolidine-2-carbonyl)-amino]-ethyl}-1,2,3,4-tetrahydro-thieno[2,3-d]pyrimidine-6-carboxylic acid 3-methoxy-benzylamide;
1-Methyl-2,4-dioxo-3-{2-[3-(5-phenyl-1H-pyrrol-2-yl)-propionylamino]-ethyl}-1,2,3,4-tetrahydro-thieno[2,3-d]pyrimidine-6-carboxylic acid 3-methoxy-benzylamide;
1-Methyl-2,4-dioxo-3-{2-[2-(pyridin-4-ylsulfanyl)-acetylamino]-ethyl}-1,2,3,4-tetrahydro-thieno[2,3-d]pyrimidine-6-carboxylic acid 3-methoxy-benzylamide;
3-(6-Amino-pyridin-3-ylmethyl)-1-methyl-2,4-dioxo-1,2,3,4-tetrahydro-thieno[2,3-d]pyrimidine-6-carboxylic acid 3-methoxy-benzylamide;
1-Methyl-2,4-dioxo-3-(3-phenyl-prop-2-ynyl)-1,2,3,4-tetrahydro-thieno[2,3-d]pyrimidine-6-carboxylic acid 4-methoxy-benzylamide;
3-(6-Amino-pyridin-3-ylmethyl)-1-methyl-2,4-dioxo-1,2,3,4-tetrahydro-thieno[2,3-d]pyrimidine-6-carboxylic acid 4-methoxy-benzylamide;
3-(6-Amino-pyridin-3-ylmethyl)-1-methyl-2,4-dioxo-1,2,3,4-tetrahydro-thieno[2,3-d]pyrimidine-6-carboxylic acid (pyridin-4-ylmethyl)-amide;
1-Methyl-2,4-dioxo-3-[2-(pyridin-2-ylamino)-ethyl]-1,2,3,4-tetrahydro-thieno[2,3-d]pyrimidine-6-carboxylic acid 4-methoxy-benzylamide;
1-Methyl-2,4-dioxo-3-[2-(pyrimidin-2-ylamino)-ethyl]-1,2,3,4-tetrahydro-thieno[2,3-d]pyrimidine-6-carboxylic acid 4-methoxy-benzylamide; and
1-Methyl-2,4-dioxo-3-[2-(pyrimidin-2-ylamino)-ethyl]-1,2,3,4-tetrahydro-thieno[2,3-d]pyrimidine-6-carboxylic acid 3-methoxy-benzylamide.

23. A pharmaceutical composition, comprising a compound of claim 1, or a pharmaceutically acceptable salt thereof, together with a pharmaceutically acceptable carrier, diluent, or excipient.

24. A pharmaceutical composition, comprising a compound of claim 2, or a pharmaceutically acceptable salt thereof, together with a pharmaceutically acceptable carrier, diluent, or excipient.

25. A method for inhibiting MMP-13 in an animal, comprising administering to the animal an MMP-13 inhibiting amount of a compound of claim 1, or a pharmaceutically acceptable salt thereof.

26. A method for inhibiting MMP-13 in an animal, comprising administering to the animal an MMP-13 inhibiting amount of a compound of claim 2, or a pharmaceutically acceptable salt thereof.

27. A method for treating a disease mediated by an MMP-13 enzyme, comprising administering to a patient suffering from such a disease an effective amount of a compound of claim 1, or a pharmaceutically acceptable salt thereof.

28. A method for treating a disease mediated by an MMP-13 enzyme, comprising administering to a patient suffering from such a disease an effective amount of a compound of claim 2, or a pharmaceutically acceptable salt thereof.

29. A method for treating a cancer, comprising administering to a patient suffering from such a disease an anticancer effective amount of a compound of claim 1, or a pharmaceutically acceptable salt thereof.

30. A method for treating breast carcinoma, comprising administering to a patient suffering from such a disease an anticancer effective amount of a compound of claim 1, or a pharmaceutically acceptable salt thereof.

31. A method for treating a rheumatoid arthritis, comprising administering to a patient suffering from such a disease an effective amount of a compound of claim 1, or a pharmaceutically acceptable salt thereof.

32. A method for treating a osteoarthritis, comprising administering to a patient suffering from such a disease an effective amount of a compound of claim 1, or a pharmaceutically acceptable salt thereof.

33. A method for treating a heart failure, comprising administering to a patient suffering from such a disease an effective amount of a compound of claim 1, or a pharmaceutically acceptable salt thereof.

34. A method for treating a inflammation, comprising administering to a patient suffering from such a disease an effective amount of a compound of claim 1, or a pharmaceutically acceptable salt thereof.

35. A method for treating a cancer, comprising administering to a patient suffering from such a disease an anticancer effective amount of a compound of claim 2, or a pharmaceutically acceptable salt thereof.

36. A method for treating breast carcinoma, comprising administering to a patient suffering from such a disease an anticancer effective amount of a compound of claim 2, or a pharmaceutically acceptable salt thereof.

37. A method for treating a rheumatoid arthritis, comprising administering to a patient suffering from such a disease an effective amount of a compound of claim 2, or a pharmaceutically acceptable salt thereof.

38. A method for treating a osteoarthritis, comprising administering to a patient suffering from such a disease an effective amount of a compound of claim 2, or a pharmaceutically acceptable salt thereof.

39. A method for treating a heart failure, comprising administering to a patient suffering from such a disease an effective amount of a compound of claim 2, or a pharmaceutically acceptable salt thereof.

40. A method for treating a inflammation, comprising administering to a patient suffering from such a disease an effective amount of a compound of claim 2, or a pharmaceutically acceptable salt thereof.

41. A compound of Formula I 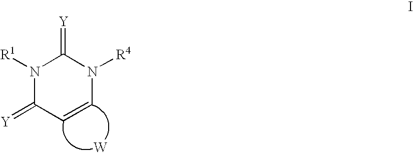
and the pharmaceutically acceptable salts thereof; wherein:
W is 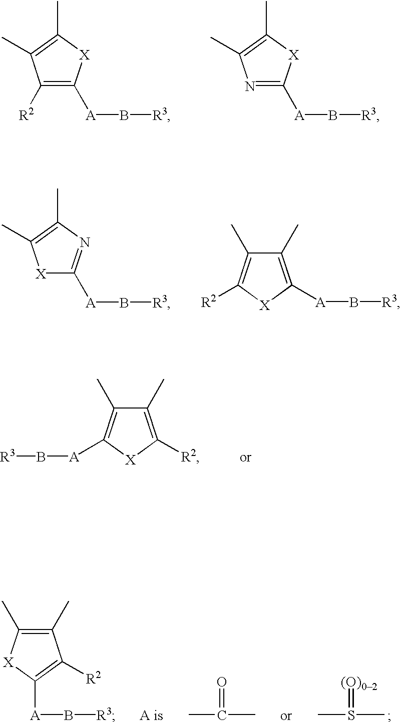
A is
B is O or NR⁵;
X is O, S, SO, SO₂, NR⁵, or CH₂;
each Y independently is O or S;
R¹, R⁴, and R⁵ independently are hydrogen, C₁-C₆ alkyl, C₂-C₆ alkenyl, C₂-C₆ alkynyl, (CH₂)ₙ cycloalkyl, (CH₂)ₙ heterocyclic, C₁-C₆ alkanoyl, (CH₂)ₙ aryl, or (CH₂)ₙ heteroaryl;
R² and R³ independently are hydrogen, halo, C₁-C₆ alkyl, C₂-C₆ alkenyl, C₂-C₆ alkynyl, CH, NO₂, NR⁴R⁵, (CH₂)ₙ cycloalkyl, (CH₂)ₙ aryl, or (CH₂)ₙ heteroaryl;
n is 0, 1, or 2;
R⁴ and R⁵ when taken together with the nitrogen to which they are attached complete a 3- to 8-membered ring optionally containing O, S, or N, and substituted or unsubstituted.

42. A compound of Formula II 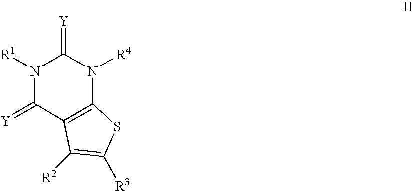
wherein:
each Y independently is O or S;
R¹, R⁴, and R⁵ independently are hydrogen, C₁-C₆ alkyl, C₂-C₆ alkenyl, C₂-C₆ alkynyl, (CH₂)ₙ cycloalkyl, (CH₂)ₙ heterocyclic, C₁-C₆ alkanoyl, (CH₂)ₙ aryl, or (CH₂)ₙ heteroaryl;
R² and R³ independently are hydrogen, halo, C₁-C₆ alkyl, C₂-C₆ alkenyl, C₂-C₆ alkynyl, CH, NO₂, NR⁴R⁵, (CH₂)ₙ cycloalkyl, (CH₂)ₙ aryl, or (CH₂)ₙ heteroaryl;
n is 0, 1, or 2;
R⁴ and R⁵ when taken together with the nitrogen to which they are attached complete a 3- to 8-membered ring optionally containing O, S, or N, and substituted or unsubstituted.

43. A compound of Formula III 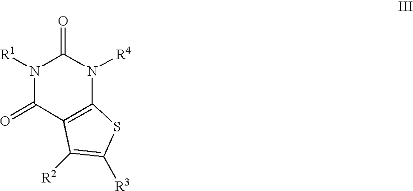
wherein:
each Y independently is O or S;
R¹, R⁴, and R⁵ independently are hydrogen, C₁-C₆ alkyl, C₂-C₆ alkenyl, C₂-C₆ alkynyl, (CH₂)ₙ cycloalkyl, (CH₂)ₙ heterocyclic, C₁-C₆ alkanoyl, (CH₂)ₙ aryl, or (CH₂)ₙ heteroaryl;
R² and R³ independently are hydrogen, halo, C₁-C₆ alkyl, C₂-C₆ alkenyl, C₂-C₆ alkynyl, CN, NO₂, NR⁴R⁵, (CH₂)ₙ cycloalkyl, (CH₂)ₙ aryl, or (CH₂)ₙ heteroaryl;
n is 0, 1, or 2;
R⁴ and R⁵ when taken together with the nitrogen to which they are attached complete a 3- to 8-membered ring optionally containing O, S, or N, and substituted or unsubstituted.

44. A compound of Formula IV 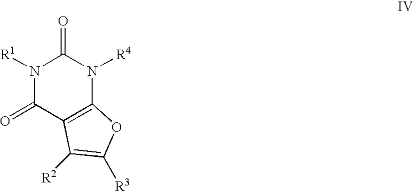
wherein:
each Y independently is O or S;
R¹, R⁴, and R⁵ independently are hydrogen, C₁-C₆ alkyl, C₂-C₆ alkenyl, C₂-C₆ alkynyl, (CH₂)ₙ cycloalkyl, (CH₂)ₙ heterocyclic, C₁-C₆ alkanoyl, (CH₂)ₙ aryl, or (CH₂)ₙ heteroaryl;
R² and R³ independently are hydrogen, halo, C₁-C₆ alkyl, C₂-C₆ alkenyl, C₂-C₆ alkynyl, CN, NO₂, NR⁴R⁵, (CH₂)ₙ cycloalkyl, (CH₂)ₙ aryl, or (CH₂)ₙ heteroaryl;
n is 0, 1, or 2;
R⁴ and R⁵ when taken together with the nitrogen to which they are attached complete a 3- to 8-membered ring optionally containing O, S, or N, and substituted or unsubstituted.

45. A compound of Formula V 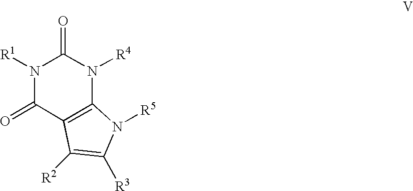
wherein R¹, R², R³, R⁴, and R⁵;
each Y independently is O or S;
R¹, R⁴, and R⁵ independently are hydrogen, C₁-C₆ alkyl, C₂-C₆ alkenyl, C₂-C₆ alkynyl, (CH₂)ₙ cycloalkyl, (CH₂)ₙ heterocyclic, C₁-C₆ alkanoyl, (CH₂)ₙ aryl, or (CH₂)ₙ heteroaryl;
R² and R³ independently are hydrogen, halo, C₁-C₆ alkyl, C₂-C₆ alkenyl, C₂-C₆ alkynyl, CN, NO₂, NR⁴R⁵, (CH₂)ₙ cycloalkyl, (CH₂)ₙ aryl, or (CH₂)ₙ heteroaryl;
n is 0, 1, or 2;
R⁴ and R⁵ when taken together with the nitrogen to which they are attached complete a 3- to 8-membered ring optionally containing O, S, or N, and substituted or unsubstituted.

46. A pharmaceutical composition comprising a compound of claim 41 together with a pharmaceutically acceptable carrier, diluent, or excipient.

47. A pharmaceutical composition comprising a compound of claim 42 together with a pharmaceutically acceptable carrier, diluent, or excipient.

48. A method for inhibiting MMP-13 enzymes in animals comprising administering to the animal an MMP-13 inhibiting amount of a compound of claim 41.

49. A method for treating cancer comprising administering to a patient having cancer and in need of treatment an anticancer effective amount of a compound of claim 41.

50. A method for treating arthritis comprising administering to a patient in need of treatment an anti-arthritic amount of a compound of claim 41.

51. The method of claim 50 wherein the arthritis is osteoarthritis.

52. The method of claim 50 wherein the arthritis is rheumatoid arthritis.
